(19) United States
(12) Patent Application Publication
(10) Pub. No.: US 2025/0171399 A1
(43) Pub. Date: May 29, 2025

(54) PYRROLIDINE COMPOUNDS

(71) Applicant: Eli Lilly and Company, Indianapolis, IN (US)

(72) Inventors: Ana Maria CASTAÑO MANSANET, Alcobendas (ES); Nuria DIAZ BUEZO, Alcobendas (ES); Timothy Barrett DURHAM, Indianapolis, IN (US); Ana Maria ESCRIBANO NIETO, Alcobendas (ES); Celia LAFUENTE BLANCO, Alcobendas (ES); Carlos LAMAS PETEIRA, San Sebastián de los Reyes (ES); Jose Alfredo MARTIN FUENTES, Alcobendas (ES); Jose Antonio MARTINEZ PEREZ, Alcobendas (ES); Carlos PEREZ MARTINEZ, Alcobendas (ES); Julian PRIEGO SOLER, Alcobendas (ES); Gema Consuelo SANZ GIL, Alcobendas (ES); James Lee TOTH, Knightstown, IN (US); Miguel Angel TOLEDO ESCRIBANO, San Diego, CA (US)

(21) Appl. No.: 18/730,436

(22) PCT Filed: Jan. 19, 2023
(86) PCT No.: PCT/US2023/011103

§ 371 Date: Jul. 19, 2024

(30) Foreign Application Priority Data
Jan. 26, 2022 (EP) 22382054.9
Aug. 5, 2022 (EP) 22382765.0

(51) Int. Cl. C07D 207/08; C07D 207/09; C07D 401/06; C07D 401/10; C07D 401/14; C07D 403/10; C07D 403/12; C07D 403/14; C07D 405/12; C07D 409/10; C07D 413/10; C07D 413/12; C07D 417/12; C07D 471/04; C07D 487/10; C07F 5/02

(52) U.S. Cl. CPC C07D 207/08; C07D 207/09; C07D 401/06; C07D 401/10; C07D 401/14; C07D 403/10; C07D 403/12; C07D 403/14; C07D 405/12; C07D 409/10; C07D 413/10; C07D 413/12; C07D 417/12; C07D 471/04; C07D 487/10; C07F 5/025

(57) ABSTRACT

The present disclosure provides compounds of the formula:

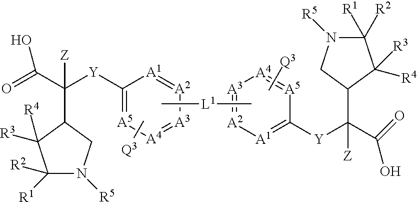

and their pharmaceutically acceptable salts, and compounds of the formula:

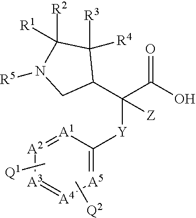

and their pharmaceutically acceptable salts, and pharmaceutical compositions comprising these compounds, as well as intermediates for preparing the compounds.

This invention relates to pyrrolidine compounds, pharmaceutically acceptable salts thereof, pharmaceutical compositions, and therapeutic uses of the compounds, in particular their use in lowering lipoprotein(a) (Lp(a)) plasma levels. The invention also relates to intermediates for use in preparing the therapeutic pyrrolidine compounds.

There have been significant advances in treating cardiovascular disease (CVD). Despite treatment advances, patients continue to experience cardiovascular disease events such as angina, myocardial infarction, and stroke, which if untreated, lead to death. Lipid disorder or dyslipidemia remains a major risk factor for CVD. Lipid disorders can be divided into four general risk factors: elevated low-density lipoprotein cholesterol (LDL-c), low high-density lipoprotein cholesterol (HDL-c), elevated triglycerides (TG), and elevated Lp(a). There are a variety of treatment regimens targeting elevated LDL-c, low HDL-c, and elevated triglycerides. There are few approved treatment options for patients with elevated Lp(a) concentrations. In some cases, apheresis may be used to filter the blood to remove LDL and Lp(a); however, the effects are temporary and typically need to be repeated every two weeks. There is no pharmaceutical treatment approved to lower Lp(a) levels. The physiological function of Lp(a) is complex; however, it is reported that elevated Lp(a) plasma level is an independent risk factor for CVD.

Lp(a) may exhibit both prothrombotic and antithrombotic properties, and atherogenic and atherothrombotic properties. Lp(a) may inhibit fibrinolysis and accumulate in the vascular wall inducing thrombogenesis and atherosclerotic lesions. Plasma levels of Lp(a) vary substantially among individuals. Unlike the other risk factors, Lp(a) plasma levels do not vary significantly with diet and exercise. Lp(a) plasma levels may be linked to genetic predisposition.

Lp(a) resembles LDL-c in that it includes an LDL lipid core with the attendant apolipoprotein B (apoB), but unlike LDL-c, Lp(a) also contains a unique apolipoprotein(a) (apo(a)) bound to the apoB via disulfide bond. Apo(a) is synthesized in the liver. The assembly of Lp(a) from apo(a) and LDL particles can occur in hepatocytes, on the cell wall, or in plasma. Inhibition of the assembly of the LDL particle with apo(a) may reduce Lp(a) levels. Additional treatment options are desired for patients suffering from cardiovascular diseases and, in particular, patients suffering from lipid disorders or dyslipidemia. There is a need for additional treatment options for patients whose cardiovascular risks are not adequately managed using current standard of care therapies, such as, diet, exercise and/or the use of one or more drugs such as statins, fibrates, and niacin. The present invention offers another treatment option for patients suffering from CVD. There is a need for pharmaceutically acceptable compounds and treatment options to reduce plasma Lp(a) levels.

In a first aspect, there is provided a compound of formula III:

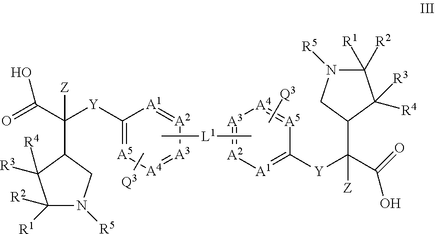

wherein
L¹ is attached at A¹, A² or A³ and is selected from a bond, —(CH₂)ₚNHC(O)NH(CH₂)ₚ—, —(CH₂)ₚC(O)NH(CH₂)ₚ—, —(CH₂)ₚS(O)₂NH(CH₂)ₚ—, —(CH₂)_{q}—, —(CH₂)ₚNH(CH₂)ₚ—,

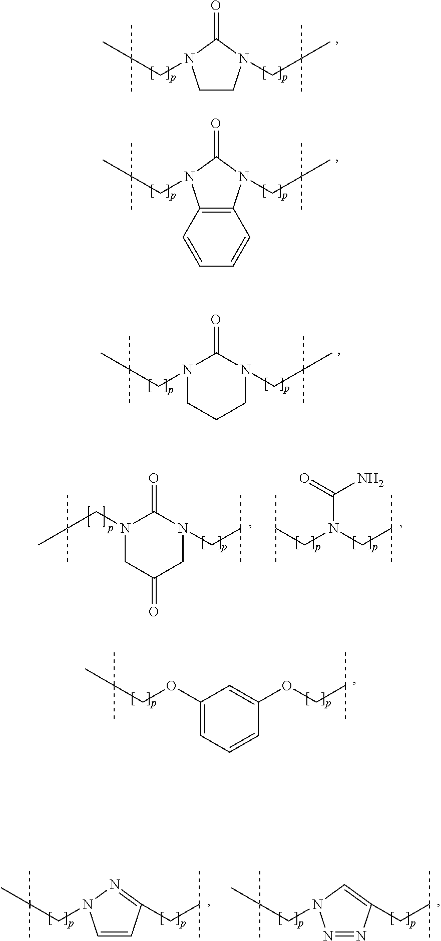

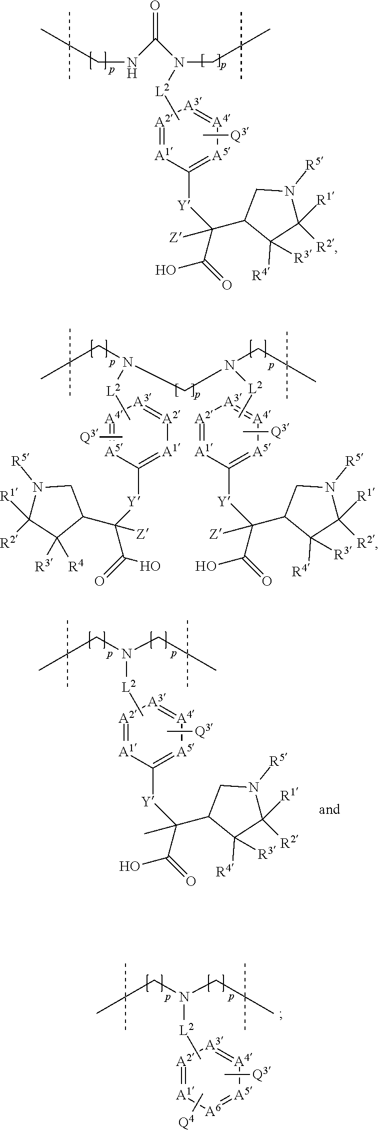

or L¹ together with the carbons at positions A² and A³ on one ring form the fused ring:

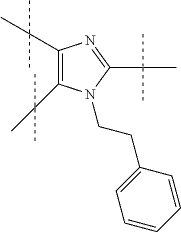

R¹, R^{1′}, R² and R^{2′} at each occurrence are independently selected from H, C₁₋₄ alkyl and F;
R³, R^{3′}, R⁴ and R^{4′} at each occurrence are independently either H or F;
R⁵ and R^{5′} at each occurrence are independently H, C₁₋₄ alkyl, or cyclopropyl;
Z and Z′ at each occurrence are independently H, C₁₋₄ alkyl, OH, cyclopropyl, CH₂OH, CH₂NH₂ or CH₂OCH₂phenyl;
Y and Y′ at each occurrence are independently CH₂, CH(CH₃), O or S; A¹, A^{1′}, A², A^{2′}, A³, A^{3′}, A⁴, A^{4′}, A⁵, A^{5′} and A⁶ at each occurrence are independently C or N, wherein no more than two of A¹, A², A³, A⁴ and A⁵ on each ring are N or no more than two of A^{1′}, A^{2′}, A^{3′}, A^{4′}, A^{5′} and A^{6′} on each ring are N;
Q³ and Q^{3′} at each occurrence are independently H; —(CH₂)ₙO(CH₂)ₙR¹⁰;
—(CH₂)ₙNR¹⁵(CH₂)ₙR¹⁰; —CN; —(CH₂)ₙCO₂R¹⁰; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; NO₂; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or

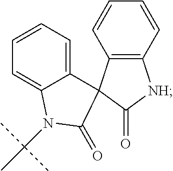

Q⁴ is R¹¹, CF₃, O—R¹¹, OCF₃, halo, or CN;
m at each occurrence is independently 0, 1, or 2;
n at each occurrence is independently 0, 1, 2, or 3;
R¹⁰ at each occurrence is independently H; halo; OH; carboxyl; —S(O)₂OH; C₁₋₄ alkyl optionally substituted with one to four OH or with OCH₃; C₃₋₆ cycloalkyl optionally substituted with one or two halo; C₁₋₄ haloalkyl; —C₂₋₆ alkynyl; 1-benzyl-4-piperidyl; 2-tert-butoxy-2-oxo-ethyl; benzyloxyphenyl optionally substituted with one or two halo; O(C₁₋₂alkyl)ᵣOCH₃; NH₂; 2,3-dihydro-1H-indene; 2,3-dihydrobenzo[b][1,4]dioxine; benzo[d][1,3]dioxole optionally substituted with one or two halo; indoline optionally substituted with C(O)CH₃; 5- or 6-membered heteroaryl or 9- or 10-membered bicyclic heteroaryl optionally substituted with C₁₋₄ alkyl, C₁₋₄ alkoxy, halo or phenyl; phenyl optionally substituted with one to three substituents independently selected from: halo, C₁₋₄ alkoxy, hydroxy, C₁₋₄ alkyl, CF₃, CN, —(CH₂)₂C(O)OH, —C(O)NHNH₂, —OCF₃, —N(CH₃)₂, C₃₋₆ cycloalkyl, —OC₃₋₆ cycloalkyl, OCH₂C₃₋₆ cycloalkyl, 5- or 6-membered heteroaryl, —(CH₂)ₙ(5- or 6-membered heterocyclyl) or —(CH₂)ₙphenyl wherein the phenyl is optionally substituted one or two halo;
R¹¹ is H, C₁₋₄ alkyl, or cyclopropyl;
L² is attached at A^{1′}, A^{2′} or A^{3′} and is C₁₋₃ alkylene or a bond; and
p at each occurrence is independently 0 to 3;
q is 1 to 5;
r is 1, 2, or 3;
R¹⁵ is H or C₁₋₃alkyl;
or a pharmaceutically acceptable salt thereof,
wherein the compound is not of the formula:

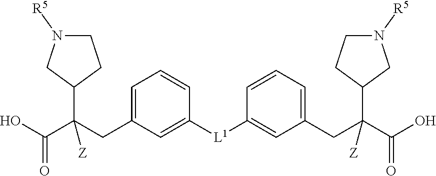

wherein
L¹ is selected from the group consisting of —CH₂NHCH₂—, —CH₂NH—, —NH—,

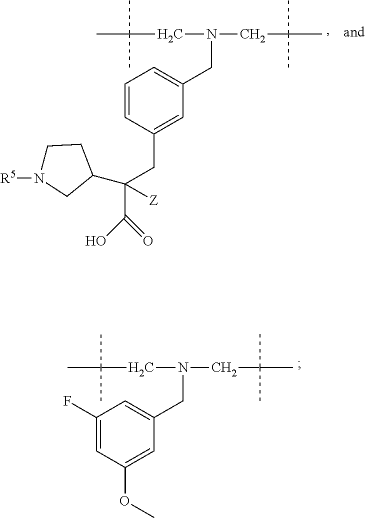

R⁵ is H or CH₃; and
Z is H or CH₃.

In a second aspect, there is provided a compound of formula IV:

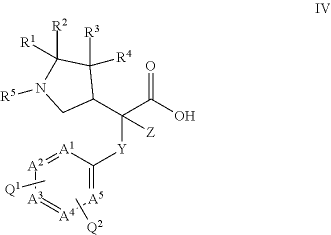

wherein
R¹ and R² are independently selected from H, C₁₋₄ alkyl, and F;
R³ and R⁴ are independently either H or F;
R⁵ is H, C₁₋₄ alkyl, or cyclopropyl;
Z is H, C₁₋₄ alkyl, OH, cyclopropyl, CH₂OH, CH₂NH₂ or CH₂OCH₂phenyl;
Y is CH₂, CH(CH₃), O or S;
A¹, A², A³, A⁴, and A⁵ are each independently C or N, wherein no more than two of A¹, A², A³, A⁴, or A⁵ are N;
Q¹ is —(CH₂)ₙO(CH₂)ₙR¹⁰; —(CH₂)ₙNR¹⁵(CH₂)ₙR¹⁰; —CN; —(CH₂)ₙCO₂R¹⁰; —B(OR¹⁰)₂; a boronic acid ethylene glycol ester; a boronic acid pinacol ester; a boronic acid propylene-1,3-diol ester; a boronic acid 2,2-dimethyl-propylene-1,3-diol ester; —N₃; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; NO₂; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or

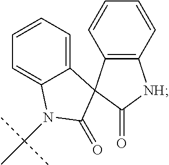

Q² is R¹¹, CF₃, O—R¹¹, OCF₃, halo, or CN;
m is 0, 1, or 2;
n is 0, 1, 2, or 3;
R¹⁰ is H; halo; OH; carboxyl; —S(O)₂OH; C₁₋₄ alkyl optionally substituted with one to four OH or with OCH₃; C₃₋₆ cycloalkyl optionally substituted with one or two halo; C₁0.4 haloalkyl; —C₂₋₆ alkynyl; 1-benzyl-4-piperidyl; 2-tert-butoxy-2-oxo-ethyl; benzyloxyphenyl optionally substituted with one or two halo; O(C₁₋₂alkyl)ᵣOCH₃; NH₂; 2,3-dihydro-1H-indene; 2,3-dihydrobenzo[b][1,4]dioxine; benzo[d][1,3]dioxole optionally substituted with one or two halo; indoline optionally substituted with C(O)CH₃; 5- or 6-membered heteroaryl or 9- or 10-membered bicyclic heteroaryl optionally substituted with C₁₋₄ alkyl, C₁₋₄ alkoxy, halo or phenyl; phenyl optionally substituted with one to three substituents independently selected from: halo, C₁₋₄ alkoxy, hydroxy, C₁₋₄ alkyl, CF₃, CN, —(CH₂)₂C(O)OH, —C(O)NHNH₂, —OCF₃, —N(CH₃)₂, C₃₋₆ cycloalkyl, —OC₃₋₆ cycloalkyl, —OCH₂C₃₋₆ cycloalkyl, 5- or 6-membered heteroaryl, —(CH₂)ₙ(5- or 6-membered heterocyclyl) or —(CH₂)ₙphenyl wherein the phenyl is optionally substituted one or two halo;
R¹¹ is H, C₁₋₄ alkyl, or cyclopropyl;
r is 1, 2 or 3; and
R¹⁵ is H or C₁₋₃alkyl;
or a pharmaceutically acceptable salt thereof.

In an alternate embodiment, there is provided a compound of formula I:

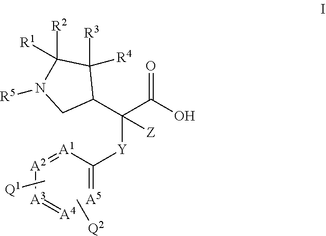

wherein
R¹ and R² are independently selected from H, C₁₋₄ alkyl, and F;
R³ and R⁴ are independently either H or F;
R⁵ is H, C₁₋₄ alkyl, or cyclopropyl;
Z is H, C₁₋₄ alkyl, or cyclopropyl;
A¹, A², A³, A⁴, and A⁵ may each independently be C or N, wherein no more than two of A¹, A², A³, A⁴, or A⁵ are N;
Q¹ is —(CH₂)ₙO(CH₂)ₙR¹⁰, —(CH₂)ₙNH(CH₂)ₙR¹⁰, —CN, —(CH₂)ₙCO₂R¹⁰, —B(OR¹⁰)₂, a boronic acid ethylene glycol ester, a boronic acid pinacol ester, a boronic acid propylene-1,3-diol ester, a boronic acid 2,2-dimethyl-propylene-1,3-diol ester, —N₃, C₁₋₆ alkyl, —C₂₋₆ alkenyl, —C₂₋₆ alkynyl, halo, —C(O)—R¹⁰, —C(O)NHR¹⁰, —NH—NH₂, —S(O)ₘ(CH₂)ₙR¹⁰, —(CH₂)ₙNHS(O)ₘ(CH₂)ₙR¹⁰, —(CH₂)ₙS(O)ₘNH(CH₂)ₙR¹⁰, —(CH₂)ₙNHCONHR¹⁰, —NHCO(CH₂)ₙR¹⁰, —NHCOOR¹⁰, or an aryl or heteroaryl selected from: phenyl, pyrrole, imidazole, pyrazole, triazole, furan, thiophene, oxazole, isoxazole, thiazole, isothiazole, pyridine, pyridazine, pyrimidine and pyrazine, wherein aryl and heteroaryl are optionally substituted with a halo or OCF₃ substituent;
Q² is R¹¹, CF₃, O—R¹¹, OCF₃, halo, or CN;
m is 0, 1, or 2;
n is 0, 1, or 2;
R¹⁰ is H; hydroxy; carboxyl; C₁₋₄ alkyl optionally substituted with OH; C₃₋₆ cycloalkyl; 4,4-difluorocyclohexyl; C₁₋₄ haloalkyl; 3-(1H)-benzimidazol-2-yl; thiazole optionally substituted with methyl; methyl(phenyl)carbamoyl; 1-benzyl-4-piperidyl; 8-quinolyl; 2-methoxyethyl; 3-methoxypropyl; 3-phenyl-1,2,4-oxadiazol-5-yl; 2-tert-butoxy-2-oxo-ethyl; benzyloxyphenyl; phenyl optionally substituted with one to three substituents independently selected from: halo, C₁₋₄ alkoxy, hydroxy, C₁₋₄ alkyl, CF₃, CN, (CH₂)₂C(O)OH, C(O)NHNH₂, OCF₃, N(CH₃)₂, C₃₋₆ cycloalkyl, —OC₃₋₆ cycloalkyl and OCH₂C₃₋₆ cycloalkyl;
and R¹¹ is H, C₁₋₄ alkyl, or cyclopropyl;
or a pharmaceutically acceptable salt thereof.

In a third aspect, there is provided a compound of the formula V:

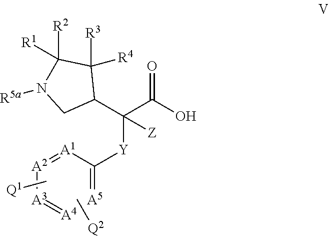

wherein
R¹ and R² are independently selected from H, C₁₋₄ alkyl, and F;
R³ and R⁴ are independently either H or F;
R^{5a} is H, C₁₋₄ alkyl, cyclopropyl, or a protecting group;
X is OH, —OR⁶, or

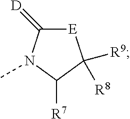

R⁶ is C₁₋₄ alkyl;
D and E are each independently O or S;
R⁷ is H, C₁₋₄ alkyl, phenyl or benzyl, wherein the phenyl and benzyl are optionally substituted with one or two substituents independently selected from: halo, C₁₋₄ alkyl, trifluoromethyl, amino, C₁₋₄ alkylamino, and di-C₁₋₄ alkylamino;
R⁸ and R⁹ are each independently H, C₁₋₄ alkyl or phenyl optionally substituted with one or two substituents independently selected from: halo, C₁₋₄ alkyl, trifluoromethyl, amino, C₁₋₄ alkylamino and di-C₁₋₄ alkylamino;
Z is H, C₁₋₄ alkyl, OH, cyclopropyl, CH₂OH, CH₂NH₂ or CH₂OCH₂phenyl;
Y is CH₂, CH(CH₃), O or S;
A¹, A², A³, A⁴, and A⁵ are each independently C or N, wherein no more than two of A¹, A², A³, A⁴, or A⁵ are N;
Q¹ is —(CH₂)ₙO(CH₂)ₙR¹⁰; —(CH₂)ₙNR¹⁵(CH₂)ₙR¹⁰; —CN; —(CH₂)ₙCO₂R¹⁰; —B(OR¹⁰)₂; a boronic acid ethylene glycol ester; a boronic acid pinacol ester; a boronic acid propylene-1,3-diol ester; a boronic acid 2,2-dimethyl-propylene-1,3-diol ester; —N₃; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; NO₂; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or

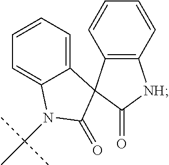

Q² is R¹¹, CF₃, O—R¹¹, OCF₃, halo, or CN;
m is 0, 1, or 2;
n is 0, 1, 2, or 3;
R¹⁰ is H; halo; OH; carboxyl; —S(O)₂OH; C₁₋₄ alkyl optionally substituted with one to four OH or with OCH₃; C₃₋₆ cycloalkyl optionally substituted with one or two halo; C₁₋₄ haloalkyl; —C₂₋₆ alkynyl; 1-benzyl-4-piperidyl; 2-tert-butoxy-2-oxo-ethyl; benzyloxyphenyl optionally substituted with one or two halo; O(C₁₋₂alkyl)ᵣOCH₃; NH₂; 2,3-dihydro-1H-indene; 2,3-dihydrobenzo[b][1,4]dioxine; benzo[d][1,3]dioxole optionally substituted with one or two halo; indoline optionally substituted with C(O)CH₃; 5- or 6-membered heteroaryl or 9- or 10-membered bicyclic heteroaryl optionally substituted with C₁₋₄ alkyl, C₁₋₄ alkoxy, halo or phenyl; phenyl optionally substituted with one to three substituents independently selected from: halo, C₁₋₄ alkoxy, hydroxy, C₁₋₄ alkyl, CF₃, CN, —(CH₂)₂C(O)OH, —C(O)NHNH₂, —OCF₃, —N(CH₃)₂, C₃₋₆ cycloalkyl, —OC₃₋₆ cycloalkyl, —OCH₂C₃₋₆ cycloalkyl, 5- or 6-membered heteroaryl, —(CH₂)ₙ(5- or 6-membered heterocyclyl) or —(CH₂)ₙphenyl wherein the phenyl is optionally substituted one or two halo; R¹¹ is H, C₁₋₄ alkyl, or cyclopropyl;
r is 1, 2 or 3; and
R¹⁵ is H or C₁₋₃alkyl;
or a salt thereof,
wherein if X is OH then R^{5a} must be a protecting group; and
wherein the compound is not:
tert-butyl-3-[1-[(3-bromophenyl)methyl]-2-methoxy-2-oxo-ethyl]pyrrolidine-1-carboxylate;
3-(3-bromophenyl)-2-[1-tert-butoxycarbonylpyrrolidin-3-yl]-2-methyl-propanoic acid;
tert-butyl-3-[2-[4-benzyl-2-oxo-oxazolidin-3-yl]-1-[(3-bromophenyl)methyl]-2-oxo-ethyl]pyrrolidine-1-carboxylate;
tert-butyl-3-[(2-[4-benzyl-2-oxo-oxazolidin-3-yl]-1-[(3-nitrophenyl)methyl]-2-oxo-ethyl]pyrrolidine-1-carboxylate;
3-(3-bromophenyl)-2-[1-tert-butoxycarbonylpyrrolidin-3-yl]propanoic acid;
2-[(1-tert-butoxycarbonylpyrrolidin-3-yl]-3-(3-nitrophenyl) propanoic acid;
tert-butyl-3-[1-[(3-bromophenyl)methyl]-2-tert-butoxy-2-oxo-ethyl]pyrrolidine-1-carboxylate;
tert-butyl-3-[1-[(3-bromophenyl)methyl]-2-tert-butoxy-1-methyl-2-oxo-ethyl]pyrrolidine-1-carboxylate;
tert-butyl-3-[(2-tert-butoxy-1-[(3-nitrophenyl)methyl]-2-oxo-ethyl]pyrrolidine-1-carboxylate;
tert-butyl-3-[2-tert-butoxy-1-[(3-formylphenyl)methyl]-2-oxo-ethyl]pyrrolidine-1-carboxylate;
tert-butyl-3-[2-tert-butoxy-1-[(3-formylphenyl)methyl]-1-methyl-2-oxo-ethyl]pyrrolidine-1-carboxylate;
tert-butyl-3-[1-[[3-(aminomethyl)phenyl]methyl]-2-tert-butoxy-2-oxo-ethyl]pyrrolidine-1-carboxylate;
tert-butyl-3-[1-[(3-aminophenyl)methyl]-2-tert-butoxy-2-oxo-ethyl]pyrrolidine-1-carboxylate;
tert-butyl-3-[2-tert-butoxy-1-[[3-(hydroxymethyl)phenyl]methyl]-2-oxo-ethyl]pyrrolidine-1-carboxylate;
[3-[3-tert-butoxy-2-[1-tert-butoxycarbonylpyrrolidin-3-yl]-3-oxo-propyl]phenyl]boronic acid;
tert-butyl-3-[2-tert-butoxy-1-[(3-hydroxyphenyl)methyl]-2-oxo-ethyl]pyrrolidine-1-carboxylate.

In an alternate embodiment, there is provided a compound of formula II:

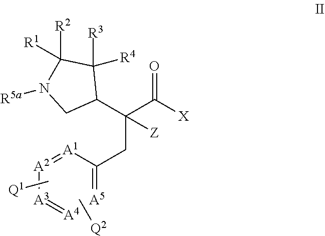

wherein
R¹ and R² are independently selected from H, C₁₋₄ alkyl, and F;
R³ and R⁴ are independently either H or F;
R^{5a} is H, C₁₋₄ alkyl, cyclopropyl, or a protecting group;

X is OH, —OR⁶, or

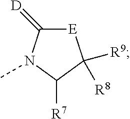

R⁶ is C₁₋₄ alkyl;
D and E are each independently O or S;
R⁷ is H, C₁₋₄ alkyl, phenyl or benzyl, wherein the phenyl and benzyl are optionally substituted with one or two substituents independently selected from: halo, C₁₋₄ alkyl, trifluoromethyl, amino, C₁₋₄ alkylamino, and di-C₁₋₄ alkylamino;
R⁸ and R⁹ are each independently H, C₁₋₄ alkyl or phenyl optionally substituted with one or two substituents independently selected from: halo, C₁₋₄ alkyl, trifluoromethyl, amino, C₁₋₄ alkylamino and di-C₁₋₄ alkylamino;
Z is H, C₁₋₄ alkyl, or cyclopropyl;
A¹, A², A³, A⁴, and A⁵ may each independently be C or N, wherein no more than two of A¹, A², A³, A⁴, or A⁵ are N;
Q¹ is —(CH₂)ₙO(CH₂)ₙR¹⁰, —(CH₂)ₙNH(CH₂)ₙR¹⁰, —CN, —(CH₂)ₙCO₂R¹⁰, —B(OR¹⁰)₂, a boronic acid ethylene glycol ester, a boronic acid pinacol ester, a boronic acid propylene-1,3-diol ester, a boronic acid 2,2-dimethyl-propylene-1,3-diol ester, —N₃, C₁₋₆ alkyl, —C₂₋₆ alkenyl, —C₂₋₆ alkynyl, halo, —C(O)—R¹⁰, —C(O)NHR¹⁰, —NH—NH₂, —S(O)ₘ(CH₂)ₙR¹⁰, —(CH₂)ₙNHS(O)ₘ(CH₂)ₙR¹⁰, —(CH₂)ₙS(O)ₘNH(CH₂)ₙR¹⁰, —(CH₂)ₙNHCONHR¹⁰, —NHCO(CH₂)ₙR¹⁰, —NHCOOR¹⁰, or an aryl or heteroaryl selected from: phenyl, pyrrole, imidazole, pyrazole, triazole, furan, thiophene, oxazole, isoxazole, thiazole, isothiazole, pyridine, pyridazine, pyrimidine and pyrazine, wherein aryl and heteroaryl are optionally substituted with a halo or OCF₃ substituent;
Q² is R¹¹, CF₃, O—R¹¹, OCF₃, halo, or CN;
m is 0, 1, or 2;
n is 0, 1, or 2;
R¹⁰ is H; hydroxy; carboxyl; C₁₋₄ alkyl optionally substituted with OH; C₃₋₆ cycloalkyl; 4,4-difluorocyclohexyl; C₁₋₄ haloalkyl; 3-(1H)-benzimidazol-2-yl; thiazole optionally substituted with methyl; methyl(phenyl)carbamoyl; 1-benzyl-4-piperidyl; 8-quinolyl; 2-methoxyethyl; 3-methoxypropyl; 3-phenyl-1,2,4-oxadiazol-5-yl; 2-tert-butoxy-2-oxo-ethyl; benzyloxyphenyl; phenyl optionally substituted with one to three substituents independently selected from: halo, C₁₋₄ alkoxy, hydroxy, C₁₋₄ alkyl, CF₃, CN, (CH₂)₂C(O)OH, C(O)NHNH₂, OCF₃, N(CH₃)₂, C₃₋₆ cycloalkyl, —OC₃₋₆ cycloalkyl and —OCH₂C₃₋₆ cycloalkyl;
and R¹¹ is H, C₁₋₄ alkyl, or cyclopropyl;
wherein if X is OH then R^{5a} must be a protecting group;
or a salt thereof.

In a fourth aspect, there is provided a compound of the formula:

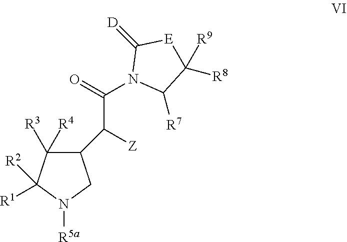

wherein
R¹ and R² are independently selected from H, C₁₋₄ alkyl, and F;
R³ and R⁴ are independently either H or F;
R^{5a} is H, C₁₋₄ alkyl, cyclopropyl, or a protecting group;
D and E are each independently O or S;
R⁷ is H, C₁₋₄ alkyl, phenyl or benzyl, wherein the phenyl and benzyl are optionally substituted with one or two substituents independently selected from: halo, C₁₋₄ alkyl, trifluoromethyl, amino, C₁₋₄ alkylamino, and di-C₁₋₄ alkylamino;
R⁸ and R⁹ are each independently H, C₁₋₄ alkyl or phenyl optionally substituted with one or two substituents independently selected from: halo, C₁₋₄ alkyl, trifluoromethyl, amino, C₁₋₄ alkylamino and di-C₁₋₄ alkylamino;
Z is H, C₁₋₄ alkyl, OH, cyclopropyl, CH₂OH, CH₂NH₂ or CH₂OCH₂phenyl;
or a salt thereof,
wherein the compound is not:

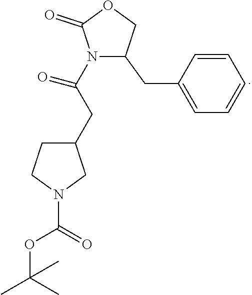 [SMILES: CC(C)(C)OC(=O)N1CCC(CC(=O)N2C(=O)OCC2Cc2ccccc2)C1 ; "."]

In a fifth aspect, there is provided the use of a compound of formula I, II, IV, V or VI in the preparation of an oligomer.

In a sixth aspect, there is provided an oligomer prepared from a compound of formula I, II, IV, V or V1.

In a seventh aspect, there is provided a pharmaceutical composition comprising the aforementioned oligomer.

In one embodiment of a compound of formula III, L¹ is attached at A¹, A² or A³ and is selected from a bond, —(CH₂)ₚNHC(O)NH(CH₂)ₚ—, —(CH₂)ₚC(O)NH(CH₂)ₚ—, —(CH₂)ₚS(O)₂NH(CH₂)ₚ—, —(CH₂)_{q}—, —(CH₂)ₚNH(CH₂)ₚ—,

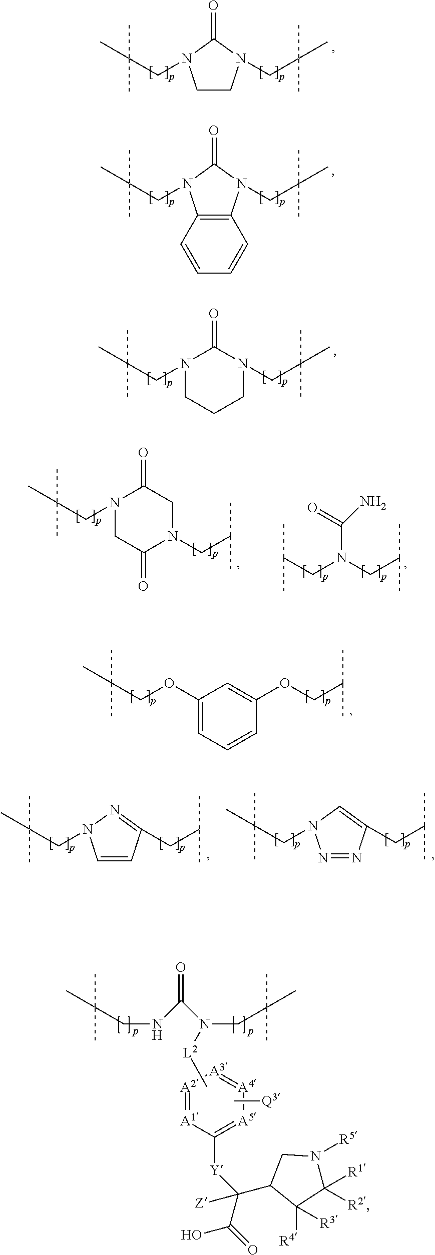

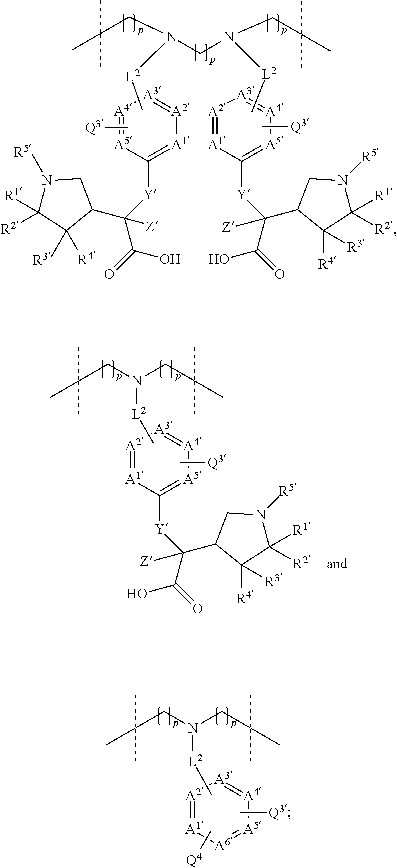

or L¹ together with the carbons at positions A² and A³ on one ring form the fused ring:

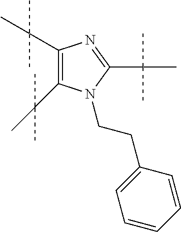

R¹, R^{1′}, R² and R^{2′} at each occurrence are independently selected from H, C₁₋₄ alkyl and F;
R³, R^{3′}, R⁴ and R^{4′} at each occurrence are independently either H or F;
R⁵ and R^{5′} at each occurrence are independently H, C₁₋₄ alkyl, or cyclopropyl;
Z and Z′ at each occurrence are independently H, C₁₋₄ alkyl, OH, or cyclopropyl;
Y and Y′ at each occurrence are independently CH₂, O or S;
A¹, A^{1′}, A², A^{2′}, A³, A^{3′}, A⁴, A^{4′}, A⁵, A^{5′} and A⁶ at each occurrence are independently C or N, wherein no more than two of A¹, A², A³, A⁴ and A⁵ on each ring are N or no more than two of A¹, A^{2′}, A^{3′}, A^{4′}, A^{5′} and A^{6′} on each ring are N;
Q³ and Q^{3′} at each occurrence are independently H, —(CH₂)ₙO(CH₂)ₙR¹⁰, —(CH₂)ₙNH(CH₂)ₙR¹⁰, —CN, —(CH₂)ₙCO₂R¹⁰, C₁₋₆ alkyl, —C₂₋₆ alkenyl, —C₂₋₆ alkynyl, halo, —C(O)—R¹⁰, —C(O)NHR¹⁰, —S(O)ₘ(CH₂)ₙR¹⁰, —(CH₂)ₙNHS(O)ₘ(CH₂)ₙR¹⁰, —(CH₂)ₙS(O)ₘNH(CH₂)ₙR¹⁰, —(CH₂)ₙNHCONHR¹⁰, —NHCO(CH₂)ₙR¹⁰, —NHCOOR¹⁰, NO₂, cyclopropyl, —O-cyclopropyl, CF₃, OCF₃, OH, or an aryl or heteroaryl selected from: phenyl, pyrrole, imidazole, pyrazole, triazole, furan, thiophene, oxazole, isoxazole, thiazole, isothiazole, pyridine, pyridazine, pyrimidine and pyrazine, wherein aryl and heteroaryl are optionally substituted with a halo or OCF₃ substituent;
Q⁴ is R¹¹, CF₃, O—R¹¹, OCF₃, halo, or CN;
m at each occurrence is independently 0, 1, or 2;
n at each occurrence is independently 0, 1, or 2;
R¹⁰ at each occurrence is independently H; halo; hydroxy; carboxyl; C₁₋₄ alkyl optionally substituted with OH; C₃₋₆ cycloalkyl; 4,4-difluorocyclohexyl; C₁₋₄ haloalkyl; 3-(1H)-benzimidazol-2-yl; thiazole optionally substituted with methyl; methyl(phenyl)carbamoyl; 1-benzyl-4-piperidyl; 8-quinolyl; 2-methoxyethyl; 3-methoxypropyl; 3-phenyl-1,2,4-oxadiazol-5-yl; 2-tert-butoxy-2-oxo-ethyl; benzyloxyphenyl; phenyl optionally substituted with one to three substituents independently selected from: halo, C₁₋₄ alkoxy, hydroxy, C₁₋₄ alkyl, CF₃, CN, (CH₂)₂C(O)OH, C(O)NHNH₂, OCF₃, N(CH₃)₂, C₃₋₆ cycloalkyl, —OC₃₋₆ cycloalkyl and —OCH₂C₃₋₆ cycloalkyl;
R¹¹ is H, C₁₋₄ alkyl, or cyclopropyl;
L² is attached at A^{1′}, A^{2′} or A^{3′} and is C₁₋₃ alkylene or a bond; and
p at each occurrence is independently 0 to 3;
q is 1 to 5;
or a pharmaceutically acceptable salt thereof,
wherein the compound is not of the formula:

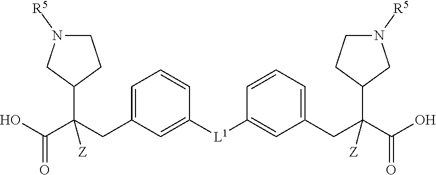

wherein
L¹ is selected from the group consisting of —CH₂NHCH₂—, —CH₂NH—, —NH—,

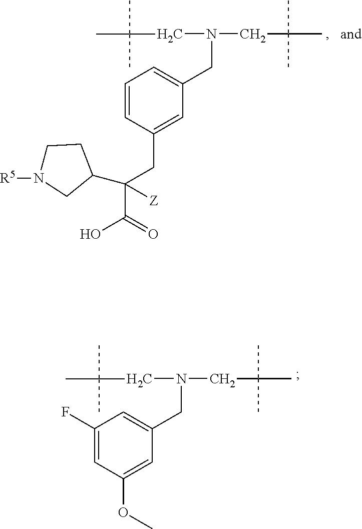

R⁵ is H or CH₃; and
Z is H or CH₃.

In a compound of formula III, L¹ may be attached at different points on each ring. In one embodiment of a compound of formula III, L¹ is attached at A² on each ring; at A² on one ring and A³ on the other; at A³ on each ring; or at A¹ on one ring and A² on the other. In a further embodiment, L¹ is attached at A² on each ring; at A² on one ring and A³ on the other; or at A³ on each ring. In a preferred embodiment, L¹ is attached at A² on each ring.

In one embodiment of a compound of formula III, L¹ is selected from: a bond, —(CH₂)ₚNHC(O)NH(CH₂)ₚ—, —(CH₂)ₚC(O)NH(CH₂)ₚ—, —(CH₂)ₚS(O)₂NH(CH₂)ₚ—, —(CH₂)_{q}—,

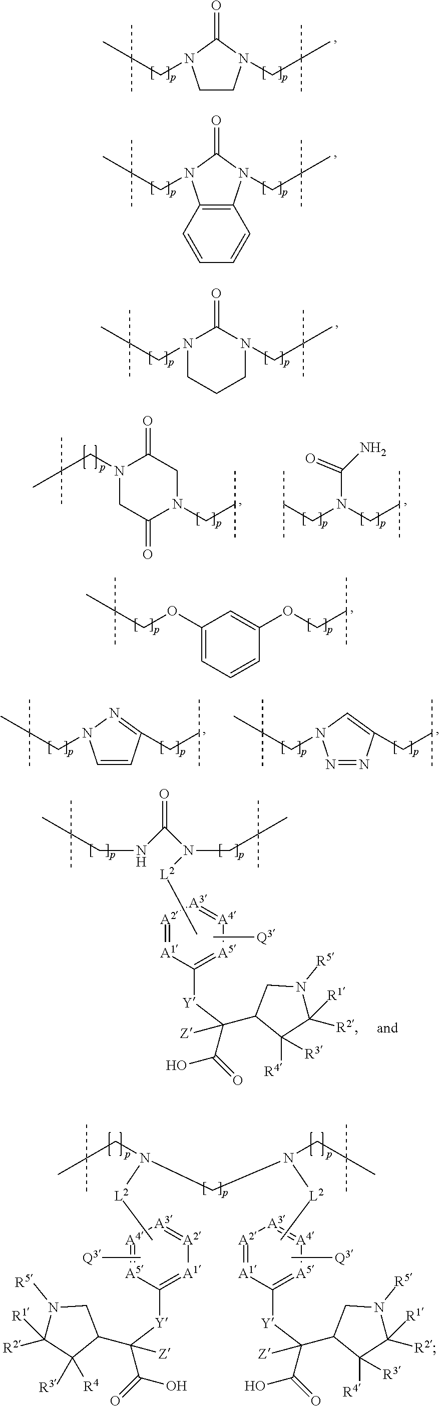

or L¹ together with the carbons at positions A² and A³ on one ring form the fused ring:

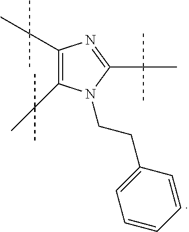

In one embodiment of a compound of formula III, L¹ is —(CH₂)ₚNH(CH₂)ₚ—,

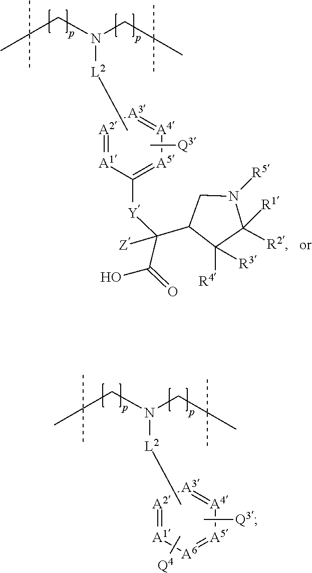

and at least one Q³ is other than H.

In one embodiment of a compound of formula III, L¹ is selected from: a bond, —(CH₂)ₚNHC(O)NH(CH₂)ₚ—, —(CH₂)ₚC(O)NH(CH₂)ₚ—, —(CH₂)ₚS(O)₂NH(CH₂)ₚ—, —(CH₂)_{q}—, —(CH₂)ₚNH(CH₂)ₚ—,

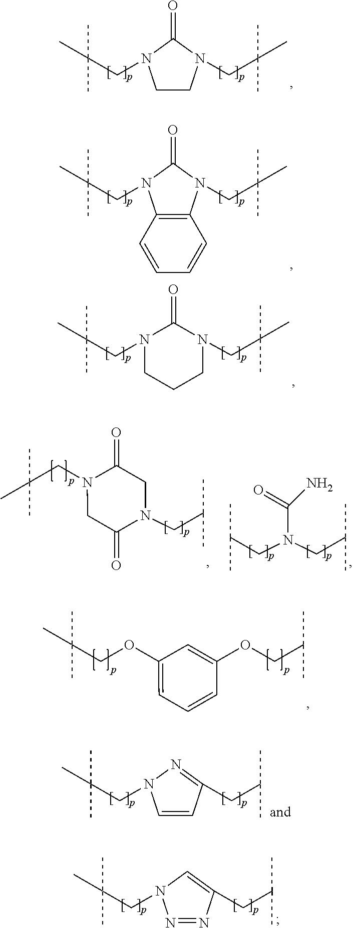

or L¹ together with the carbons at positions A² and A³ on one ring form the fused ring:

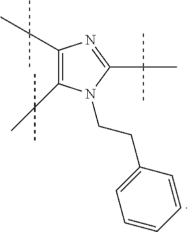

In a further embodiment, L¹ is selected from: a bond, —NHC(O)NH—, —NHC(O)NHCH₂—, —C(O)NH—, —S(O)₂NH—, —CH₂CH₂CH₂—, —NH—,

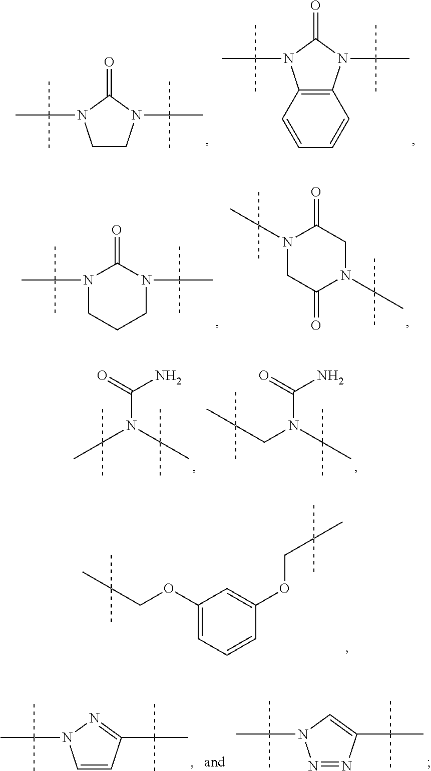

or L¹ together with the carbons at positions A² and A³ on one ring form the fused ring:

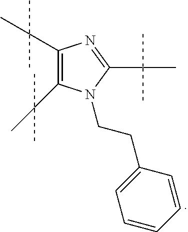

In a preferred embodiment, L¹ is selected from: a bond, —NHC(O)NH—, —NHC(O)NHCH₂—, —NH—,

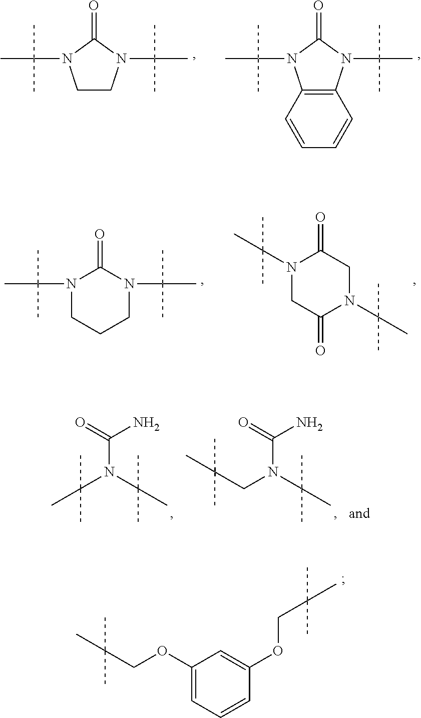

or L¹ together with the carbons at positions A² and A³ on one ring form the fused ring:

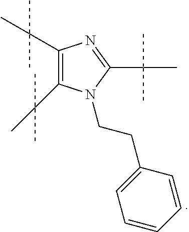

In one embodiment of a compound of formula III, L¹ is selected from:

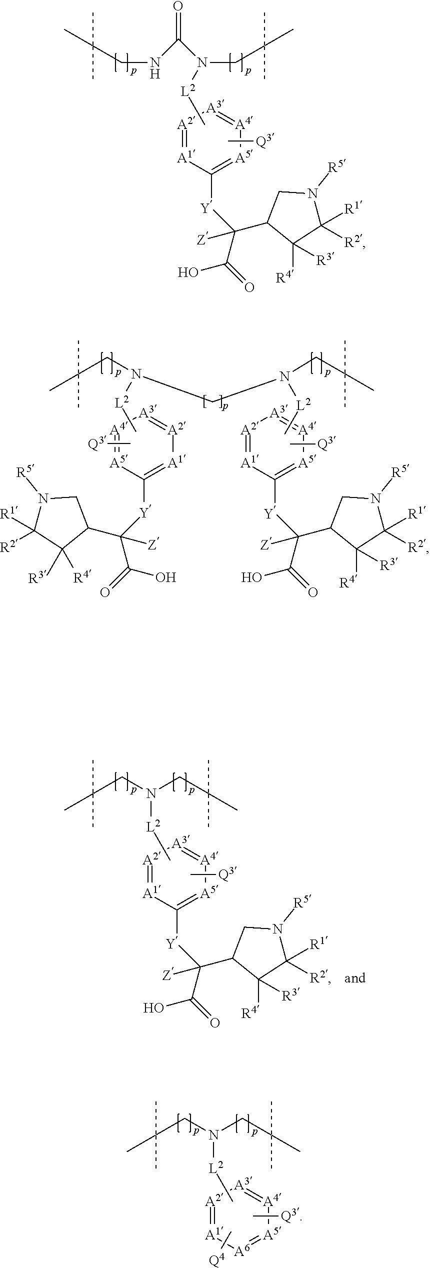

In a further embodiment, L¹ is selected from:

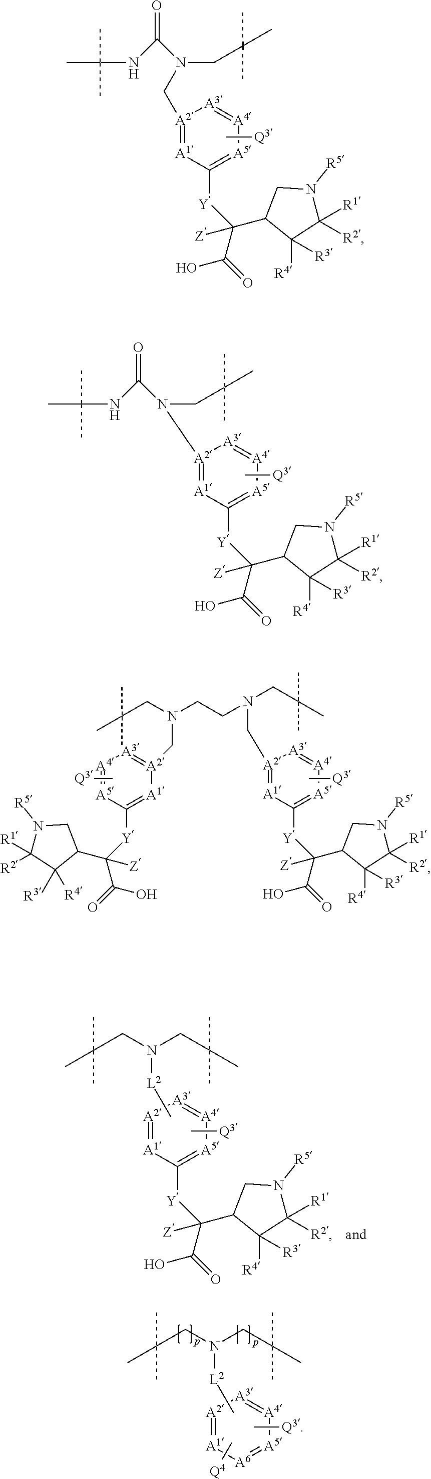

In yet a further embodiment, L¹ is selected from:

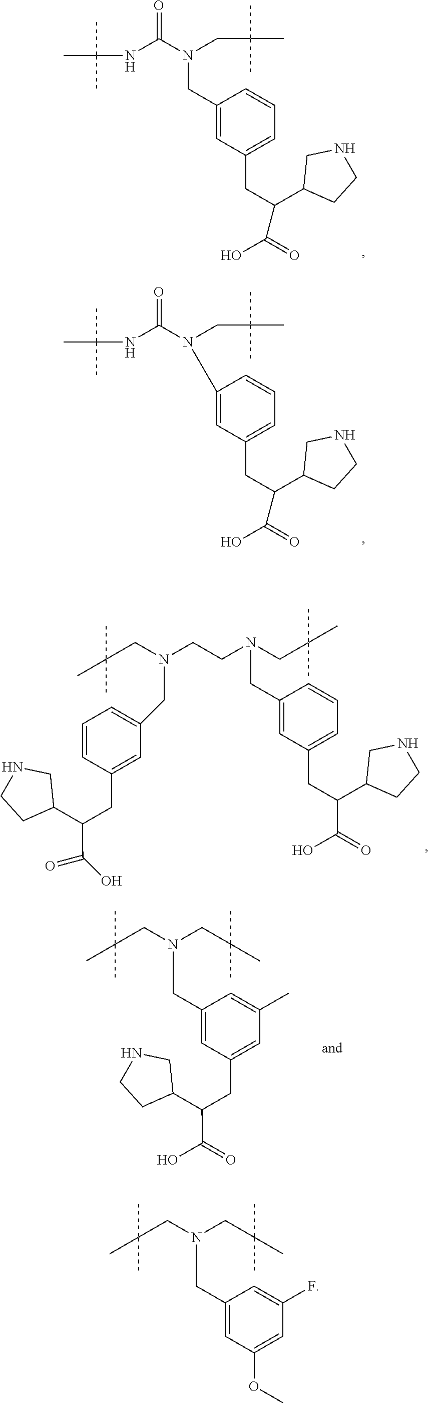

In a preferred embodiment, L¹ is selected from:

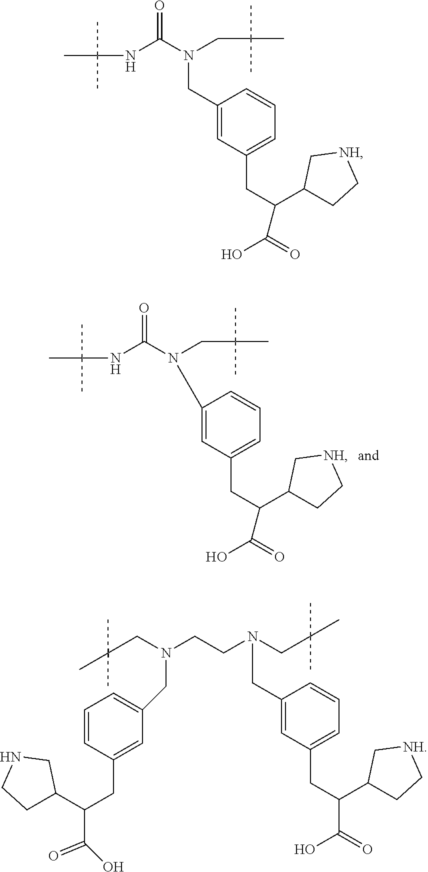

In one embodiment of a compound of formula III, on each ring R¹ and R² are both H or one of R¹ and R² is H and the other is CH₃.

In one embodiment of a compound of formula III, on each ring R³ and R⁴ are both H or R³ and R⁴ are both F.

In one embodiment of a compound of formula III, at each occurrence R⁵ is H.

In one embodiment of a compound of formula III, at each occurrence Z is H, CH₃ or OH.

In one embodiment of a compound of formula III, on each ring A¹, A², A³, A⁴ and A⁵ are all C; or on one ring two of A¹, A², A³, A⁴ and A⁵ are N and on the other A¹, A², A³, A⁴ and A⁵ are all C; or on each ring A³ is N and all others are C. In a preferred embodiment, on each ring A¹, A², A³, A⁴ and A⁵ are all C.

In one embodiment of a compound of formula III, at each occurrence Q³ is H, F, CF₃ or CN. In a preferred embodiment, at each occurrence Q³ is H, F or CF₃.

In one embodiment, the compound of formula III is selected from:

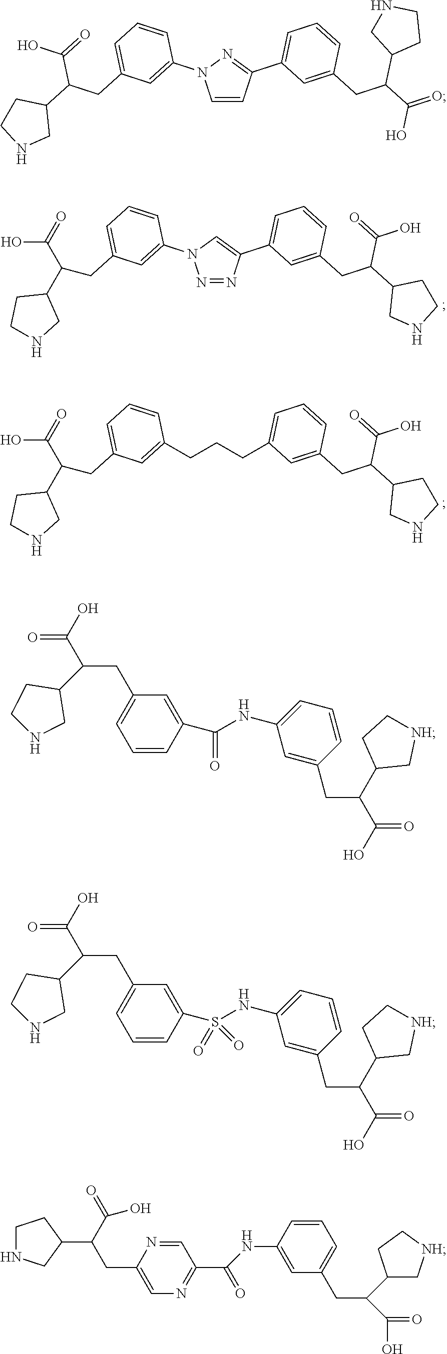 [SMILES: O=C(O)C(Cc1cccc(-c2ccn(-c3cccc(CC(C(=O)O)C4CCNC4)c3)n2)c1)C1CCNC1 ; O=C(O)C(Cc1cccc(-c2cn(-c3cccc(CC(C(=O)O)C4CCNC4)c3)nn2)c1)C1CCNC1 ; O=C(O)C(Cc1cccc(CCCc2cccc(CC(C(=O)O)C3CCNC3)c2)c1)C1CCNC1 ; O=C(Nc1cccc(CC(C(=O)O)C2CCNC2)c1)c1cccc(CC(C(=O)O)C2CCNC2)c1 ; O=C(O)C(Cc1cccc(NS(=O)(=O)c2cccc(CC(C(=O)O)C3CCNC3)c2)c1)C1CCNC1 ; O=C(Nc1cccc(CC(C(=O)O)C2CCNC2)c1)c1cnc(CC(C(=O)O)C2CCNC2)cn1]

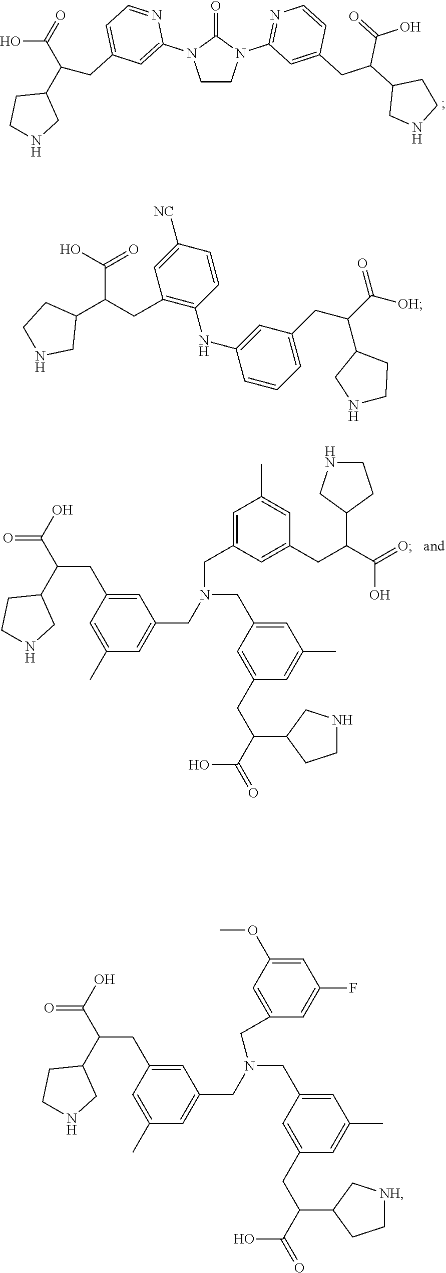 [SMILES: O=C(O)C(Cc1ccnc(N2CCN(c3cc(CC(C(=O)O)C4CCNC4)ccn3)C2=O)c1)C1CCNC1 ; N#Cc1ccc(Nc2cccc(CC(C(=O)O)C3CCNC3)c2)c(CC(C(=O)O)C2CCNC2)c1 ; Cc1cc(CC(C(=O)O)C2CCNC2)cc(CN(Cc2cc(C)cc(CC(C(=O)O)C3CCNC3)c2)Cc2cc(C)cc(CC(C(=O)O)C3CCNC3)c2)c1 ; "and" ; COc1cc(F)cc(CN(Cc2cc(C)cc(CC(C(=O)O)C3CCNC3)c2)Cc2cc(C)cc(CC(C(=O)O)C3CCNC3)c2)c1]

or a pharmaceutically acceptable salt thereof.

In one embodiment of the compound of Formula IV:
R¹ and R² are independently selected from H, C₁₋₄ alkyl, and F;
R³ and R⁴ are independently either H or F;
R⁵ is H, C₁₋₄ alkyl, or cyclopropyl;
Z is H, C₁₋₄ alkyl, OH, or cyclopropyl;
Y is CH₂, O or S;
A¹, A², A³, A⁴, and A⁵ are each independently C or N, wherein no more than two of A¹, A², A³, A⁴, or A⁵ are N;
Q¹ is —(CH₂)ₙO(CH₂)ₙR¹⁰, —(CH₂)ₙNH(CH₂)ₙR¹⁰, —CN, —(CH₂)ₙCO₂R¹⁰, —B(OR¹⁰)₂, a boronic acid ethylene glycol ester, a boronic acid pinacol ester, a boronic acid propylene-1,3-diol ester, a boronic acid 2,2-dimethyl-propylene-1,3-diol ester, —N₃, C₁₋₆ alkyl, —C₂₋₆ alkenyl, —C₂₋₆ alkynyl, halo, —C(O)—R¹⁰, —C(O)NHR¹⁰, —NH—NH₂, —S(O)ₘ(CH₂)ₙR¹⁰, —(CH₂)ₙNHS(O)ₘ(CH₂)ₙR¹⁰, —(CH₂)ₙS(O)ₘNH(CH₂)ₙR¹⁰, —(CH₂)ₙNHCONHR¹⁰, —NHCO(CH₂)ₙR¹⁰, —NHCOOR¹⁰, NO₂, or an aryl or heteroaryl selected from: phenyl, pyrrole, imidazole, pyrazole, triazole, furan, thiophene, oxazole, isoxazole, thiazole, isothiazole, pyridine, pyridazine, pyrimidine and pyrazine, wherein aryl and heteroaryl are optionally substituted with a halo or OCF₃ substituent;
Q² is R¹¹, CF₃, O—R¹¹, OCF₃, halo, or CN;
m is 0, 1, or 2;
n is 0, 1, or 2;
R¹⁰ is H; halo; hydroxy; carboxyl; C₁₋₄ alkyl optionally substituted with OH; C₃₋₆ cycloalkyl; 4,4-difluorocyclohexyl; C₁₋₄ haloalkyl; 3-(1H)-benzimidazol-2-yl; thiazole optionally substituted with methyl; methyl(phenyl)carbamoyl; 1-benzyl-4-piperidyl; 8-quinolyl; 2-methoxyethyl; 3-methoxypropyl; 3-phenyl-1,2,4-oxadiazol-5-yl; 2-tert-butoxy-2-oxo-ethyl; benzyloxyphenyl; phenyl optionally substituted with one to three substituents independently selected from: halo, C₁₋₄ alkoxy, hydroxy, C₁₋₄ alkyl, CF₃, CN, —(CH₂)₂C(O)OH, C(O)NHNH₂, OCF₃, N(CH₃)₂, C₃₋₆ cycloalkyl, —OC₃₋₆ cycloalkyl and —OCH₂C₃₋₆ cycloalkyl;
and R¹¹ is H, C₁₋₄ alkyl, or cyclopropyl.

In one further embodiment, the compound according to formula IV is a compound of formula IV′:

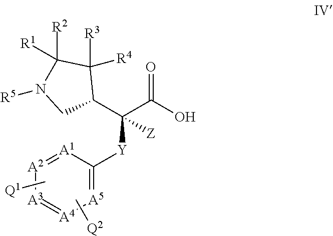

or a pharmaceutically acceptable salt thereof.

In an embodiment, the compound according to formula I is a compound of formula I′:

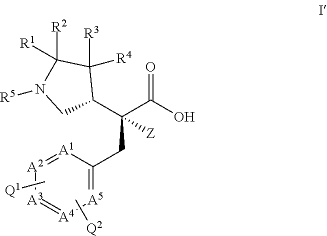

or a pharmaceutically acceptable salt thereof.

In another embodiment, the compound according to formula I or IV is selected from:

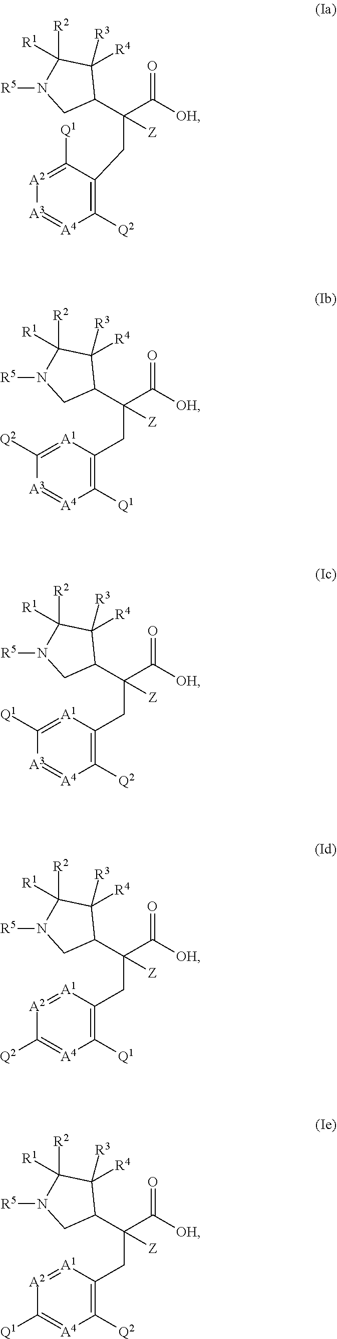

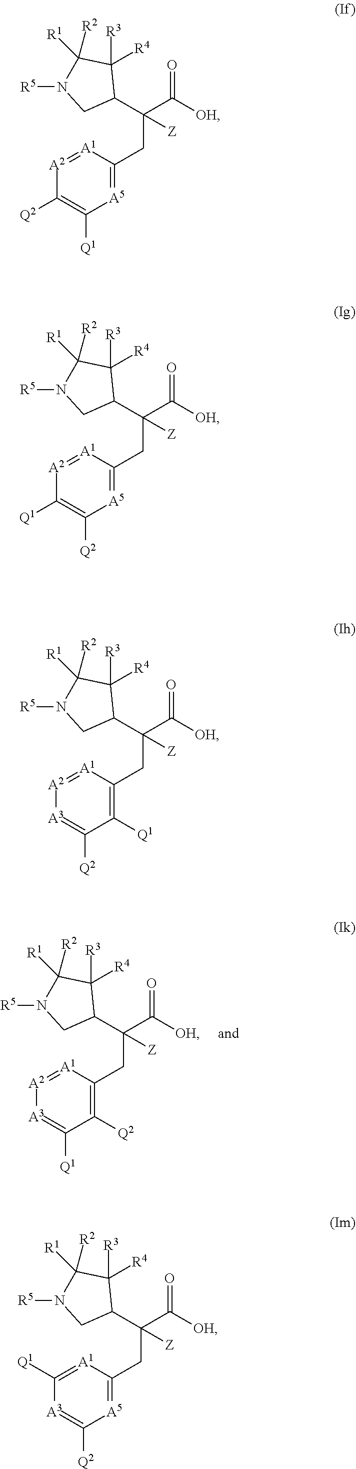

or a pharmaceutically acceptable salt thereof.

In yet one further embodiment, the compound of formula I or IV is selected from:

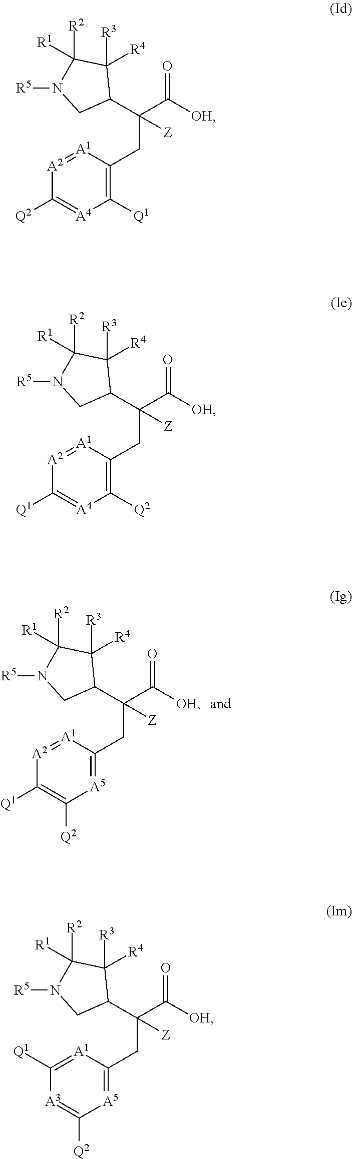

or a pharmaceutically acceptable salt of thereof.

In one embodiment, the compound of formula I or IV is selected from:

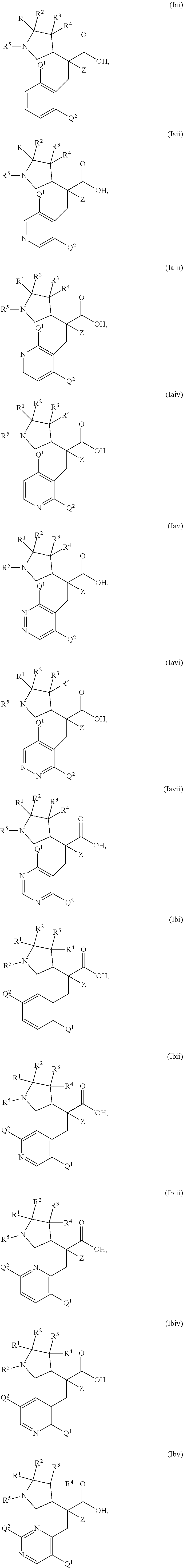

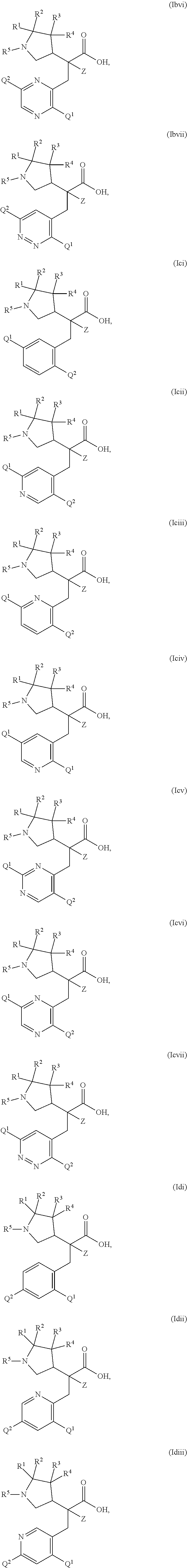

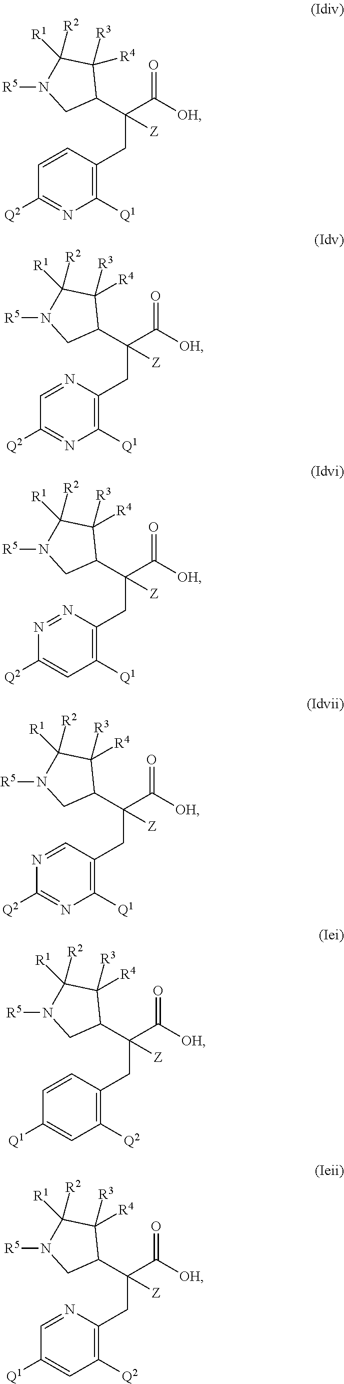

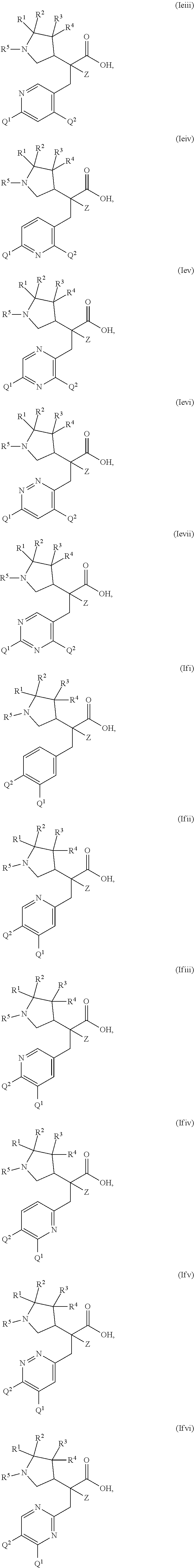

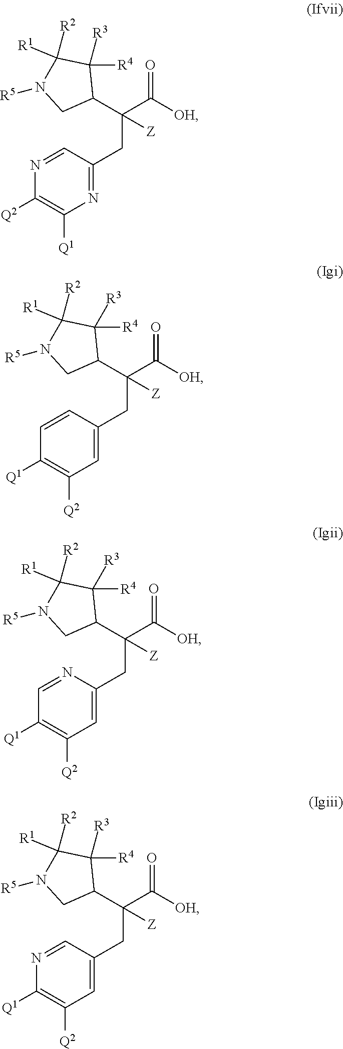

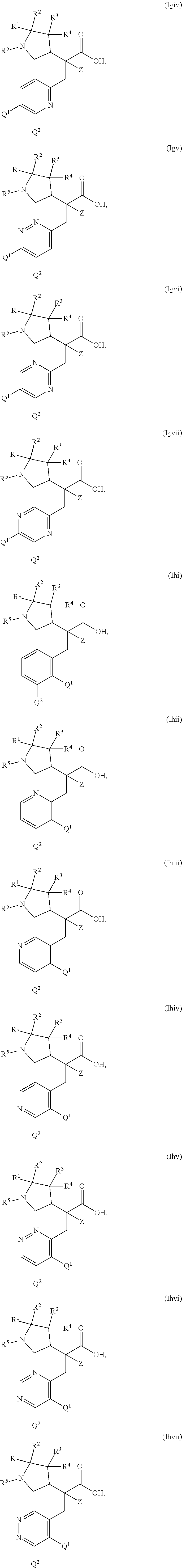

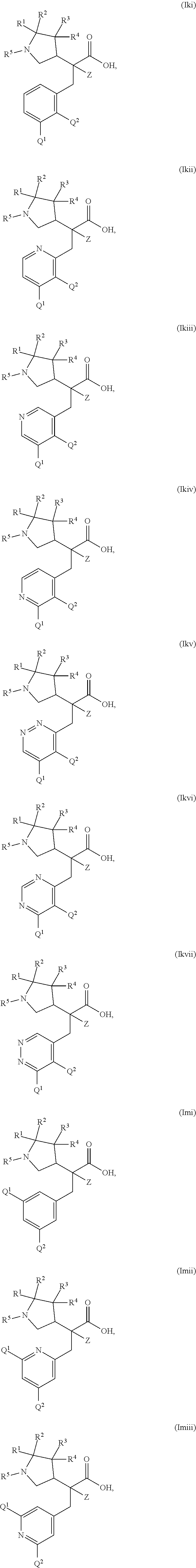

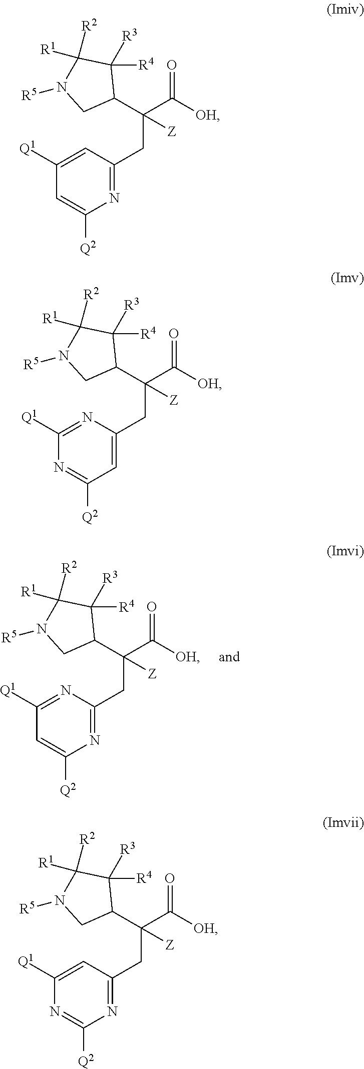

or a pharmaceutically acceptable salt thereof.

In yet another embodiment, the compound of formula I or IV is selected from:

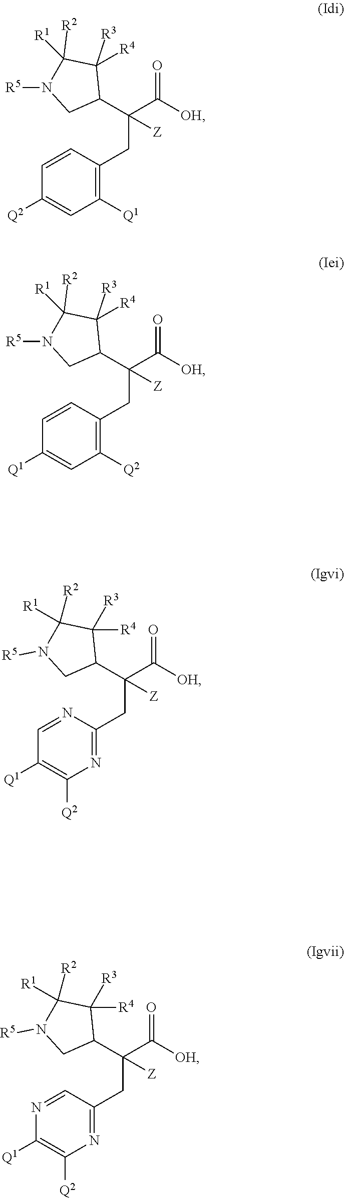

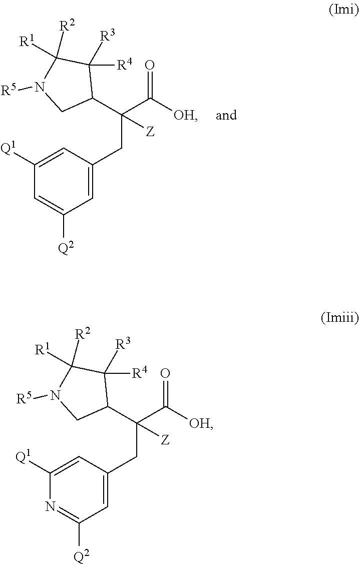

or a pharmaceutically acceptable salt thereof.

In a further embodiment, the compound of formula I or IV is selected from:

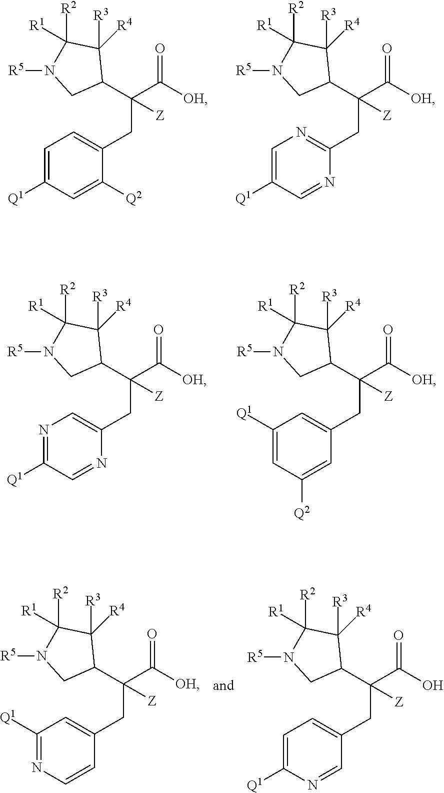

or a pharmaceutically acceptable salt thereof.

In one embodiment of a compound of formula I, I′, IV or IV′, Q¹ is selected from: —(CH₂)ₙO(CH₂)ₙR¹⁰; —(CH₂)ₙNR¹⁵(CH₂)ₙR¹⁰; —CN; —(CH₂)ₙCO₂R¹⁰; —N₃; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; CF₃; C₃₋₆cycloalkyl; —NH(C═NH)CH₂CN; 5- or 6-membered heterocyclyl optionally substituted with one to four halo or with a phenyl; indoline optionally substituted with one or two CH₃; imidazolidinone or imidazolidin-2,5-dione optionally substituted with (CH₂)CF₃, or (CH₂)ₙphenyl, which phenyl is optionally substituted with OH; indolin-2-one or benzimidazol-2-one optionally substituted with CH₃; phenyl, 5- or 6-membered heteroaryl or 9-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆cycloalkyl, C₁₋₄ alkoxy, C₁₋₆alkyl optionally substituted with OH or halo, phenyl or benzyl which phenyl or benzyl is optionally substituted with one to three substituents independently selected from —OCH₃ and halo; or

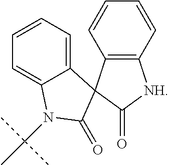

In one embodiment of a compound of formula I, I′, IV or IV′, Q² is H, CH₃, F or Br.

In one embodiment of a compound of formula I, I′, IV or IV′, R⁵ is H. In another embodiment of a compound of formula I, I′, IV or IV′, at least one of R¹, R², R³, and R⁴ is F. In yet another embodiment of a compound of formula I, I′, IV or IV′, at least two of R¹, R², R³, and R⁴ are F. In one further embodiment of a compound of formula I, I′, IV or IV′, R¹, R², R³, and R⁴ are all H. In one further embodiment of a compound of formula I, I′, IV or IV′, R¹ and R² are H and R³ and R⁴ are F.

In one embodiment of a compound of formula I, I′, IV or IV′, Z is selected from H, methyl, CH₂OH, CH₂NH₂ and CH₂OCH₂phenyl. In yet another embodiment of a compound of formula I, I′, IV or IV′, Z is selected from H, methyl, ethyl, n-propyl, and i-propyl.

In one embodiment, the compound according to formula I or IV is selected from:

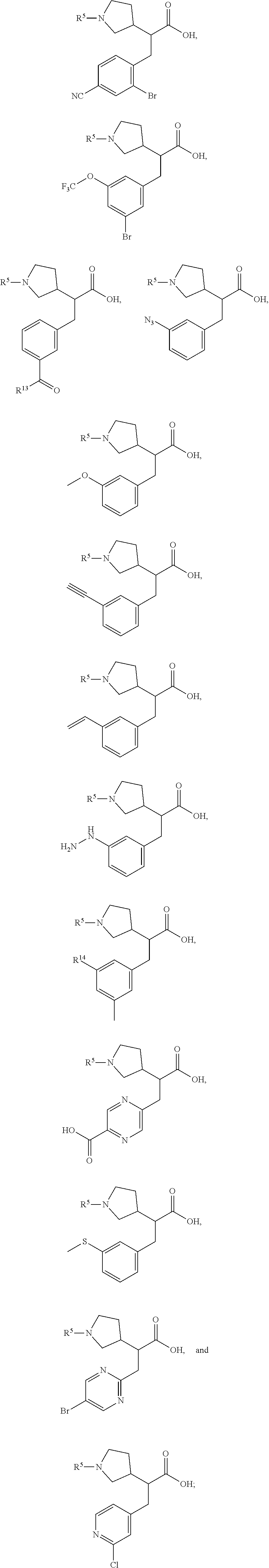

wherein R¹³ is selected from OH, OCH₃, CH₃, and NH₂;
wherein R¹⁴ is selected from Br, OH, C(O)H, CH₂OH, CH₂NH₂, and B(OH)₂, or a pharmaceutically acceptable salt thereof.

In one embodiment of a compound of formula V:
R¹ and R² are independently selected from H, C₁₋₄ alkyl, and F;
R³ and R⁴ are independently either H or F;
R^{5a} is H, C₁₋₄ alkyl, cyclopropyl, or a protecting group;
X is OH, —OR⁶, or

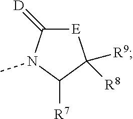

R⁶ is C₁₋₄ alkyl;
D and E are each independently O or S;
R⁷ is H, C₁₋₄ alkyl, phenyl or benzyl, wherein the phenyl and benzyl are optionally substituted with one or two substituents independently selected from: halo, C₁₋₄ alkyl, trifluoromethyl, amino, C₁₋₄ alkylamino, and di-C₁₋₄ alkylamino;
R⁸ and R⁹ are each independently H, C₁₋₄ alkyl or phenyl optionally substituted with one or two substituents independently selected from: halo, C₁₋₄ alkyl, trifluoromethyl, amino, C₁₋₄ alkylamino and di-C₁₋₄ alkylamino;
Z is H, C₁₋₄ alkyl, OH, or cyclopropyl;
Y is CH₂, O or S;
A¹, A², A³, A⁴, and A⁵ are each independently C or N, wherein no more than two of A¹, A², A³, A⁴, or A⁵ are N;
Q¹ is —(CH₂)ₙO(CH₂)ₙR¹⁰, —(CH₂)ₙNH(CH₂)ₙR¹⁰, —CN, —(CH₂)ₙCO₂R¹⁰, —B(OR¹⁰)₂, a boronic acid ethylene glycol ester, a boronic acid pinacol ester, a boronic acid propylene-1,3-diol ester, a boronic acid 2,2-dimethyl-propylene-1,3-diol ester, —N₃, C₁₋₆ alkyl, —C₂₋₆ alkenyl, —C₂₋₆ alkynyl, halo, —C(O)—R¹⁰, —C(O)NHR¹⁰, —NH—NH₂, —S(O)ₘ(CH₂)ₙR¹⁰, —(CH₂)ₙNHS(O)ₘ(CH₂)ₙR¹⁰, —(CH₂)ₙS(O)ₘNH(CH₂)ₙR¹⁰, —(CH₂)ₙNHCONHR¹⁰, —NHCO(CH₂)ₙR¹⁰, —NHCOOR¹⁰, NO₂, or an aryl or heteroaryl selected from: phenyl, pyrrole, imidazole, pyrazole, triazole, furan, thiophene, oxazole, isoxazole, thiazole, isothiazole, pyridine, pyridazine, pyrimidine and pyrazine, wherein aryl and heteroaryl are optionally substituted with a halo or OCF₃ substituent;
Q² is R¹¹, CF₃, O—R¹¹, OCF₃, halo, or CN;
m is 0, 1, or 2;
n is 0, 1, or 2;
R¹⁰ is H; halo; hydroxy; carboxyl; C₁₋₄ alkyl optionally substituted with OH; C₃₋₆ cycloalkyl; 4,4-difluorocyclohexyl; C₁₋₄ haloalkyl; 3-(1H)-benzimidazol-2-yl; thiazole optionally substituted with methyl; methyl(phenyl)carbamoyl; 1-benzyl-4-piperidyl; 8-quinolyl; 2-methoxyethyl; 3-methoxypropyl; 3-phenyl-1,2,4-oxadiazol-5-yl; 2-tert-butoxy-2-oxo-ethyl; benzyloxyphenyl; phenyl optionally substituted with one to three substituents independently selected from: halo, C₁₋₄ alkoxy, hydroxy, C₁₋₄ alkyl, CF₃, CN, (CH₂)₂C(O)OH, C(O)NHNH₂, OCF₃, N(CH₃)₂, C₃₋₆ cycloalkyl, —OC₃₋₆ cycloalkyl and —OCH₂C₃₋₆ cycloalkyl;
and R¹¹ is H, C₁₋₄ alkyl, or cyclopropyl;
wherein if X is OH then R^{5a} must be a protecting group; and
wherein the compound is not:
tert-butyl-3-[1-[(3-bromophenyl)methyl]-2-methoxy-2-oxo-ethyl]pyrrolidine-1-carboxylate;
3-(3-bromophenyl)-2-[1-tert-butoxycarbonylpyrrolidin-3-yl]-2-methyl-propanoic acid;
tert-butyl-3-[2-[4-benzyl-2-oxo-oxazolidin-3-yl]-1-[(3-bromophenyl)methyl]-2-oxo-ethyl]pyrrolidine-1-carboxylate;
tert-butyl-3-[(2-[4-benzyl-2-oxo-oxazolidin-3-yl]-1-[(3-nitrophenyl)methyl]-2-oxo-ethyl]pyrrolidine-1-carboxylate;
3-(3-bromophenyl)-2-[1-tert-butoxycarbonylpyrrolidin-3-yl]propanoic acid;
2-[(1-tert-butoxycarbonylpyrrolidin-3-yl]-3-(3-nitrophenyl) propanoic acid;
tert-butyl-3-[1-[(3-bromophenyl)methyl]-2-tert-butoxy-2-oxo-ethyl]pyrrolidine-1-carboxylate;
tert-butyl-3-[1-[(3-bromophenyl)methyl]-2-tert-butoxy-1-methyl-2-oxo-ethyl]pyrrolidine-1-carboxylate;
tert-butyl-3-[(2-tert-butoxy-1-[(3-nitrophenyl)methyl]-2-oxo-ethyl]pyrrolidine-1-carboxylate;
tert-butyl-3-[2-tert-butoxy-1-[(3-formylphenyl)methyl]-2-oxo-ethyl]pyrrolidine-1-carboxylate;
tert-butyl-3-[2-tert-butoxy-1-[(3-formylphenyl)methyl]-1-methyl-2-oxo-ethyl]pyrrolidine-1-carboxylate;
tert-butyl-3-[1-[[3-(aminomethyl)phenyl]methyl]-2-tert-butoxy-2-oxo-ethyl]pyrrolidine-1-carboxylate;
tert-butyl-3-[1-[(3-aminophenyl)methyl]-2-tert-butoxy-2-oxo-ethyl]pyrrolidine-1-carboxylate;
tert-butyl-3-[2-tert-butoxy-1-[[3-(hydroxymethyl)phenyl]methyl]-2-oxo-ethyl]pyrrolidine-1-carboxylate;
[3-[3-tert-butoxy-2-[1-tert-butoxycarbonylpyrrolidin-3-yl]-3-oxo-propyl]phenyl]boronic acid;
tert-butyl-3-[2-tert-butoxy-1-[(3-hydroxyphenyl)methyl]-2-oxo-ethyl]pyrrolidine-1-carboxylate.

In one embodiment of a compound of formula V: Q¹ is —CN; —(CH₂)˜CO₂R¹⁰; a boronic acid ethylene glycol ester; a boronic acid pinacol ester; a boronic acid propylene-1,3-diol ester; a boronic acid 2,2-dimethyl-propylene-1,3-diol ester; —N₃; C₁₋₆ alkyl; —C₂₋₆ alkenyl; —C₂₋₆ alkynyl; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or

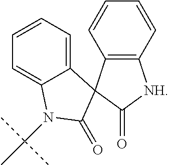

In one embodiment of a compound of formula II, V or VI, R^{5a} can represent a protecting group (“PG”) for the pyrrolidine nitrogen. The following protecting groups can be considered (PG abbreviation expressed in parentheses): carbamates, such as tert-butyloxycarbonyl (Boc), carboxybenzyl (Cbz), 9-fluorenylmethoxycarbonyl (Fmoc), allyloxycarbonyl (Alloc), trimethylsilylethoxycarbonyl (Teoc), trichloroethoxycarbonyl (Troc); amides, such as trifluoroacetamide (Tfa) benzamide (Bz); amines, such as benzylamine (Bn), triphenylmethylamines (Tphm), sulfonamides, such as p-toluenesulfonamide. In particular, the PG may be selected from: Boc, Cbz, Fmoc, Alloc, Teoc, Troc, Tfa, Bz, Bn, Tphm, and p-toluenesulfonamide. In a particular embodiment, R^{5a} is Boc.

In another embodiment of a compound of formula II or V, X is selected from:

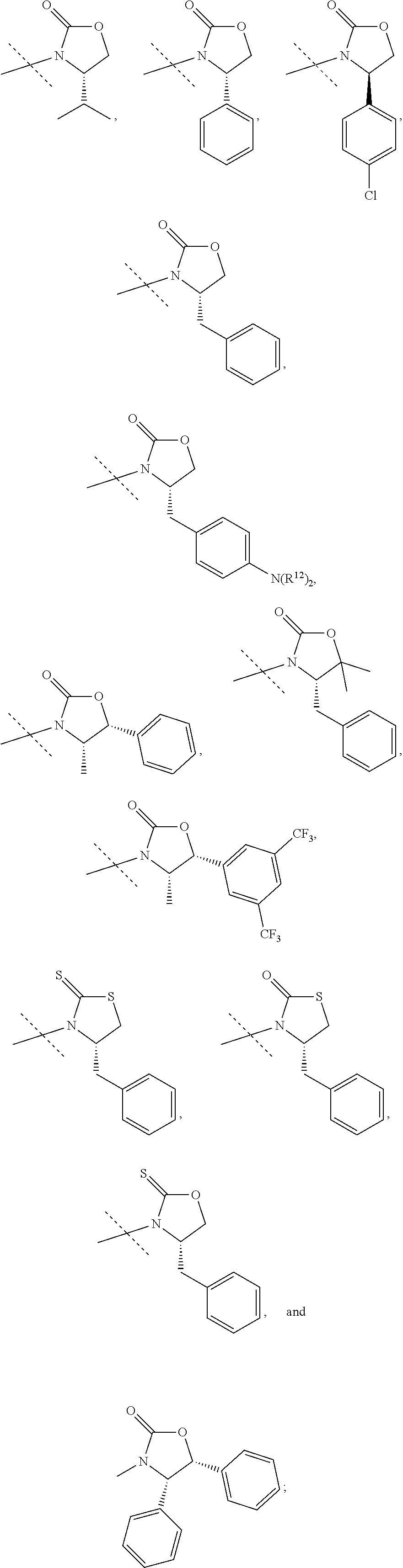

wherein R¹² at each occurrence is independently H or C₁₋₄ alkyl.

The compounds of formula VI are alternative intermediates which may be used in the preparation of compounds of formula I, I′, III, IV or IV′ and other oligomer compounds. The chirality of the compound of formula VI may be selected to direct the stereoselectivity of the alkylation step (Scheme 2). In an embodiment of a compound of formula VI, when R⁷ is benzyl then at least one of R¹, R², R³, R⁴, R⁵, R⁸ and R⁹ is other than H. In an alternate embodiment, either D or E is other than O.

Preferred aryl and heteroaryl groups: phenyl, pyrrole, imidazole, pyrazole, triazole, furan, thiophene, oxazole, isoxazole, thiazole, isothiazole, pyridine, pyridazine, pyrimidine, pyrazine, benzothiophene, indazole, indole, imidazopyridine, benzothiazole and benzimidazole

As mentioned above, one aspect of the present disclosure includes the use of a compound according to formula I, I′, II, IV, IV′, V or VI in the preparation of an oligomer. In one further aspect, the present disclosure includes an oligomer prepared from a compound according to formula, I′, II, IV, IV′, V or VI. In one embodiment, the oligomer comprises two pyrrolidine moieties. In yet another embodiment, the oligomer comprises three pyrrolidine moieties. Accordingly, one further aspect of the present disclosure includes a pharmaceutical composition comprising such oligomer.

In another embodiment, there is provided a pharmaceutically acceptable composition comprising a compound of formula I, I′, III, IV or IV′, or a pharmaceutically acceptable salt thereof, and at least one of a pharmaceutically acceptable carrier, diluent or excipient.

In an embodiment, there is provided a method of treating a patient in need of treatment for cardiovascular disease, comprising administering an effective amount of a compound of formula I, I′, III, IV or IV′, or a pharmaceutically acceptable salt thereof.

In an embodiment, there is provided a method of treating a patient in need of treatment for elevated Lp(a) plasma levels, comprising administering an effective amount of a compound of formula I, I′, III, IV or IV′, or a pharmaceutically acceptable salt thereof.

In an embodiment, there is provided a compound of formula I, I′, III, IV or IV′, or a pharmaceutically acceptable salt thereof, for use in therapy.

In an embodiment, there is provided a compound of formula I, I′, III, IV or IV′, or a pharmaceutically acceptable salt thereof, for use in the treatment of cardiovascular disease. In an embodiment, there is provided a compound of formula I, I′, III, IV or IV′, or a pharmaceutically acceptable salt thereof, for use in treating elevated Lp(a) plasma levels.

In an embodiment, there is provided the use of a compound of formula I, I′, III, IV or IV′, or a pharmaceutically acceptable salt thereof, in the manufacture of a medicament for the treatment of cardiovascular disease. In an embodiment, there is provided the use of a compound of formula I, I′, III, IV or IV′, or a pharmaceutically acceptable salt thereof, in the manufacture of a medicament for the treatment of elevated Lp(a) plasma levels.

The term “halogen” or “halo” refers to fluorine, chlorine, bromine, or iodine.

The term “C₁₋ₙ alkyl” refers to a straight or branched chain saturated hydrocarbon containing 1 to n carbon atoms. Examples of a C₁₋₄ alkyl group include, but are not limited to, methyl, ethyl, propyl, butyl, and tert-butyl. Examples of a C₁₋₃ alkyl group include, but are not limited to, methyl, ethyl and propyl. A C₁₋₂ alkyl group is methyl or ethyl.

The term “C₁₋₃ alkylene” refers to a bivalent C₁₋₃ alkyl group.

The term “C₁₋₄haloalkyl” refers to a C₁₋₄ alkyl group, as defined herein, which is substituted with one or more halogen. Examples of C₁₋₄ haloalkyl groups include, but are not limited to, trifluoromethyl, difluoromethyl and pentafluoroethyl.

The term “C₁₋₄ alkoxy” refers to a straight, or branched chain saturated hydrocarbon containing 1 to 4 carbon atoms containing a terminal “O” in the chain, i.e., —O(alkyl). Examples of C₁₋₄ alkoxy groups include, but are not limited to, methoxy, ethoxy, propoxy and butoxy.

The term “C₂₋₆ alkenyl” refers to a straight or branched chain hydrocarbon containing 2 to 6 carbon atoms and at least one double bond.

The term “C₂₋₆ alkynyl” refers to a straight or branched chain hydrocarbon containing 2 to 6 carbon atoms and at least one triple bond.

The term “C₃₋₆ cycloalkyl” refers to a monocyclic saturated carbon ring containing between 3 and 6 carbon atoms. Specifically, it refers to cyclopropyl, cyclobutyl, cyclopentyl or cyclohexyl.

The term “heteroaryl” refers to a monocyclic aromatic ring containing one or more heteroatoms, preferably selected from: N, S and O. Examples of 5-membered heteroaryls include, but are not limited to, pyrazole, triazole and thiazole. Examples of 6-membered heteroaryls include, but are not limited to, pyridine and pyridazine.

The term “bicyclic heteroaryl” refers to a bicyclic aromatic ring containing one or more heteroatoms, preferably selected from: N, S and O. Examples of 9-membered bicyclic heteroaryls include, but are not limited to, indole, isoindole, indazole and pyrazolopyridine. Examples of 10-membered bicyclic heteroaryls include, but are not limited to, quinoline and chromene.

The term “5- or 6-heterocyclyl” refers to a 5 or 6 membered monocyclic saturated ring containing one or more heteroatoms, for example, pyrrolidine and piperidine.

As used herein, the term “oligomer” means compounds that have at least two of the pyrrolidine moieties set out in formula I, I′, II, IV, IV′ or V. As used herein, “pyrrolidine moiety” refers to an optionally substituted pyrrolidine. The pyrrolidine moieties in an oligomer may be the same or different.

As used herein, the term “elevated Lp(a) plasma levels” means a plasma level of Lp(a) that is equal to or above about 50 mg/dL. A compound or oligomer provided herein may be used in treatment to reduce Lp(a) plasma levels.

The term “pharmaceutically acceptable salt” as used herein refers a salt of a compound that is acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19. In particular, the compounds of Formulae I, I′, IV and IV′ may be a zwitterion, a mono-, di, or tri-acid addition salt.

The pharmaceutical compositions for the present invention may be prepared using pharmaceutically acceptable additives. The term “pharmaceutically acceptable” refers to one or more carriers, diluents, and/or excipients that are compatible with the other components of the composition and not pharmaceutically deleterious to the patient.

Examples of pharmaceutical compositions and processes for their preparation are well known to the skilled artisan, and can be found, for example, in “Remington: The Science and Practice of Pharmacy”, Loyd, V., et al. Eds., 22nd Ed., Mack Publishing Co., 2012.

As used herein, the term “effective amount” refers to a dosage amount that is effective in treating a disorder. The effective amount for a particular patient can be determined by a skilled health professional.

As used herein, the terms “treating”, “to treat”, or “treatment”, includes slowing, reducing, preventing, or reversing the progression or severity of an existing symptom, disorder, condition, or disease. As used herein, “treating cardiovascular disease” means slowing, reducing, preventing, or reversing the progression of heart or blood vessel disease.

As used herein, the term “patient” refers to a mammal. Preferably, the patient is a human.

Pharmaceutical compositions can be formulated as a tablet or capsule for oral administration, a solution for oral administration, or an injectable solution. In an embodiment the composition is suitable for oral administration.

Certain abbreviations are defined as follows: “ACN” refers to acetonitrile; “Apo” refers to Apolipoprotein; “BOC” refers to tert-butoxycarbonyl; “Bn” refers to benzyl; “BSA” refers to Bovine Serum Albumin; “CDI” refers to carbonyldiimidazole; “DAD” refers to diode array detector; “DCM” refers to dichloromethane or methylene chloride; “de” refers to diasteriomeric excess; “DMA” refers to N,N-dimethylacetamide; “DMAP” refers to 4-dimethylaminopyridine; “DMEA” refers to dimethylethylamine; “DMEM” refers to Dulbecco's Modified Eagle's Medium; “DMF” refers to N,N-dimethylformamide; “DMSO” refers to dimethyl sulfoxide; “ee” refers to enantiomeric excess; “EACA” refers to epsilon-aminocaproic acid or 6-aminocaproic acid; “ELISA” refers to enzyme-linked immunosorbent assay; “equiv” refers to equivalents; “Et₂O” refers to diethyl ether; “EtOAc” refers to ethyl acetate; “EtOH” refers to ethanol or ethyl alcohol; “Ex” refers to example; “FBS” refers to Fetal Bovine Serum; “h” refers to hour or hours; “HATU” refers to 1-[bis(dimethylamino)methylene]-1H-1,2,3-triazolo[4,5-b]pyridinium 3-oxid hexafluorophosphate; “HEC” refers to hydroxy ethyl cellulose; “HEK” refers to human embryonic kidney; “HepG2” refers to a human hepatoma cell line; “HEPES” refers to 4-(2-hydroxyethyl)-1-piperazineethanesulfonic acid; “HLB” refers to hydrophilic lipophilic balance; “HPLC” refers to high-performance liquid chromatography; “IHRP” refers to Horseradish Peroxidase; “IC₅₀” refers to the concentration of an agent that produces 50% of the maximal inhibitory response possible for that agent; “IPA” refers to isopropanol; “min” refers to minute or minutes; “MeOH” refers to methanol or methyl alcohol; “MTBE” refers to methyl tert-butyl ether; “RP-HPLC/MS” refers to reverse-phase high performance liquid chromatography with mass spectrometry; “RT” refers to room temperature; “SFC” refers to supercritical fluid chromatography; “SPA” refers to scintillation proximity assay; “t_{(R)}” refers to retention time; “TEA” refers to triethylamine; “THF” refers to tetrahydrofuran; “TMB” refers to 3,3′,5,5′-teramethylbenzidine and “Tris” refers to tris(hydroxymethyl)aminomethane.

Individual isomers, enantiomers, and diastereomers may be separated or resolved by one of ordinary skill in the art at any convenient point in the synthesis of compounds listed below, by methods known to the artisan, such as selective crystallization techniques or chiral chromatography.

A compound of formula I, I′, II, III, IV, IV′ or V, or any depicted formulae is readily converted to and may be isolated as a pharmaceutically acceptable salt. Salt formation can occur upon the addition of a pharmaceutically acceptable acid to form the acid addition salt or by the addition of a pharmaceutically acceptable base to form a base addition salt. Salts can also form simultaneously upon deprotection of a nitrogen or oxygen, i.e., removing the protecting group. Examples, reactions and conditions for salt formation are known to the skilled artisan.

The compounds of formula I, I′, II, III, IV, IV′ or V or any depicted formulae, or salts thereof, may be prepared by a variety of procedures, some of which are illustrated in the Preparations and Examples below. The specific synthetic steps for each of the routes described may be combined in different ways, or in conjunction with steps from different routes, to prepare compounds or salts of the present invention. The products of each step in the Preparations below can be recovered by conventional methods, including extraction, evaporation, precipitation, chromatography, filtration, trituration, and crystallization.

In the schemes below, all substituents unless otherwise indicated, are as previously defined. The reagents and starting materials are readily available to one of ordinary skill in the art. Without limiting the scope of the invention, the following schemes, preparations, and examples are provided to further illustrate the invention. Compounds of afore-depicted formulae, or salts thereof may be prepared by using starting materials or intermediates with the corresponding desired stereochemical configuration.

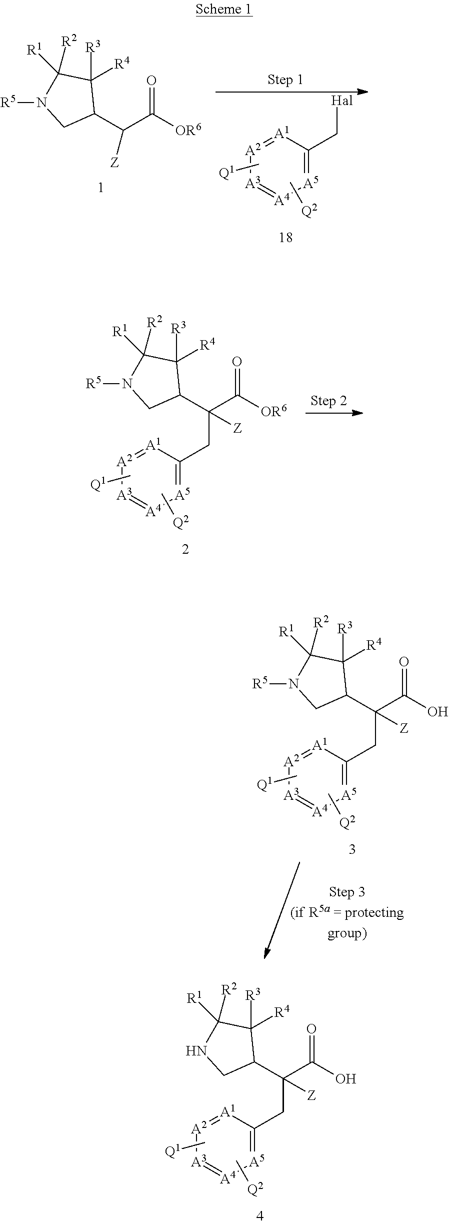

Scheme 1 depicts the preparation of compounds of the present invention starting with ester intermediate 1, which is treated with a strong organic base such as lithium diisopropylamide or potassium bis(trimethylsilyl)amide at −78° C. followed by the addition of halide 18 to give 2 in Step 1. The ester can be hydrolyzed in Step 2 using a strong inorganic base such as lithium or sodium hydroxide to give 3, and if R^{5a} is a protecting group it can be removed in Step 3 to give the acid 4 (for example, if R^{5a}=Boc it can be removed under acidic conditions).

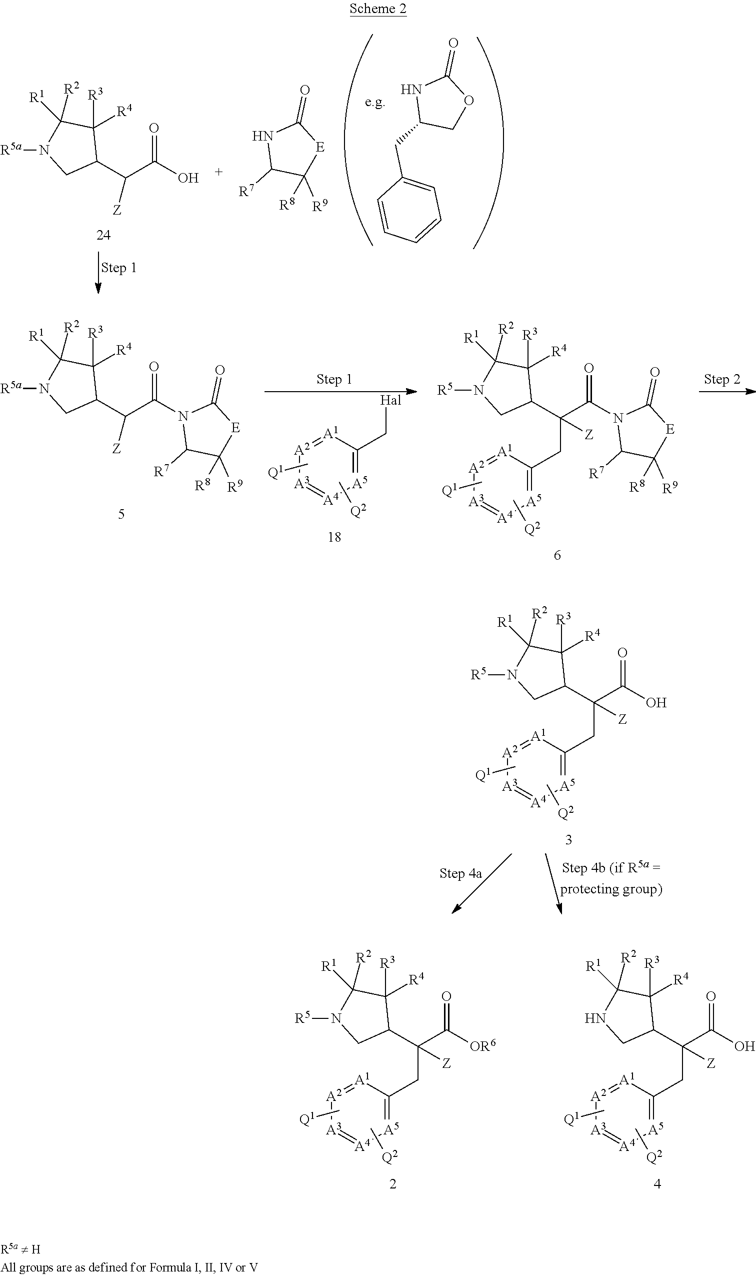

Scheme 2 depicts preparation of compounds of the present invention using a chiral auxiliary to direct the stereoselectivity of the alkylation of intermediate 5, which is prepared from acid 24 via the acid chloride and heterocycle 25 at 0° C. in Step 1. In Step 2, intermediate 5 is treated with a strong organic base such as lithium bis(trimethylsilyl)amide at a cold temperature (−78-0° C.) and then halide 18 is added to give 6. In Step 3, the chiral auxiliary is hydrolyzed using aqueous H₂O₂ and LiOH at 0° C. followed by quenching the reaction with NaHSO₃ to give acid 3. Optionally, 3 is esterified in Step 4a to give 2, or if R^{5a} is a protecting group it can be removed to give 4 (Step 4b).

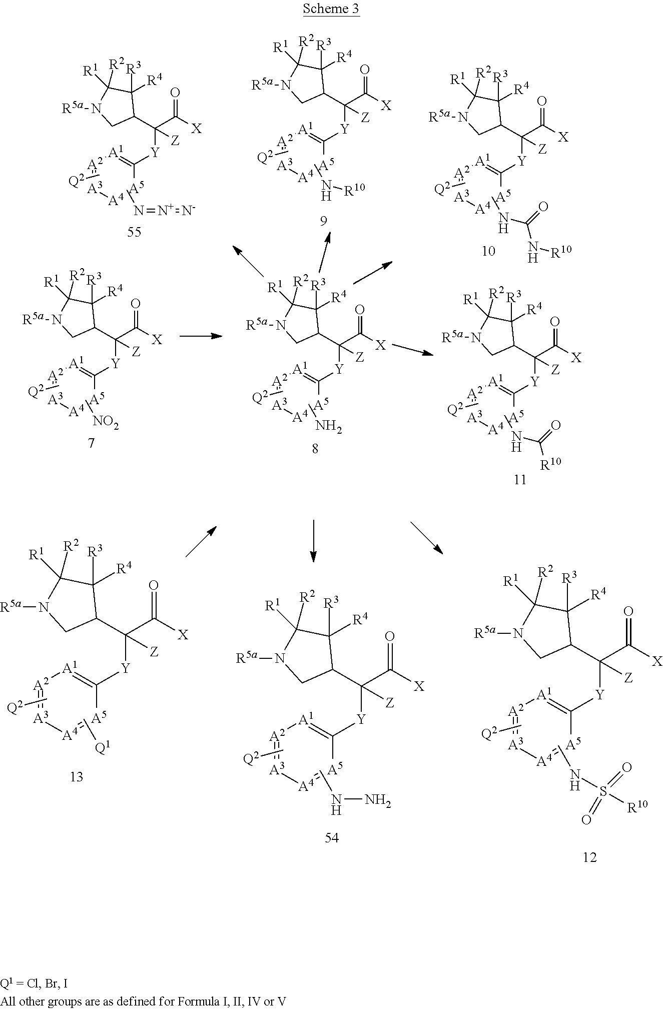

Scheme 3 shows functional groups which can be prepared from amino compound 8, which can be prepared either by reduction of nitro compound 7 with hydrogen gas using a palladium on carbon, or by coupling 13 with ammonium hydroxide using a copper catalyst such as copper (II) acetylacetonate, 2,2,6,6-tetramethyl-3,5-heptanedione, and a carbonate base at elevated temperature. Amino compound 8 can then be reacted with alkyl halides and a carbonate base to give 9. Additionally, 9 is accessible by reductive amination on compound 8 (e.g. using an aldehyde and a reducing agent such as sodium triacetoxyborohydride or sodium cyanoborohydride). Urea compound 10 can be prepared by treating 8 with potassium cyanate (giving 10 where R¹⁰=H), or 8 can be treated with 1,1′-carbonyldiimidazole followed by an amine H₂NR¹⁰. Amide compound 11 can be prepared by reacting 8 with an acid chloride (R¹⁰C(O)Cl) and an organic base such as triethylamine, or by reacting 8 with a carboxylic acid (R¹⁰CO₂H) under amide coupling conditions (e.g. using HATU and an organic base). Reacting compound 8 with a sulfonyl chloride (R¹⁰SO₂Cl) and an organic base gives sulfonamide 12. Oxidizing aniline 8 with sodium nitrite and HCl followed by reduction with stannous chloride gives hydrazine compound 54. Reacting 8 with tert-butyl nitrite and azidotrimethylsilane gives azide 55.

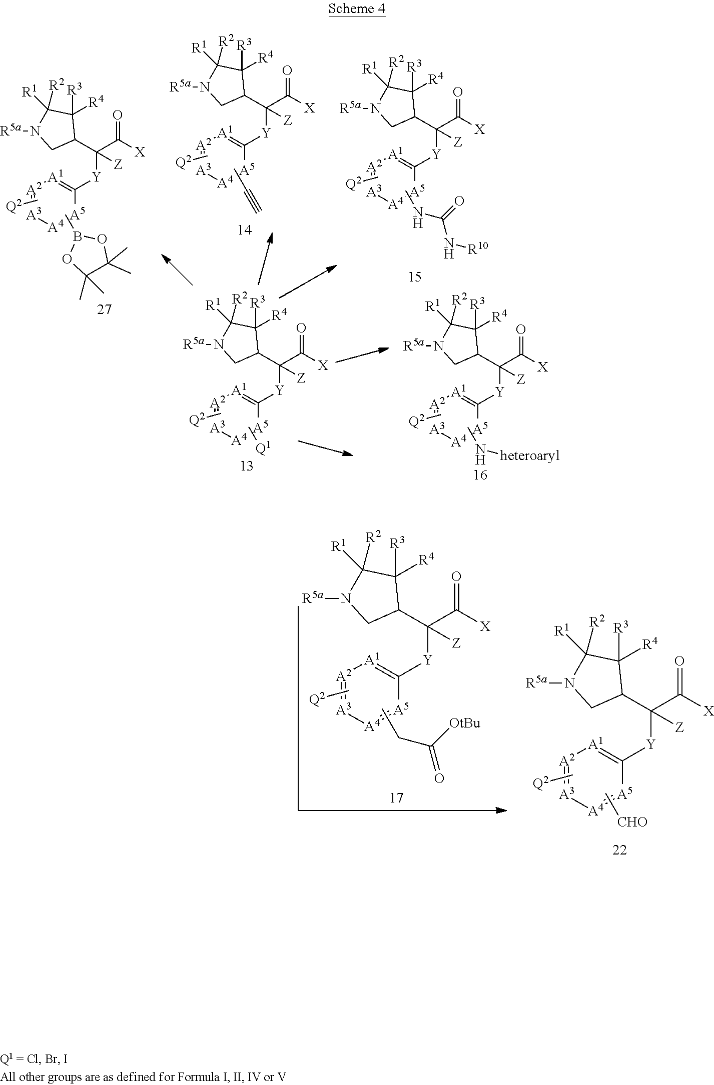

When Q¹ is a group capable of undergoing metal-halogen exchange (e.g. Cl, Br, I, or —OSO₂CF₃— compound 13), a number of derivatives are accessible through a variety of functional group transformations. Sonogashira coupling with an alkyne, palladium catalyst, and an organic base at elevated temperature gives aryl alkynes, e.g. 14 (product of coupling with ethynyl(trimethyl)silane followed by removal of the trimethylsilyl group under basic conditions). Ureas and amino heterocycles can be coupled onto the aryl ring using a palladium catalyst and sodium tert-butoxide to give 15 and 16, respectively, when Q¹ of 13 is bromine. Negishi coupling on 13 is accomplished using an organozinc compound and palladium catalyst at elevated temperature, e.g. with (2-tert-butoxy-2-oxoethyl)(chloro)zinc to give 17. Aldehyde 22 can be prepared from 13 via catalytic carbonylation, and boronate 27 can be prepared by coupling 13 with e.g. bis(pinacolao)diboron, potassium acetate, and a palladium catalyst at elevated temperature.

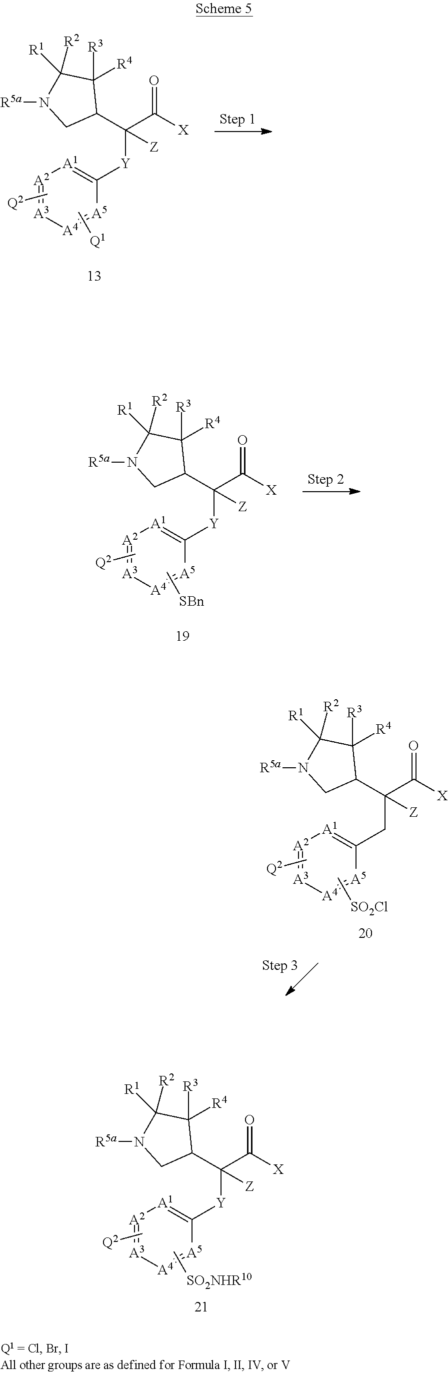

Compound 13 can also be used to prepare sulfonamides 21 as shown in Scheme 5. In Step 1, compound 13 is coupled with benzyl mercaptan using a palladium catalyst and organic base at elevated temperature to give thioether 19, which is converted in Step 2 to sulfonyl chloride 20 using 1,3-dichloro-5,5-dimethylhydantoin at 0° C. in a mixture of ACN, water, and acetic acid. Sulfonamide 21 is then prepared using an amine (H₂NR¹⁰) and an organic base.

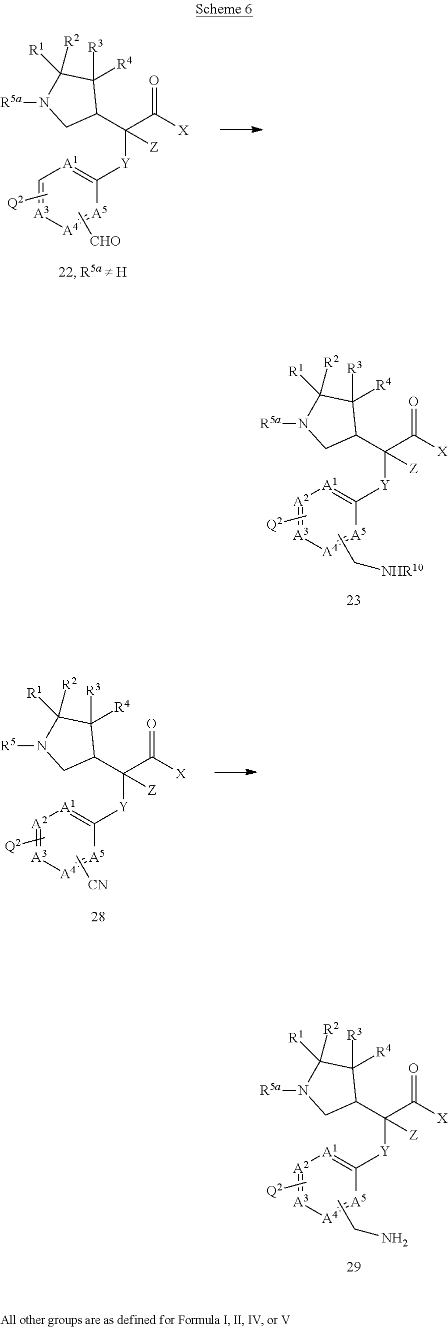

All other groups are as defined for Formula I, II, IV, or V Scheme 6 shows the preparation of amine 23 from aldehyde 22, which is accomplished by reductive amination with an amine (H₂NR¹⁰) and a reducing agent such as sodium triacetoxyborohydride. Amine 29 can be prepared from nitrile 28 by hydrogenation in the presence of a palladium on carbon catalyst.

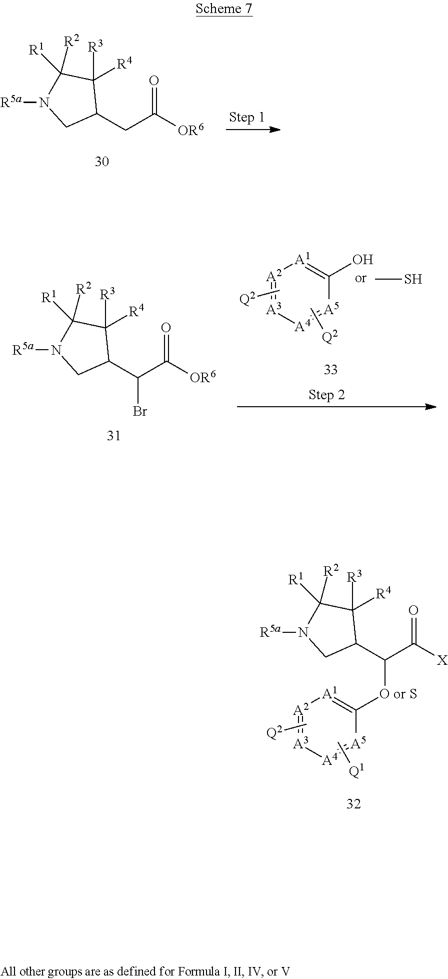

Scheme 7 shows the preparation of compound 32, which can be either an ether or a thioether. In Step 1, intermediate 30 is brominated via the silyl enol ether (prepared by treating intermediate 30 with lithium diisopropylamide at −78° C. and adding chlorotrimethylsilane) and N-bromosuccinimide to give bromide 31. In Step 2, bromide 31 is reacted with 33 and a base (carbonate base if 33 is a phenol, or sodium methoxide if 33 is a thiophenol).

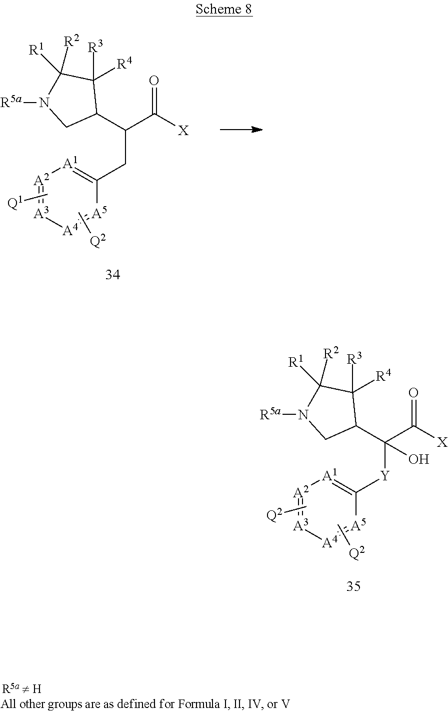

Scheme 8 shows the hydroxylation of intermediate 34. Intermediate 34 is first treated with lithium diisopropylamide at −78° C. and then with 3-phenyl-2-(phenylsulfonyl)-1,2-oxaziridine to give hydroxy compound 35.

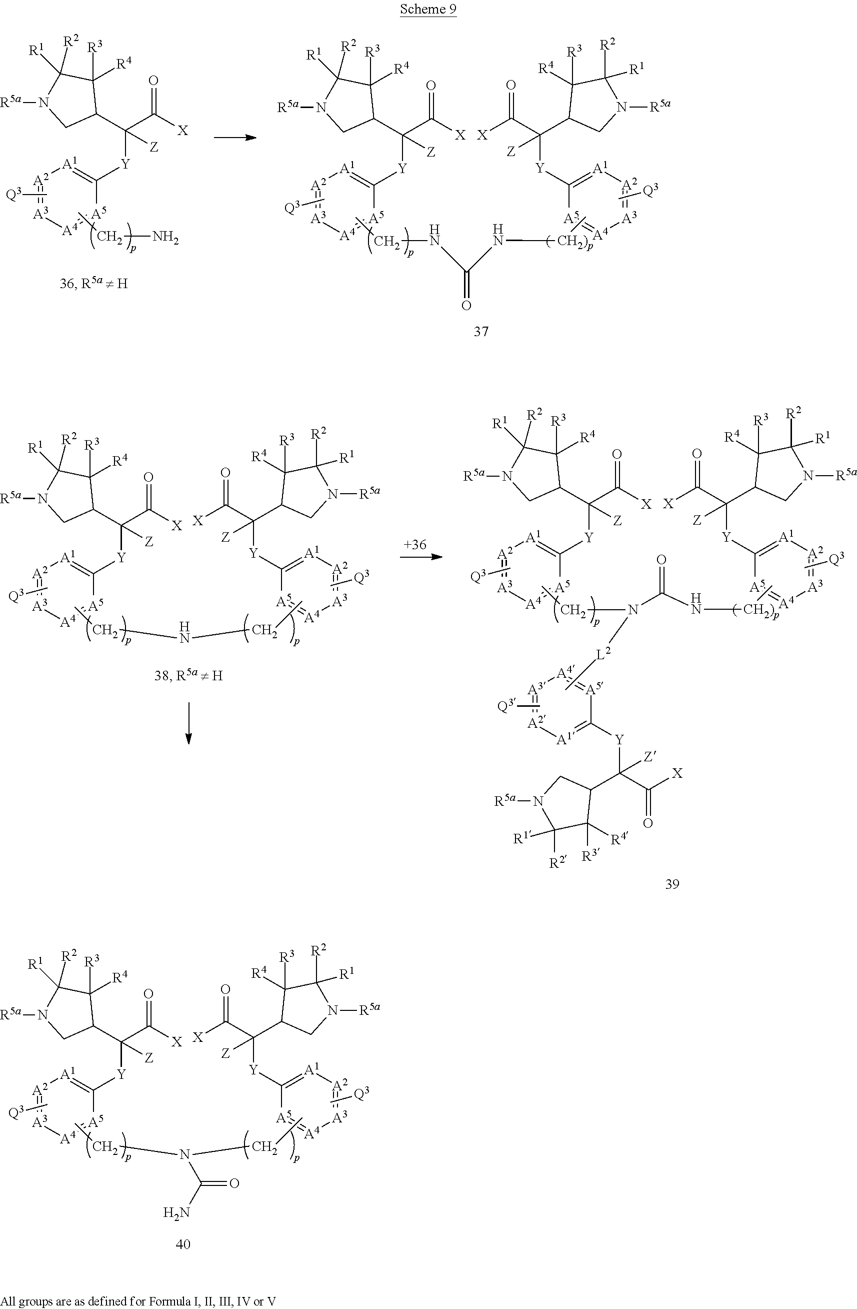

Scheme 9 shows the preparation of multimeric urea compounds 37, 39, and 40. Compound 37 can be prepared by reacting amino compound 36 with CDI. Disubstituted amino compound 38 [prepared by reductive amination of 36 with aldehyde 22 (Scheme 4) or, if p=0, Buchwald coupling of aniline intermediate 8 (Scheme 3) with intermediate 13 (Scheme 4)] is reacted with amino compound 36 and CDI to give trisubstituted urea 39. Amine 38 can also be reacted with sodium cyanate followed by 1-2 equivalents of TFA to give urea 40.

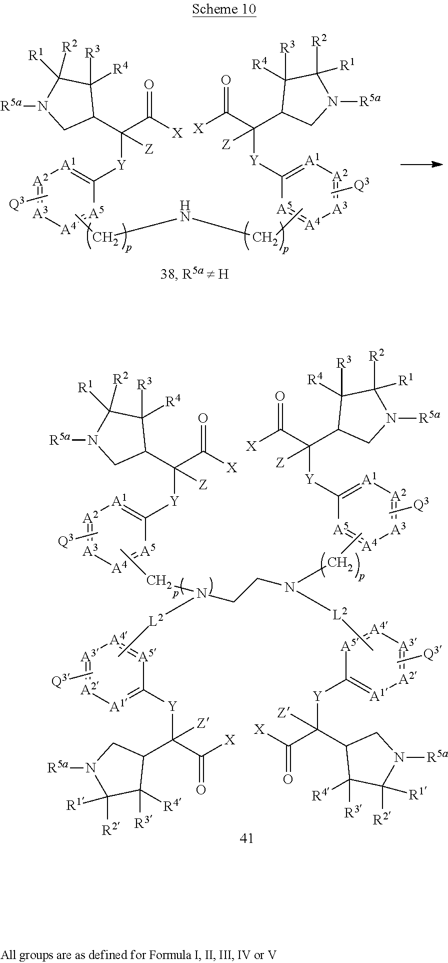

Scheme 10 shows the preparation of tetrameric compound 41, in which amine 38 undergoes reductive amination with oxaldehyde and a reducing agent such as sodium triacetoxyborohydride.

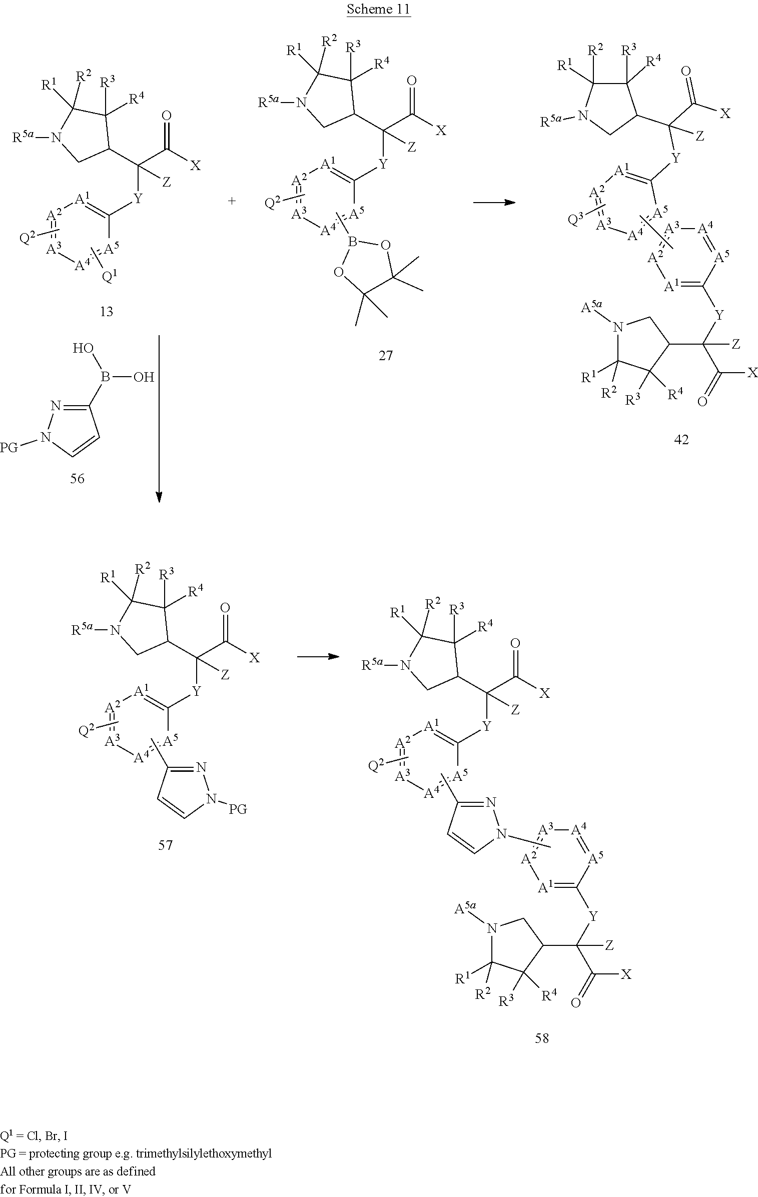

Scheme 11 shows the preparation of dimeric compound 42, wherein intermediate 13 is coupled with boronate 27 using a palladium catalyst and a carbonate base at elevated temperature. Intermediate 13 can also be coupled with a protected pyrazole boronic acid (56) using a palladium catalyst and a carbonate base at elevated temperature to give intermediate 57, which can be deprotected and coupled with another equivalent of intermediate 13 using copper(IJ) acetate and pyridine as solvent at elevated temperature to give 58.

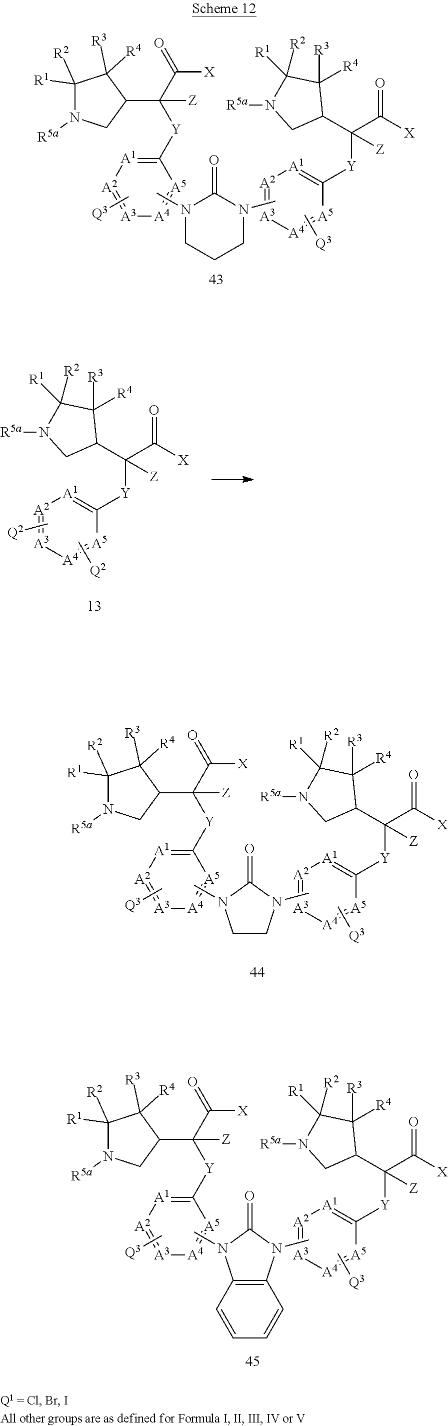

Scheme 12 shows the preparation of cyclic urea compounds 43, 44, and 45. Intermediate 13 is coupled using a palladium catalyst and sodium tert-butoxide at elevated temperature with either tetrahydro-2(1H)-pyrimidinone to give 43, or imidazolidine-2-one to give 44. Intermediate 13 is also coupled with 1,3-dihydrobenzimidazol-2-one using cuprous iodide, N,N′-dimethylethylenediamine, and a carbonate base at elevated temperature to give 45.

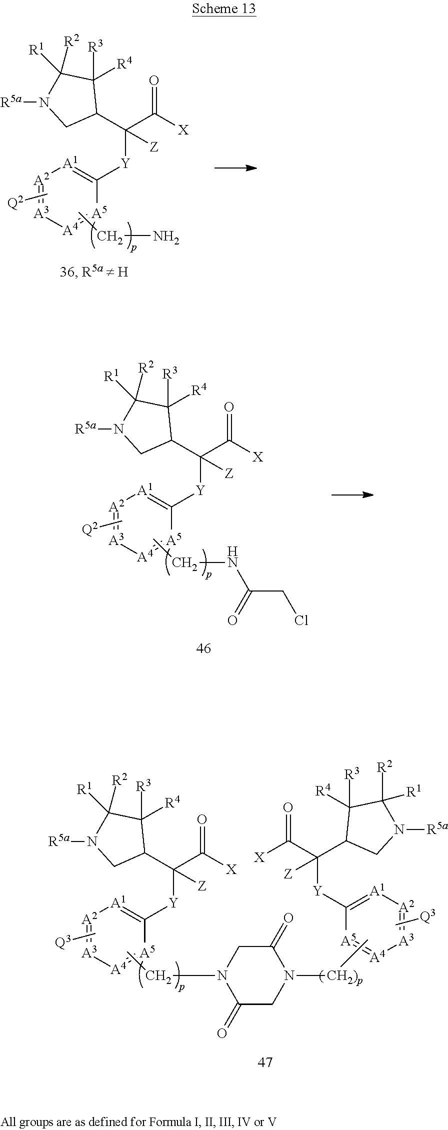

Scheme 13 shows the preparation of compound 47. Intermediate 36 is reacted with chloroacetyl chloride and an organic base to give chloroacetyl intermediate 46, which is then dimerized using a carbonate base at elevated temperature to give dioxopiperazine compound 47.

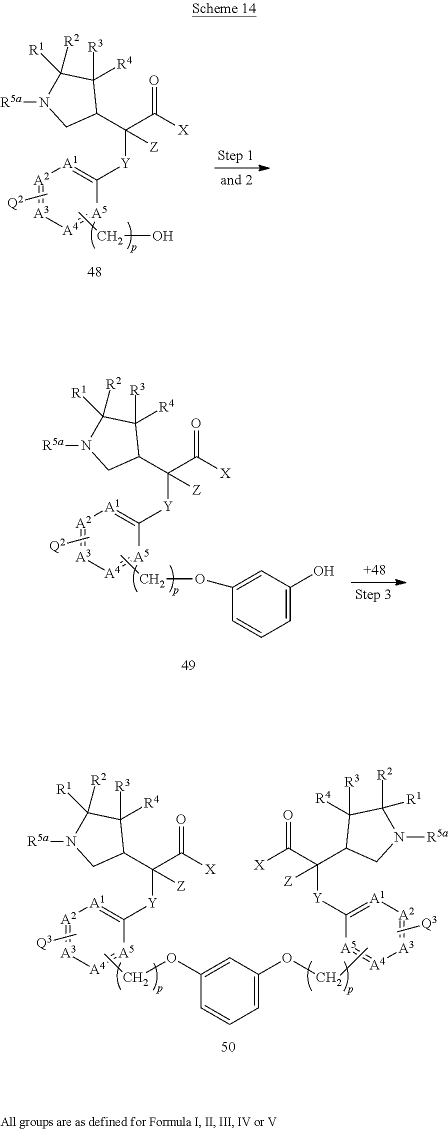

Scheme 14 shows the preparation of compound 50. Alcohol compound 48 first undergoes a Mitsunobu reaction with (3-hydroxyphenyl)acetate, diethyl azodicarboxylate, and triphenylphosphine, and the acetyl group is removed using a carbonate base in MeOH to give intermediate 49. Mitsunobu reaction again with intermediate 48 and phenol 49 gives intermediate 50.

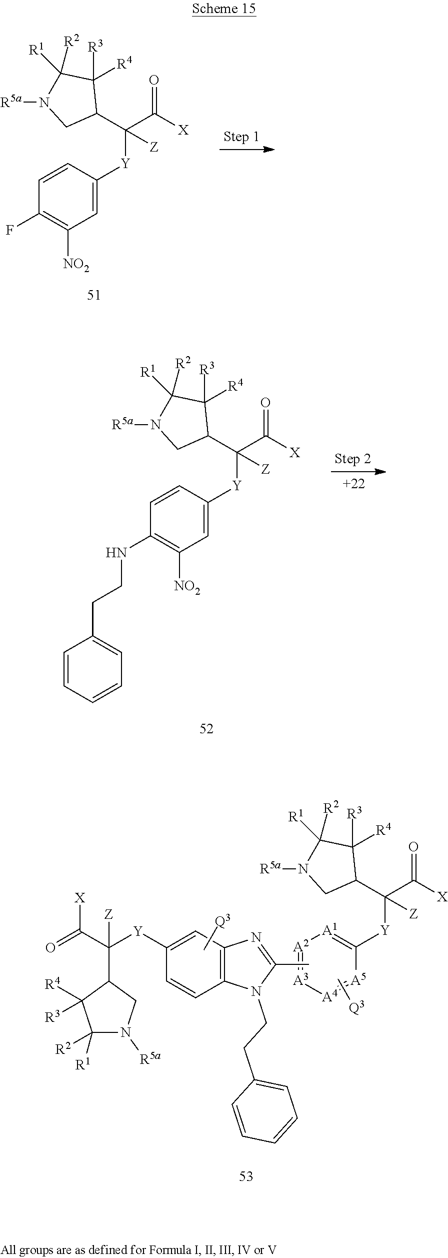

Scheme 15 shows the preparation of compound 53 via a one-pot procedure from intermediates 51 and 22. In Step 1, fluoro-nitro compound 51 undergoes a S_{N}Ar reaction with 2-phenylethanamine at elevated temperature to give intermediate 52. Na₂S₂O₄ is then added to the reaction followed by aldehyde intermediate 22 (See Scheme 4) and heating is continued to give cyclic compound 53.

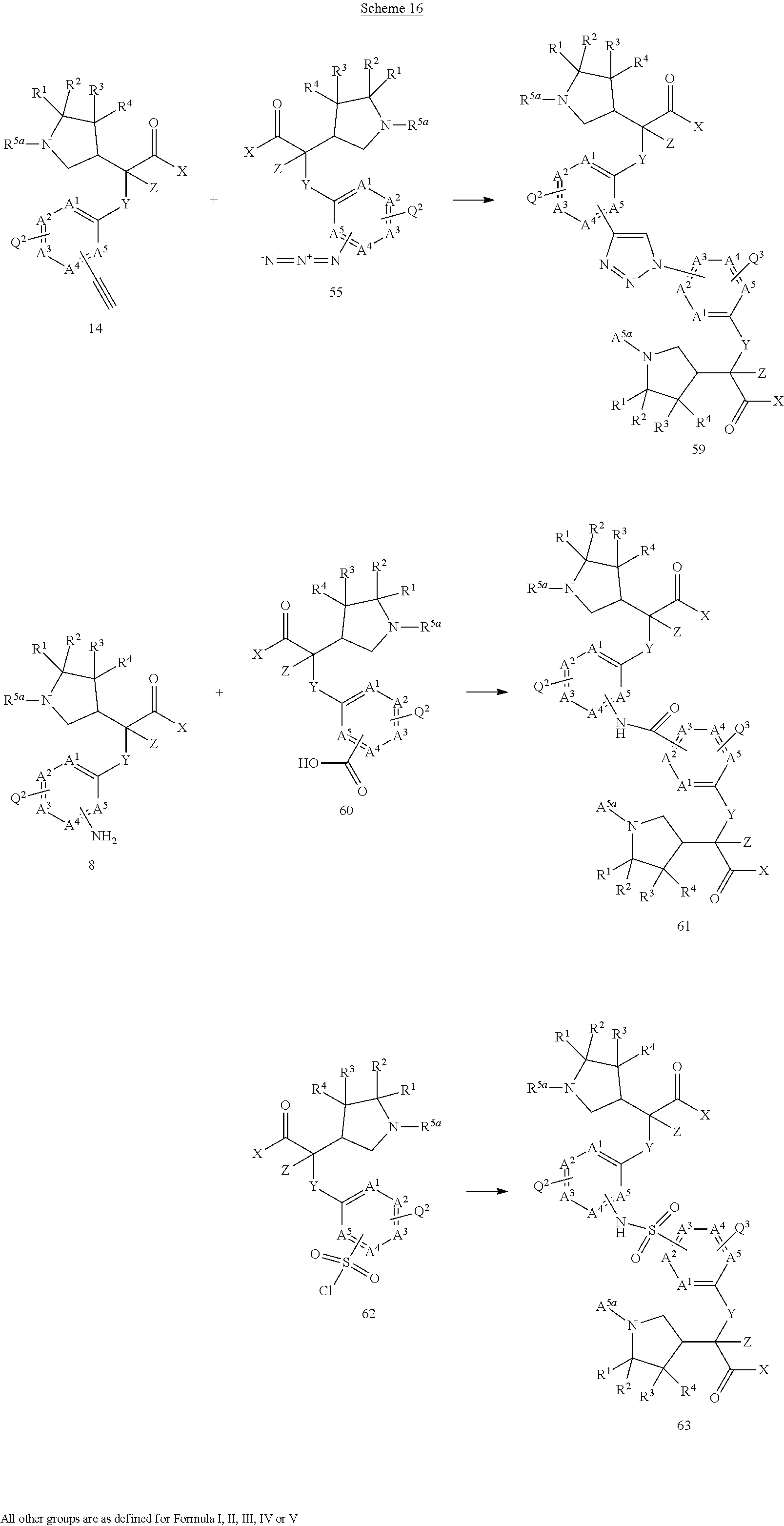

Scheme 16 shows the preparation of compound 59 (prepared by click chemistry in which alkyne 14 and azide 55 are cyclized to the 1,2,3-triazole using cupric sulfate, sodium ascorbate, and benzoic acid in a mixture of tert-butanol and water), compound 61 (prepared by coupling of aniline 8 with acid 60 under amide coupling conditions, e.g. with HATU in the presence of an organic base), and compound 63 (prepared by reacting aniline 8 with sulfonyl chloride 62 in the presence of an organic base).

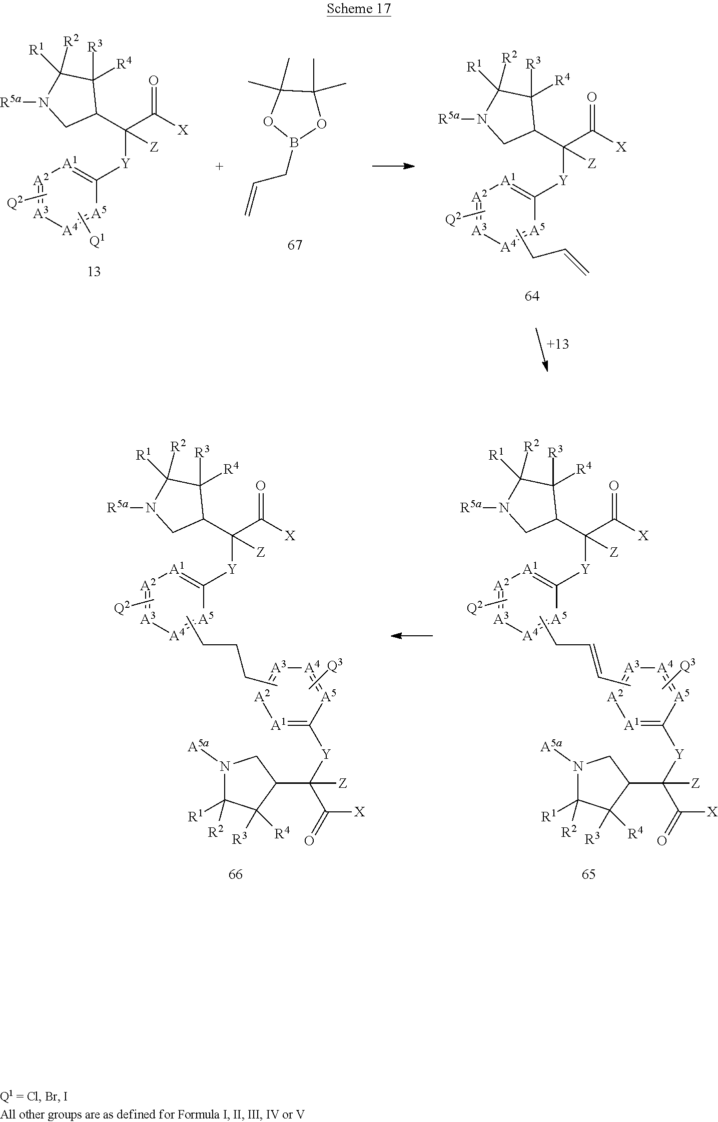

Scheme 17 shows the preparation of propyl-linked dimeric compound 66. Aryl halide 13 is first coupled with boronate 67 using a palladium catalyst and a carbonate base at elevated temperature to give compound 64, which then undergoes a Heck coupling with another equivalent of halide 13 using a palladium catalyst and an organic base at elevated temperature to give alkene 65. Reduction of the alkene under hydrogen gas using a palladium on carbon catalyst then gives 66.

### Preparation 1 tert-Butyl (3R)-3-[2-methoxy-2-oxo-1-[[3-(2-trimethylsilylethynyl)phenyl]methyl]ethyl]pyrrolidine-1-carboxylate

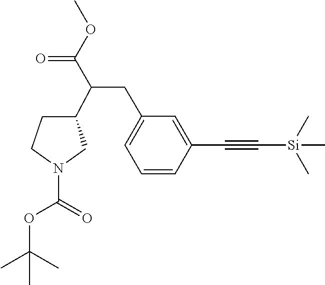 [SMILES: COC(=O)C(Cc1cccc(C#C[Si](C)(C)C)c1)[C@H]1CCN(C(=O)OC(C)(C)C)C1]

To a mixture of tert-butyl (3R)-3-[1-[(3-bromophenyl)methyl]-2-methoxy-2-oxo-ethyl]pyrrolidine-1-carboxylate (prepared essentially as described in WO 2020/247429, 1.95 g, 4.73 mmol), cuprous iodide (0.90 g, 0.47 mmol), 1,3-bis(diphenylphosphino)propane (390 mg, 0.94 mmol) and tris(dibenzylideneacetone)dipalladium(0) (433 mg, 0.47 mmol) in TEA (39 mL) was added ethynyl(trimethyl)silane (2.67 mL, 1.86 g, 18.9 mmol). The resulting mixture was stirred at 120° C. overnight. The mixture was cooled to RT, filtered through Celite® and washed with EtOAc. The solvent was removed in vacuo to give 4.2 g of a brown oil, which was then purified via silica gel chromatography using a gradient of 10 to 40% MTBE in hexanes to give the title compound (1.80 g, 84%) as a brown oil. ES-MS m/z 374 (M-tBu+H)

### Preparation 2

### 2-[(3R)-1-tert-Butoxycarbonylpyrrolidin-3-yl]-3-(3-ethynylphenyl)propanoic acid—Isomer 1 and Isomer 2

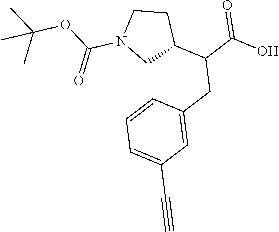 [SMILES: C#Cc1cccc(CC(C(=O)O)[C@H]2CCN(C(=O)OC(C)(C)C)C2)c1]

Aqueous NaOH (2 M, 21 mL, 42 mmol) was added to a solution of tert-butyl (3R)-3-[2-methoxy-2-oxo-1-[[3-(2-trimethylsilylethynyl)phenyl]methyl]ethyl]pyrrolidine-1-carboxylate (1.8 g, 4.2 mmol) in THE (21 mL) and MeOH (10 mL). The mixture was stirred at RT 3 days, then EtOAc was added, and then aqueous HCl (1M) was added until pH=3. The aqueous phase was extracted with EtOAc and the organic layer was dried over Na₂SO₄. The solvent was removed under reduced pressure then the residue was loaded onto a HLB column. The column was eluted with water, then ACN, and then the ACN fraction was concentrated to give a brown oil containing the title compound as a mixture of diastereomers.

The diastereomers were separated by SFC [column: 20×250 mm, SFC 2-ethylpyridine stationary phase, 5 μm; mobile phase: 10% (10 mM NH₄HCO₃ in MeOH, pH 8) in CO₂] to give Isomer 1 (610 mg, 42%, first-eluting isomer) as a yellow solid and Isomer 2 (454 mg, 27%, second-eluting isomer) as a pale brown solid. Both Isomer 1 and Isomer 2: ES-MS m/z 288 (M-tBu+H).

### Preparation 3

### tert-Butyl (3R)-3-[1-[(6-chloro-3-pyridyl)methyl]-2-methoxy-2-oxo-ethyl]pyrrolidine-1-carboxylate

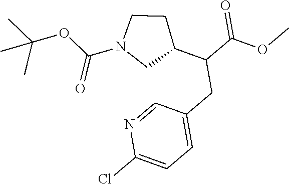 [SMILES: COC(=O)C(Cc1ccc(Cl)nc1)[C@H]1CCN(C(=O)OC(C)(C)C)C1]

Lithium diisopropylamide (2.0 M in THF/heptane/ethylbenzene, 0.49 mL, 0.99 mmol) was added dropwise under N₂ to a −78° C. solution of tert-butyl (3R)-3-(2-methoxy-2-oxo-ethyl)pyrrolidine-1-carboxylate (prepared essentially as described in WO 2020/247429, 200 mg, 0.82 mmol) in THE (4 mL) and the reaction was stirred at −78° C. for 1 h. A solution of 5-(bromomethyl)-2-chloropyridine (357 mg, 1.64 mmol) in THE (2 mL) was then added and the reaction was stirred at −78° C. for 30 min. The reaction was quenched with saturated aqueous NH₄Cl and extracted with EtOAc. The organic layer was separated, dried over MgSO₄ and concentrated in vacuo. The residue was purified by silica gel chromatography using a gradient of 25 to 100% EtOAc in hexanes to give the title compound (mixture of diastereomers, 256 mg, 84%) as a colorless oil. ES-MS m/z 313 (M-tBu+H).

### Preparation 4

### 2-[(3R)-1-tert-butoxycarbonylpyrrolidin-3-yl]-3-(6-chloro-3-pyridyl)propanoic acid—Isomer1 and Isomer 2

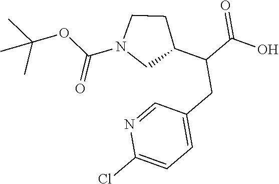 [SMILES: CC(C)(C)OC(=O)N1CC[C@H](C(Cc2ccc(Cl)nc2)C(=O)O)C1]

To a solution of tert-butyl (3R)-3-[1-[(6-chloro-3-pyridyl)methyl]-2-methoxy-2-oxo-ethyl]pyrrolidine-1-carboxylate (256 mg, 0.69 mmol) in THE (4 mL) was added LiOH (1 M in water, 4 mL, 4 mmol). The reaction was stirred at RT overnight, then at 50° C. for 1 h. The reaction was quenched with 1 N HCl and extracted with EtOAc. The organic layer was separated, dried over MgSO₄ and concentrated in vacuo. The residue was purified via silica gel chromatography using 10% MeOH in DCM, and then the diastereomers were separated by chiral SFC [column: Chiralpak AD 20×250 mm, 5 μm; mobile phase: 25% (MeOH+0.2% DMEA) in CO₂, 65 mL/min] to give Isomer 1 (107 mg, 43%, first-eluting isomer) and Isomer 2 (70 mg, 28%, second-eluting isomer) of the title compound. Both Isomer 1 and Isomer 2: ES-MS m/z 299 (M-tBu+H).

### Preparation 5

### (2S)-3-(3-Bromo-5-methyl-phenyl)-2-[(3R)-1-tert-butoxycarbonylpyrrolidin-3-yl]propanoic acid

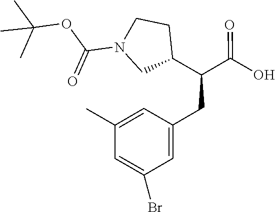 [SMILES: Cc1cc(Br)cc(C[C@H](C(=O)O)[C@H]2CCN(C(=O)OC(C)(C)C)C2)c1]

Lithium bis(trimethylsilyl)amide (1.0 M in THF, 6.8 mL, 6.8 mmol) was added to a solution of tert-butyl (3R)-3-[2-[(4S)-4-benzyl-2-oxo-oxazolidin-3-yl]-2-oxo-ethyl]pyrrolidine-1-carboxylate (prepared essentially as described in WO 2020/247429, 2.2 g, 5.7 mmol) in THE (40 mL) at 0° C. The mixture was stirred for 30 min, then a solution of 1-bromo-3-(bromomethyl)-5-methyl-benzene (1.6 g, 6.2 mmol) in THE (6.6 mL) was added dropwise. The resulting mixture was allowed to reach RT and stirred overnight. The reaction was quenched with saturated aqueous NH₄Cl and extracted with EtOAc. The organic phases were combined and washed with saturated aqueous NaCl, dried over Na₂SO₄, filtered and concentrated to dryness. To the residue was added THF (39 mL) and water (7.4 mL), and the mixture was cooled to 0° C. Aqueous hydrogen peroxide (3.1 M, 4.8 mL, 57 mmol) was added followed by a solution of LiOH (0.24 g, mmol) in water (11 mL). The reaction was stirred at 0° C. for 50 min, then the reaction was quenched by adding NaHSO₃ and the mixture was warmed to RT. The mixture was extracted with EtOAc, then the organics were washed with saturated aqueous NaCl, dried over Na₂SO₄, filtered and evaporated to dryness. The residue was purified via silica gel chromatography using a gradient of 10 to 40% EtOAc in hexanes+1% acetic acid to give the title compound (1.4 g, 45%) as a white solid. ES-MS m/z 355,357 (M-tBu+H).

### Preparation 6

### (2S)-3-(3-Amino-5-methyl-phenyl)-2-[(3R)-1-tert-butoxycarbonylpyrrolidin-3-yl]propanoic acid

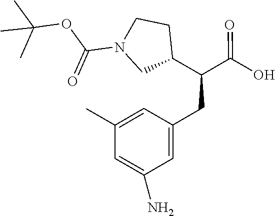 [SMILES: Cc1cc(N)cc(C[C@H](C(=O)O)[C@H]2CCN(C(=O)OC(C)(C)C)C2)c1]

To a mixture of (2S)-3-(3-bromo-5-methyl-phenyl)-2-[(3R)-1-tert-butoxycarbonylpyrrolidin-3-yl]propanoic acid (0.37 g, 0.68 mmol), sodium azide (0.088 g, 1.4 mmol) and L-proline (0.10 g, 0.88 mmol) in DMSO (3.4 mL) was added Cu₂O (97 mg, 0.68 mmol). The mixture was purged with nitrogen and heated at 100° C. overnight. The mixture was cooled to RT, saturated aqueous NH₄Cl was added and the mixture was extracted with EtOAc. The organic layer was dried over Na₂SO₄, filtered and concentrated under reduced pressure. The residue was purified via reverse phase chromatography using a gradient of 20 to 50% ACN in water to give the title compound (0.11 g, 46%) as a pale yellow solid. ES-MS m/z 249 (M-Boc+H)

### Preparation 7

### (2S)-2-[(3R)-1-tert-butoxycarbonylpyrrolidin-3-yl]-3-(3-methyl-5-ureido-phenyl)propanoic acid

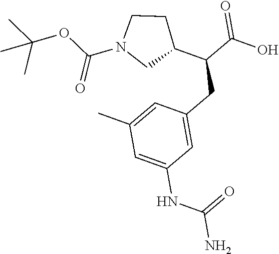 [SMILES: Cc1cc(C[C@H](C(=O)O)[C@H]2CCN(C(=O)OC(C)(C)C)C2)cc(NC(N)=O)c1]

To a solution of (2S)-3-(3-amino-5-methyl-phenyl)-2-[(3R)-1-tert-butoxycarbonylpyrrolidin-3-yl]propanoic acid (90 mg, 0.26 mmol) in acetic acid (2.1 mL) was added potassium cyanate (0.105 g, 1.29 mmol) in water (1.7 mL). The mixture was stirred at RT for 30 min and then concentrated. The reaction was acidified with HCl (1 N aqueous) and extracted with EtOAc. The organic layer was dried over Na₂SO₄, filtered, and concentrated under reduced pressure. The crude product was passed through a pad of silica gel eluting with EtOH, then purified by reverse phase HPLC (column: XBridge® C18, 19×100 mm, 5 μm; mobile phase gradient: 20 to 40% ACN in 20 mM aqueous NH₄HCO₃, pH 9) to give the title compound (62 mg, 59%) as a white solid. ES-MS m/z 392 (M+H).

### Preparation 8

### tert-Butyl (4-(cyanomethyl)-3,3-difluoro-pyrrolidine-1-carboxylate

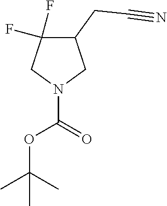 [SMILES: CC(C)(C)OC(=O)N1CC(CC#N)C(F)(F)C1]

Methanesulfonyl chloride (8.7 mL, 110 mmol) was added slowly to a solution of tert-butyl-3,3-difluoro-4-(hydroxymethyl)pyrrolidine-1-carboxylate (prepared essentially as described in McAlpine, I.; et al. J. Org. Chem. 2015, 80, 7266-7274; 13.6 g, 57.3 mmol) in TEA (16 mL, 115 mmol) at 0° C. under nitrogen atmosphere and the mixture was stirred for 1.5 h at 0° C. Water was added at 0° C. and the mixture was extracted with EtOAc. The combined organic layers were dried over Na₂SO₄, filtered and concentrated. The residue was dissolved in DMF (260 mL) and potassium cyanide (14.5 g, 218 mmol) was added. The mixture was heated under nitrogen at 75° C. for 3 days and then allowed to cool to RT. The mixture was then quenched with water and extracted with EtOAc. The combined organics were dried over Na₂SO₄, filtered and concentrated. The residue was purified by silica gel chromatography using a gradient of 10 to 40% acetone in hexanes to give the title compound (10.2 g, 54%). ES-MS (m/z): 191 (M+H-tert-butyl).

### Preparation 9

### 2-[1-tert-butoxycarbonyl-4,4-difluoro-pyrrolidin-3-yl]acetic acid

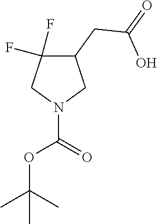 [SMILES: CC(C)(C)OC(=O)N1CC(CC(=O)O)C(F)(F)C1]

Potassium hydroxide (40% mass in water, 63.5 mL, 628 mmol) was added to a solution of tert-butyl (4-(cyanomethyl)-3,3-difluoro-pyrrolidine-1-carboxylate (10.3 g, 31.4 mmol) in MeOH (125 mL) and the resulting mixture was heated to reflux for 17 h.

The reaction was cooled to RT, HCl (1 M aqueous) was added to bring the pH to 7, then citric acid (5% in water) was added to bring pH to 3. The mixture was extracted with DCM, washed with saturated aqueous NaCl, then the organic layer was passed through a phase separator cartridge. The eluate was concentrated to obtain the title compound (8.32 g, 90%). ES-MS (m/z): 264 (M−H).

### Preparation 10

### tert-Butyl 4-[2-[(4S)-4-benzyl-2-oxo-oxazolidin-3-yl]-2-oxo-ethyl]-3,3-difluoro-pyrrolidine-1-carboxylate

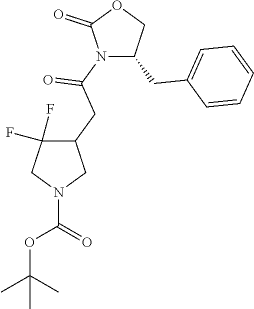 [SMILES: CC(C)(C)OC(=O)N1CC(CC(=O)N2C(=O)OC[C@@H]2Cc2ccccc2)C(F)(F)C1]

To a solution of 2-[1-tert-butoxycarbonyl-4,4-difluoro-pyrrolidin-3-yl]acetic acid (7.51 g, 28.3 mmol) in anhydrous THE (75 mL) at 0° C. was added TEA (9.9 mL), then pivaloyl chloride (4.58 mL, 36.8 mmol) was added dropwise. The reaction was stirred at 0° C. for 1 h and a solution of lithium chloride (1.52 g, 35.5 mmol) in anhydrous THE (82.6 mL) was added dropwise followed by a solution of (S)-4-benzyl-2-oxazolidinone (5.07 g, 28.3 mmol) in anhydrous THE (82.6 mL). The mixture was stirred at 0° C. for 1.5 h, then at RT for 20 h. HCl (1M, 70 mL) was added and the aqueous layer was extracted with EtOAc. The organic layer was separated and dried over anhydrous Na₂SO₄, then filtered and concentrated under reduce pressure. The residue was purified via silica gel chromatography using a gradient of 0 to 100% EtOAc in hexanes to give the title compound (7.27 g, 61%). ES-MS m/z 369 (M+H-tert-butyl).

### Preparation 11

### tert-Butyl 4-[(1S)-2-[(4S)-4-benzyl-2-oxo-oxazolidin-3-yl]-1-[(3-bromophenyl)methyl]-2-oxo-ethyl]-3,3-difluoro-pyrrolidine-1-carboxylate

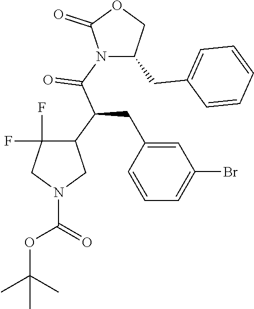 [SMILES: CC(C)(C)OC(=O)N1CC([C@H](Cc2cccc(Br)c2)C(=O)N2C(=O)OC[C@@H]2Cc2ccccc2)C(F)(F)C1]

To a solution of tert-butyl 4-[2-[(4S)-4-benzyl-2-oxo-oxazolidin-3-yl]-2-oxo-ethyl]-3,3-difluoro-pyrrolidine-1-carboxylate (7.44 g, 17.5 mmol) in anhydrous THE (37 mL) at −78° C. was added lithium bis(trimethylsilyl)amide (1 M) in toluene (21 mL) dropwise. The reaction was stirred at −78° C. for 2 h and a solution of 3-bromobenzyl bromide (4.92 g, 19.3 mmol) in anhydrous THE (14.9 mL) was added dropwise. The reaction was stirred at RT overnight, then saturated aqueous NH₄Cl was added and the aqueous layer was extracted with EtOAc. The organic layer was washed with saturated aqueous NaCl, dried over anhydrous Na₂SO₄, filtered and concentrated under reduced pressure. The residue was purified via silica gel chromatography using a gradient of 10 to 40% EtOAc in hexanes to give the title compound (7.32 g, 70%). ES-MS m/z 537/539 (M+H-tert-butyl).

### Preparation 12

### (2S)-3-(3-Bromophenyl)-2-(1-tert-butoxycarbonyl-4,4-difluoro-pyrrolidin-3-yl)propanoic acid—Isomer 1 and Isomer 2

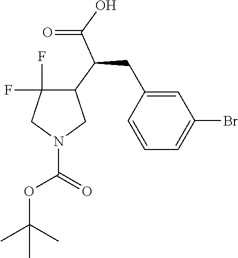 [SMILES: CC(C)(C)OC(=O)N1CC([C@H](Cc2cccc(Br)c2)C(=O)O)C(F)(F)C1]

To a stirred solution of tert-butyl 4-[(1S)-2-[(4S)-4-benzyl-2-oxo-oxazolidin-3-yl]-1-[(3-bromophenyl)methyl]-2-oxo-ethyl]-3,3-difluoro-pyrrolidine-1-carboxylate (7.32 g, 12.3 mmol) in THF (123 mL) at 0° C. was added hydrogen peroxide (35.5 mass % in water, 22.5 mL, 263 mmol) followed by a solution of lithium hydroxide (1 M in water, 18.5 mL, 18.5 mmol). The mixture was stirred at 0° C. for 6.5 h, then a solution of sodium bisulfite (15% in water) was added and the reaction warmed to RT. The pH was adjusted to 12 with the addition of NaOH (5 N aqueous) and the aqueous mixture was extracted with EtOAc. The organic layer was washed with saturated aqueous NaCl, dried over Na₂SO₄, filtered and concentrated under reduced pressure. The residue was purified via silica gel chromatography using a gradient of 0 to 50% EtOAc in hexanes+1% acetic acid to give the title compound (3.58 g, 65.5%). ES-MS m/z 378/380 (M+H-tert-butyl) as a mixture of diastereomers.

A portion (3.44 g) of the diastereomeric mixture was separated using chiral SFC chromatography (column: Chiralcel OJ (25×2 cm, 5 μm) at 40° C.; flow rate: 80 mL/min; mobile phase: isocratic 7% (IPA+0.2% DMEA) in CO₂) to give Isomer 1 (first-eluting isomer, 1.58 g, 46%, >98% de) and Isomer 2 (second-eluting isomer, 1.93 g, 56%, >95% de. Both isomers—ES-MS m/z 378/380 (M+H-tert-butyl).

### Preparation 13

### tert-Butyl (3R)-3-[(1S)-2-tert-butoxy-1-[[3-[(3-fluoro-5-methoxy-phenyl)methylamino]phenyl]methyl]-2-oxo-ethyl]pyrrolidine-1-carboxylate

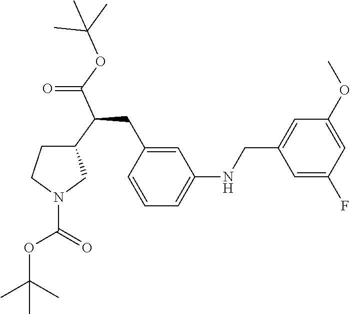 [SMILES: COc1cc(F)cc(CNc2cccc(C[C@H](C(=O)OC(C)(C)C)[C@H]3CCN(C(=O)OC(C)(C)C)C3)c2)c1]

To a solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429, 153 mg, 0.39 mmol) in DCM (4 mL) was added 3-fluoro-5-methoxy-benzaldehyde (0.091 g, 0.59 mmol) and sodium triacetoxyborohydride (0.17 g, 0.78 mmol). The reaction was stirred at RT for 1 h, then saturated aqueous NaHCO₃ was added and the aqueous layer was extracted with DCM. The organic layer was washed with water and then with saturated aqueous NaCl, dried over MgSO₄, filtered and then evaporated under reduced pressure. The residue was purified via reverse phase HPLC [column: XBridge™C18 (19×100 mm, 5 μm); mobile phase: solvent A=20 mM ammonium bicarbonate in water (pH 9), solvent B=ACN; flow rate: 25 mL/min] to give the title compound (155 mg, 75%). ES-MS m/z 529 (M+H).

The following were prepared essentially as described in Preparation 13 using the appropriate aldehyde:

| | | | | | Physical |
|---|---|---|---|---|---|
| | | | | | Data |
| | Chemical | | Procedure | Purification | ES-MS |
| Preparation | name | Structure | modifications | modifications | m/z |
| 14 | tert-Butyl (3R)-3-[(1S)- 2-tert-butoxy-1-[[3- (cyclobutylmethylamino) phenyl]methyl]-2-oxo- ethyl]pyrrolidine-1- carboxylate | 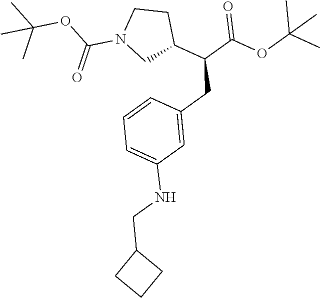 [SMILES: CC(C)(C)OC(=O)[C@@H](Cc1cccc(NCC2CCC2)c1)[C@H]1CCN(C(=O)OC(C)(C)C)C1] | A | B | 459 (M + H) |
| 15 | tert-Butyl (3R)-3-[(1S)- 2-tert-butoxy-1-[[3-[(4,4- difluorocyclohexyl) methylamino] phenyl]methyl]- 2-oxo-ethyl]pyrrolidine- 1-carboxylate | 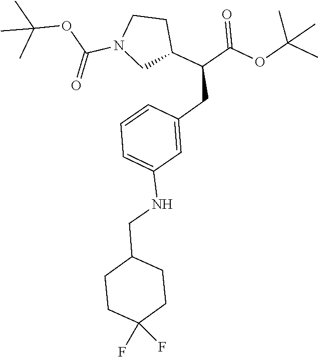 [SMILES: CC(C)(C)OC(=O)[C@@H](Cc1cccc(NCC2CCC(F)(F)CC2)c1)[C@H]1CCN(C(=O)OC(C)(C)C)C1] | A | B + C | 523 (M + H) |
| 16 | tert-Butyl (3R)-3-[(1S)- 2-tert-butoxy-1-[[3-(2- cyclopropylethylamino) phenyl]methyl]-2-oxo- ethyl]pyrrolidine-1- carboxylate | 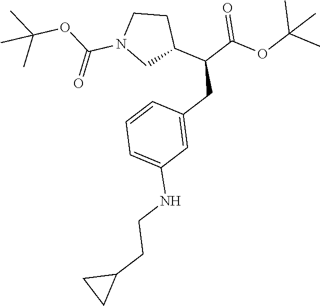 [SMILES: CC(C)(C)OC(=O)[C@@H](Cc1cccc(NCCC2CC2)c1)[C@H]1CCN(C(=O)OC(C)(C)C)C1] | A | B + D | 459 (M + H) |
| 17 | tert-Butyl (3R)-3-[(1S)- 2-tert-butoxy-1-[[3- (cyclopropylmethylamino) phenyl]methyl]-2-oxo- ethyl]pyrrolidine-1- carboxylate | 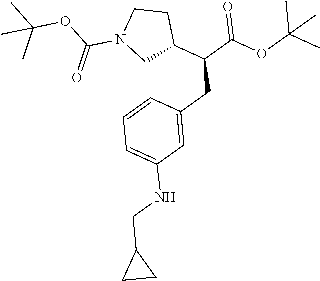 [SMILES: CC(C)(C)OC(=O)[C@@H](Cc1cccc(NCC2CC2)c1)[C@H]1CCN(C(=O)OC(C)(C)C)C1] | A | B | 445 (M + H) |
| 18 | tert-Butyl (3R)-3-[(1S)- 2-tert-butoxy-1-[[3- (cyclohexylmethylamino) phenyl]methyl]-2-oxo- ethyl]pyrrolidine-1- carboxylate | 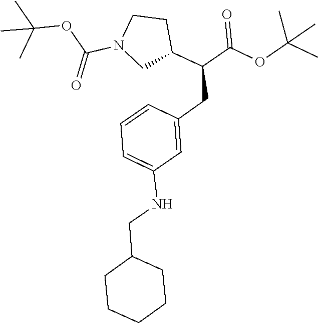 [SMILES: CC(C)(C)OC(=O)[C@@H](Cc1cccc(NCC2CCCCC2)c1)[C@H]1CCN(C(=O)OC(C)(C)C)C1] | A | B | 487 (M + H) |
| 19 | tert-Butyl (3R)-3-[(1S)- 2-tert-butoxy-2-oxo-1- [[3-(3,3,3- trifluoropropylamino) phenyl]methyl]ethyl] pyrrolidine-1- carboxylate | 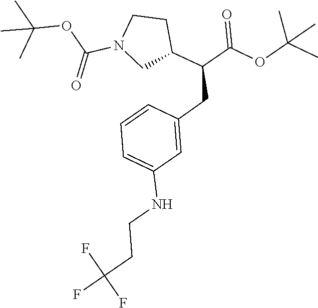 [SMILES: CC(C)(C)OC(=O)[C@@H](Cc1cccc(NCCC(F)(F)F)c1)[C@H]1CCN(C(=O)OC(C)(C)C)C1] | A | B | 387 (M + H − Boc) |

A. Sodium cyanoborohydride was used as reducing agent and MeOH or MeOH/THF as solvent. Temperature either RT or 60-65° C.
B. Silica gel chromatography using a gradient of EtOAc in hexanes
C. Reverse phase chromatography (column: Claricep C-series) using a gradient of ACN in aqueous NH₄CO₃ (pH9)

### Preparation 20

### tert-Butyl (3R)-3-[(1S)-2-tert-butoxy-1-[[3-[(3-fluoro-5-methoxy-anilino)methyl]phenyl]methyl]-2-oxo-ethyl]pyrrolidine-1-carboxylate

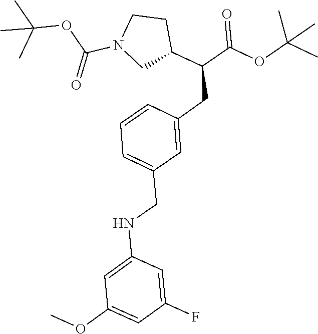 [SMILES: COc1cc(F)cc(NCc2cccc(C[C@H](C(=O)OC(C)(C)C)[C@H]3CCN(C(=O)OC(C)(C)C)C3)c2)c1]

To solution of tert-butyl (3R)-3-[(1S)-2-tert-butoxy-1-[(3-formylphenyl)methyl]-2-oxo-ethyl]pyrrolidine-1-carboxylate (prepared essentially as described in WO 2020/247429, 425 mg, 1.05 mmol) in DCM (10 mL) was added 3-fluoro-5-methoxy-aniline (0.223 g, 1.58 mmol) and one drop of acetic acid. The reaction was stirred at RT for 1 h, then sodium triacetoxyborohydride (0.456 g, 2.11 mmol) was added stirring continued for 1 h. Saturated aqueous NaHCO₃ was added and the aqueous layer was extracted with DCM. The organic layer was washed with water, then with saturated aqueous NaCl, then dried over MgSO₄, filtered and evaporated under reduced pressure. The residue was purified via reverse phase chromatography [column: Claricep C-series; mobile phase: gradient of 70 to 100% ACN in aqueous NH₄CO₃ (pH9)] to give the title compound (433 mg, 78%). ES-MS m/z 417 [M+H-(2×tert-butyl)]

The following were prepared essentially as described in Preparation 20 using the appropriate amine:

| | | | | | Physical |
|---|---|---|---|---|---|
| | | | | | Data |
| | Chemical | | Procedure | Purification | ES-MS |
| Preparation | name | Structure | modifications | modifications | m/z |
| 21 | tert-Butyl (3R)-3-[(1S)- 2-tert-butoxy-1-[[3-[[(3- fluoro-5-methoxy- phenyl)methylamino] methyl]phenyl]methyl]-2- oxo-ethyl]pyrrolidine-1- carboxylate | 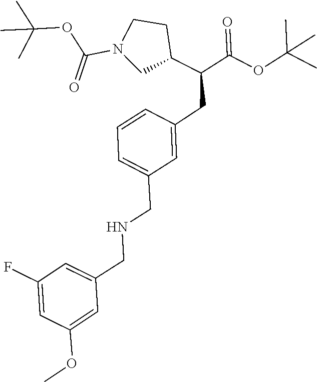 [SMILES: COc1cc(F)cc(CNCc2cccc(C[C@H](C(=O)OC(C)(C)C)[C@H]3CCN(C(=O)OC(C)(C)C)C3)c2)c1] | | | 543 (M + H) |
| 22 | tert-Butyl (3R)-3-[(1S)- 1-[[3-[(1H- benzimidazol-2- ylamino)methyl]phenyl] methyl]-2-tert-butoxy-2- oxo-ethyl]pyrrolidine-1- carboxylate | 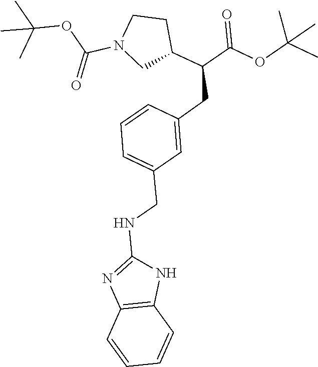 [SMILES: CC(C)(C)OC(=O)[C@@H](Cc1cccc(CNc2nc3ccccc3[nH]2)c1)[C@H]1CCN(C(=O)OC(C)(C)C)C1] | A | B | 521 (M + H) |

A. Aldehyde + amine + catalytic toluenesulfonic acid in toluene heated at reflux with a Dean Stark trap for 4 h. The solvent was eliminated, the residue was re-dissolved in MeOH and sodium borohydride was added
B. RP-HPLC/MS; column XBridge ™ C18 (10 × 100 mm, 5 μm); mobile phase- solvent A = 20 mM ammonium bicarbonate is water (pH 9); solvent B = ACN; flow rate- 25 mL/min

### Preparation 23

### (2S)-2-[(3R)-1-tert-Butoxycarbonylpyrrolidin-3-yl]-3-[3-[(2-fluoro-3-methoxy-phenyl) carbamoylamino]phenyl]propanoic acid

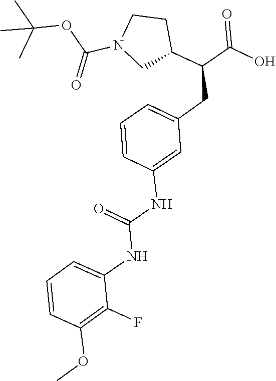 [SMILES: COc1cccc(NC(=O)Nc2cccc(C[C@H](C(=O)O)[C@H]3CCN(C(=O)OC(C)(C)C)C3)c2)c1F]

To a solution of (2S)-3-(3-bromophenyl)-2-[(3R)-1-tert-butoxycarbonylpyrrolidin-3-yl]propanoic acid (prepared essentially as described in WO 2020/247429, 200 mg, 0.50 mmol) in 1,4-dioxane (0.1 M, 5.021 mL) was added (2-fluoro-3-methoxy-phenyl)urea (0.277 g, 1.51 mmol), tBuXPhos-Pd-G3 (0.040 g, 0.050 mmol) and sodium tert-butoxide (0.149 g, 1.51 mmol). The mixture was heated at 100° C. under nitrogen atmosphere for 4 h, then diluted with NaOH (1 N aqueous) and extracted with EtOAc. The organic layer was discarded and the aqueous layer was then acidified with HCl (1 N aqueous) and extracted with EtOAc. The organic layer was dried over Na₂SO₄, then filtered and concentrated under reduced pressure. The residue was purified via silica gel chromatography using a gradient of 50 to 100% EtOAc in hexanes to give the title compound (88 mg, 32%). ES-MS m/z 502 (M+H).

The following were prepared essentially as described in Preparation 23 using the appropriate amine or urea:

| | | | | | Physical |
|---|---|---|---|---|---|
| | | | | | Data |
| | Chemical | | Procedure | Purification | ES-MS |
| Preparation | name | Structure | modifications | modifications | m/z |
| 24 | (2S)-2-[(3R)-1-tert- Butoxycarbonylpyrrolidin-3- yl]-3-[3-[(4-methylthiazol-2- yl)amino]phenyl]propanoic acid | 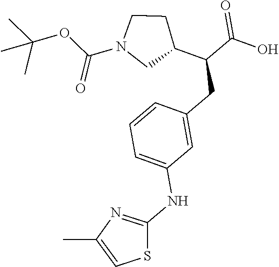 [SMILES: Cc1csc(Nc2cccc(C[C@H](C(=O)O)[C@H]3CCN(C(=O)OC(C)(C)C)C3)c2)n1] | A | B | 432 (M + H) |
| 25 | (2S)-2-[(3R)-1-tert- Butoxycarbonylpyrrolidin-3- yl]-3-[3-(tert- butylcarbamoylamino)phenyl] propanoic acid | 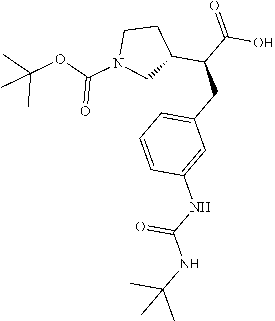 [SMILES: CC(C)(C)NC(=O)Nc1cccc(C[C@H](C(=O)O)[C@H]2CCN(C(=O)OC(C)(C)C)C2)c1] | | C | 434 (M + H) |
| 26 | (2S)-2-[(3R)-1-tert- Butoxycarbonylpyrrolidin-3- yl]-3-[3- [[methyl(phenyl)carbamoyl] amino]phenyl]propanoic acid | 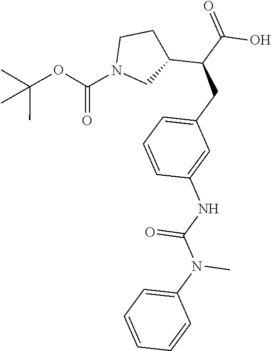 [SMILES: CN(C(=O)Nc1cccc(C[C@H](C(=O)O)[C@H]2CCN(C(=O)OC(C)(C)C)C2)c1)c1ccccc1] | | C | 468 (M + H) |
| 27 | (2S)-3-[3-[(1-Benzyl-4- piperidyl)carbamoylamino] phenyl]-2-[(3R)-1-tert- butoxycarbonylpyrrolidin-3- yl]propanoic acid | 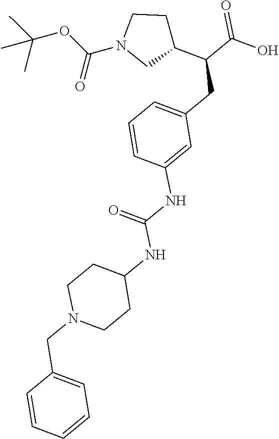 [SMILES: CC(C)(C)OC(=O)N1CC[C@H]([C@H](Cc2cccc(NC(=O)NC3CCN(Cc4ccccc4)CC3)c2)C(=O)O)C1] | | C | 551 (M + H) |
| 28 | (2S)-2-[(3R)-1-tert- Butoxycarbonylpyrrolidin-3- yl]-3-[3-(8- quinolylcarbamoylamino) phenyl]propanoic acid | 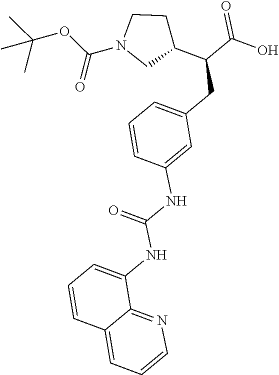 [SMILES: CC(C)(C)OC(=O)N1CC[C@H]([C@H](Cc2cccc(NC(=O)Nc3cccc4cccnc34)c2)C(=O)O)C1] | | C | 505 (M + H) |
| 29 | (2S)-2-[(3R)-1-tert- Butoxycarbonylpyrrolidin-3- yl]-3-[3-[(3,4- dichlorophenyl) carbamoylamino] phenyl]propanoic acid | 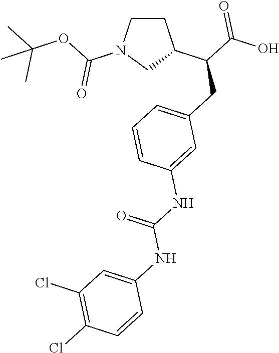 [SMILES: CC(C)(C)OC(=O)N1CC[C@H]([C@H](Cc2cccc(NC(=O)Nc3ccc(Cl)c(Cl)c3)c2)C(=O)O)C1] | | C | 522/524 (M + H) |
| 30 | (2S)-3-[3-[(4- Benzyloxyphenyl)carbamoyl amino]phenyl]-2-[(3R)-1- tert- butoxycarbonylpyrrolidin-3- yl]propanoic acid | 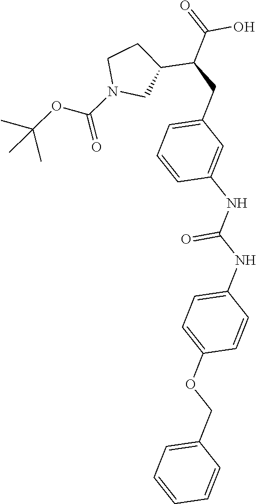 [SMILES: CC(C)(C)OC(=O)N1CC[C@H]([C@H](Cc2cccc(NC(=O)Nc3ccc(OCc4ccccc4)cc3)c2)C(=O)O)C1] | | C | 560 (M + H) |
| 31 | (2S)-2-[(3R)-1-tert- Butoxycarbonylpyrrolidin-3- yl]-3-[3- (isopropylcarbamoylamino) phenyl]propanoic acid | 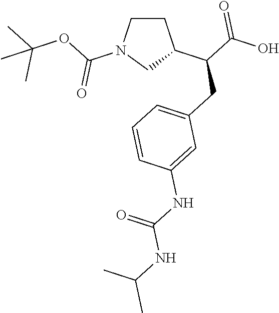 [SMILES: CC(C)NC(=O)Nc1cccc(C[C@H](C(=O)O)[C@H]2CCN(C(=O)OC(C)(C)C)C2)c1] | | C | 420 (M + H) |
| 32 | (2S)-2-[(3R)-1-tert- Butoxycarbonylpyrrolidin-3- yl]-3-[3-(2- methoxyethylcarbamoylamino) phenyl]propanoic acid | 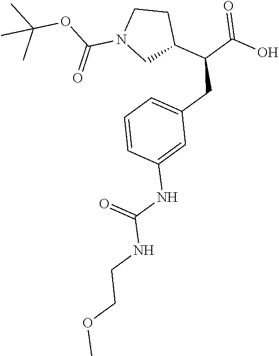 [SMILES: COCCNC(=O)Nc1cccc(C[C@H](C(=O)O)[C@H]2CCN(C(=O)OC(C)(C)C)C2)c1] | | C | 436 (M + H) |
| 33 | (2S)-2-[(3R)-1-tert- Butoxycarbonylpyrrolidin-3- yl]-3-[3-[(3-phenyl-1,2,4- oxadiazol-5- yl)carbamoylamino]phenyl] propanoic acid | 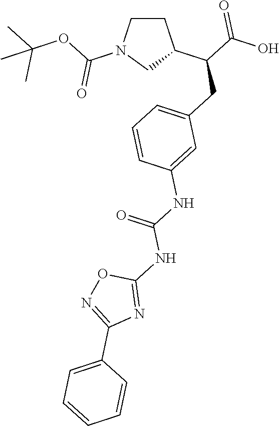 [SMILES: CC(C)(C)OC(=O)N1CC[C@H]([C@H](Cc2cccc(NC(=O)Nc3nc(-c4ccccc4)no3)c2)C(=O)O)C1] | | C | 522 (M + H) |

A. Alternative reagents were used: tris(dibenzylideneacetone)dipalladium(0) (0.1 equiv), 2-(di-tert-butylphosphino)-2′,4′,6′-triisopropyl-3,6-dimethoxy-1,1′-biphenyl (0.15 equiv), potassium phosphate tribasic (1.5 equiv), and tert-butyl alcohol (0.1 M); reaction heated to 120° C.; started with ammonium salt of (2S)-3-(3-bromophenyl)-2-[(3R)-1-tert-butoxycarbonylpyrrolidin-3-yl]propanoic acid, which was prepared as follows: (2S)-3-(3-bromophenyl)-2-[(3R)-1-tert-butoxycarbonylpyrrolidin-3-yl]propanoic acid in MTBE was treated with 2 equivalents of ammonia (7 M in MeOH), stirred for 1 h, filtered, washed with MTBE, then dried in vacuo
B. Silica gel chromatography using a gradient of 5 to 20% acetone in hexanes + 1% acetic acid
C. RP-HPLC/MS [column: XBridge ™ C18 (10 x 100 mm, 5 μm); mobile phase: solvent A = 20 mM aqueous NH₄HCO₃ (pH 9); solvent B = ACN; flow rate: 25 mL/min]

### Preparation 34

### tert-butyl (3R)-3-[(1S)-1-[[3-[[2-(2-Bromophenyl)acetyl]amino]phenyl]methyl]-2-tert-butoxy-2-oxo-ethyl]pyrrolidine-1-carboxylate

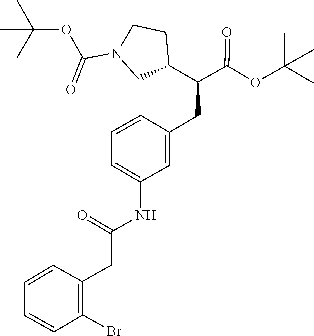 [SMILES: CC(C)(C)OC(=O)[C@@H](Cc1cccc(NC(=O)Cc2ccccc2Br)c1)[C@H]1CCN(C(=O)OC(C)(C)C)C1]

To a solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429, 1 g, 2.56 mmol) and TEA (1.04 g, 1.43 mL, 10.2 mmol) in DCM (12.8 mL) was added 2-bromophenylacetyl chloride (0.671 g, 0.427 mL, 2.82 mmol) slowly. The mixture was stirred at RT for 3.5 h, then more 2-bromophenylacetyl chloride (0.305 g, 0.194 mL, 1.28 mmol) was added and the mixture stirred at RT for 2.5 h. Saturated aqueous NaHCO₃ was added and the aqueous layer was extracted with DCM. The organic layer was washed with saturated aqueous NaCl, dried over MgSO₄, filtered and concentrated under reduced pressure. The reside was purified via silica gel chromatography using a gradient of 0 to 50% EtOAc in hexanes to give the title compound (860 mg, 57%). ES-MS m/z 587/589 (M+H).

### Preparation 35

### tert-Butyl (3R)-3-[(1S)-2-tert-butoxy-1-[[3-(3-hydroxypropanoylamino)phenyl]methyl]-2-oxo-ethyl]pyrrolidine-1-carboxylate

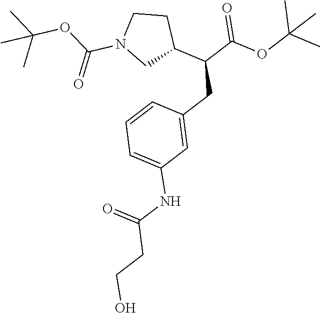 [SMILES: CC(C)(C)OC(=O)[C@@H](Cc1cccc(NC(=O)CCO)c1)[C@H]1CCN(C(=O)OC(C)(C)C)C1]

To a solution of 3-hydroxypropanoic acid (0.127 g, 0.42 mmol) in DMF (4.3 mL) was added HATU (0.298 g, 0.77 mmol) and 2,4,6-trimethylpyridine (0.047 g, 0.051 mL, 0.38 mmol). The mixture was stirred for 30 min and tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429, 150 mg, 0.38 mmol) was added. The reaction was stirred at RT for 18 h, then the reaction was concentrated and the residue purified via reverse phase chromatography (column: Claricep C-series) using a gradient of 20 to 70% ACN in aqueous NH₄CO₃ (pH9). A second purification was performed by RP-HPLC/MS [column: XBridge™ C18 (19×100 mm, 5 μm); mobile phase: solvent A=20 mM aqueous NH₄HCO₃ (pH 9); solvent B=ACN; flow rate: 25 mL/min] to give the title compound (33.6 mg, 19%). ES-MS m/z 463 (M+H).

The following were prepared essentially as described in Preparation 35 using the appropriate carboxylic acid:

| | | | | Physical |
|---|---|---|---|---|
| | | | | Data |
| | Chemical | | Purification | ES-MS |
| Preparation | name | Structure | modifications | m/z |
| 36 | tert-Butyl (3R)-3- [(1S)-2-tert-butoxy- 1-[3-[(2- hydroxyacetyl)amino] phenyl]methyl]-2- oxo- ethyl]pyrrolidine-1- carboxylate | 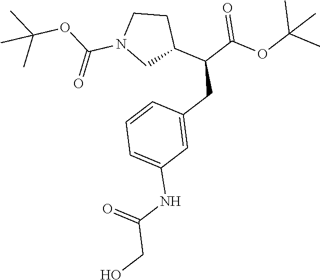 [SMILES: CC(C)(C)OC(=O)[C@@H](Cc1cccc(NC(=O)CO)c1)[C@H]1CCN(C(=O)OC(C)(C)C)C1] | A | 449 (M + H) |

A. Reverse phase flash chromatography was performed using a gradient of 20 to 70% ACN in aqueous NH₄CO₃, pH9. A second purification was performed by basic SFC [stationary phase: ethyl pyridine; column size: 30 × 150 mm, 5 μm; mobile phase: 10 mM NH₄CO₃ in MeOH/CO₂ (pH8)]; flow rate: 100 mL/min]

### Preparation 37

### tert-Butyl (3R)-3-[(1S)-2-tert-butoxy-1-[[3-(3-hydroxypropylcarbamoylamino)phenyl]methyl]-2-oxo-ethyl]pyrrolidine-1-carboxylate

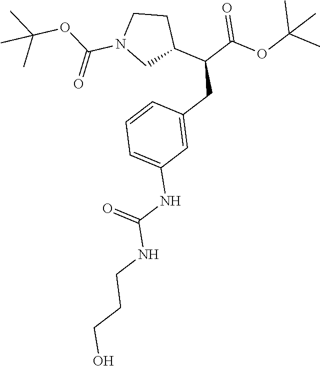 [SMILES: CC(C)(C)OC(=O)[C@@H](Cc1cccc(NC(=O)NCCCO)c1)[C@H]1CCN(C(=O)OC(C)(C)C)C1]

To a solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429, 200 mg, 0.49 mmol) in THE (5 mL) was added 1,1′-carbonyldiimidazole (0.164 g, 0.98 mmol). The mixture was stirred at RT for 18 h, then 3-amino-1-propanol (0.0569 mL, 0.0556 g, 0.74 mmol). The mixture was stirred at 110° C. for 1 h, then diluted with DCM and concentrated to dryness. The residue was purified via silica gel chromatography using a gradient of 10 to 40% acetone in DCM to give the title compound (176 mg, 73%). ES-MS m/z 492 (M+H).

### Preparation 38

### tert-Butyl (3R)-3-[(1S)-2-tert-butoxy-2-oxo-1-[[3-(2,2,2-trifluoroethylsulfonylamino)phenyl]methyl]ethyl]pyrrolidine-1-carboxylate

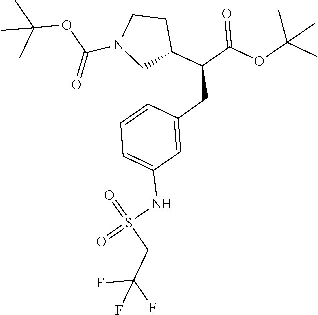 [SMILES: CC(C)(C)OC(=O)[C@@H](Cc1cccc(NS(=O)(=O)CC(F)(F)F)c1)[C@H]1CCN(C(=O)OC(C)(C)C)C1]

To a solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429, 130 mg, 0.33 mmol) and under nitrogen atmosphere in anhydrous DCM (3 mL) was added TEA (0.101 g, 0.139 mL, 1.00 mmol). The mixture was cooled to −78° C. and 2,2,2-trifluoroethanesulfonyl chloride (0.0608 g, 0.33 mmol) was added. The mixture warmed to RT and stirred for 18 h. Saturated aqueous NaHCO₃ was added and the aqueous layer was extracted with DCM. The layers were separated and the organic layer was concentrated under reduced pressure. The residue was purified via silica gel chromatography using a gradient of 15 to 60% acetone in hexanes to give the title compound (123 mg, 66%). ES-MS m/z 425 (M+H-(2×tert-butyl)).

The following were prepared essentially as described in Preparation 38 using the appropriate sulfonyl chloride:

| | | | Procedure | Purification | Physical Data |
|---|---|---|---|---|---|
| Preparation | Chemical name | Structure | modifications | modifications | ES-MS m/z |
| 39 | tert-Butyl (3R)-3-[(1S)- 2-tert-butoxy-1-[[3- ((cyclohexylmethyl) sulfonylamino)phenyl] methyl]-2-oxo- ethyl]pyrrolidine-1- carboxylate | 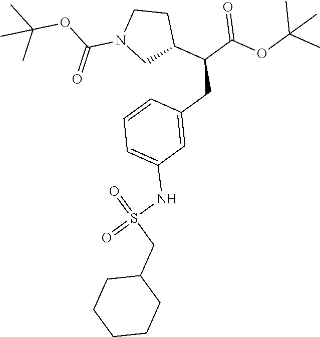 [SMILES: CC(C)(C)OC(=O)[C@@H](Cc1cccc(NS(=O)(=O)CC2CCCCC2)c1)[C@H]1CCN(C(=O)OC(C)(C)C)C1] | A | B | 451 (M + H—Boc) |
| 40 | tert-Butyl (3R)-3-[(1S)- 2-tert-butoxy-1-[[3- (cyclopropylsulfonylamino) phenyl]methyl]-2- oxo-ethyl]pyrrolidine-1- carboxylate | 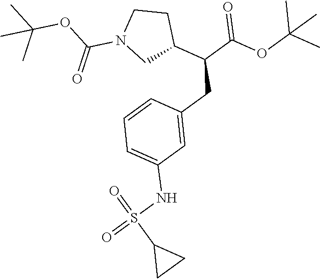 [SMILES: CC(C)(C)OC(=O)[C@@H](Cc1cccc(NS(=O)(=O)C2CC2)c1)[C@H]1CCN(C(=O)OC(C)(C)C)C1] | | | 395 (M + H—Boc) |

A Added 4-dimethylaminopyridine (0.1 equiv.)
B Reverse phase chromatography (column: Claricep C-series) using a gradient of ACN in aqueous NH₄CO₃ (pH 9).

### Preparation 41

### tert-Butyl (3R)-3-[(1S)-1-[(3-benzylsulfanylphenyl)methyl]-2-tert-butoxy-2-oxo-ethyl]pyrrolidine-1-carboxylate

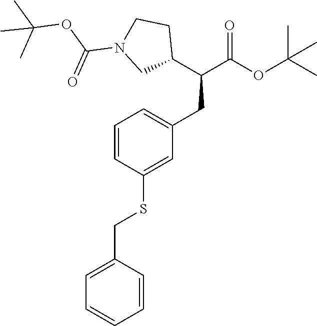 [SMILES: CC(C)(C)OC(=O)[C@@H](Cc1cccc(SCc2ccccc2)c1)[C@H]1CCN(C(=O)OC(C)(C)C)C1]

Nitrogen was bubbled into a suspension of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429, 7.8 g, 17.2 mmol) in toluene (10 mL). To the suspension was added tris(dibenzylideneacetone)dipalladium(0) (1.59 g, 1.72 mmol), 1,1′-bis(diphenylphosphino)ferrocene (1.99 g, 3.45 mmol), N,N-diisopropylethylamine (3.31 mL, 2.45 g, 19.0 mmol) and benzyl mercaptan (2.06 mL, 2.18 mL, 17.4 mmol) and the mixture was stirred at 100° C. for 16 h. EtOAc was added and the mixture was filtered. Water was added to the filtrate and the aqueous layer was extracted with EtOAc. The organic layer was separated, dried over MgSO₄, filtered and concentrated in vacuo. The residue was purified by silica gel chromatography using a gradient of 5 to 20% EtOAc in hexanes to give the title compound (6.89 g, 80%). ES-MS m/z 398 (M+H-Boc).

### Preparation 42

### tert-Butyl (3R)-3-[(1S)-2-tert-butoxy-1-[(3-chlorosulfonylphenyl)methyl]-2-oxo-ethyl]pyrrolidine-1-carboxylate

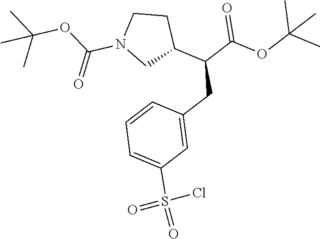 [SMILES: CC(C)(C)OC(=O)[C@@H](Cc1cccc(S(=O)(=O)Cl)c1)[C@H]1CCN(C(=O)OC(C)(C)C)C1]

To a 0° C. solution of tert-butyl (3R)-3-[(1S)-1-[(3-benzylsulfanylphenyl)methyl]-2-tert-butoxy-2-oxo-ethyl]pyrrolidine-1-carboxylate (1500 mg, 3.01 mmol) in ACN:acetic acid:water 40:1.5:1 (30 mL) was added 1,3-dichloro-5,5-dimethylhydantoin (1.76 g, 6.03 mmol) slowly in portions. The mixture was stirred at 0° C. for 20 min then concentrated in vacuo. The residue was dissolved in DCM, cooled to 0° C. and NaHCO₃ (5% aqueous) was added. The mixture was stirred for 5 min and the organic layer was separated. The organics were dried over MgSO₄ and concentrated in vacuo to give the title compound, which was used immediately without purification in the next step. ES-MS m/z 364 (M+H-Boc).

### Preparation 43

### tert-Butyl (3R)-3-[(1S)-2-tert-butoxy-1-[[3-[[(3-chlorophenyl)methyl]sulfamoyl]phenyl]methyl]-2-oxo-ethyl]pyrrolidine-1-carboxylate

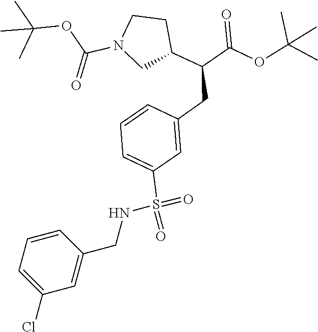 [SMILES: CC(C)(C)OC(=O)[C@@H](Cc1cccc(S(=O)(=O)NCc2cccc(Cl)c2)c1)[C@H]1CCN(C(=O)OC(C)(C)C)C1]

To solution of tert-butyl (3R)-3-[(1S)-2-tert-butoxy-1-[(3-chlorosulfonylphenyl)methyl]-2-oxo-ethyl]pyrrolidine-1-carboxylate (0.446 g, 0.60 mmol, 64 mass %) in DCM (3 mL) was added TEA (0.122 g, 0.168 mL, 1.21 mmol) and 3-chlorobenzylamine (0.102 g, 0.0888 mL, 0.72 mmol). The reaction was stirred at RT for 16 h, then HCl (1 N aqueous) was added and the aqueous layer was extracted with DCM. The organic layer was separated and dried over MgSO₄, filtered and concentrated in vacuo. The residue was purified via RP-HPLC/MS [column: XBridge™ C18 (10×100 mm, 5 μm); mobile phase: solvent A=20 mM aqueous NH₄HCO₃ (pH 9); solvent B=ACN; flow rate: 25 mL/min] to give the title compound (156 mg, 45%). ES-MS m/z 479 (M+H-Boc).

The following were prepared essentially as described in Preparation 43 using the appropriate amine:

| | | | Physical Data |
|---|---|---|---|
| Preparation | Chemical name | Structure | ES-MS m/z |
| 44 | tert-Butyl (3R)-3-[(1S)- 2-tert-butoxy-1-[[3-[(2- tert-butoxy-2-oxo- ethyl)sulfamoyl]phenyl] methyl]-2-oxo- ethyl]pyrrolidine-1- carboxylate | 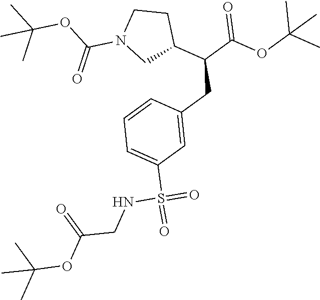 [SMILES: CC(C)(C)OC(=O)CNS(=O)(=O)c1cccc(C[C@H](C(=O)OC(C)(C)C)[C@H]2CCN(C(=O)OC(C)(C)C)C2)c1] | 469 (M + H—Boc) |
| 45 | tert-Butyl (3R)-3-[(1S)- 2-tert-butoxy-1-[[3-[2- (3- methoxyphenyl) ethylsulfamoyl] phenyl]methyl]- 2-oxo-ethyl]pyrrolidine- 1-carboxylate | 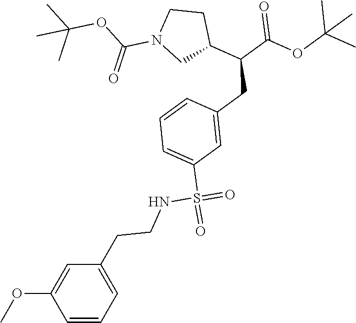 [SMILES: COc1cccc(CCNS(=O)(=O)c2cccc(C[C@H](C(=O)OC(C)(C)C)[C@H]3CCN(C(=O)OC(C)(C)C)C3)c2)c1] | 489 (M + H—Boc) |
| 46 | tert-Butyl (3R)-3-[(1S)- 2-tert-butoxy-1-[[3- [[(3,5- dimethoxyphenyl) methyl]sulfamoyl]phenyl] methyl]-2-oxo- ethyl]pyrrolidine-1- carboxylate | 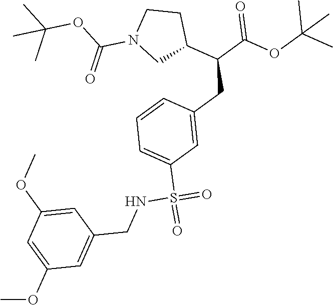 [SMILES: COc1cc(CNS(=O)(=O)c2cccc(C[C@H](C(=O)OC(C)(C)C)[C@H]3CCN(C(=O)OC(C)(C)C)C3)c2)cc(OC)c1] | 505 (M + H—Boc) |

### Preparation 47

### tert-Butyl (3R)-3-[2-methoxy-1-[(3-nitrophenyl)methyl]-2-oxo-ethyl]pyrrolidine-1-carboxylate

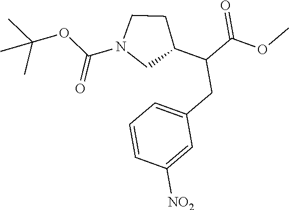 [SMILES: COC(=O)C(Cc1cccc([N+](=O)[O-])c1)[C@H]1CCN(C(=O)OC(C)(C)C)C1]

To a solution of tert-butyl (3R)-3-(2-methoxy-2-oxo-ethyl)pyrrolidine-1-carboxylate (prepared essentially as described in WO 2020/247429, 6 g, 24.7 mmol) in THE (170 mL) at −78° C. under nitrogen was added potassium bis(trimethylsilyl)amide (0.5 M in tolune, 60 mL, 30 mmol). The mixture was stirred at −78° C. for 45 min and then a solution of 1-(bromomethyl)-3-nitro-benzene (5.9 g, 27 mmol) in THE (25 mL) was added. The mixture was warmed to RT and stirred for 1.75 h, then quenched with saturated NH₄Cl and extracted with MTBE. The combined organic layers were dried over MgSO₄ and concentrated under reduced pressure. The residue was purified via silica gel chromatography using a gradient of 7 to 30% acetone in hexanes to give the title compound (3.4 g, 36%) as a yellow oil. ES-MS m/z 279 (M-Boc+H).

### Preparation 48

### tert-Butyl (3R)-3-[1-[(3-aminophenyl)methyl]-2-methoxy-2-oxo-ethyl]pyrrolidine-1-carboxylate

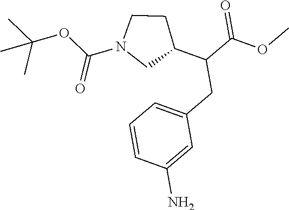 [SMILES: COC(=O)C(Cc1cccc(N)c1)[C@H]1CCN(C(=O)OC(C)(C)C)C1]

A mixture of tert-butyl (3R)-3-[2-methoxy-1-[(3-nitrophenyl)methyl]-2-oxo-ethyl]pyrrolidine-1-carboxylate (3.4 g, 9.0 mmol) and palladium (10% on activated carbon, 340 mg, 0.32 mmol) in MeOH (45 mL) was stirred at RT for 4 h under a balloon of hydrogen. The reaction mixture was filtered through a pad of Celite® and the filtrate was concentrated to dryness to give the title compound (2.9 g, 93%) as a colorless oil. ES-MS m/z 249 (M-Boc+H).

### Preparation 49

### tert-Butyl (3R)-3-[2-methoxy-2-oxo-1-[[3-(prop-2-ynylamino)phenyl]methyl]ethyl]pyrrolidine-1-carboxylate

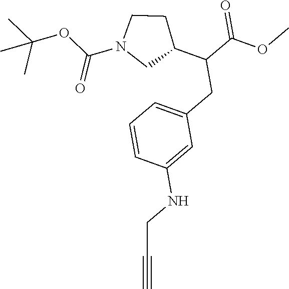 [SMILES: C#CCNc1cccc(CC(C(=O)OC)[C@H]2CCN(C(=O)OC(C)(C)C)C2)c1]

tert-Butyl (3R)-3-[1-[(3-aminophenyl)methyl]-2-methoxy-2-oxo-ethyl]pyrrolidine-1-carboxylate (710 mg, 2.04 mmol) was dissolved in DMF (7 mL), then K₂CO₃ (0.285 g, 2.04 mmol) and 3-bromoprop-1-yne (80% by mass in toluene, 0.242 mL, 0.333 g, 2.24 mmol) was added. The mixture was stirred at RT overnight. Water was added and the aqueous layer was extracted with EtOAc. The organic layer was dried over Na₂SO₄, filtered, and concentrated under reduced pressure. The residue was purified via silica gel chromatography using a gradient of 10 to 40% EtOAc in hexanes to give the title compound (0.252 g, 31%) as a colorless oil. ES-MS m/z 287 (M-Boc+H).

### Preparation 50

### 2-[(3R)-1-tert-butoxycarbonylpyrrolidin-3-yl]-3-[3-(prop-2-ynylamino)phenyl]propanoic acid

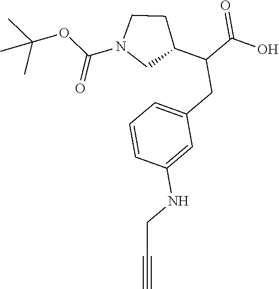 [SMILES: C#CCNc1cccc(CC(C(=O)O)[C@H]2CCN(C(=O)OC(C)(C)C)C2)c1]

To a solution of tert-butyl (3R)-3-[2-methoxy-2-oxo-1-[[3-(prop-2-ynylamino)phenyl]methyl]ethyl]pyrrolidine-1-carboxylate (252 mg, 0.65 mmol) in MeOH (4 mL) and THF (8 mL) was added NaOH (2M in water, 2 mL, 4 mmol). The mixture was stirred at RT overnight, then HCl (1 N aqueous) was added until the solution pH=4. The aqueous layer was extracted three times with EtOAc and the combined organics were dried over Na₂SO₄, filtered, and concentrated. The residue was loaded onto an HLB column and eluted first with water, then 1:1 water:ACN, then ACN. Appropriate fractions were concentrated and the resulting solid dried in vacuo at 40° C. to give the title compound (237 mg, 98%) as a brown solid, which is a mixture of diastereomers. ES-MS m/z 273 (M-Boc+H).

### Preparation 51

### tert-Butyl (3R)-3-[(1S)-2-tert-butoxy-1-[[3-(2-tert-butoxy-2-oxo-ethyl)phenyl]methyl]-2-oxo-ethyl]pyrrolidine-1-carboxylate

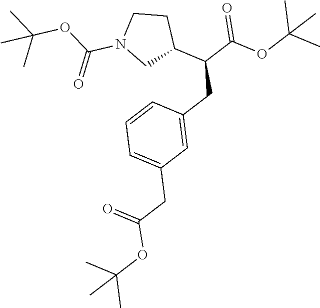 [SMILES: CC(C)(C)OC(=O)Cc1cccc(C[C@H](C(=O)OC(C)(C)C)[C@H]2CCN(C(=O)OC(C)(C)C)C2)c1]

To a 2-necked round-bottom flask with a reflux condenser was added: bis(dibenzylideneacetone)palladium (15.8 mg, 0.028 mmol) and 1,2,3,4,5-pentaphenyl-1′-(di-tert-butylphosphino)ferrocene (39.5 mg, 0.055 mmol) and the flask was purged with nitrogen. A solution of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429, 250 mg, 0.55 mmol) in THE (3 mL) was then added followed by (2-tert-butoxy-2-oxoethyl)(chloro)zinc (0.5M in Et₂O, 3 mL, 1.38 mmol). The reaction was heated at gentle reflux for 1.5 h, and once cooled saturated aqueous NH₄Cl was added and the mixture was extracted with EtOAc. The organic layer was separated, dried over MgSO₄, filtered, and concentrated. The residue was purified via silica gel chromatography using a gradient of 5 to 20% EtOAc in hexanes to give the title compound (212 mg, 79%) as a colorless oil. ES-MS m/z 390 (M-Boc+H).

### Preparation 52

### tert-Butyl (3S)-3-[2-[(4R)-4-benzyl-2-oxo-oxazolidin-3-yl]-2-oxo-ethyl]pyrrolidine-1-carboxylate

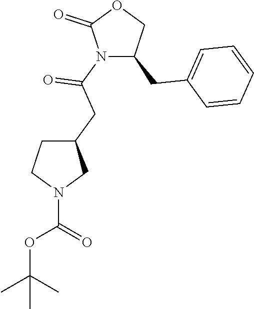 [SMILES: CC(C)(C)OC(=O)N1CC[C@@H](CC(=O)N2C(=O)OC[C@H]2Cc2ccccc2)C1]

Prepare the title compound essentially as described in Preparation 10 using (S)—N-BOC-3-pyrrolidineacetic acid and (R)-4-benzyl-2-oxazolidinone. Purify the crude product by silica gel chromatography using a gradient of 10 to 50% EtOAc in hexanes to give the title compound as a pale yellow oil. ES/MS (m/z): 333 (M+H-tert-butyl).

### Preparation 53

### (3R,7aS)-3-Phenyl-3,6,7,7a-tetrahydro-1H-pyrrolo[1,2-c]oxazol-5-one

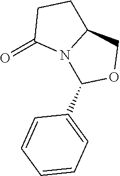 [SMILES: O=C1CC[C@H]2CO[C@H](c3ccccc3)N12]

A mixture of (5S)-5-(hydroxymethyl)pyrrolidin-2-one (20.0 g, 174 mmol), benzaldehyde (1.3 equiv., 226 mmol), p-toluenesulfonic acid (0.01 equiv., 1.74 mmol), and toluene (200 mL) was heated to reflux for 2 days in a round-bottom flask equipped with a Dean-Stark trap. The reaction was cooled to RT and washed with saturated aqueous NaHCO₃, saturated aqueous NaCl, dried over Na₂SO₄, filtered and concentrated. The residue was purified by silica gel chromatography eluting with a gradient of 0 to 70% EtOAc in hexanes to give the title compound (29.0 g, 82%) as a light yellow oil. ¹H NMR (399.80 MHz, CDCl₃) δ 7.48-7.45 (m, 2H), 7.40-7.35 (m, 3H), 6.35 (s, 1H), 4.24 (dd, J=6.3, 8.0 Hz, 1H), 4.19-4.12 (m, 1H), 3.50 (t, J=8.0 Hz, 1H), 2.87-2.78 (m, 1H), 2.56 (ddd, J=17.3, 10.0, 3.8 Hz, 1H), 2.43-2.37 (m, 1H), 2.00-1.93 (m, 1H).

### Preparation 54

### tert-butyl 2-[(3R,6R,7aS)-5-oxo-3-phenyl-3,6,7,7a-tetrahydro-1H-pyrrolo[1,2-c]oxazol-6-yl]acetate

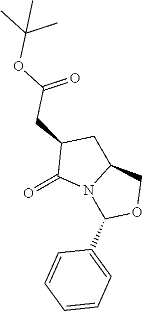 [SMILES: CC(C)(C)OC(=O)C[C@H]1C[C@H]2CO[C@H](c3ccccc3)N2C1=O]

To a solution of n-butyllithium (2.5 M in hexanes, 86 mL, 214 mmol) under nitrogen at 0° C. was added diisopropylamine (1.7 equiv., 243 mmol) slowly. After completion of the addition, the red slurry was stirred for 15 min and then slowly diluted with THE (200 mL). The resulting orange solution as cooled to −76° C., and then a solution of (3R,7aS)-3-phenyl-3,6,7,7a-tetrahydro-1H-pyrrolo[1,2-c]oxazol-5-one (29.0 g, 143 mmol) in THE (200 mL) was added dropwise at such a rate that the internal temperature did not rise above −68° C. The resulting brown solution was stirred for 45 min. A solution of tert-butyl bromoacetate (64.1 mL, 85.2 g, 428 mmol) in THE (100 mL) was added dropwise, keeping the internal temperature below −72° C. Upon completion of the addition, the mixture was stirred for 2 h at −78° C. Saturated aqueous NaHCO₃, was added and the reaction was diluted with Et₂O. The mixture was warmed to RT slowly overnight. The layers were separated, then the organic layer was washed with saturated aqueous NaHCO₃, saturated aqueous NaCl, dried over MgSO₄, filtered and concentrated. The residue was purified by silica gel chromatography using a gradient of 0 to 70% EtOAc in hexanes to give the title compound (40.7 g, 90%) as a yellow oil. ¹H NMR (399.80 MHz, CDCl₃) δ 7.48-7.45 (m, 2H), 7.40-7.36 (m, 3H), 6.34 (s, 1H), 4.26 (dd, J=6.3, 8.1 Hz, 1H), 4.18-4.11 (m, 1H), 3.63-3.59 (m, 1H), 3.33-3.27 (m, 1H), 2.84 (dd, J=4.4, 16.7 Hz, 1H), 2.73-2.66 (m, 1H), 2.41 (dd, J=8.7, 16.7 Hz, 1H), 1.74-1.65 (m, 1H), 1.48 (s, 9H).

### Preparation 55

### tert-Butyl (2S,4R)-2-(hydroxymethyl)-4-(2-methoxy-2-oxo-ethyl)pyrrolidine-1-carboxylate

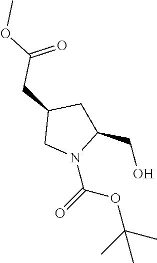 [SMILES: COC(=O)C[C@H]1C[C@@H](CO)N(C(=O)OC(C)(C)C)C1]

A solution of tert-butyl 2-[(3R,6R,7aS)-5-oxo-3-phenyl-3,6,7,7a-tetrahydro-1H-pyrrolo[1,2-c]oxazol-6-yl]acetate (11.30 g, 35.60 mmol) in dry THE (200 mL) under nitrogen was cooled to 0° C., then borane-tetrahydrofuran complex (1 M in THF, 71 mL, 71 mmol). The resulting mixture was heated to reflux for 3 h then the concentrated under reduced pressure. The residue was slowly and carefully dissolved in MeOH (50 mL), resulting in evolution of a gas. After the reaction had stopped generating gas, HCl (4 M solution in 1,4-dioxane, 50 mL, 200 mmol) was added, resulting in more evolution of gas. The resulting mixture was heated to reflux for 2 h, then cooled to ambient temperature and concentrated under reduced pressure. The residue was dissolved in EtOAc and washed with saturated aqueous NaHCO₃ twice, then saturated aqueous NaCl, then dried over MgSO₄, filtered and concentrated to give methyl 2-[(3R,5S)-1-benzyl-5-(hydroxymethyl)pyrrolidin-3-yl]acetate (8.78 g, 36.7 mmol).

The product was suspended under nitrogen in dry THE (300 mL), then tert-butoxycarbonyl tert-butyl carbonate (8.01 g, 36.7 mmol) and palladium on carbon (5% wt, 4.0 g) were added and the reaction vessel was purged with hydrogen (1 ATM) and stirred overnight. The flask was purged with nitrogen and then filtered through a pad of diatomaceous earth, washing with THF. The filtrate was concentrated and the residue purified by silica gel chromatography using a gradient of 0 to 80% EtOAc in hexanes to give the title compound (5.90 g, 65%) as a colorless oil. ¹H NMR (399.80 MHz, CDCl₃) δ 4.00-3.94 (m, 1H), 3.89-3.81 (m, 1H), 3.71 (m, 4H), 3.59 (dd, J=7.2, 11.6 Hz, 1H), 2.88 (t, J=10.5 Hz, 1H), 2.48-2.39 (m, 3H), 2.31-2.24 (m, 1H), 1.49 (s, 9H).

### Preparation 56

### tert-Butyl (2R,4R)-4-[2-[(4S)-4-benzyl-2-oxo-oxazolidin-3-yl]-2-oxo-ethyl]-2-methyl-pyrrolidine-1-carboxylate

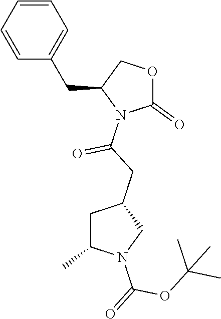 [SMILES: C[C@@H]1C[C@H](CC(=O)N2C(=O)OC[C@@H]2Cc2ccccc2)CN1C(=O)OC(C)(C)C]

To a mixture of imidazole (2.94 g, 43.2 mmol) and triphenylphosphine (8.49 g, 32.4 mmol) in dry THE (100 mL) under nitrogen at 0° C. was added iodine (8.22 g, 32.4 mmol) dropwise as a solution in THE (50 mL). The resulting mixture was stirred for 1 h, then a solution of tert-butyl (2S,4R)-2-(hydroxymethyl)-4-(2-methoxy-2-oxo-ethyl)pyrrolidine-1-carboxylate (5.90 g, 21.6 mmol) in THE (100 mL) was added dropwise. The cooling bath was allowed to slowly warm up and the reaction stirred at RT overnight. The reaction mixture was diluted with ether and washed with 10% aqueous sodium thiosulfate solution, saturated aqueous NaCl, dried over Na₂SO₄, filtered and concentrated. The residue was purified by silica gel chromatography using a gradient of 0 to 100% MTBE in hexanes to give tert-butyl (2S,4R)-2-(iodomethyl)-4-(2-methoxy-2-oxo-ethyl)pyrrolidine-1-carboxylate (7.30 g, 88%).

The product was suspended along with palladium on carbon (5 mass %, 4.0 g) in a mixture of TEA (3.19 mL, 2.31 g, 22.9 mmol) in dry THE (300 mL) under nitrogen. The reaction vessel was purged with hydrogen (1 ATM) and stirred overnight. The reaction was re-charged with additional Pd-C (200 mg) and stirred for 24 h. The reaction vessel was purged with nitrogen and the reaction mixture was filtered through a pad of diatomaceous earth, washing with THF. The filtrate was concentrated, and the residue was dissolved in ether and washed with water, 10% aqueous citric acid, and saturated aqueous NaCl. The organics were dried over MgSO₄, filtered and concentrated to give tert-butyl (2R,4R)-4-(2-methoxy-2-oxo-ethyl)-2-methyl-pyrrolidine-1-carboxylate (5.36 g, quantitative yield).

A mixture of the product in THE (30 mL), water (30 mL), lithium hydroxide (0.748 g, 31.2 mmol) was stirred for 2 h, then the reaction mixture was acidified with citric acid to pH=3-4 and the mixture was extracted with ether. The combined organics were dried over MgSO₄, filtered, and concentrated to give 2-[(3R,5R)-1-tert-butoxycarbonyl-5-methyl-pyrrolidin-3-yl]acetic acid.

The resulting product in dry THE (150 mL) under nitrogen was cooled to 0° C. TEA (3.16 g, 4.35 mL, 31.2 mmol) was added, and after 5 min, pivaloyl chloride (3.14 g, 3.18 mL, 26.0 mmol) was added drop-wise over 30 min. The resulting slurry was warmed to RT and stirred for 1 h. The reaction mixture was cooled to 0° C., then a solution of LiCl (5.29 g, 125 mmol) in a minimum amount of THF was added dropwise, followed by (S)-4-benzyl-2-oxazolidinone (4.16 g, 26.0 mmol). The reaction was warmed to RT overnight, then diluted with ether. The organics were washed with water, saturated aqueous NaHCO₃, and saturated aqueous NaCl, then dried over Na₂SO₄, filtered, and concentrated. The residue was purified by silica gel chromatography using a gradient of 0 to 100% MTBE in to give the title compound (6.80 g, 81%) as a yellow oil. ¹H NMR (399.80 MHz, CDCl3) δ 7.39-7.31 (m, 3H), 7.23-7.21 (m, 2H), 4.70 (ddd, J=13.0, 7.2, 3.4 Hz, 1H), 4.27-4.19 (m, 2H), 4.03-4.01 (m, 2H), 3.31 (dd, J=3.3, 13.4 Hz, 1H), 3.11-2.96 (m, 3H), 2.83-2.77 (m, 1H), 2.60-2.53 (m, 1H), 2.48-2.37 (m, 1H), 1.49-1.48 (m, 9H), 1.33-1.24 (m, 3H).

The following were prepared essentially as described in Preparation 11 using the appropriate starting material:

| | | | Procedure | Physical Data |
|---|---|---|---|---|
| Preparation | Chemical name | Structure | modifications | ES-MS m/z |
| 57 | tert-Butyl (3S)-3-[(1R)- 2-[(4R)-4-benzyl-2-oxo- oxazolidin-3-yl]-1-[(3- bromophenyl)methyl]-2- oxo-ethyl]pyrrolidine-1- carboxylate | 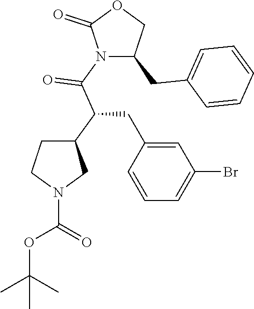 [SMILES: CC(C)(C)OC(=O)N1CC[C@@H]([C@@H](Cc2cccc(Br)c2)C(=O)N2C(=O)OC[C@H]2Cc2ccccc2)C1] | A | 501, 503 (M + H- tert-butyl) |
| 58 | tert-Butyl (3R)-3-[(1S)- 2-[(4S)-4-benzyl-2-oxo- oxazolidin-3-yl]-1-[(3- bromo-5-fluoro- phenyl)methyl]-2-oxo- ethyl]pyrrolidine-1- carboxylate | 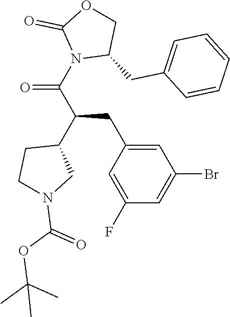 [SMILES: CC(C)(C)OC(=O)N1CC[C@H]([C@H](Cc2cc(F)cc(Br)c2)C(=O)N2C(=O)OC[C@@H]2Cc2ccccc2)C1] | A, B | 519, 521 (M + H- tert-butyl) |
| 59 | tert-Butyl (3R)-3-[(1S)- 2-[(4S)-4-benzyl-2-oxo- oxazolidin-3-yl]-1-[(4- fluoro-3-nitro- phenyl)methyl]-2-oxo- ethyl]pyrrolidine-1- carboxylate | 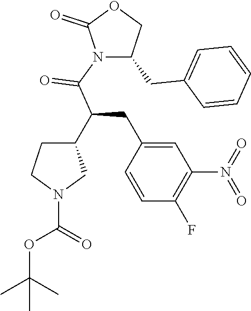 [SMILES: CC(C)(C)OC(=O)N1CC[C@H]([C@H](Cc2ccc(F)c([N+](=O)[O-])c2)C(=O)N2C(=O)OC[C@@H]2Cc2ccccc2)C1] | | 486 (M + H—tBu) |
| 60 | tert-Butyl (3R)-3-[(1S)- 2-[(4S)-4-benzyl-2-oxo- oxazolidin-3-yl]-1-[[3- bromo-5- (trifluoromethyl)phenyl] methyl]-2-oxo- ethyl]pyrrolidine-1- carboxylate | 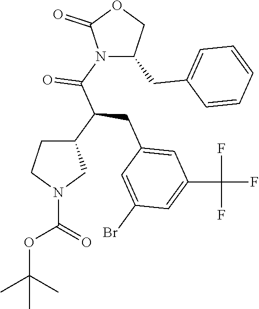 [SMILES: CC(C)(C)OC(=O)N1CC[C@H]([C@H](Cc2cc(Br)cc(C(F)(F)F)c2)C(=O)N2C(=O)OC[C@@H]2Cc2ccccc2)C1] | A, B | 569, 571 (M + H- tert-butyl) |
| 61 | tert-Butyl (3R)-3-[(1S)- 2-[(4S)-4-benzyl-2-oxo- oxazolidin-3-yl]-1-[(4- bromophenyl)methyl]-2- oxo-ethyl]pyrrolidine-1- carboxylate | 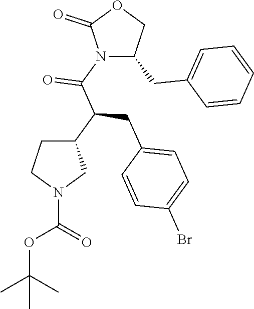 [SMILES: CC(C)(C)OC(=O)N1CC[C@H]([C@H](Cc2ccc(Br)cc2)C(=O)N2C(=O)OC[C@@H]2Cc2ccccc2)C1] | B, C | 501, 503 (M + H- tert-butyl) |
| 62 | tert-Butyl (2R,4R)-4- [(1S)-2-[(4S)-4-benzyl- 2-oxo-oxazolidin-3-yl]- 1-[(3- nitrophenyl)methyl]-2- oxo-ethyl]-2-methyl- pyrrolidine-1- carboxylate | 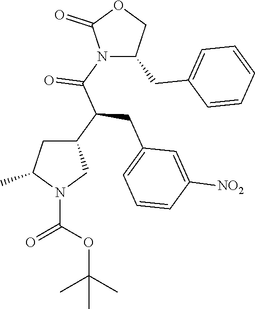 [SMILES: C[C@@H]1C[C@H]([C@H](Cc2cccc([N+](=O)[O-])c2)C(=O)N2C(=O)OC[C@@H]2Cc2ccccc2)CN1C(=O)OC(C)(C)C] | | 482 (M + H- tert-butyl) |

A product was used without purification
B starting material (tert-butyl (3R)-3-[2-[(4S)-4-benzyl-2-oxo-oxazolidin-3-yl]-2-oxo-ethyl]pyrrolidine-1-carboxylate) was prepared essentially as described in WO 2020/247429
C reaction was stirred at 0° C. for 20 min and prior to bromide addition

The following were prepared essentially as described in Preparation 12 using the appropriate starting material:

| | | | Procedure | Physical Data |
|---|---|---|---|---|
| Preparation | Chemical name | Structure | modifications | ES-MS m/z |
| 63 | (2R)-3-(3- Bromophenyl)-2-[(3S)- 1-tert- butoxycarbonylpyrrolidin- 3-yl]propanoic acid ammonium salt | 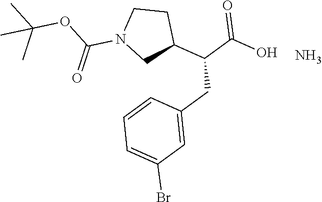 | A, B | 342, 344 (M + H- tert-butyl) |
| 64 | (2S)-3-(3-Bromo-5- fluoro-phenyl)-2-[(3R)- 1-tert- butoxycarbonylpyrrolidin- 3-yl]propanoic acid | 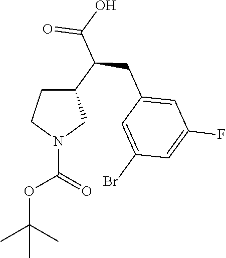 [SMILES: CC(C)(C)OC(=O)N1CC[C@H]([C@H](Cc2cc(F)cc(Br)c2)C(=O)O)C1] | B | 360, 362 (M + H- tert-butyl) |
| 65 | (2S)-2-[(3R)-1-tert- Butoxycarbonylpyrrolidin- 3-yl]-3-(4-fluoro-3- nitro-phenyl)propanoic acid | 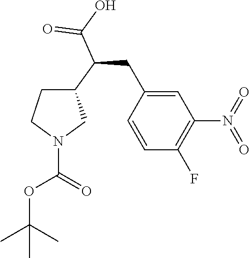 [SMILES: CC(C)(C)OC(=O)N1CC[C@H]([C@H](Cc2ccc(F)c([N+](=O)[O-])c2)C(=O)O)C1] | B | 327 (M + H- tert-butyl) |
| 66 | (2S)-3-[3-Bromo-5- (trifluoromethyl)phenyl]- 2-[(3R)-1-tert- butoxycarbonylpyrrolidin- 3-yl]propanoic acid | 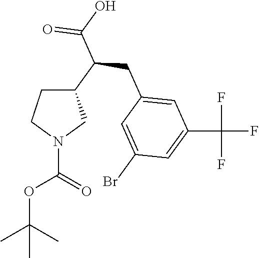 | B | 409, 411 (M + H- tert-butyl) |
| 67 | (2S)-3-(4- Bromophenyl)-2-[(3R)- 1-tert- butoxycarbonylpyrrolidin- 3-yl]propanoic acid | 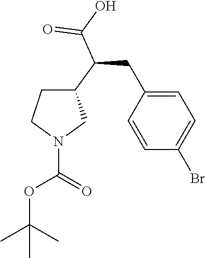 [SMILES: CC(C)(C)OC(=O)N1CC[C@H]([C@H](Cc2ccc(Br)cc2)C(=O)O)C1] | B | 342, 344 (M + H- tert-butyl) |
| 68 | (2S)-2-[(3R,5R)-1-tert- Butoxycarbonyl-5- methyl-pyrrolidin-3-yl]- 3-(3- nitrophenyl)propanoic acid | 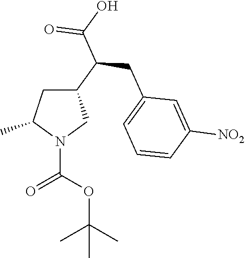 [SMILES: C[C@@H]1C[C@H]([C@H](Cc2cccc([N+](=O)[O-])c2)C(=O)O)CN1C(=O)OC(C)(C)C] | B | 323 (M + H- tert-butyl) |

A Upon completion of the reaction and aqueous workup, the crude product was dissolved in MTBE and ammonia (7M in MeOH, 2 equiv) was added, stirred for 1 h, filtered the resulting solid and washed with MTBE, then dried in vacuo.
B No chiral separation was performed

### Preparation 69

### tert-Butyl (3S)-3-[(1R)-1-[(3-bromophenyl)methyl]-2-tert-butoxy-2-oxo-ethyl]pyrrolidine-1-carboxylate

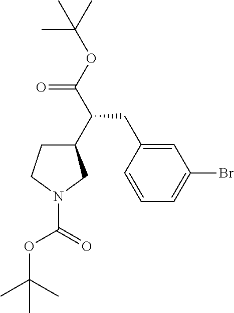 [SMILES: CC(C)(C)OC(=O)[C@H](Cc1cccc(Br)c1)[C@@H]1CCN(C(=O)OC(C)(C)C)C1]

(2R)-3-(3-Bromophenyl)-2-[(3S)-1-tert-butoxycarbonylpyrrolidin-3-yl]propanoic acid (ammonium salt, 0.530 g, 1.28 mmol) was converted to its free carboxylic acid by acidification with KHSO₄ (1N) and extraction with DCM. The organics were evaporated and the residue was dissolved in 2-methyltetrahydrofuran (4 mL). 2-tert-butyl-1,3-diisopropylisourea (1.0 mL, 0.902 g, 3.83 mmol, 85 mass %) was added and the reaction mixture was heated and stirred at 65° C. for 4 h. The reaction was cooled to RT, the solid in suspension was filtered and discarded, and the filtrate was concentrated in vacuo. The residue was purified by silica gel chromatography using a gradient of 10 to 40% EtOAc in hexanes to give 0.464 g (80%) of the title compound as a colorless oil. ES/MS m/z 476,478 (M+Na).

The following were prepared essentially as described in Preparation 69 using the appropriate acid:

| | | | Procedure | Physical Data |
|---|---|---|---|---|
| Preparation | Chemical name | Structure | modifications | ES-MS m/z |
| 70 | tert-Butyl (3R)-3-[(1S)-1- [(3-bromo-5-fluoro- phenyl)methyl]-2-tert- butoxy-2-oxo- ethyl]pyrrolidine-1- carboxylate | 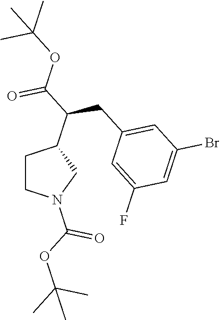 | A | 360, 362 (M + H-2x tert-butyl) |
| 71 | tert-Butyl (3R)-3-[(1S)-2- tert-butoxy-1-[(4-fluoro-3- nitro-phenyl)methyl]-2- oxo-ethyl]pyrrolidine-1- carboxylate | 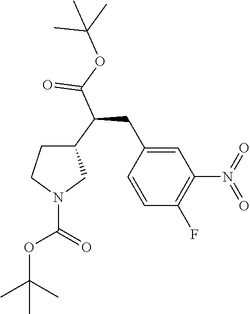 | A, B | 327 (M + H-2x tert-butyl) |
| 72 | tert-Butyl (3R)-3-[(1S)-1- [[3-bromo-5- (trifluoromethyl)phenyl] methyl]-2-tert-butoxy-2- oxo-ethyl]pyrrolidine-1- carboxylate | 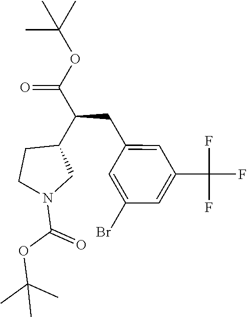 | A | 409, 411 (M + H-2x tert-butyl) |
| 73 | tert-Butyl (3R)-3-[1-[(3- bromophenyl)methyl]-2- tert-butoxy-1-methyl-2- oxo-ethyl]pyrrolidine-1- carboxylate (Isomer 2) | 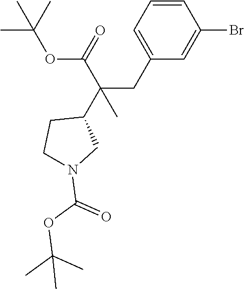 | C | 356, 358 (M + H-2x tert-butyl). |

A Starting material was used as the free acid and the neutralization step was not needed
B Purification by silica gel chromatography eluting with acetone:hexanes
C Isomer 2 of the starting material was used

### Preparation 74

### tert-Butyl-4-[(1S)-1-[(3-bromophenyl)methyl]-2-tert-butoxy-2-oxo-ethyl]-3,3-difluoro-pyrrolidine-1-carboxylate (Isomer 1 and Isomer 2)

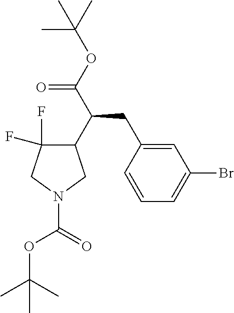 [SMILES: CC(C)(C)OC(=O)[C@@H](Cc1cccc(Br)c1)C1CN(C(=O)OC(C)(C)C)CC1(F)F]

A mixture of (2S)-3-(3-bromophenyl)-2-(1-tert-butoxycarbonyl-4,4-difluoro-pyrrolidin-3-yl)propanoic acid (1.09 g, 2.31 mmol) and 2-tert-butyl-1,3-diisopropylisourea (1.6 mL, 6.9 mmol) in 2-methyltetrahydrofuran (24 mL) was heated at 60° C. for 30 min. After 1 h, more 2-tert-butyl-1,3-diisopropylisourea (0.8 mL, 3 mmol) was added and the mixture was heated at 60° C. for 1 h. The reaction was cooled to RT. The white solid was filtered and washed with MTBE. The filtrate was evaporated to dryness. The residue was purified by silica gel chromatography using a gradient of 0 to 60% MTBE in hexanes to give Isomer 1 (280 mg, 25%, first-eluting isomer) as a colorless oil and Isomer 2 (367 mg, 32%, second-eluting isomer) as a colorless oil. Both Isomer 1 and Isomer 2: ES/MS m/z 378, 380 (M−2×tBu+H).

### Preparation 75

### tert-Butyl (3S)-3-[1-[(3-bromophenyl)methyl]-2-tert-butoxy-1-hydroxy-2-oxo-ethyl]pyrrolidine-1-carboxylate (Isomer 1 and Isomer 2)

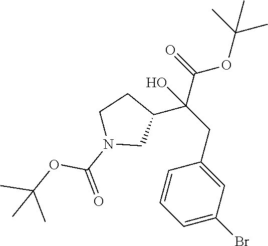 [SMILES: CC(C)(C)OC(=O)N1CC[C@H](C(O)(Cc2cccc(Br)c2)C(=O)OC(C)(C)C)C1]

Diisopropylamine (4.4 mL, 31.5 mmol) was added to a reaction vessel, chilled on an ice bath (0° C.) under inert atmosphere and then n-butyl lithium (2.5M solution in hexanes, 12 mL, 29 mmol) was added, forming a white slurry after a few minutes. To the mixture was added anhydrous THE (50 mL) and the reaction was cooled −78° C. tert-Butyl (3R)-3-[(1S)-1-[(3-bromophenyl)methyl]-2-tert-butoxy-2-oxo-ethyl]pyrrolidine-1-carboxylate (prepared essentially as described in WO 2020/247429, 5.50 g, 12.1 mmol) was added as a solution in THE (50 mL), and the mixture was stirred for 2 h at −78° C. A solution of 3-phenyl-2-(phenylsulfonyl)-1,2-oxaziridine (5.87 g, 21.8 mmol) in THE (50 mL) was added at −78° C. and the reaction mixture was allowed to warm to RT and stirred for 18 h. The reaction mixture was poured into ice with citric acid and extracted with DCM (2×100 mL). The organic extracts were combined, dried over MgSO₄, filtered and concentrated to dryness under reduced pressure. The residue was purified by silica gel chromatography using a gradient of 0 to 40% EtOAc in hexanes to give the title compound as a mixture of diastereomers (2.92 g). ES/MS (m/z): 358/360 (M+H-2×tert-butyl).

The diastereomeric mixture was separated by chiral HPLC [column: Lux Amylose-1 5×25 cm; mobile phase: 85:15 CO₂: IPA; mixture dissolved in IPA @50 mg/mL, 350 mg injections every 6 min) to give Isomer 1 (1.136 g, 20%) and Isomer 2 (0.956 g, 17%).

### Preparation 76

### tert-Butyl (3S)-3-[1-bromo-2-methoxy-2-oxo-ethyl]pyrrolidine-1-carboxylate

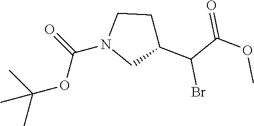 [SMILES: COC(=O)C(Br)[C@H]1CCN(C(=O)OC(C)(C)C)C1]

To a solution of tert-butyl (3R)-3-(2-methoxy-2-oxo-ethyl)pyrrolidine-1-carboxylate (prepared essentially as described in WO 2020/247429, 2 g, 8.2 mmol) in THE (16 mL) under N₂ atmosphere at −78° C. was added lithium bis(trimethylsilyl)amide (1.0 M in hexane (9.9 mL, 9.9 mmol). The mixture was stirred at −78° C. for 30 min and then chlorotrimethylsilane (1.57 mL, 1.34 g, 12.3 mmol) was added. The mixture was stirred at −78° C. for 30 min. Then a solution of N-bromosuccinimide (1.81 g, 9.86 mmol) in THE (22 mL) was added at −78° C. The reaction was stirred at RT overnight then saturated aqueous NH₄Cl was added. The mixture was extracted with EtOAc, the combined organic phases were washed with saturated aqueous NaCl, dried over MgSO₄, filtered, and concentrated. The residue was purified by silica gel chromatography using a gradient of 0 to 100% EtOAc in hexanes to give the title compound (mixture of diastereomers) as a yellow oil. ES/MS m/z 266, 268 (M-tBu+H).

### Preparation 77

### tert-Butyl (3S)-3-[1-(3-bromophenyl)sulfanyl-2-methoxy-2-oxo-ethyl]pyrrolidine-1-carboxylate

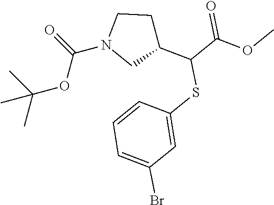 [SMILES: COC(=O)C(Sc1cccc(Br)c1)[C@H]1CCN(C(=O)OC(C)(C)C)C1]

To sodium methoxide (0.5 M solution in MeOH, 20 mL, 8.75 mmol) was added a solution of 3-bromothiophenol (0.956 mL, 1.65 g, 8.75 mmol) in THE (3 mL) and the mixture was heated at 80° C. for 1 h. A solution of tert-butyl (3S)-3-[1-bromo-2-methoxy-2-oxo-ethyl]pyrrolidine-1-carboxylate (1.88 g, 5.83 mmol) in THE (6 mL) was added and the mixture was heated at 80° C. for 30 min. Water was added to the reaction mixture and extracted with EtOAc. The combined organic phases were washed with saturated aqueous NaCl, dried over MgSO₄, filtered, and concentrated. The residue was purified by silica gel chromatography using a gradient of 50 to 100% MTBE in hexanes to give the title compound (2.2 g, 88%, mixture of diastereomers) as a pale yellow oil. ES/MS m/z 374, 376 (M-tBu+H).

### Preparation 78

### 2-(3-Bromophenyl)sulfanyl-2-[(3S)-1-tert-butoxycarbonylpyrrolidin-3-yl]acetic acid

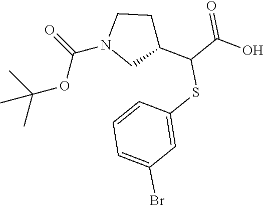 [SMILES: CC(C)(C)OC(=O)N1CC[C@H](C(Sc2cccc(Br)c2)C(=O)O)C1]

A mixture of tert-butyl (3S)-3-[1-(3-bromophenyl)sulfanyl-2-methoxy-2-oxo-ethyl]pyrrolidine-1-carboxylate (2.2 g, 5.1 mmol) and NaOH (5 N aqueous solution, 15 mL, 77 mmol) in MeOH (25 mL) and THF (25 mL) was stirred at RT 2.5 h. Organic solvents were evaporated to dryness and 1 N aqueous HCl was added to the residue bringing the mixture pH=2-3. The mixture was extracted with EtOAc, then the combined organic phases were washed with saturated aqueous NaCl, dried over MgSO₄, filtered and concentrated to give the title compound (1.84 g, 86%) as a white solid (mixture of diastereomers). ES/MS m/z 360, 362 (M-tBu+H).

### Preparation 79

### tert-Butyl (3S)-3-[1-(3-bromophenyl)sulfanyl-2-tert-butoxy-2-oxo-ethyl]pyrrolidine-1-carboxylate

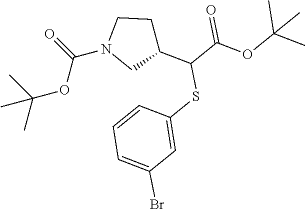 [SMILES: CC(C)(C)OC(=O)C(Sc1cccc(Br)c1)[C@H]1CCN(C(=O)OC(C)(C)C)C1]

A mixture of 2-(3-bromophenyl)sulfanyl-2-[(3S)-1-tert-butoxycarbonylpyrrolidin-3-yl]acetic acid (1.84 g, 4.42 mmol) and 2-tert-butyl-1,3-diisopropylisourea (4.6 mL, 4.07 g, 19.9 mmol) in 2-methyltetrahydrofuran (44 mL) was heated at 55° C. for 3 h. A white solid was filtered and discarded, and the filtrate was evaporated to dryness. The residue was purified by silica gel chromatography using a gradient of 0 to 50% MTBE in hexanes to give the title compound (2 g, 96%, mixture of diastereomers) as a colorless oil. ES/MS m/z 494, 496 (M+Na).

### Preparation 80

### 3-(3-Bromophenyl)-2-[(3R)-1-tert-butoxycarbonylpyrrolidin-3-yl]-2-methyl-propanoic acid (Isomer 1 and Isomer 2)

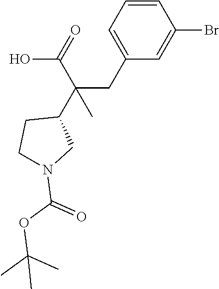 [SMILES: CC(C)(C)OC(=O)N1CC[C@H](C(C)(Cc2cccc(Br)c2)C(=O)O)C1]

To a solution of tert-butyl (3R)-3-[1-[(3-bromophenyl)methyl]-2-methoxy-2-oxo-ethyl]pyrrolidine-1-carboxylate (prepared essentially as described in WO 2020/247429, 8.4 g, 20 mmol) in THE (100 mL) under N₂ and at −78° C. was added lithium bis(trimethylsilyl)amide (1 M solution in THF, 41 mmol, 41 mL). The reaction was stirred at −78° C. for 2 h, then iodomethane (58 g, 25 mL, 410 mmol) was added and the reaction warmed to RT. The mixture was stirred overnight, then saturated aqueous NH₄Cl was added and extracted with EtOAc. The organic layer was washed with saturated aqueous NaCl and dried over MgSO₄, filtered and concentrated to give tert-butyl (3R)-3-[1-[(3-bromophenyl)methyl]-2-methoxy-1-methyl-2-oxo-ethyl]pyrrolidine-1-carboxylate (9.2 g, 98%) as a brown oil.

tert-Butyl (3R)-3-[1-[(3-bromophenyl)methyl]-2-methoxy-1-methyl-2-oxo-ethyl]pyrrolidine-1-carboxylate (9.2 g, 20 mmol) was dissolved in MeOH (80 mL) and THE (80 mL), then sodium hydroxide (5 M solution in water, 81 mL, 410 mmol) was added and the resulting mixture was heated at 60° C. for 3 days. The mixture was allowed to cool down to RT, then HCl (1N aqueous solution) was added until pH=2-3. The aqueous layer was extracted with EtOAc, and the organic layer was dried over MgSO₄, filtered and concentrated. The residue was purified by chiral SFC [column: Chiralpak AD 25×3 cm, 5 μm; mobile phase: solvent A—CO₂, solvent B—MeOH+0.2% DMEA; gradient: isocratic 80/20 A/B; flow rate: 120 mL/min] to obtain Isomer 1 (first-eluting isomer, 1.7 g, 27%, >98% de) as a white solid and Isomer 2 (second-eluting isomer, 3.4 g, 41%, >98% de) as a white solid. Both isomers: ES/MS (m/z): 356, 358 (M+H-tert-butyl).

### Preparation 81

### tert-Butyl (2R,4R)-4-[(1S)-1-[(3-aminophenyl)methyl]-2-tert-butoxy-2-oxo-ethyl]-2-methyl-pyrrolidine-1-carboxylate

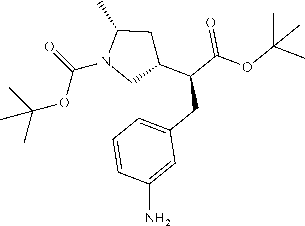 [SMILES: C[C@@H]1C[C@H]([C@H](Cc2cccc(N)c2)C(=O)OC(C)(C)C)CN1C(=O)OC(C)(C)C]

To a solution of (2S)-2-[(3R,5R)-1-tert-butoxycarbonyl-5-methyl-pyrrolidin-3-yl]-3-(3-nitrophenyl)propanoic acid (3.78 g, 9.99 mmol) in toluene (100 mL) at 80° C. was added N,N-dimethylformamide di-tert-butyl acetal (90 mass %, 26.7 mL, 22.6 g, 99.9 mmol), and the reaction was heated at 80° C. overnight. The reaction mixture was concentrated in vacuo and the residue was purified by silica gel chromatography using 40% EtOAc in hexanes to give tert-butyl (2R,4R)-4-[(1S)-2-tert-butoxy-1-[(3-nitrophenyl)methyl]-2-oxo-ethyl]-2-methyl-pyrrolidine-1-carboxylate (2.8 g, 60%) as a yellow oil.

The oil was dissolved in THE (70 mL) and palladium on carbon (5 mass %, 2.9 g) was added under nitrogen. The reaction vessel was purged with hydrogen and stirred under 1 ATM of hydrogen for 6 h. The resulting slurry was filtered through a pad of Celite® and washed with THF. The filtrate was concentrated to give a yellow oil, which was dissolved again in THE (70 mL) and slurried with palladium on carbon (5 mass %, 2.9 g) and stirred under 1 ATM of hydrogen for 24 h. The reaction mixture was filtered through a pad of Celite and washed with THF. The filtrate was concentrated to give the title compound (2.2 g, 84%) as a dark yellow oil, which contains minor diastereomeric impurities. ES/MS m/z 403 (M−H).

A portion of the product (1 g) was further purified by chiral SFC [column: Chiralpak AD-H 21×150 mm; mobile phase: 20% IPA in CO₂; flow rate: 80 mL/min; column temperature: 40° C.] to give the title compound (691 mg, third-eluting isomer, 96.7% de)

### Preparation 82

### tert-Butyl (3S)-3-[(1R)-1-[(3-aminophenyl)methyl]-2-tert-butoxy-2-oxo-ethyl]pyrrolidine

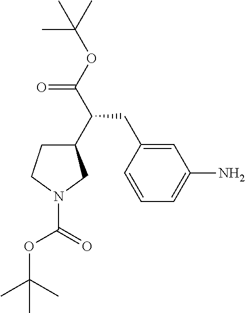 [SMILES: CC(C)(C)OC(=O)[C@H](Cc1cccc(N)c1)[C@@H]1CCN(C(=O)OC(C)(C)C)C1]

A mixture of tert-butyl (3S)-3-[(1R)-1-[(3-bromophenyl)methyl]-2-tert-butoxy-2-oxo-ethyl]pyrrolidine-1-carboxylate (0.464 g, 1.02 mmol), copper (II) acetylacetonate (0.027 g, 0.102 mmol), 2,2,6,6-tetramethyl-3,5-heptanedione (0.087 mL, 0.408 mmol), Cs₂CO₃ (0.665 g, 2.04 mmol), DMA (2 mL) and ammonium hydroxide (28 mass % in water, 0.816 mL, 6 mmol) was heated to 110° C. overnight, then cooled to RT. MTBE was added and the mixture was washed with water. The organics were dried over MgSO₄, filtered, and concentrated under reduced pressure. The residue was purified by silica gel chromatography using a gradient of 20 to 80% EtOAc in hexanes to give the title compound (0.271 g, 68%). ES/MS (m/z): 291 (M+H-BOC).

The following were prepared essentially as described in Preparation 82 using the appropriate aryl bromide:

| | | | Procedure | Physical Data |
|---|---|---|---|---|
| Preparation | Chemical name | Structure | modifications | ES-MS m/z |
| 83 | tert-Butyl (3R)-3-[(1S)- 1-[(3-amino-5-fluoro- phenyl)methyl]-2-tert- butoxy-2-oxo- ethyl]pyrrolidine-1- carboxylate | 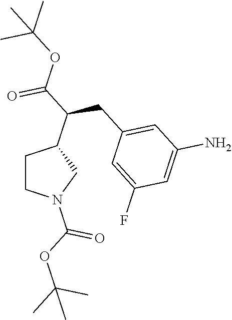 [SMILES: CC(C)(C)OC(=O)[C@@H](Cc1cc(N)cc(F)c1)[C@H]1CCN(C(=O)OC(C)(C)C)C1] | A | 309 (M + H—BOC) |
| 84 | tert-Butyl (3R)-3-[(1S)- 1-[[3-amino-5- (trifluoromethyl)phenyl] methyl]-2-tert-butoxy-2- oxo-ethyl]pyrrolidine-1- carboxylate | 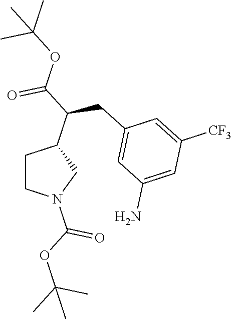 [SMILES: CC(C)(C)OC(=O)[C@@H](Cc1cc(N)cc(C(F)(F)F)c1)[C@H]1CCN(C(=O)OC(C)(C)C)C1] | A | 359 (M + H—BOC) |
| 85 | tert-Butyl (3S)-3-[1-[(3- aminophenyl)methyl]-2- tert-butoxy-1-hydroxy- 2-oxo-ethyl]pyrrolidine- 1-carboxylate (Isomer 1) | 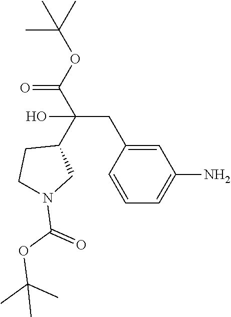 [SMILES: CC(C)(C)OC(=O)N1CC[C@H](C(O)(Cc2cccc(N)c2)C(=O)OC(C)(C)C)C1] | B, E | not available |
| 86 | tert-Butyl (3S)-3-[1-(3- aminophenyl)sulfanyl-2- tert-butoxy-2-oxo- ethyl]pyrrolidine-1- carboxylate (Isomer 1 and Isomer 2) | 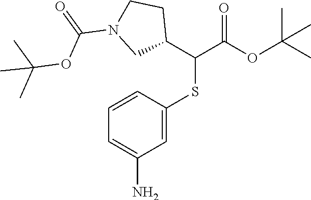 [SMILES: CC(C)(C)OC(=O)C(Sc1cccc(N)c1)[C@H]1CCN(C(=O)OC(C)(C)C)C1] | C, D | 431 (M + Na) |
| 87 | tert-Butyl (3R)-3-[1-[(3- tert-butoxy-1-methyl-2- aminophenyl)methyl]-2- oxo-ethyl]pyrrolidine-1- carboxylate | 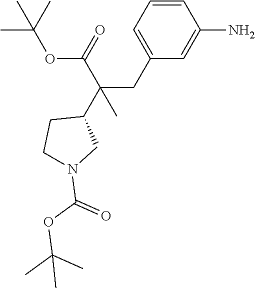 [SMILES: CC(C)(C)OC(=O)N1CC[C@H](C(C)(Cc2cccc(N)c2)C(=O)OC(C)(C)C)C1] | | 305 (M + H—BOC) |

A Reagents used—Copper (I) oxide (1 equiv.), Sodium azide (2 equiv), L-Proline (1.3 equiv.) in DMSO (0.2M, 9.6 mL); Reaction temperature—100° C.
B Upon completion, the reaction mixture was absorbed onto silica gel and carried forward to purification without aqueous workup.
C Purification by silica gel chromatography eluting with a mixture of MTBE and hexanes
D Diastereomers were separated by chiral SFC [column: Chiralcel OD 25 × 2 cm, 5 μm; mobile phase: CO₂—solvent A), MeOH + 0.5% DMEA—solvent B; isocratic 85:15 solvent A:solvent B; flow rate: 80 mL/min; column temperature: 40° C.], Isomer 1 is the first-eluting isomer and Isomer 2 is the second-eluting isomer.
E Prepared from Isomer 1 of the starting aryl bromide

### Preparation 88

### tert-Butyl-4-[1-[(3-aminophenyl)methyl]-2-tert-butoxy-2-oxo-ethyl]-3,3-difluoro-pyrrolidine-1-carboxylate (Isomers 1, 2, 3, and 4)

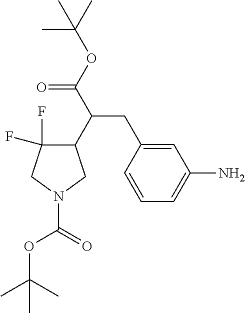 [SMILES: CC(C)(C)OC(=O)C(Cc1cccc(N)c1)C1CN(C(=O)OC(C)(C)C)CC1(F)F]

To a mixture of tert-butyl-4-[(1S)-1-[(3-bromophenyl)methyl]-2-tert-butoxy-2-oxo-ethyl]-3,3-difluoro-pyrrolidine-1-carboxylate (Isomer 1, 280 mg, 0.5710 mmol), bis(2,4-pentanedionato)copper(II) (15 mg, 0.057 mmol), 2,2,6,6-tetramethyl-3,5-heptanedione (50 μL, 0.24 mmol), Cs₂CO₃ (372 mg, 1.14 mmol) and DMA (1.2 mL, 13 mmol) NH40H (28 mass % in water, 1.12 mL, 6 mmol) was added and immediately gas evolution was observed. The reaction was sealed and heated at 110° C. overnight. The mixture was quenched with water and extracted with EtOAc. The combined organic phases were washed with saturated aqueous NaCl, dried over anhydrous MgSO₄, filtered and concentrated. The residue was purified by silica gel chromatography using a gradient of 70 to 90% MTBE in hexanes to give two diastereomeric products, likely resulting from epimerization during the reaction: First-eluting isomer—Isomer 1 (100 mg, 41%), colorless oil; second-eluting isomer—Isomer 2 (34 mg, 11%), colorless oil. Both Isomer 1 and Isomer 2: ES/MS m/z 449 (M+Na).

This procedure was repeated on tert-butyl-4-[(1S)-1-[(3-bromophenyl)methyl]-2-tert-butoxy-2-oxo-ethyl]-3,3-difluoro-pyrrolidine-1-carboxylate (Isomer 2) essentially as described above on the same scale, purifying the diastereomeric products by silica gel chromatography using 80% MTBE in hexanes to give: first-eluting isomer—Isomer 3 (77 mg, 28%), colorless oil; second-eluting isomer—Isomer 4 (93 mg, 36%), colorless oil. Both Isomer 3 and Isomer 4: ES/MS m/z 449 (M+Na).

### Preparation 89

### (2S)-2-[(3R)-1-tert-Butoxycarbonylpyrrolidin-3-yl]-3-(3-cyanophenyl)propanoic acid

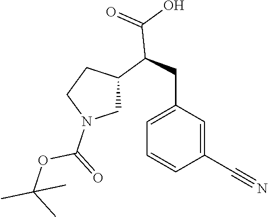 [SMILES: CC(C)(C)OC(=O)N1CC[C@H]([C@H](Cc2cccc(C#N)c2)C(=O)O)C1]

A mixture of (2S)-3-(3-bromophenyl)-2-[(3R)-1-tert-butoxycarbonylpyrrolidin-3-yl]propanoic acid (prepared essentially as described in WO 2020/247429; 600 mg, 1.506 mmol), potassium ferrocyanide trihydrate (277 mg, 0.753 mmol, 0.5 equiv), [(2-di-tert-butylphosphino-2′,4′,6′-triisopropyl-1,1′-biphenyl)-2-(2′-amino-1,1′-biphenyl)]palladium(II) methanesulfonate (tBuXPhos Pd G3, 24 mg, 0.030 mmol) and 2-di-tert-butylphosphino-2′,4′,6′-triisopropylbiphenyl in a sealed reaction vessel was evacuated and backfilled with nitrogen, repeating this process 3 times. To the vessel was then added 1,4-dioxane (2.5 mL) and a solution of potassium acetate (20 mg, 0.188 mmol) in degassed water (2.5 mL) via syringe. The mixture was heated to 100° C. for 1 h in a microwave. The reaction was filtered and concentrated in vacuo, and the residue to silica gel flash chromatography using a gradient of 50 to 100% EtOAc in hexanes to give the title compound (235 mg, 45%) as a colorless oil. ES/MS (m/z): 343 (M−H).

### Preparation 90

### (2S)-3-[3-(aminomethyl)phenyl]-2-[(3R)-1-tert-butoxycarbonylpyrrolidin-3-yl]propanoic acid

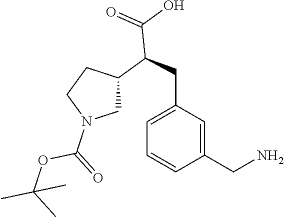 [SMILES: CC(C)(C)OC(=O)N1CC[C@H]([C@H](Cc2cccc(CN)c2)C(=O)O)C1]

Palladium (10% on carbon, 680 mg, 0.638 mmol) was added to a solution of (2S)-2-[(3R)-1-tert-butoxycarbonylpyrrolidin-3-yl]-3-(3-cyanophenyl)propanoic acid (220 mg, 0.638 mmol) in acetic acid (10 mL) in a pressure vessel. The mixture was purged with hydrogen and stirred under 551.7 Kpa of hydrogen at RT for 2 days. The mixture was filtered through diatomaceous earth and rinsed with MeOH. The filtrate was collected, and the solvent was evaporated under reduced pressure. The residue was purified by strong cation exchange (SCX) purification eluting with ammonia (2M in MeOH) to give the title compound (123 mg, 39%) which is 70% pure and used without further purification. ES/MS (m/z): 349 (M+H).

### Preparation 91

### tert-Butyl (3S)-3-[2-methoxy-1-(3-nitrophenoxy)-2-oxo-ethyl]pyrrolidine-1-carboxylate

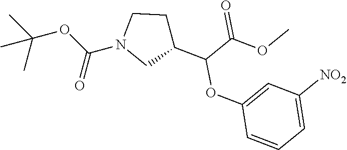 [SMILES: COC(=O)C(Oc1cccc([N+](=O)[O-])c1)[C@H]1CCN(C(=O)OC(C)(C)C)C1]

Diisopropylamine (1.4 mL, 9.9 mmol, 1.2 equiv) was added to a reaction vessel, chilled on an ice bath (0° C.) under inert atmosphere and n-butyl lithium (2.5 M solution in hexanes, 3.9 mL, 9.9 mmol) was slowly added. A white slurry formed after a few minutes. The slurry was diluted with anhydrous THE (20 mL). The solution was chilled to −78° C., tert-butyl (3R)-3-(2-methoxy-2-oxo-ethyl)pyrrolidine-1-carboxylate (prepared essentially as described in WO 2020/247429, 2.0 g, 8.2 mmol, 1 equiv) in a solution of THE (20 mL) was added and the mixture was stirred for 40 min at −78° C. Chlorotrimethylsilane (1.1 mL, 8.6 mmol) was added at −78° C. and the reaction was stirred for 20 min. N-bromosuccinimide (1.60 g, 8.6 mmol) was then added as a solid. The cooling bath was allowed to warm to RT over 18 h, then the reaction was diluted with EtOAc and washed with sodium thiosulfate (10% aqueous solution). The organic phase was dried over MgSO₄, filtered and concentrated to dryness under reduced pressure. The residue was purified by silica gel chromatography using a gradient of 0 to 50% EtOAc in hexanes to give the bromide intermediate [tert-butyl (3S)-3-(1-bromo-2-methoxy-2-oxo-ethyl)pyrrolidine-1-carboxylate] as a yellow oil. The bromide intermediate (1.84 g, 5.71 mmol) was dissolved in acetone (50 mL) and potassium carbonate (4.74 g, 34.3 mmol) and 3-nitrophenol (3.76 mL, 34.0 mmol) were added. The reaction mixture was stirred under inert atmosphere for 18 h, then diluted with EtOAc and washed with water and saturated aqueous NaCl. The organic phase was dried over MgSO₄, filtered and concentrated to dryness under reduced pressure. The residue was purified by silica gel chromatography eluting with 0 to 50% EtOAc in hexanes to give the title compound (300 mg, 14%) as a mixture of diastereomers. ES/MS (m/z): 325 (M+H-tert-butyl).

### Preparation 92

### tert-Butyl (3S)-3-[2-tert-butoxy-1-(3-nitrophenoxy)-2-oxo-ethyl]pyrrolidine-1-carboxylate (Isomer 1 and Isomer 2)

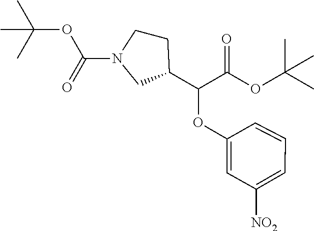 [SMILES: CC(C)(C)OC(=O)C(Oc1cccc([N+](=O)[O-])c1)[C@H]1CCN(C(=O)OC(C)(C)C)C1]

Sodium hydroxide (5 N aqueous solution, 8 mL, 40 mmol) was added to a solution of tert-butyl (3S)-3-[2-methoxy-1-(3-nitrophenoxy)-2-oxo-ethyl]pyrrolidine-1-carboxylate (1.24 g, 3.27 mmol) in MeOH (50 mL) and stirred for 18 h. The reaction mixture was concentrated under reduced pressure and the residue was diluted with DCM (100 mL) and citric acid (10% aqueous solution, 50 mL). The organic phase was collected, extracted with DCM (100 mL), and the combined organic extractions were concentrated under reduced pressure. To the residue was added toluene (20 mL) and the mixture was heated to 75° C. N,N-Dimethylformamide di-tert-butyl acetal (10 mL, 41 mmol) was added over 30 min and the reaction was allowed to stir at 75° C. for 4 h. The reaction was cooled to RT and concentrated under reduced pressure. The residue was purified by silica gel chromatography using a gradient of 0 to 100% EtOAc in hexanes to give the title compound (1.05 g) as a mixture of diastereomers. ES/MS (m/z): 311 (M+H-2×t-butyl).

The mixture of diastereomers was purified by chiral HPLC [column: Lux Cellulose-4 5×25 cm; mobile phase: 85:15 CO₂/IPA; 1.05 g dissolved in IPA to give a 50 mg/mL solution, 50 mg injected every 1.9 min] to give Isomer 1 (first-eluting isomer, 376 mg, 27%, >99% de) and Isomer 2 (second-eluting isomer, 487 mg, 35%, >99% de)

### Preparation 93

### tert-Butyl (3S)-3-[1-(3-aminophenoxy)-2-tert-butoxy-2-oxo-ethyl]pyrrolidine-1-carboxylate (Isomer 2)

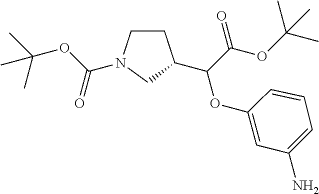 [SMILES: CC(C)(C)OC(=O)C(Oc1cccc(N)c1)[C@H]1CCN(C(=O)OC(C)(C)C)C1]

To a mixture of tert-butyl (3S)-3-[2-tert-butoxy-1-(3-nitrophenoxy)-2-oxo-ethyl]pyrrolidine-1-carboxylate (Isomer 2, 486 mg, 1.15 mmol) in EtOH (20 mL) was added Pd (5 mass % on carbon, 250 mg, 0.117 mmol). The slurry was stirred under hydrogen gas for 4 h, then filtered and concentrated to give the title compound (410 mg, 91%) as an oil which was carried forward without characterization.

### Preparation 94

### tert-Butyl (3R)-3-[(1S)-2-tert-butoxy-1-[[3-[[3-[(2S)-3-tert-butoxy-2-[(3R)-1-tert-butoxycarbonylpyrrolidin-3-yl]-3-oxo-propyl]phenyl]carbamoylamino]phenyl]methyl]-2-oxo-ethyl]pyrrolidine-1-carboxylate

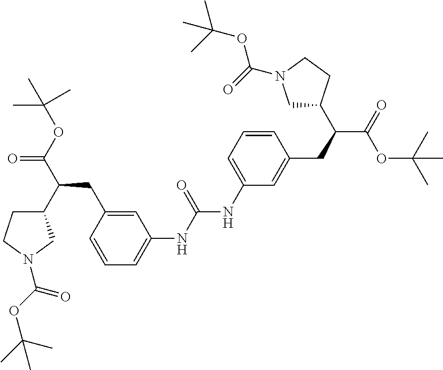 [SMILES: CC(C)(C)OC(=O)[C@@H](Cc1cccc(NC(=O)Nc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

A mixture of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 57.80 g, 148.0 mmol) and 1,1′-carbonyldiimidazole (12 g, 74 mmol) in 2-methyltetrahydrofuran (240 mL) was stirred at 70° C. overnight. The mixture was cooled to RT and then water (250 mL) and 2-methyltetrahydrofuran (250 mL) were added. The layers were separated, and the organic layer was dried over MgSO₄, filtered, and concentrated. The residue was triturated at 60° C. in a mixture of 1:1 IPA/water (600 mL) for 4 h and then at RT overnight. The resulting solid was filtered, washed with 1:1 IPA/water, and dried under reduced pressure at 40° C. to give the title compound (58 g, 97%). ES/MS (m/z): 706 (M+H-BOC). Analytical chiral SFC [column: Chiralpak IB 4.6×100 mm, 5 μm; mobile phase: solvent A=MeOH+0.2% isopropylamine; flow rate 4 mL/min; column temperature 40° C.] shows de >98%.

The following were prepared essentially as described in Preparation 94 using the appropriate amine:

| | | | | Purifi- | Physical |
|---|---|---|---|---|---|
| | | | Proce- | cation | Data |
| Prepa- | | | dure | modifi- | ES-MS |
| ration | Chemical name | Structure | notes | cations | m/z |
| 95 | tert-Butyl (3S)-3-[(1R)- 2-tert-butoxy-1-[[3-[[3- [(2R)-3-tert-butoxy-2- [(3S)-1-tert- butoxycarbonylpyrrolidin- 3-yl]-3-oxo- propyl]phenyl] carbamoylamino] phenyl]methyl]- 2-oxo-ethyl]pyrrolidine- 1-carboxylate | 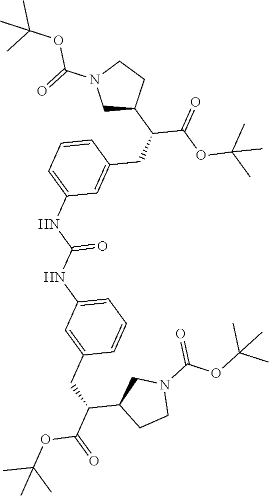 [SMILES: CC(C)(C)OC(=O)[C@H](Cc1cccc(NC(=O)Nc2cccc(C[C@@H](C(=O)OC(C)(C)C)[C@@H]3CCN(C(=O)OC(C)(C)C)C3)c2)c1)[C@@H]1CCN(C(=O)OC(C)(C)C)C1] | | A, B | 707 (M + H—BOC) |
| 96 | (2S)-3-[3-[[3-[(2S)-3- tert-Butoxy-2-[(3R)-1- tert- butoxycarbonylpyrrolidin- 3-yl]-3-oxo- propyl]phenyl] carbamoylamino] methyl]phenyl]- 2-[(3R)-1-tert- butoxycarbonylpyrrolidin- 3-yl]propanoic acid | 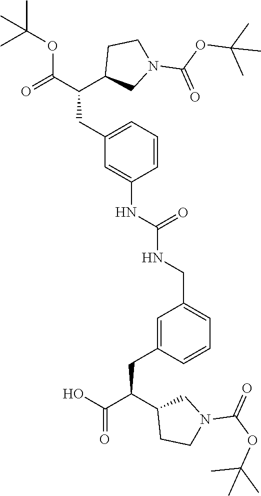 [SMILES: CC(C)(C)OC(=O)[C@@H](Cc1cccc(NC(=O)NCc2cccc(C[C@H](C(=O)O)[C@H]3CCN(C(=O)OC(C)(C)C)C3)c2)c1)[C@H]1CCN(C(=O)OC(C)(C)C)C1] | C | B | 765 (M + H) |
| 97 | tert-Butyl (3R)-3-[(1S)- 2-tert-butoxy-1-[[3-[[3- [(2S)-3-tert-butoxy-2- [(3R)-1-tert- butoxycarbonylpyrrolidin- 3-yl]-3-oxo-propyl]-5- fluoro-phenyl] carbamoylamino]- 5-fluoro- phenyl]methyl]-2-oxo- ethyl]pyrrolidine-1- carboxylate | 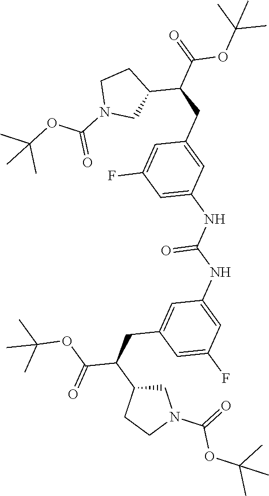 [SMILES: CC(C)(C)OC(=O)[C@@H](Cc1cc(F)cc(NC(=O)Nc2cc(F)cc(C[C@H](C(=O)OC(C)(C)C)[C@H]3CCN(C(=O)OC(C)(C)C)C3)c2)c1)[C@H]1CCN(C(=O)OC(C)(C)C)C1] | D | A | 743 (M + H—BOC) |
| 98 | tert-Butyl (3R)-3-[(1S)- 2-tert-butoxy-1-[[3-[[3- [(2S)-3-tert-butoxy-2- [(3R)-1-tert- butoxycarbonylpyrrolidin- 3-yl]-3-oxo-propyl]- N-[[3-[(2S)-3-tert- butoxy-2-[(3R)-1-tert- butoxycarbonylpyrrolidin- 3-yl]-3-oxo- propyl]phenyl]carbamoyl] anilino]methyl]phenyl] methyl]-2-oxo- ethyl]pyrrolidine-1- carboxylate | 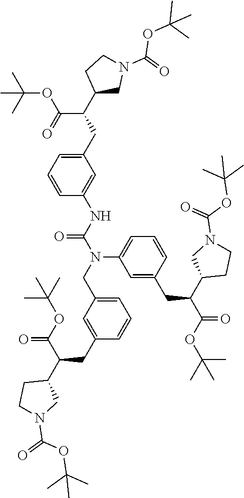 [SMILES: CC(C)(C)OC(=O)[C@@H](Cc1cccc(CN(C(=O)Nc2cccc(C[C@H](C(=O)OC(C)(C)C)[C@H]3CCN(C(=O)OC(C)(C)C)C3)c2)c2cccc(C[C@H](C(=O)OC(C)(C)C)[C@H]3CCN(C(=O)OC(C)(C)C)C3)c2)c1)[C@H]1CCN(C(=O)OC(C)(C)C)C1] | C | E | M |
| 99 | tert-Butyl 4-[2-tert- butoxy-1-[[3-[[3-[3-tert- butoxy-2-(1-tert- butoxycarbonyl-4,4- difluoro-pyrrolidin-3- yl)-3-oxo- propyl]phenyl] carbamoylamino] phenyl]methyl]- 2-oxo-ethyl]-3,3- difluoro-pyrrolidine-1- carboxylate (Isomer 1) | 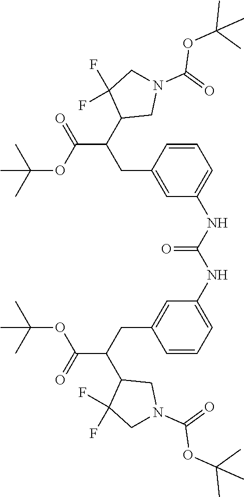 [SMILES: CC(C)(C)OC(=O)C(Cc1cccc(NC(=O)Nc2cccc(CC(C(=O)OC(C)(C)C)C3CN(C(=O)OC(C)(C)C)CC3(F)F)c2)c1)C1CN(C(=O)OC(C)(C)C)CC1(F)F] | G, D | A | 779 (M + H—BOC) |
| 100 | tert-Butyl 4-[2-tert- butoxy-1-[[3-[3-[3-tert- butoxy-2-(1-tert- butoxycarbonyl-4,4- difluoro-pyrrolidin-3- yl)-3-oxo- propyl]phenyl] carbamoylamino] phenyl]methyl]- 2-oxo-ethyl]-3,3- difluoro-pyrrolidine-1- carboxylate (Isomer 2) | 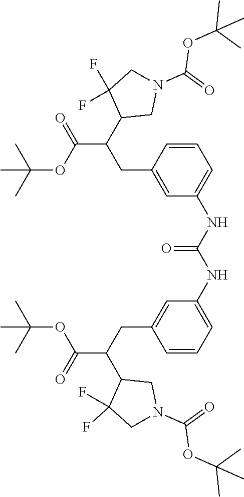 [SMILES: CC(C)(C)OC(=O)C(Cc1cccc(NC(=O)Nc2cccc(CC(C(=O)OC(C)(C)C)C3CN(C(=O)OC(C)(C)C)CC3(F)F)c2)c1)C1CN(C(=O)OC(C)(C)C)CC1(F)F] | H, D | A | 779 (M + H—BOC) |
| 101 | tert-Butyl 4-[2-tert- butoxy-1-[[3-[[3-[3-tert- butoxy-2-(1-tert- butoxycarbonyl-4,4- difluoro-pyrrolidin-3- yl)-3-oxo- propyl]phenyl] carbamoylamino] phenyl]methyl]- 2-oxo-ethyl]-3,3- difluoro-pyrrolidine-1- carboxylate (Isomer 3) | 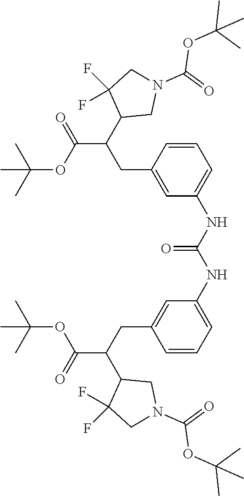 [SMILES: CC(C)(C)OC(=O)C(Cc1cccc(NC(=O)Nc2cccc(CC(C(=O)OC(C)(C)C)C3CN(C(=O)OC(C)(C)C)CC3(F)F)c2)c1)C1CN(C(=O)OC(C)(C)C)CC1(F)F] | I, D | A | 779 (M + H—BOC) |
| 102 | tert-Butyl 4-[2-tert- butoxy-1-[3-[[3-[3-tert- butoxy-2-(1-tert- butoxycarbonyl-4,4- difluoro-pyrrolidin-3- yl)-3-oxo- propyl]phenyl] carbamoylamino] phenyl]methyl]- 2-oxo-ethyl]-3,3- difluoro-pyrrolidine-1- carboxylate (Isomer 4) | 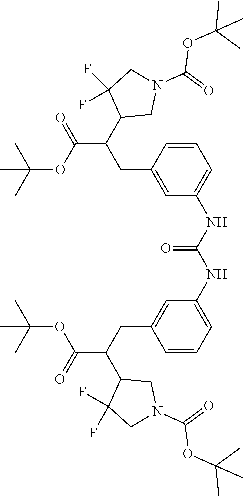 [SMILES: CC(C)(C)OC(=O)C(Cc1cccc(NC(=O)Nc2cccc(CC(C(=O)OC(C)(C)C)C3CN(C(=O)OC(C)(C)C)CC3(F)F)c2)c1)C1CN(C(=O)OC(C)(C)C)CC1(F)F] | J, D | A | 779 (M + H—BOC) |
| 103 | tert-Butyl (3S)-3-[2-tert- butoxy-1-[3-[[3-[2-tert- butoxy-1-[(3S)-1-tert- butoxycarbonylpyrrolidin- 3-yl]-2-oxo- ethyl]sulfanylphenyl] carbamoylamino]phenyl] sulfanyl-2-oxo- ethyl]pyrrolidine-1- carboxylate (Isomer 1) | 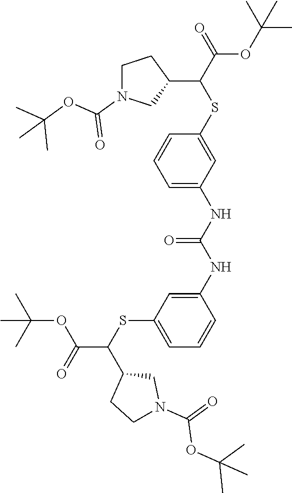 [SMILES: CC(C)(C)OC(=O)C(Sc1cccc(NC(=O)Nc2cccc(SC(C(=O)OC(C)(C)C)[C@H]3CCN(C(=O)OC(C)(C)C)C3)c2)c1)[C@H]1CCN(C(=O)OC(C)(C)C)C1] | G | F | 743 (M + H—BOC) |
| 104 | tert-Butyl (3S)-3-[2-tert- butoxy-1-[3-[[3-[2-tert- butoxy-1-[(3S)-1-tert- butoxycarbonylpyrrolidin- 3-yl]-2-oxo- ethyl]sulfanylphenyl] carbamoylamino]phenyl] sulfanyl-2-oxo- ethyl]pyrrolidine-1- carboxylate (Isomer 2) | 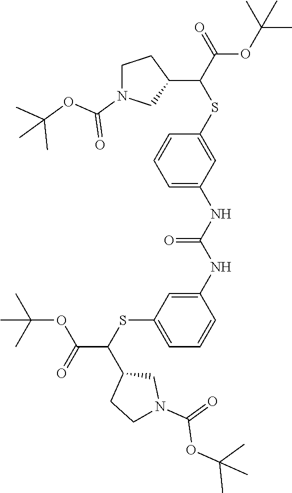 [SMILES: CC(C)(C)OC(=O)C(Sc1cccc(NC(=O)Nc2cccc(SC(C(=O)OC(C)(C)C)[C@H]3CCN(C(=O)OC(C)(C)C)C3)c2)c1)[C@H]1CCN(C(=O)OC(C)(C)C)C1] | H | B | 743 (M + H—BOC) |
| 105 | tert-Butyl (3R)-3-[2-tert- butoxy-1-[3-[[3-[3-tert- butoxy-2-[(3R)-1-tert- butoxycarbonylpyrrolidin- 3-yl]-2-methyl-3-oxo- propyl]phenyl] carbamoylamino] phenyl]methyl]- 1-methyl-2-oxo- ethyl]pyrrolidine-1- carboxylate (Isomer 2) | 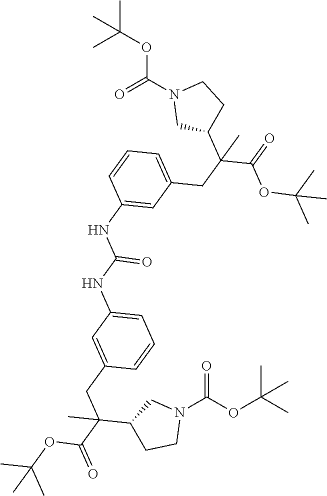 [SMILES: CC(C)(C)OC(=O)N1CC[C@H](C(C)(Cc2cccc(NC(=O)Nc3cccc(CC(C)(C(=O)OC(C)(C)C)[C@H]4CCN(C(=O)OC(C)(C)C)C4)c3)c2)C(=O)OC(C)(C)C)C1] | H, D | | 735 (M + H—BOC) |
| 106 | tert-butyl (3R)-3-[(1S)- 2-tert-butoxy-1-[[3-[[[3- [(2S)-3-tert-butoxy-2- [(3R)-1-tert- butoxycarbonylpyrrolidin- 3-yl]-3-oxo- propyl]phenyl]carbamoyl- [[3-[(2S)-3-tert- butoxy-2-[(3R)-1-tert- butoxycarbonylpyrrolidin- 3-yl]-3-oxo- propyl]phenyl]methyl] amino]methyl]phenyl] methyl]-2-oxo- ethyl ]pyrrolidine-1- carboxylate | 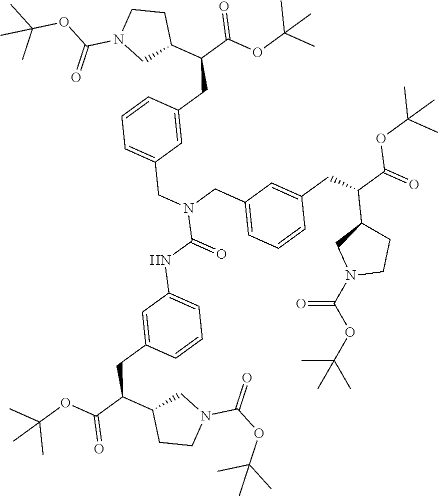 [SMILES: CC(C)(C)OC(=O)[C@@H](Cc1cccc(CN(Cc2cccc(C[C@H](C(=O)OC(C)(C)C)[C@H]3CCN(C(=O)OC(C)(C)C)C3)c2)C(=O)Nc2cccc(C[C@H](C(=O)OC(C)(C)C)[C@H]3CCN(C(=O)OC(C)(C)C)C3)c2)c1)[C@H]1CCN(C(=O)OC(C)(C)C)C1] | K, D | A, B | L |

A Purification by silica gel chromatography eluting with EtOAc:hexanes
B Purification by reverse-phase flash chromatography; mobile phase: ACN in aqueous NH₄CO₃ pH 9
C tert-butyl (3R)-3-[(1S)-1-[(3-aminophenyl)methyl]-2-tert-butoxy-2-oxo-ethyl]pyrrolidine-1-carboxylate prepared essentially as described in WO 2020/247429
D Upon completion the reaction mixture was concentrated in vacuo and no aqueous workup was performed.
E Purification by silica gel chromatography eluting with acetone:DCM
F Purification by silica gel chromatography eluting with acetone:hexanes
G Prepared using Isomer 1 of the amine starting material
H Prepared using Isomer 2 of the amine starting material
I Prepared using Isomer 3 of the amine starting material
J Prepared using Isomer 4 of the amine starting material
K Starting material tert-butyl (3R)-3-[(1S)-2-tert-butoxy-1-[[3-[[[3-[(2S)-3-tert-butoxy-2-[(3R)-1-tert-butoxycarbonylpyrrolidin-3-yl]-3-oxo-propyl]phenyl]methylamino]methyl]phenyl]
methyl]-2-oxo-ethyl]pyrrolidine-1-carboxylate prepared essentially as described in WO 2020/247429
L No MS data available; ¹H NMR (400.21 MHz, CDCl₃) δ 7.28 (m, 12H), 4.60-4.56 (m, 4H), 3.72-3.62 (m, 6H), 3.31-3.22 (m, 3H), 3.05-2.95 (m, 9H), 2.51-2.43 (m, 6H), 1.99-1.91 (m, 3H), 1.74-1.70 (m, 3H), 1.48 (s, 27H), 1.30-1.28 (m, 27H).
M No MS data, ¹H NMR (400.13 MHz, DMSO-d₆) δ 7.55-7.48 (m, 1H), 7.32-7.23 (m, 2H), 7.22-7.14 (m, 2H), 7.14-6.98 (m, 6H), 6.77 (d, J = 7.7 Hz, 1H), 5.76 (d, J = 1.3 Hz, 1H), 4.89-4.76 (m, 2H), 3.56-3.41 (m, 3H), 3.39-3.27 (m, 3H), 3.20-3.06 (m, 3H), 2.99-2.88 (m, 3H), 2.80-2.61 (m, 6H), 2.58-2.37 (m, 3H), 2.35-2.17 (m, 3H), 1.90-1.76 (m, 3H), 1.66-1.49 (m, 3H), 1.39 (s, 27H), 1.24-1.21 (m, 27H)

### Preparation 107

### tert-Butyl (3S)-3-[(1S)-2-tert-butoxy-1-[3-[[3-[(1S)-2-tert-butoxy-1-[(3S)-1-tert-butoxycarbonylpyrrolidin-3-yl]-2-oxo-ethoxy]phenyl]carbamoylamino]phenoxy]-2-oxo-ethyl]pyrrolidine-1-carboxylate, Isomer 2

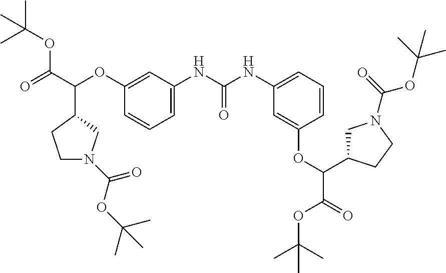 [SMILES: CC(C)(C)OC(=O)C(Oc1cccc(NC(=O)Nc2cccc(OC(C(=O)OC(C)(C)C)[C@H]3CCN(C(=O)OC(C)(C)C)C3)c2)c1)[C@H]1CCN(C(=O)OC(C)(C)C)C1]

tert-Butyl (3S)-3-[1-(3-aminophenoxy)-2-tert-butoxy-2-oxo-ethyl]pyrrolidine-1-carboxylate (Isomer 2, 410 mg, 1.045 mmol) was dissolved in THE (12 mL), then CDI (102 mg, 0.63 mmol) and DMAP (26 mg, 0.20 mmol) were added to the mixture. The reaction was stirred at RT for 56 h, then additional CDI (160 mg, 0.987 mmol) was added and the mixture was stirred at 40° C. for 18 h. The reaction mixture was concentrated to dryness under reduced pressure and the residue was purified by silica gel chromatography using a gradient of 0 to 50% EtOAc in hexanes to give the title compound (200 mg, 24%) as a clear glassy solid, which was carried forward without characterization.

The following were prepared essentially as described in Preparation 107 using the appropriate amine:

| | | | Procedure | Purification | Physical Data |
|---|---|---|---|---|---|
| Preparation | Chemical name | Structure | notes | modifications | ES-MS m/z |
| 108 | tert-Butyl (3S)-3-[2-tert- butoxy-1-[[3-[[3-[3-tert- butoxy-2-[(3S)-1-tert- butoxycarbonylpyrrolidin- 3-yl]-2-hydroxy-3- oxo- propyl]phenyl] carbamoylamino] phenyl]methyl]- 1-hydroxy-2-oxo- ethyl]pyrrolidine-1- carboxylate (Isomer 1) | 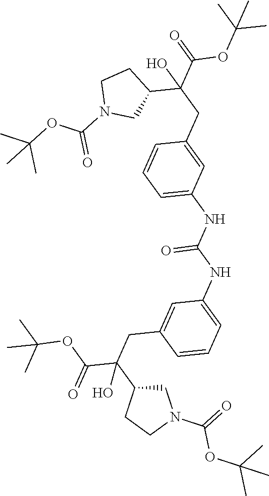 [SMILES: CC(C)(C)OC(=O)N1CC[C@H](C(O)(Cc2cccc(NC(=O)Nc3cccc(CC(O)(C(=O)OC(C)(C)C)[C@H]4CCN(C(=O)OC(C)(C)C)C4)c3)c2)C(=O)OC(C)(C)C)C1] | A | | not available |
| 109 | tert-Butyl (2R,4R)-4- [(1S)-2-tert-butoxy-1- [[3-[[3-[(2S)-3-tert- butoxy-2-[(3R,5R)-1- tert-butoxycarbonyl-5- methyl-pyrrolidin-3-yl]- 3-oxo- propyl]phenyl] carbamoylamino] phenyl]methyl]- 2-oxo-ethyl]-2-methyl- pyrrolidine-1- carboxylate | 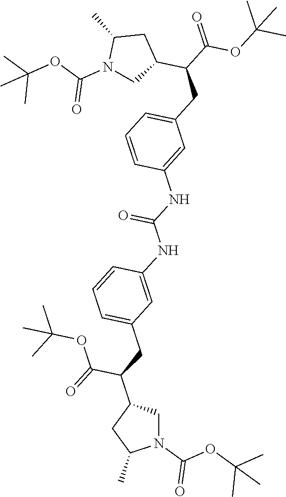 [SMILES: C[C@@H]1C[C@H]([C@H](Cc2cccc(NC(=O)Nc3cccc(C[C@H](C(=O)OC(C)(C)C)[C@H]4C[C@@H](C)N(C(=O)OC(C)(C)C)C4)c3)c2)C(=O)OC(C)(C)C)CN1C(=O)OC(C)(C)C] | B | | 835.5 (M + H) |

A A: Prepared using Isomer 1 of the amine starting material
B B: Upon completion, the reaction was diluted with EtOAc, washed with saturated aqueous NaHCO₃, saturated aqueous NaCl, dried over Na₂SO₄, filtered, and concentrated before purification.

### Preparation 110

(2 S)-2-[(3R)-1-tert-Butoxycarbonylpyrrolidin-3-yl]-3-[3-[3-[3-[(2 S)-2-[(3R)-1-tert-butoxycarbonylpyrrolidin-3-yl]-2-carboxy-ethyl]phenyl]-2-oxo-hexahydropyrimidin-1l-yl]phenyl]propanoic acid

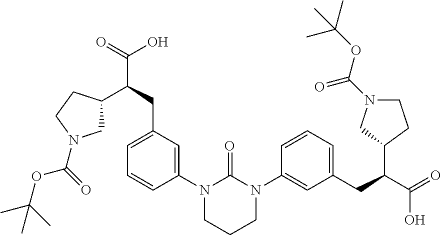 [SMILES: CC(C)(C)OC(=O)N1CC[C@H]([C@H](Cc2cccc(N3CCCN(c4cccc(C[C@H](C(=O)O)[C@H]5CCN(C(=O)OC(C)(C)C)C5)c4)C3=O)c2)C(=O)O)C1]

A mixture of (2S)-3-(3-bromophenyl)-2-[(3R)-1-tert-butoxycarbonylpyrrolidin-3-yl]propanoic acid (prepared essentially as described in WO 2020/247429; 347 mg, 0.872 mmol), tetrahydro-2(1H)-pyrimidinone (45 mg, 0.436 mmol), sodium tert-butoxide (126 mg, 1.31 mmol), tBuXPhos Pd G3 (35 mg, 0.043 mmol) and 1,4-dioxane (4.36 mL) was purged with nitrogen for 10 min, then the reaction vessel was sealed and heated at 100° C. for 16 h. Additional (2S)-3-(3-bromophenyl)-2-[(3R)-1-tert-butoxycarbonylpyrrolidin-3-yl]propanoic acid (174 mg, 0.436 mmol), sodium tert-butoxide (126 mg, 1.31 mmol) and tBuXPhos Pd G3 (35 mg, 0.043 mmol) were added, then the mixture was purged with nitrogen for 10 min, sealed and heated at 100° C. for 16 h. Additional sodium tert-butoxide (126 mg, 1.31 mmol) and tBuXPhos Pd G3 (35 mg, 0.043 mmol) were added, then the mixture was purged with nitrogen for 10 min, seal and heated at 100° C. for 2.5 h. The reaction was filtered through a pad of diatomaceous earth, washing with DCM and MeOH. The filtrate was concentrated under reduced pressure and the residue was purified by reverse phase flash chromatography using a gradient of 20 to 50% ACN in aqueous NH₄CO₃ (pH 9) to give the title compound (155 mg, 48%). ES/MS (m/z): 735 (M+H).

The following were prepared essentially as described in Preparation 110 using the appropriate aryl halide and imidazolidin-2-one.

| | | | Procedure | Purification | Physical Data |
|---|---|---|---|---|---|
| Preparation | Chemical name | Structure | notes | modifications | ES-MS m/z |
| 111 | (2S)-2-[(3R)-1-tert- Butoxycarbonylpyrrolidin- 3-yl]-3-[3-[3-[3-[(2S)- 2-[(3R)-1-tert- butoxycarbonylpyrrolidin- 3-yl]-2-carboxy- ethyl]phenyl]-2-oxo- imidazolidin-1- yl]phenyl]propanoic acid | 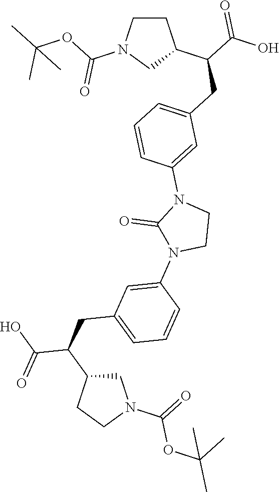 [SMILES: CC(C)(C)OC(=O)N1CC[C@H]([C@H](Cc2cccc(N3CCN(c4cccc(C[C@H](C(=O)O)[C@H]5CCN(C(=O)OC(C)(C)C)C5)c4)C3=O)c2)C(=O)O)C1] | | | 621 (M + H—BOC) |
| 112 | (2S)-2-(1-tert- Butoxycarbonyl-4,4- difluoro-pyrrolidin-3- yl)-3-[3-[3-[3-[(2S)-2- (1-tert-butoxycarbonyl- 4,4-difluoro-pyrrolidin- 3-yl)-2-carboxy- ethyl]phenyl]-2-oxo- imidazolidin-1- yl]phenyl]propanoic acid (Isomer 2) | 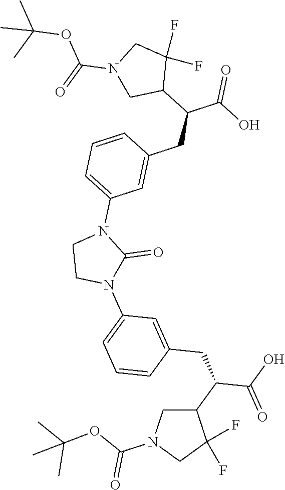 [SMILES: CC(C)(C)OC(=O)N1CC([C@H](Cc2cccc(N3CCN(c4cccc(C[C@H](C(=O)O)C5CN(C(=O)OC(C)(C)C)CC5(F)F)c4)C3=O)c2)C(=O)O)C(F)(F)C1] | A, B | C | 693 (M + H—BOC) |

A Isomer 2 of the starting aryl bromide was used
B Product precipitated from the reaction mixture using a mixture of MTBE and hexane
C Purified by silica gel chromatography using a gradient of 10 to 40% EtOH in hexanes + 1% acetic acid

### Preparation 113

### tert-butyl (3R)-3-[(1S)-2-tert-butoxy-1-[[3-[(2-chloroacetyl)amino]phenyl]methyl]-2-oxo-ethyl]pyrrolidine-1-carboxylate

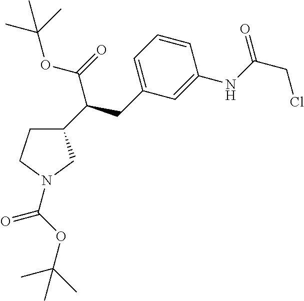 [SMILES: CC(C)(C)OC(=O)[C@@H](Cc1cccc(NC(=O)CCl)c1)[C@H]1CCN(C(=O)OC(C)(C)C)C1]

TEA (1.5 equiv., 0.107 mL, 0.7682 mmol) was added at 0° C. to a mixture of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429, 200 mg, 0.5122 mmol) in DCM (0.16 M, 3.201 mL). This was stirred at this temperature for 10 min and then chloroacetyl chloride (1.1 equiv., 0.04478 mL, 0.5634 mmol) was added. The mixture was stirred at RT overnight. NaHCO₃ (sat) (5 mL) was added, the aqueous layer was extracted with DCM. The organic layer was washed with saturated aqueous NaCl and filtered through diatomaceous earth. The solution was dried over MgSO₄ and the solvent was eliminated under reduced pressure to obtain the title compound (238 g, 99%). ES/MS (m/z): 368 (M+H-BOC).

### Preparation 114

### tert-Butyl (3R)-3-[(1S)-2-tert-butoxy-1-[[3-[4-[3-[(2S)-3-tert-butoxy-2-[(3R)-1-tert-butoxycarbonylpyrrolidin-3-yl]-3-oxo-propyl]phenyl]-2,5-dioxo-piperazin-1-yl]phenyl]methyl]-2-oxo-ethyl]pyrrolidine-1-carboxylate

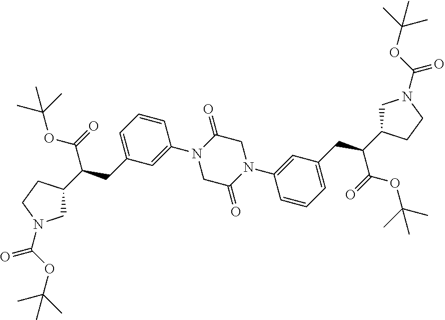 [SMILES: CC(C)(C)OC(=O)[C@@H](Cc1cccc(N2CC(=O)N(c3cccc(C[C@H](C(=O)OC(C)(C)C)[C@H]4CCN(C(=O)OC(C)(C)C)C4)c3)CC2=O)c1)[C@H]1CCN(C(=O)OC(C)(C)C)C1]

Potassium carbonate (0.1409 g, 1.019 mmol) was added to a solution of tert-butyl (3R)-3-[(1S)-2-tert-butoxy-1-[[3-[(2-chloroacetyl)amino]phenyl]methyl]-2-oxo-ethyl]pyrrolidine-1-carboxylate (238 mg, 0.5096 mmol) in ACN (5 mL) and the mixture was stirred at 80° C. overnight. The reaction was cooled to RT, quenched with water, then extracted EtOAc. The organic layer was washed with saturated aqueous NaCl and filtered through diatomaceous earth. The solution was dried over MgSO₄, filtered and concentrated under reduced pressure. The residue was purified by reverse phase HPLC [column: XBridge® C18 19×100 mm, 5 μm; mobile phase: solvent A—aqueous NH₄HCO₃ (20 mM, pH9), solvent B—ACN; flow rate: 25 mL/min] to give the title compound (63 mg, 14%) as a colorless oil. ES/MS (m/z): 762 (M+H-BOC).

### Preparation 115

### tert-Butyl (3R)-3-[(1S)-2-tert-butoxy-1-[[3-[[3-[(2S)-3-tert-butoxy-2-[(3R)-1-tert-butoxycarbonylpyrrolidin-3-yl]-3-oxo-propyl]-N-carbamoyl-anilino]methyl]phenyl]methyl]-2-oxo-ethyl]pyrrolidine-1-carboxylate

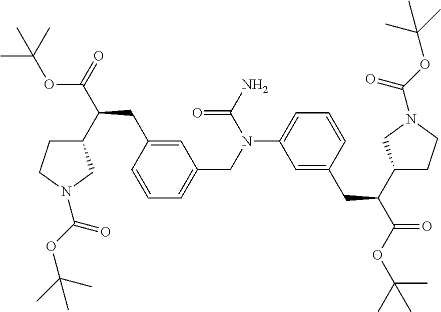 [SMILES: CC(C)(C)OC(=O)[C@@H](Cc1cccc(CN(C(N)=O)c2cccc(C[C@H](C(=O)OC(C)(C)C)[C@H]3CCN(C(=O)OC(C)(C)C)C3)c2)c1)[C@H]1CCN(C(=O)OC(C)(C)C)C1]

A solution of potassium cyanate (115 mg, 1.420 mmol) in water (1.82 mL) was added to a solution of tert-butyl (3R)-3-[(1S)-2-tert-butoxy-1-[[3-[[3-[(2S)-3-tert-butoxy-2-[(3R)-1-tert-butoxycarbonylpyrrolidin-3-yl]-3-oxo-propyl]anilino]methyl]phenyl]methyl]-2-oxo-ethyl]pyrrolidine-1-carboxylate (prepared essentially as described in WO 2020/247429; 260 mg, 0.284 mmol) in acetic acid (2.27 mL) at RT and the mixture was stirred for 30 min. Water was added and the aqueous layer was extracted with EtOAc. The organic phase was washed with saturated aqueous NaHCO₃. The organic layer was dried over MgSO₄, filtered and concentrated under reduced pressure. The residue was purified by silica gel chromatography using a gradient of 15 to 100% MTBE in hexanes to give the title compound (212 mg, 87%). ES/MS (m/z): 821 (M+H).

### Preparation 116

### (2S)-2-[(3R)-1-tert-Butoxycarbonylpyrrolidin-3-yl]-3-[3-[3-[3-[(2S)-2-[(3R)-1-tert-butoxycarbonylpyrrolidin-3-yl]-2-carboxy-ethyl]phenyl]-2-oxo-benzimidazol-1-yl]phenyl]propanoic acid

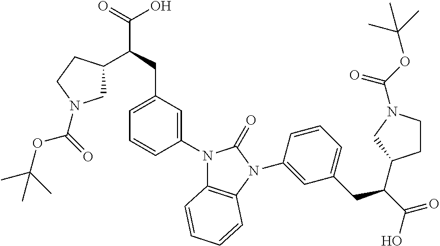 [SMILES: CC(C)(C)OC(=O)N1CC[C@H]([C@H](Cc2cccc(-n3c(=O)n(-c4cccc(C[C@H](C(=O)O)[C@H]5CCN(C(=O)OC(C)(C)C)C5)c4)c4ccccc43)c2)C(=O)O)C1]

A mixture of (2S)-3-(3-bromophenyl)-2-[(3R)-1-tert-butoxycarbonylpyrrolidin-3-yl]propanoic acid (prepared essentially as described in WO 2020/247429; 400 mg, 1.004 mmol), 1,3-dihydrobenzimidazol-2-one (67 mg, 0.5021 mmol, 0.5 equiv), potassium carbonate (486 mg, 3.515 mmol, 3.5 equiv), cuprous iodide (29 mg, 1.506 mmol, 1.5 equiv), N,N′-dimethylethylenediamine (0.322 mL, 3.013 mmol, 3 equiv) and anhydrous toluene (1.5 mL, 1.5 mL/mmol) was purged with nitrogen for 10 min, then the reaction vessel was sealed and heated at 100° C. for 4 h. The reaction mixture was filtered through a pad of diatomaceous earth, then the pad was washed with DCM and MeOH. The filtrate was concentrated under reduced pressure and the resulting residue was purified by reverse phase chromatography using a gradient of 20 to 50% ACN in aqueous NH₄CO₃ (pH 9) to give a blue solid. The solid was dissolved in MeOH (12 mL), then SiliaMetS® Thiol metal scavenger (1.2 g) was added and the mixture was stirred at RT for 18 h. The suspension was filtered and washed with MeOH. The filtrate was concentrated under reduced pressure to give the title compound (183 mg, 24%). ES/MS (m/z): 769 (M+H).

### Preparation 117

### (2S)-2-[(3R)-1-tert-Butoxycarbonylpyrrolidin-3-yl]-3-[3-(4,4,5,5-tetramethyl-1,3,2-dioxaborolan-2-yl)phenyl]propanoic acid

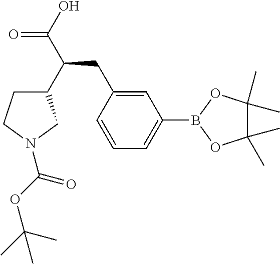

A mixture of (2S)-3-(3-bromophenyl)-2-[(3R)-1-tert-butoxycarbonylpyrrolidin-3-yl]propanoic acid ammonium salt (see Preparation 24; 2 g, 4.82 mmol), bis(pinacolato)diboron (1.85 g, 7.22 mmol), potassium acetate (0.945 g, 9.63 mmol), 1,1′-bis(diphenylphosphino)ferrocene-palladium(II)dichloride dichloromethane complex (0.286 g, 0.337 mmol) and anhydrous 1,4-dioxane (12 mL) was purged with nitrogen for 10 min and stirred at 90° C. overnight. The reaction was cooled to RT, then water was added and the aqueous layer was extracted with EtOAc. The organic layer was washed with saturated aqueous NaCl and filtered through a pad of diatomaceous earth. The filtrate was dried over MgSO₄, filtered, concentrated under reduced pressure, then purified by silica gel chromatography using a gradient of 0 to 100% acetone in hexanes to give the title compound (2.14 g, 100%). ES/MS (m/z): 346 (M+H-BOC).

### Preparation 118

### (2R)-2-[(3S)-1-tert-Butoxycarbonylpyrrolidin-3-yl]-3-[3-(4,4,5,5-tetramethyl-1,3,2-dioxaborolan-2-yl)phenyl]propanoic acid

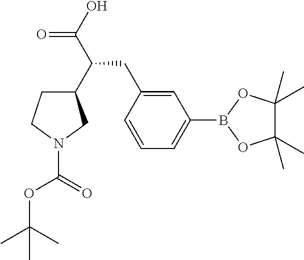 [SMILES: CC(C)(C)OC(=O)N1CC[C@@H]([C@@H](Cc2cccc(B3OC(C)(C)C(C)(C)O3)c2)C(=O)O)C1]

The title compound was prepared essentially as described in Preparation 117 using (2R)-3-(3-bromophenyl)-2-[(3S)-1-tert-butoxycarbonylpyrrolidin-3-yl]propanoic acid ammonium salt. ES/MS (m/z): 346 (M+H-BOC).

### Preparation 119

### (2S)-2-[(3R)-1-tert-Butoxycarbonylpyrrolidin-3-yl]-3-[3-[3-[(2S)-2-[(3R)-1-tert-butoxycarbonylpyrrolidin-3-yl]-2-carboxy-ethyl]phenyl]phenyl]propanoic acid

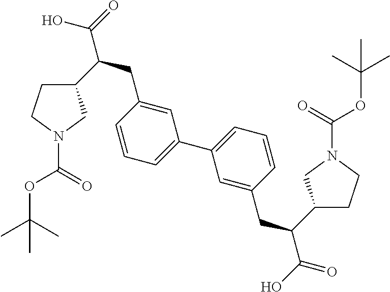 [SMILES: CC(C)(C)OC(=O)N1CC[C@H]([C@H](Cc2cccc(-c3cccc(C[C@H](C(=O)O)[C@H]4CCN(C(=O)OC(C)(C)C)C4)c3)c2)C(=O)O)C1]

A mixture of (2S)-3-(3-bromophenyl)-2-[(3R)-1-tert-butoxycarbonylpyrrolidin-3-yl]propanoic acid ammonium salt (see Preparation 24; 500 mg, 1.20 mmol) in 1,4-dioxane (12 mL) and water (1.2 mL), (2S)-2-[(3R)-1-tert-butoxycarbonylpyrrolidin-3-yl]-3-[3-(4,4,5,5-tetramethyl-1,3,2-dioxaborolan-2-yl)phenyl]propanoic acid (0.804 g, 1.81 mmol), 1,1′-bis(diphenylphosphino)ferrocene-palladium(II)dichloride dichloromethane complex (0.201 g, 0.241 mmol) and K₂CO₃ (0.499 g, 3.61 mmol) was stirred at 110° C. overnight under nitrogen. The reaction was cooled, filtered through a pad of diatomaceous earth and washed with DCM and MeOH. The filtrate was concentrated under reduced pressure and the residue was purified by reverse phase HPLC [column: XBridge® C18 (19×100 mm, 5 m); mobile phase: solvent A—aqueous NH₄HCO₃ (20 mM, pH9), solvent B—ACN, 1:3 B:A; flow rate: 25 mL/min] to give the title compound (442 mg, 58%). ES/MS (m/z): 537 (M+H-BOC).

### Preparation 120

### (2R)-2-[(3S)-1-tert-Butoxycarbonylpyrrolidin-3-yl]-3-[3-[3-[(2R)-2-[(3S)-1-tert-butoxycarbonylpyrrolidin-3-yl]-2-carboxy-ethyl]phenyl]phenyl]propanoic acid

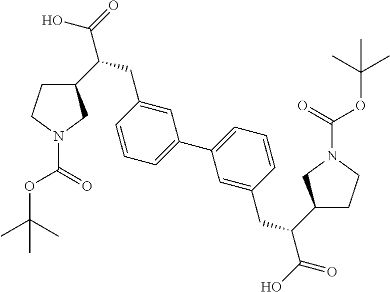 [SMILES: CC(C)(C)OC(=O)N1CC[C@@H]([C@@H](Cc2cccc(-c3cccc(C[C@@H](C(=O)O)[C@@H]4CCN(C(=O)OC(C)(C)C)C4)c3)c2)C(=O)O)C1]

Prepare the title compound essentially as described in Preparation 119 using (2R)-3-(3-bromophenyl)-2-[(3S)-1-tert-butoxycarbonylpyrrolidin-3-yl]propanoic acid ammonium salt and (2R)-2-[(3S)-1-tert-butoxycarbonylpyrrolidin-3-yl]-3-[3-(4,4,5,5-tetramethyl-1,3,2-dioxaborolan-2-yl)phenyl]propanoic acid. Upon completion of the reaction, the reaction mixture was quenched with water and extracted with EtOAc. The organic phase was washed with saturated aqueous NaCl, dried over MgSO₄, filtered, and concentrated. The residue was purified by silica gel chromatography using 90 to 100% EtOAc in hexanes to give the title compound as a colorless oil. ES/MS (m/z): 537 (M+H-BOC).

### Preparation 121

### tert-Butyl (3R)-3-[(1S)-2-tert-butoxy-1-[[3-[3-[(2S)-3-tert-butoxy-2-[(3R)-1-tert-butoxycarbonylpyrrolidin-3-yl]-3-oxo-propyl]-5-(trifluoromethyl)anilino]phenyl]methyl]-2-oxo-ethyl]pyrrolidine-1-carboxylate

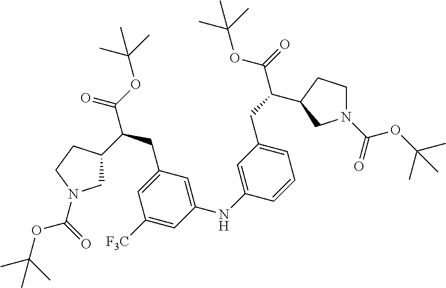 [SMILES: CC(C)(C)OC(=O)[C@@H](Cc1cccc(Nc2cc(C[C@H](C(=O)OC(C)(C)C)[C@H]3CCN(C(=O)OC(C)(C)C)C3)cc(C(F)(F)F)c2)c1)[C@H]1CCN(C(=O)OC(C)(C)C)C1]

tert-Butyl (3R)-3-[(1S)-1-[[3-bromo-5-(trifluoromethyl)phenyl]methyl]-2-tert-butoxy-2-oxo-ethyl]pyrrolidine-1-carboxylate (250 mg, 0.479 mmol), tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429, 0.224 g, 0.574 mmol), cesium carbonate (468 mg, 1.44 mmol), and methanesulfonato(2-dicyclohexylphosphino-2′,6′-di-1-propoxy-1,1′-biphenyl)(2′-amino-1,1′-biphenyl-2-yl)palladium(II) (0.3 equiv., 123 mg, 0.144 mmol) in 1,4-dioxane (3.35 mL) mixed and heated with stirring at 110° C. for 16 h, and then filtered through a pad of diatomaceous earth, washing with DCM and MeOH. The filtrate was concentrated under reduced pressure. The material was purified by RP-HPLC/MS [column: XBridge® C18 (19×100 mm, 5 μm); mobile phase: 20 mM NH₄HCO₃ in water (pH9)—solvent A; ACN—solvent B; elution conditions: gradient from 74% to 95% B; flow rate: 25 mL/min; column at RT) to give the title compound (173 mg, 43%). ES/MS (m/z): 732 (M+H-BOC).

The following were prepared essentially as described in Preparation 121 using the appropriate starting materials:

| | | | Procedure | Physical Data |
|---|---|---|---|---|
| Preparation | Chemical name | Structure | modifications | ES-MS m/z |
| 122 | tert-Butyl (3R)-3-[(1S)-2- tert-butoxy-1-[[4-[3-[(2S)- 3-tert-butoxy-2-[(3R)-1- tert- butoxycarbonylpyrrolidin- 3-yl]-3-oxo- propyl]anilino]phenyl]methyl]- 2-oxo-ethyl]pyrrolidine- 1-carboxylate | 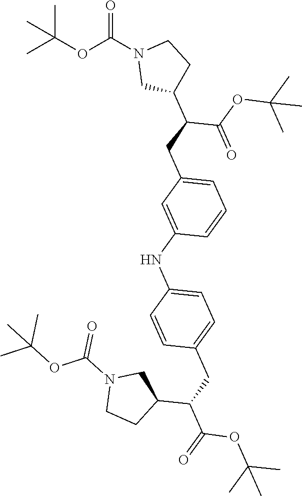 [SMILES: CC(C)(C)OC(=O)[C@@H](Cc1ccc(Nc2cccc(C[C@H](C(=O)OC(C)(C)C)[C@H]3CCN(C(=O)OC(C)(C)C)C3)c2)cc1)[C@H]1CCN(C(=O)OC(C)(C)C)C1] | | 664 (M + H—BOC) |
| 123 | tert-Butyl (3R)-3-[(1S)-2- tert-butoxy-1-[3-[3-[(2S)- 3-tert-butoxy-2-[(3R)-1- tert- butoxycarbonylpyrrolidin- 3-yl]-3-oxo- propyl]anilino]phenyl]methyl]- 2-oxo-ethyl]pyrrolidine- 1-carboxylate | 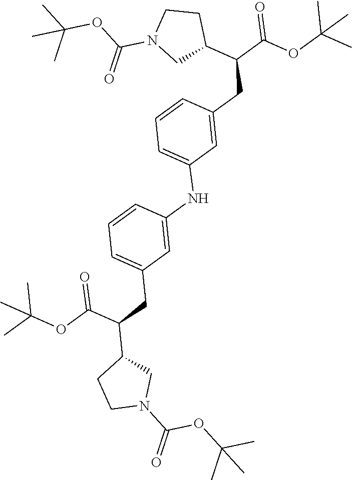 [SMILES: CC(C)(C)OC(=O)[C@@H](Cc1cccc(Nc2cccc(C[C@H](C(=O)OC(C)(C)C)[C@H]3CCN(C(=O)OC(C)(C)C)C3)c2)c1)[C@H]1CCN(C(=O)OC(C)(C)C)C1] | A | 665 (M + H—BOC) |

A Purified on a hydrophobic lipophilic balance (HLB) column eluted with a gradient of 0 to 100% ACN in aqueous NH₄HCO₃ (pH 9)

### Preparation 124

### tert-Butyl (3R)-3-[(1S)-1-[(4-bromophenyl)methyl]-2-tert-butoxy-2-oxo-ethyl]pyrrolidine-1-carboxylate

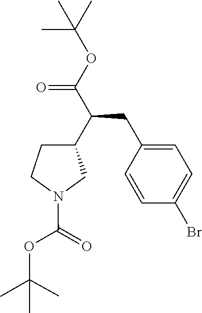 [SMILES: CC(C)(C)OC(=O)[C@@H](Cc1ccc(Br)cc1)[C@H]1CCN(C(=O)OC(C)(C)C)C1]

(2S)-3-(4-Bromophenyl)-2-[(3R)-1-tert-butoxycarbonylpyrrolidin-3-yl]propanoic acid (2.73 g, 6.85 mmol) was dissolved in toluene (68 mL), heated at 80° C., and 1,1-di-tert-butoxy-N,N-dimethyl-methanamine (12.8 mL, 48.0 mmol) was added. The mixture was stirred and heated at 80° C. for 3 h. Additional 1,1-di-tert-butoxy-N,N-dimethyl-methanamine (3.0 mL, 11 mmol) was added and the reaction was stirred at 80° C. for 16 h. The reaction was cooled to RT and concentrated under reduced pressure. The residue was purified with silica gel chromatography eluting with a 0 to 40% gradient of EtOAc in hexanes to give the title compound (3.0 g, 90%). ES/MS (m/z): 342/344 (M+H-2×tert-butyl).

### Preparation 125

### tert-Butyl (3R)-3-[(1S)-1-[(4-aminophenyl)methyl]-2-tert-butoxy-2-oxo-ethyl]pyrrolidine-1-carboxylate

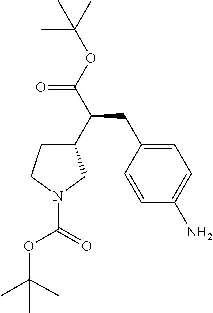 [SMILES: CC(C)(C)OC(=O)[C@@H](Cc1ccc(N)cc1)[C@H]1CCN(C(=O)OC(C)(C)C)C1]

A suspension of tert-butyl (3R)-3-[(1S)-1-[(4-bromophenyl)methyl]-2-tert-butoxy-2-oxo-ethyl]pyrrolidine-1-carboxylate (1.51 g, 3.32 mmol), copper(I) oxide (0.490 g, 3.32 mmol), L-proline (0.586 g, 4.98 mmol), and sodium azide (0.655 g, 9.97 mmol) in DMSO (16 mL) was degassed with a positive argon stream. The reaction mixture was heated to 100° C. for 36 h in a microwave reactor. The reaction was carefully vented, poured into EtOAc (100 mL), and washed with saturated aqueous NaHCO₃ (2×50 mL). The organic extract was dried over MgSO₄, filtered, and concentrated under reduced pressure to give the title compound (quantitative yield, 3.32 mmol) ES/MS (m/z): 291 (M+H-BOC).

### Preparation 126

### tert-butyl (3R)-3-[(1S)-1-[[3-[(3-acetoxyphenoxy)methyl]phenyl]methyl]-2-tert-butoxy-2-oxo-ethyl]pyrrolidine-1-carboxylate

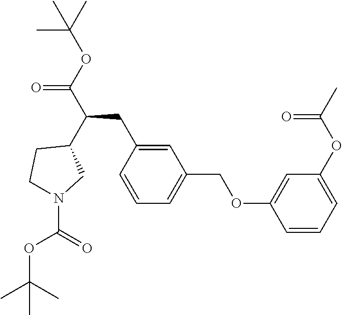 [SMILES: CC(=O)Oc1cccc(OCc2cccc(C[C@H](C(=O)OC(C)(C)C)[C@H]3CCN(C(=O)OC(C)(C)C)C3)c2)c1]

Diethyl azodicarboylate (0.23 mL, 1.5 mmol) was added to a solution of tert-butyl (3R)-3-[(1S)-2-tert-butoxy-1-[[3-(hydroxymethyl)phenyl]methyl]-2-oxo-ethyl]pyrrolidine-1-carboxylate (prepared essentially as described in WO 2020/247429; 200 mg, 0.493 mmol), triphenylphosphine (196 mg, 0.739 mmol), and (3-hydroxyphenyl)acetate (82 mg, 0.54 mmol) in THE (2 mL) under nitrogen atmosphere. The mixture was stirred at RT for 16 h and saturated aq NaHCO₃ was added. The organic phase was separated from the aqueous phase, washed with saturated aq NaCl (3+), dried over MgSO₄, and concentrated under reduced pressure. Subjected the resulting residue to silica gel chromatography eluting with EtOAc: hexanes (gradient: 10-60%) to give the title compound (123 mg, 46% yield). ES/MS (m/z): 538 (M−H).

### Preparation 127

### tert-Butyl (3R)-3-[(1S)-2-tert-butoxy-1-[[3-[(3-hydroxyphenoxy)methyl]phenyl]methyl]-2-oxo-ethyl]pyrrolidine-1-carboxylate

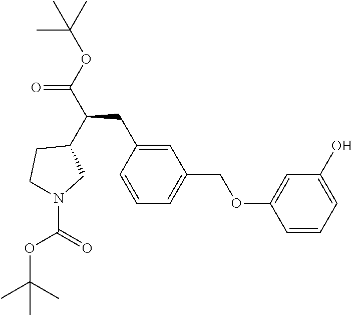 [SMILES: CC(C)(C)OC(=O)[C@@H](Cc1cccc(COc2cccc(O)c2)c1)[C@H]1CCN(C(=O)OC(C)(C)C)C1]

Potassium carbonate (67 mg, 0.68 mmol) was added to a solution of tert-butyl (3R)-3-[(1S)-1-[[3-[(3-acetoxyphenoxy)methyl]phenyl]methyl]-2-tert-butoxy-2-oxo-ethyl]pyrrolidine-1-carboxylate (123 mg, 0.227 mmol) in MeOH (4 mL). The mixture was stirred at RT for 15 min and evaporated the solvent under reduced pressure. 0.5N aq HCl and DCM were added. The organic phase was separated, washed with saturated aq NaCl, dried over MgSO₄, and concentrated under reduced pressure to give the title compound (117 mg, 92% yield). ES/MS (m/z): 398 (M+H-Boc).

### Preparation 128

### tert-Butyl (3R)-3-[(1S)-2-tert-butoxy-1-[[3-[[3-[[3-[(2S)-3-tert-butoxy-2-[(3R)-1-tert-butoxycarbonylpyrrolidin-3-yl]-3-oxo-propyl]phenyl]methoxy]phenoxy]methyl]phenyl]methyl]-2-oxo-ethyl]pyrrolidine-1-carboxylate

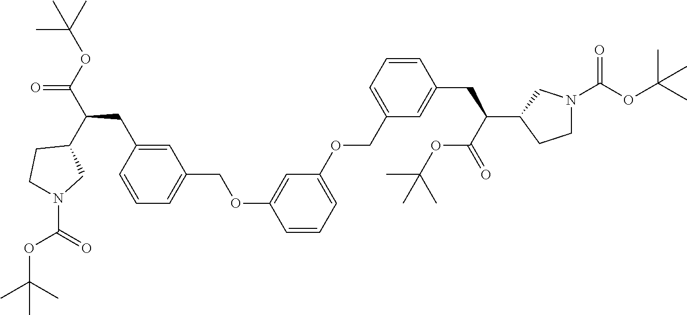 [SMILES: CC(C)(C)OC(=O)[C@@H](Cc1cccc(COc2cccc(OCc3cccc(C[C@H](C(=O)OC(C)(C)C)[C@H]4CCN(C(=O)OC(C)(C)C)C4)c3)c2)c1)[C@H]1CCN(C(=O)OC(C)(C)C)C1]

Diethyl azodicarboylate (0.11 mL, 0.71 mmol) was added to a solution of tert-butyl (3R)-3-[(1S)-2-tert-butoxy-1-[[3-[(3-hydroxyphenoxy)methyl]phenyl]methyl]-2-oxo-ethyl]pyrrolidine-1-carboxylate (117 mg, 0.253 mmol), triphenylphospine (93 mg, 0.352 mmol., 1.5 equiv.), and tert-butyl (3R)-3-[(1S)-2-tert-butoxy-1-[[3-(hydroxymethyl)phenyl]methyl]-2-oxo-ethyl]pyrrolidine-1-carboxylate (prepared essentially as described in WO 2020/247429; 105 mg, 0.259 mmol) in THE (2 mL) under nitrogen atmosphere. The mixture was stirred at RT for 16 h. More triphenylphosphine (93 mg, 0.352 mmol) and diethyl azodicarboylate (0.11 mL, 0.705 mmol) were added. After 3 h saturated aq NaHCO₃ was added. The organic phase was separated, washed with saturated aqueous NaCl, dried over MgSO₄, and concentrated under reduced pressure. The residue was purified by reverse phase HPLC [column: XBridge® C18 5™, 19×100 mm; mobile phase: solvent A—aqueous NH₄HCO₃ (20 mM, pH 9.0), solvent B—ACN; gradient: 75-95% solvent B in solvent A; flow rate: 25 mL/min; column temperature: 50° C.] to afford the title compound (50 mg, 24%). ES/MS (m/z): 785 (M+H-Boc).

### Preparation 129

### tert-butyl (3R)-3-[(1S)-2-tert-butoxy-1-[[3-[5-[(2S)-3-tert-butoxy-2-[(3R)-1-tert-butoxycarbonylpyrrolidin-3-yl]-3-oxo-propyl]-1-(2-phenylethyl)benzimidazol-2-yl]phenyl]methyl]-2-oxo-ethyl]pyrrolidine-1-carboxylate

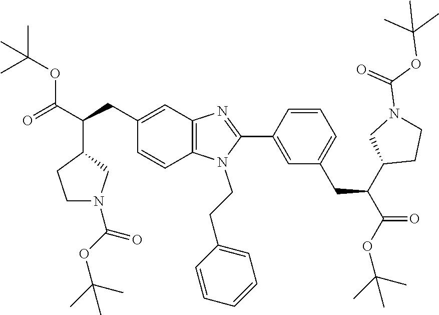 [SMILES: CC(C)(C)OC(=O)[C@@H](Cc1cccc(-c2nc3cc(C[C@H](C(=O)OC(C)(C)C)[C@H]4CCN(C(=O)OC(C)(C)C)C4)ccc3n2CCc2ccccc2)c1)[C@H]1CCN(C(=O)OC(C)(C)C)C1]

tert-Butyl (3R)-3-[(1S)-2-tert-butoxy-1-[(4-fluoro-3-nitro-phenyl)methyl]-2-oxo-ethyl]pyrrolidine-1-carboxylate (50 mg, 0.114 mmol) from a freshly prepared stock solution (0.2 M in DMA) was added to 2-phenylethanamine (0.118 mmol) in a tube. DMA (980 μL) was then added, and the mixture was stirred at 90° C. for 2 h. Na₂S₂O₄ (295 μL, 0.59 mmol, 2 M) was added followed by tert-butyl (3R)-3-[(1S)-2-tert-butoxy-1-[(3-formylphenyl)methyl]-2-oxo-ethyl]pyrrolidine-1-carboxylate (prepared essentially as described in WO 2020/247429, 118 mol, 0.118 mmol) from stock solution (0.6 M in DMA). The mixture was stirred at 90° C. for 2 h and then stirred at RT overnight. The mixture was loaded onto a HLB cartridge (6 g) and then eluted with water (2 column volumes) and ACN (2 column volumes). The solvent of the organic fraction was evaporated under nitrogen. The crude material was purified by RP-HPLC/MS [column: XBridge® C18 (19×100 mm, 5 μm); mobile phase: solvent A—20 mM NH₄HCO₃ in water (pH9), solvent B—ACN, gradient from 74% to 95% solvent B in solvent A; flow rate: 25 mL/min; column at RT] to obtain the title compound (45 mg, 44% yield).

### Preparation 130

### tert-Butyl (3R)-3-[(1S)-2-tert-butoxy-1-[[3-[3-[(2S)-3-tert-butoxy-2-[(3R)-1-tert-butoxycarbonylpyrrolidin-3-yl]-3-oxo-propyl]-N-carbamoyl-anilino]phenyl]methyl]-2-oxo-ethyl]pyrrolidine-1-carboxylate

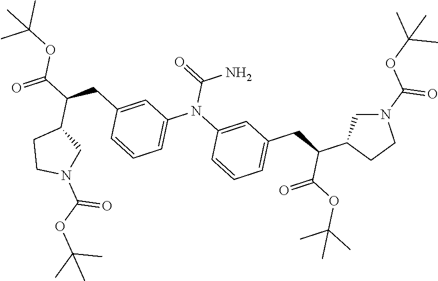 [SMILES: CC(C)(C)OC(=O)[C@@H](Cc1cccc(N(C(N)=O)c2cccc(C[C@H](C(=O)OC(C)(C)C)[C@H]3CCN(C(=O)OC(C)(C)C)C3)c2)c1)[C@H]1CCN(C(=O)OC(C)(C)C)C1]

To a solution of tert-butyl (3R)-3-[(1S)-2-tert-butoxy-1-[[3-[3-[(2S)-3-tert-butoxy-2-[(3R)-1-tert-butoxycarbonylpyrrolidin-3-yl]-3-oxo-propyl]anilino]phenyl]methyl]-2-oxo-ethyl]pyrrolidine-1-carboxylate (150 mg, 0.2 mmol) in toluene (12 mL/mmol, 2.4 mL) was added sodium cyanate (30 mg, 0.5 mmol). Then, TFA (0.072 mL, 0.9 mmol) was added and the mixture was stirred at RT overnight. The reaction mixture was concentrated and the residue was purified by silica gel chromatography using a gradient of 0 to 100% acetone in hexanes to give the title compound (76 mg, 42%). ES/MS (m/z): 708 (M+H-BOC).

### Preparation 131

### tert-Butyl (3R)-3-[(1S)-1-[[3-[[2-[bis[[3-[(2S)-3-tert-butoxy-2-[(3R)-1-tert-butoxycarbonylpyrrolidin-3-yl]-3-oxo-propyl]phenyl]methyl]amino]ethyl-[[3-[(2S)-3-tert-butoxy-2-[(3R)-1-tert-butoxycarbonylpyrrolidin-3-yl]-3-oxo-propyl]phenyl]methyl]amino]methyl]phenyl]methyl]-2-tert-butoxy-2-oxo-ethyl]pyrrolidine-1-carboxylate

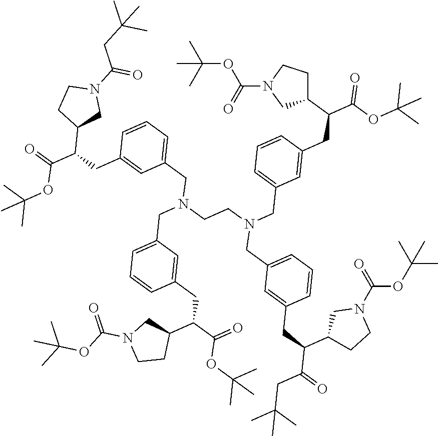 [SMILES: CC(C)(C)CC(=O)[C@@H](Cc1cccc(CN(CCN(Cc2cccc(C[C@H](C(=O)OC(C)(C)C)[C@H]3CCN(C(=O)CC(C)(C)C)C3)c2)Cc2cccc(C[C@H](C(=O)OC(C)(C)C)[C@H]3CCN(C(=O)OC(C)(C)C)C3)c2)Cc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

To a mixture of tert-butyl (3R)-3-[(1S)-2-tert-butoxy-1-[[3-[[[3-[(2S)-3-tert-butoxy-2-[(3R)-1-tert-butoxycarbonylpyrrolidin-3-yl]-3-oxo-propyl]phenyl]methylamino]methyl]phenyl]methyl]-2-oxo-ethyl]pyrrolidine-1-carboxylate (prepared essentially as described in WO 2020/247429, 185 mg, 0.334 mmol) in EtOH (5 mL) was added oxaldehyde (16 mg, 13 μL, 0.1667 mmol) and MgSO₄. The mixture was stirred at RT for 30 min, then sodium triacetoxyborohydride (101 mg, 0.467 mmol) was added and the reaction was stirred for 5 h. Additional oxaldehyde (6 mg, 5 μL, 0.0467 mmol) was added followed by sodium triacetoxyborohydride (25 mg, 0.117 mmol) and the reaction was stirred for 1 h. The reaction mixture was filtered and concentrated. The residue was dissolved in DCM and washed with saturated aqueous NaHCO₃ and saturated aqueous NaCl, then dried over MgSO₄, filtered, and concentrated. The residue was purified by silica gel chromatography using a gradient of 10 to 90% EtOAc in hexanes to give the title compound (89 mg, 24%). ¹H NMR (400.13 MHz, CDCl₃) δ 7.17 (s, 8H), 7.04 (s, 8H), 3.73-3.64 (m, 2H), 3.59-3.39 (m, 14H), 3.24 (d, J=6.1 Hz, 4H), 3.07-2.94 (m, 4H), 2.86-2.72 (m, 8H), 2.64-2.55 (m, 4H), 2.52-2.45 (m, 4H), 2.38-2.34 (m, 4H), 1.97-1.90 (m, 4H), 1.72-1.62 (m, 4H), 1.48 (s, 36H), 1.26-1.24 (m, 36H).

### Preparation 132

### (2S)-2-[(3R)-1-tert-Butoxycarbonylpyrrolidin-3-yl]-3-(3-phenylphenyl)propanoic acid

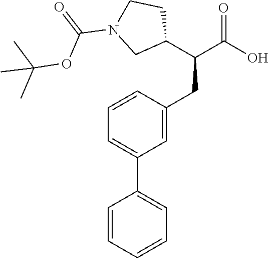 [SMILES: CC(C)(C)OC(=O)N1CC[C@H]([C@H](Cc2cccc(-c3ccccc3)c2)C(=O)O)C1]

A mixture of (2S)-3-(3-bromophenyl)-2-[(3R)-1-tert-butoxycarbonylpyrrolidin-3-yl]propanoic acid (prepared essentially as described in WO 2020/247429, 500 mg, 1.26 mmol), phenylboronic acid (138 mg, 1.13 mmol) and K₂CO₃ (521 mg, 3.77 mmol) in 1,4-dioxane (12.6 mL) and water (1.3 mL) was purged with N₂ for 5 min. 1,1′-bis(diphenylphosphino)ferrocene-palladium(II)dichloride DCM complex (107 mg, 0.126 mmol) was added and the reaction mixture was purged with N₂ for 5 min and heated overnight at 100° C. The reaction mixture was shaken between water and DCM and the layers were separated. The aqueous layer was washed three times with DCM. The organic layers were combined, dried over MgSO₄, filtered, and concentrated under reduce pressure. The residue was purified by silica gel chromatography using a gradient of 10 to 50% acetone in hexanes to give the title compound (310 mg, 62%). ES-MS m/z 340 (M-tBu).

### Preparation 133

### (2S)-2-[(3R)-1-tert-butoxycarbonylpyrrolidin-3-yl]-3-[3-[2-(3-cyclopropylphenyl)ethoxy]phenyl]propanoic acid

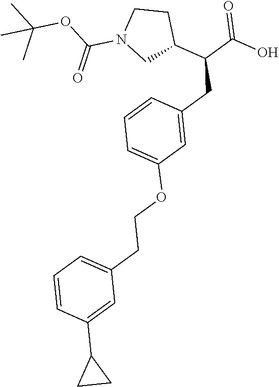 [SMILES: CC(C)(C)OC(=O)N1CC[C@H]([C@H](Cc2cccc(OCCc3cccc(C4CC4)c3)c2)C(=O)O)C1]

A reaction vessel containing a mixture of (2S)-3-(3-bromophenyl)-2-[(3R)-1-tert-butoxycarbonylpyrrolidin-3-yl]propanoic acid (ammonium salt, 415 mg, 1.00 mmol), 8-hydroxyquinoline (19 mg, 0.13 mmol), CuI (14 mg, 0.072 mmol), and potassium phosphate tribasic (440 mg, 2.03 mmol) was purged with nitrogen, then 2-(3-cyclopropylphenyl)ethanol (500 mg, 2.90 mmol) was added and the reaction mixture was stirred at 110° C. for 24 h. The mixture was shaken with water, Et₂O, and HCl (1 M aqueous, 5 mL). The organic layer was washed with water, dried over Na₂SO₄, filtered and concentrated under reduced pressure. The residue was purified by silica gel chromatography using a gradient of 0 to 17% EtOAc in DCM. Fractions containing the title compound were concentrated and combined with impure product from a reaction run on a similar scale and re-purified by reverse-phase HPLC [column: XBridge® C18 19×100 mm, 5 μm; mobile phase: 45 to 65% ACN in aqueous NH₄HCO₃ (20 mM, pH 9); flow rate 25 mL/min] to give 71 mg (7%) of the title compound as a pale yellow oil. ES-MS m/z 424 (M-tBu).

### Preparation 134

### [3-[(2S)-3-tert-butoxy-2-[(3R)-1-tert-butoxycarbonylpyrrolidin-3-yl]-3-oxo-propyl]phenyl]boronic acid

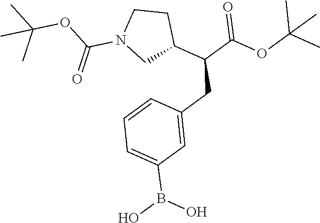 [SMILES: CC(C)(C)OC(=O)[C@@H](Cc1cccc(B(O)O)c1)[C@H]1CCN(C(=O)OC(C)(C)C)C1]

A mixture of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 1.00 g, 2.20 mmol), tetrahydroxydiboron (0.302 g, 3.30 mmol), chloro(2-dicyclohexylphosphino-2′,4′,6′-triisopropyl-1,1′-biphenyl)[2-(2′-amino-1,1′-biphenyl)]palladium(II) (8.8 mg, 0.011 mmol), X-PHOS (0.01 equiv., 0.02201 mmol, 98 mass %, 0.01071 g), potassium acetate (0.65 g, 6.6 mmol) and ethylene glycol (0.37 mL, 0.41 g, 6.6 mmol) in EtOH (22 mL) was stirred while purging with nitrogen for 10 min. The mixture was heated to 100° C. for 3.5 h, then stirred at RT overnight. The mixture was filtered through a short plug of diatomaceous earth and washed with EtOH. The filtrate was concentrated to dryness, then taken up in a mixture of water and EtOAc, the phases were separated, and the aqueous phase was extracted twice more with EtOAc. The combined organic phases were washed with saturated aqueous NaCl, dried over MgSO₄, filtered and evaporated to dryness. Half of the residue was purified by silica gel chromatography using a gradient of 25 to 100% EtOAc in hexanes to give the title compound (294 mg, 64% yield based on the portion which was purified). ES-MS m/z 280 (M+H-2^{t}Bu).

### Preparation 135

### (2S)-2-[(3R)-1-tert-Butoxycarbonylpyrrolidin-3-yl]-3-[3-(5-chloro-2-thienyl)phenyl]propanoic acid

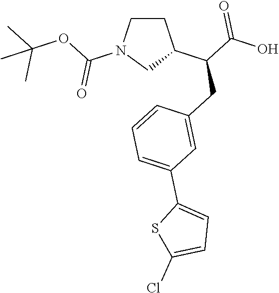 [SMILES: CC(C)(C)OC(=O)N1CC[C@H]([C@H](Cc2cccc(-c3ccc(Cl)s3)c2)C(=O)O)C1]

A mixture of (2S)-2-[(3R)-1-tert-butoxycarbonylpyrrolidin-3-yl]-3-[3-(4,4,5,5-tetramethyl-1,3,2-dioxaborolan-2-yl)phenyl]propanoic acid (150 mg, 0.32 mmol), 2-bromo-5-chlorothiophene (98 mg, 0.48 mmol) in anhydrous DMF (3.2 mL) was purged with N₂ for 10 min, and then potassium phosphate dibasic (1 M solution in water, 1.6 mL, 1.6 mmol) and tetrakis(triphenylphosphine)palladium(0) (27 mg, 0.022 mmol) were added. The reaction mixture was stirred at 100° C. for 4 h then cooled to RT. The mixture was purged with N₂ for 5 min, then tetrakis(triphenylphosphine)palladium(0) (27 mg, 0.022 mmol) and potassium phosphate dibasic (1 M solution in water, 1.6 mL, 1.6 mmol) were added. The reaction was heated at 100° C. for 15.5 h, then cooled to RT. The reaction was purged with N₂ for 5 min, then 2-bromo-5-chlorothiophene (98 mg, 0.48 mmol), tetrakis(triphenylphosphine)palladium(0) (27 mg, 0.022 mmol) and potassium phosphate dibasic (1 M solution in water, 1.6 mL, 1.6 mmol) were added and the reaction mixture was stirred at 100° C. for 2 h and at RT for 3 days. The reaction mixture was combined with the reaction mixture run in a similar manner on 0.10 mmol of the starting boronic acid. The combined mixture was filtered over a pad of diatomaceous earth, rinsing with MeOH. To the filtrate was added aqueous citric acid (5%) and the mixture was extracted with EtOAc. The organic layer was washed with saturated aqueous NaCl, dried over anhydrous Na₂SO₄, filtered, and concentrated under reduce pressure. The residue was purified by silica gel chromatography using a gradient of 10 to 40% acetone in DCM, then re-purified by reverse-phase HPLC [column: XBridge® C18 19×100 mm, 5 μm; mobile phase: 35 to 55% ACN in aqueous NH₄HCO₃ (20 mM, pH 9), flow rate: 25 mL/min] to give the title compound (22.3 mg, 16%) as a white solid. ES-MS m/z 434 (M−H).

### Preparation 136

### (2S)-2-[(3R)-1-tert-Butoxycarbonylpyrrolidin-3-yl]-3-(3-fluoro-5-thiazol-4-yl-phenyl)propanoic acid

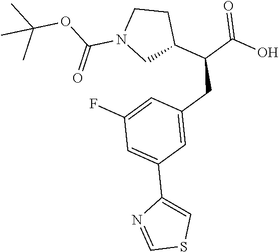 [SMILES: CC(C)(C)OC(=O)N1CC[C@H]([C@H](Cc2cc(F)cc(-c3cscn3)c2)C(=O)O)C1]

A mixture of (2S)-3-(3-bromo-5-fluoro-phenyl)-2-[(3R)-1-tert-butoxycarbonylpyrrolidin-3-yl]propanoic acid (0.25 g, 0.60 mmol), bis(pinacolato)diboron (0.18 g, 0.72 mmol), potassium acetate (0.15 g, 1.5 mmol) and [1,1′-bis(diphenylphosphino)ferrocene]dichloropalladium(II) (0.027 g, 0.036 mmol) in 1,4-dioxane (4.8 mL) was heated at 100° C. overnight. The mixture was cooled to 80° C. and 4-bromothiazole (0.11 g, 0.061 mL, 0.66 mmol), Na₂CO₃ (2 M solution in water, 1.8 mL, 1.8 mmol) and [1,1′-bis(diphenylphosphino)ferrocene]dichloropalladium(II) (0.013 g, 0.018 mmol) were added. The mixture was heated at 80° C. for 5 h then cooled to RT. The mixture was quenched with HCl (1 N aqueous) and extracted with EtOAc. The combined organic phases were filtered (diatomaceous earth), washed with saturated aqueous NaCl, dried over MgSO₄, filtered and evaporated to dryness. The residue was purified via silica gel chromatography using a gradient of 0 to 80% EtOAc+1% acetic acid, then re-purified using reversed-phase HPLC [column: Bonus RP 21×100 mm, 5 μm; mobile phase: 30 to 80% ACN in aqueous NH₄HCO₃ (20 mM, pH 8), flow rate: 25 mL/min] to give 88 mg (35%) of the title compound as a white solid. ES-MS m/z 421 (M+H).

### Preparation 137

### tert-Butyl (3R)-3-[(1S)-2-tert-butoxy-2-oxo-1-[(3-tetrahydropyran-4-ylphenyl)methyl]ethyl]pyrrolidine-1-carboxylate

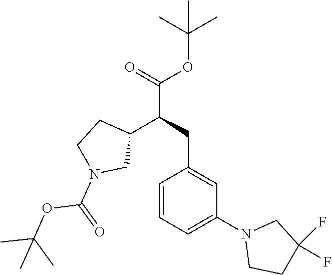 [SMILES: CC(C)(C)OC(=O)[C@@H](Cc1cccc(N2CCC(F)(F)C2)c1)[C@H]1CCN(C(=O)OC(C)(C)C)C1]

A mixture of (2S)-3-(3-bromophenyl)-2-[(3R)-1-tert-butoxycarbonylpyrrolidin-3-yl]propanoic acid (prepared essentially as described in WO 2020/247429, 200 mg, 0.440 mmol), Cs₂CO₃ (0.431 g, 1.32 mmol) in toluene (3 mL) was purged with N₂, then 3,3-difluoropyrrolidine hydrochloride (75.8 mg, 0.528 mmol) and methanesulfonato(2-dicyclohexylphosphino-2′,6′-di-1-propoxy-1,1′-biphenyl)(2′-amino-1,1′-biphenyl-2-yl)palladium(II) (RuPhos Palladacycle Gen. 4, 113 mg, 0.132 mmol) were added and the mixture was heated to 85° C. for 2 days. The reaction mixture was then filtered over diatomaceous earth and concentrated under reduced pressure. The residue was purified via silica gel chromatography using a gradient of 5 to 25% EtOAc in hexanes to give the title compound (139 mg, 64%) as a brown oil. ES-MS m/z 381 (M+H-BOC).

### Preparation 138

### (2S)-2-[(3R)-1-tert-Butoxycarbonylpyrrolidin-3-yl]-3-[3-(5-methyl-2-oxo-indolin-1-yl)phenyl]propanoic acid

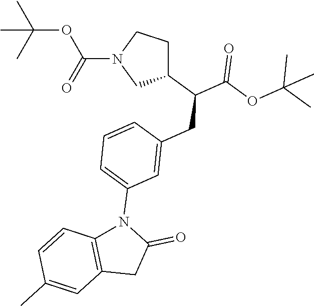 [SMILES: Cc1ccc2c(c1)CC(=O)N2c1cccc(C[C@H](C(=O)OC(C)(C)C)[C@H]2CCN(C(=O)OC(C)(C)C)C2)c1]

To a mixture of 5-methylindolin-2-one (202 mg, 1.35 mmol), (2S)-3-(3-bromophenyl)-2-[(3R)-1-tert-butoxycarbonylpyrrolidin-3-yl]propanoic acid (ammonium salt, 500 mg, 1.204 mmol) and K₂CO₃ (545 mg, 3.90 mmol) was added ACN (4 mL). A steady stream of N₂ was bubbled through the suspension as it was heated to 40° C. over 15 min. Cuprous iodide (34.4 mg, 0.181 mmol) and DMF (39 L) were added. The reaction vessel was sealed and the mixture was heated at 80° C. for 15.5 h. The reaction mixture was cooled to RT, quenched with 1N HCl and extracted with EtOAc. The combined organic phases were washed with saturated aqueous NaCl, dried over MgSO₄, filtered, and evaporated to dryness. The residue was purified by silica gel chromatography using a gradient of 12 to 50% (1% acetic acid in acetone) in hexanes. To the purified product was added toluene and the mixture was concentrated, then mixtures of MTBE and hexanes were added to the residue and concentrated to give the title compound (277 mg, 50%) as a pale yellow solid. ES-MS m/z 521 (M+H).

### Preparation 139

### 1-(3-Benzyloxyphenyl)imidazolidin-2-one

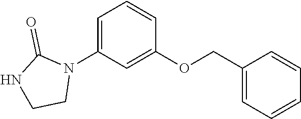 [SMILES: O=C1NCCN1c1cccc(OCc2ccccc2)c1]

A mixture of ethyleneurea (1.4 g, 16 mmol), 1-benzyloxy-3-iodobenzene (4.9 g, 16 mmol), cuprous iodide (0.61 g, 3.1 mmol), and potassium dihydrogen phosphate (4.2 g, 31 mmol) under argon atmosphere was suspended in DMF (argon sparged, 20 mL), then N,N′-dimethylethylenediamine (0.28 g, 0.33 mL, 3.1 mmol) was added. The suspension was heated to 120° C. for 3 h. The mixture was cooled and filtered through a pad of silica gel, flushing the pad with EtOAc. The filtrate was concentrated under reduced pressure, and the residue was purified by silica gel chromatography using 1:4:15 MeOH:acetone:EtOAc to give the title compound (1.10 g, 26%). ES-MS m/z 269 (M+H).

### Preparation 140

### (2S)-3-[3-[3-(3-Benzyloxyphenyl)-2-oxo-imidazolidin-1-yl]phenyl]-2-[(3R)-1-tert-butoxycarbonylpyrrolidin-3-yl]propanoic acid

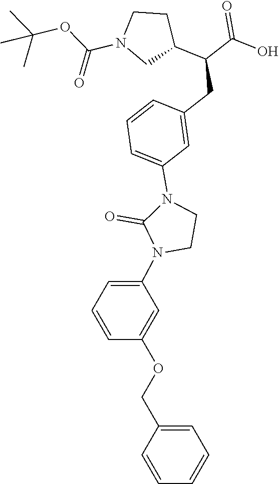 [SMILES: CC(C)(C)OC(=O)N1CC[C@H]([C@H](Cc2cccc(N3CCN(c4cccc(OCc5ccccc5)c4)C3=O)c2)C(=O)O)C1]

To a mixture of 1-(3-benzyloxyphenyl)imidazolidin-2-one (1.09 g, 4.05 mmol) and (2S)-3-(3-bromophenyl)-2-[(3R)-1-tert-butoxycarbonylpyrrolidin-3-yl]propanoic acid (ammonium salt, 1.40 g, 3.37 mmol) under N₂ was added ACN (16.9 mL) and DMF (3 mL). A steady stream of argon was bubbled through the suspension over 15 min, then K₂CO₃ (1.52 g, 10.9 mmol), CuI (128 mg, 0.674 mmol) and N,N′-dimethylethylenediamine (120 mg, 0.147 mL, 1.35 mmol) were added and the mixture was heated to 100° C. for 18 h in a microwave reactor. The mixture was quenched with water and extracted twice with EtOAc. The pH of the aqueous phase was acidified with 0.5 N aqueous HCl and extracted with EtOAc, DCM, and then again with EtOAc. The combined organic phases were dried over MgSO₄, filtered, and evaporated to dryness. The residue was purified by silica gel chromatography using a gradient of 10 to 60% (acetone+1% acetic acid) in hexanes to give 1.10 g (56%) of a yellow oil which slowly crystallized. ES-MS m/z 486 (M+H-BOC).

### Preparation 141

### tert-Butyl (3R)-3-[(1S)-2-tert-butoxy-1-[[3-(3,3-dimethylindolin-1-yl)phenyl]methyl]-2-oxo-ethyl]pyrrolidine-1-carboxylate

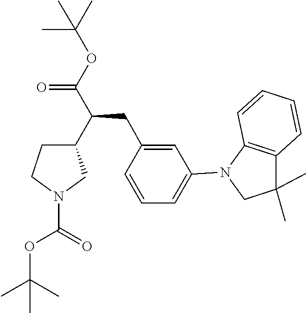 [SMILES: CC(C)(C)OC(=O)[C@@H](Cc1cccc(N2CC(C)(C)c3ccccc32)c1)[C@H]1CCN(C(=O)OC(C)(C)C)C1]

The title compound was prepared essentially as described in Preparation 137 using 3,3-dimethylindoline. ES-MS m/z 521 (M+H).

### Preparation 142

### N,N′-Bis[(4-methoxyphenyl)methyl]-N,N′-diphenyl-propanediamide

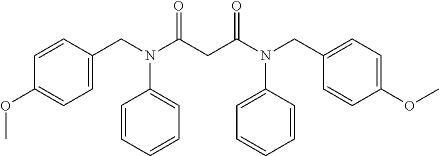 [SMILES: COc1ccc(CN(C(=O)CC(=O)N(Cc2ccc(OC)cc2)c2ccccc2)c2ccccc2)cc1]

To a solution of N-[(4-methoxyphenyl)methyl]aniline (2.05 g, 9.61 mmol) in DCM (48 mL) was added malonic acid (500 mg, 0.308 mL, 4.80 mmol) and N,N′-dicyclohexylcarbodiimide (1.98 g, 9.60 mmol). The resulting mixture was stirred at RT overnight. The reaction was filtered through diatomaceous earth and the filtrate was concentrated to dryness. The residue was purified by silica gel chromatography using a gradient of 0 to 10% (ammonia in MeOH) in DCM to give 1 g (42%) of the title compound as a pale grey solid. ES-MS m/z 495 (M+H).

### Preparation 143

### 1,1′-Bis[(4-methoxyphenyl)methyl]-3,3′-spirobi[indoline]-2,2′-dione

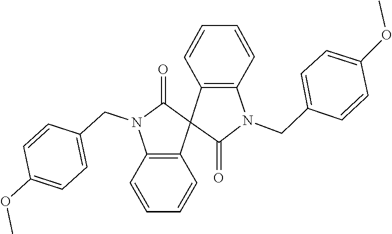 [SMILES: COc1ccc(CN2C(=O)C3(C(=O)N(Cc4ccc(OC)cc4)c4ccccc43)c3ccccc32)cc1]

To a solution of N,N′-bis[(4-methoxyphenyl)methyl]-N,N′-diphenyl-propanediamide (1 g, 2.02 mmol) in 2,2,2-trifluoroethanol (55.2 g, 40 mL, 552 mmol) was added [bis(trifluoroacetoxy)iodo]benzene (1.9 g, 4.4 mmol) portion-wise. The resulting mixture was stirred at RT for 3 days, then diluted with MeOH, and concentrated to dryness. The residue was purified by silica gel chromatography using a gradient of 0 to 10% (ammonia in MeOH) in DCM to give 746 mg (74%) of the title compound as a pale yellow solid. ES-MS m/z 491 (M+H).

### Preparation 144

### 3,3′-Spirobi[indoline]-2,2′-dione

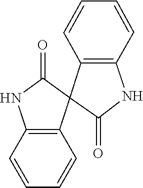 [SMILES: O=C1Nc2ccccc2C12C(=O)Nc1ccccc12]

To a solution of 1,1′-bis[(4-methoxyphenyl)methyl]-3,3′-spirobi[indoline]-2,2′-dione (746 mg, 1.52 mmol) in DCM (15 mL) was added TFA (15 mL, 22.6 g, 198 mmol) and trifluoromethanesulfonic acid (8 mL) drop-wise. The resulting mixture was stirred at RT and then concentrated under a stream of N₂. The residue was dissolved in DCM, then water was added and phases separated. The organic phase was washed with saturated aqueous NaHCO₃, then dried over Na₂SO₄, filtered and concentrated. The residue was purified by silica gel chromatograph using a gradient of 0 to 10% EtOAc in hexanes to give 210 mg (55%) of the title compound as a pale yellow solid. ES-MS m/z 251 (M+H).

### Preparation 145

### (2S)-3-[3-(2,2′-Dioxo-3,3′-spirobi[indoline]-1′-yl)phenyl]-2-[(3R)-pyrrolidin-3-yl]propanoic acid

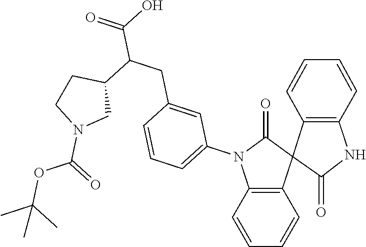 [SMILES: CC(C)(C)OC(=O)N1CC[C@H](C(Cc2cccc(N3C(=O)C4(C(=O)Nc5ccccc54)c4ccccc43)c2)C(=O)O)C1]

The title compound was prepared essentially as described in Preparation 140 using 3,3′-spirobi[indoline]-2,2′-dione. Upon completion, the reaction mixture was diluted with MeOH, filtered through diatomaceous earth, and concentrated to dryness. The residue was purified on an HLB column using a gradient of 0 to 70% ACN in aqueous NH₄HCO₃ (pH 9). ES-MS m/z 590 (M+H).

### Preparation 147

### (2S)-2-[(3R)-1-tert-Butoxycarbonylpyrrolidin-3-yl]-3-[3-(2-oxo-3H-benzimidazol-1-yl)phenyl]propanoic acid

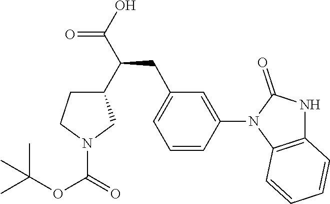 [SMILES: CC(C)(C)OC(=O)N1CC[C@H]([C@H](Cc2cccc(-n3c(=O)[nH]c4ccccc43)c2)C(=O)O)C1]

A suspension of (2S)-3-(3-bromophenyl)-2-[(3R)-1-tert-butoxycarbonylpyrrolidin-3-yl]propanoic acid (ammonium salt, 0.20 g, 0.48 mmol), 1,3-dihydrobenzimidazol-2-one (0.13 g, 0.96 mmol), cuprous iodide (0.14 g, 0.72 mmol), potassium carbonate (0.23 g, 1.69 mmol), and N,N′-dimethylethane-1,2-diamine (0.13 g, 1.45 mmol) in toluene (2.4 mL) in a pressure tube was sonicated for 5 min. The tube was sealed and the mixture was heated at 110° C. for 16 h. After cooling to RT, saturated aqueous NH₄Cl was added and the reaction extracted with EtOAc. The organic layer was dried over MgSO₄, filtered, and concentrated in vacuo. The residue was purified by reversed phase chromatography eluted with a gradient of 20% to 50% ACN in aqueous NH₄HCO₃ giving the title compound (68.2 mg, 31%) as a white solid. ES-MS m/z 352 (M+H-tBu).

### Preparation 148

### (2S)-2-[(3R)-1-tert-Butoxycarbonylpyrrolidin-3-yl]-3-[3-[2-oxo-3-(2,2,2-trifluoroethyl)imidazolidin-1-yl]phenyl]propanoic acid

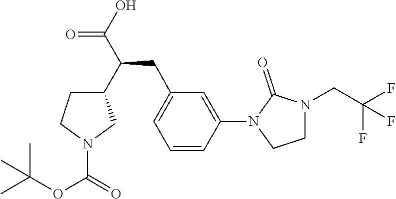 [SMILES: CC(C)(C)OC(=O)N1CC[C@H]([C@H](Cc2cccc(N3CCN(CC(F)(F)F)C3=O)c2)C(=O)O)C1]

A sealed tube was charged with (2S)-3-(3-bromophenyl)-2-[(3R)-1-tert-butoxycarbonylpyrrolidin-3-yl]propanoic acid (prepared essentially as described in WO 2020/247429, 0.15 g, 0.38 mmol), 1-(2,2,2-trifluoroethyl)imidazolidin-2-one (0.19 g, 1.13 mmol), t-BuXPhos Pd G3 (0.03 g, 0.04 mmol), and sodium tert-butoxide (0.22 g, 2.26 mmol) in anhydrous 1,4-dioxane (3.8 mL), purged with nitrogen for 5 min and stirred at 100° C. overnight. After cooling to RT, the reaction was filtered over a pad of diatomaceous earth and rinsed with EtOAc. The filtrate was extracted twice with 1M aqueous NaOH, the combined aqueous extracts were acidified to pH 3-4 with aqueous 1M HCl, and extracted twice with EtOAc. The combined organics were dried over Na₂SO₄, filtered, and concentrated under reduced pressure. The residue was purified by silica gel chromatography eluted with a gradient of 20% to 80% acetone in hexanes to give the title compound (75 mg, 40%) as a white solid. ES-MS m/z 386 (M+H-BOC).

### Preparation 149

### (2S)-2-[(3R)-1-tert-Butoxycarbonylpyrrolidin-3-yl]-3-[3-fluoro-5-(2-oxoindolin-1-yl)phenyl]propanoic acid

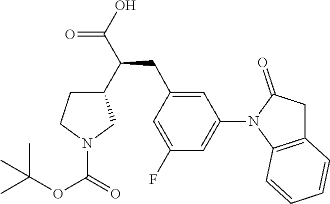 [SMILES: CC(C)(C)OC(=O)N1CC[C@H]([C@H](Cc2cc(F)cc(N3C(=O)Cc4ccccc43)c2)C(=O)O)C1]

A pressure tube was charged with 2-oxindole (0.11 g, 0.81 mmol), (2S)-3-(3-bromo-5-fluoro-phenyl)-2-[(3R)-1-tert-butoxycarbonylpyrrolidin-3-yl]propanoic acid (0.30 g, 0.72 mmol), and ACN (2.4 mL). A stream of N₂ was bubbled through the solution as it was heated to 40° C. over 15 min. The reaction was then treated with K₂CO₃ (0.33 g, 2.34 mmol), cuprous iodide (0.014 g, 0.072 mmol), and N,N′-dimethylethylenediamine (0.013 g, 0.14 mmol) giving a bright blue suspension. The tube was sealed and the mixture heated at 80° C. overnight. After 16 h, the reaction was cooled to RT, quenched with 1N aqueous HCl, and extracted with EtOAc. The combined organic phases were washed with saturated aqueous NaCl, dried over MgSO₄, filtered, and concentrated under reduced pressure. The residue was purified by silica gel chromatography eluted with a gradient of 12% to 50% acetone (with 1% HOAc) in hexanes giving the title compound (0.22 g, 89 mass %, 57%) as yellow solid. ES-MS m/z 369 (M+H-BOC).

### Preparation 150

### (2S)-2-[(3R)-1-tert-Butoxycarbonylpyrrolidin-3-yl]-3-[4-(2,5-dioxoimidazolidin-1-yl)phenyl]propanoic acid

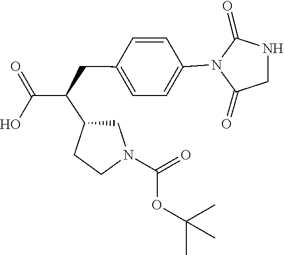 [SMILES: CC(C)(C)OC(=O)N1CC[C@H]([C@H](Cc2ccc(N3C(=O)CNC3=O)cc2)C(=O)O)C1]

Hydantoin (0.25 g, 2.4 mmol), (2S)-3-(4-bromophenyl)-2-[(3R)-1-tert-butoxycarbonylpyrrolidin-3-yl]propanoic acid (0.32 g, 0.80 mmol), cuprous iodide (0.46 g, 2.4 mmol), K₂CO₃ (0.33 g, 2.4 mmol), and DMF (5 mL) were combined in a microwave vial under nitrogen. N,N′-Dimethylethylenediamine (0.22 g, 2.4 mmol) was added and the vial sealed. The suspension was heated at 100° C. for 1 h. The reaction was cooled, diluted with EtOAc, washed with 10% aqueous citric acid, washed with water (×3), and then saturated aqueous NaCl. Dried the organics over Na₂SO₄, filtered, and concentrated under reduced pressure. The residue was purified by silica gel chromatography eluted with a gradient of 0% to 100% acetone (with 2% AcOH) in hexanes. The title compound (23 mg, 7%) was isolated as a clear wax. ES-MS m/z 362 (M+H-tBu).

### Preparation 151

### tert-Butyl (3R)-3-[(1S)-2-tert-butoxy-2-oxo-1-[[3-(4-phenyl-1-piperidyl)phenyl]methyl]ethyl]pyrrolidine-1-carboxylate

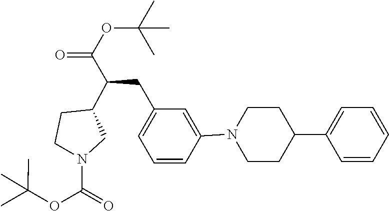 [SMILES: CC(C)(C)OC(=O)[C@@H](Cc1cccc(N2CCC(c3ccccc3)CC2)c1)[C@H]1CCN(C(=O)OC(C)(C)C)C1]

A tube was charged with (2S)-3-(3-bromophenyl)-2-[(3R)-1-tert-butoxycarbonylpyrrolidin-3-yl]propanoic acid (prepared essentially as described in WO 2020/247429, 0.20 g, 0.44 mmol), cesium carbonate (0.57 g, 1.76 mmol), and toluene (3 mL). The reaction was purged with N₂, then 4-phenylpiperidine hydrochloride (0.10 g, 0.53 mmol) and methanesulfonato(2-dicyclohexylphosphino-2′,6′-di-1-propoxy-1,1′-biphenyl)(2′-amino-1,1′-biphenyl-2-yl)palladium(II) (0.11 g, 0.13 mmol) were added. The tube was capped and the reaction stirred at 85° C. for 16 h. The reaction was allowed to cool, water was added, and the reaction extracted with EtOAc. The organic layer was collected, dried over MgSO₄, and concentrated under reduced pressure. The residue was taken up in EtOAc, filtered through celite, and the solids washed with EtOAc. The filtrate was concentrated under reduced pressure affording a yellow oil which was purified by silica gel chromatography eluted with a gradient of 0% to 35% EtOAc in hexanes to afford the title compound (0.19 g, 77%) as a colourless oil which was taken on without analysis.

### Preparation 153

### (2S)-2-[(3R)-1-tert-Butoxycarbonylpyrrolidin-3-yl]-3-[3-(2-fluoro-3-methoxy-phenyl)phenyl]propanoic acid

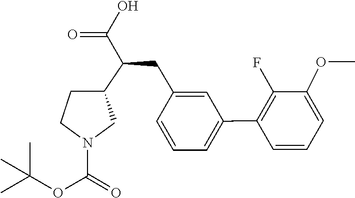 [SMILES: COc1cccc(-c2cccc(C[C@H](C(=O)O)[C@H]3CCN(C(=O)OC(C)(C)C)C3)c2)c1F]

A pressure tube was charged with (2S)-3-(3-bromophenyl)-2-[(3R)-1-tert-butoxycarbonylpyrrolidin-3-yl]propanoic acid (ammonium salt, 0.25 g, 0.60 mmol), 2-fluoro-3-methoxyphenylboronic acid (0.16 g, 0.90 mmol), potassium carbonate (0.25 g, 1.81 mmol), 1,4-dioxane (4.2 mL), water (0.60 mL), and 1,1′-bis(diphenylphosphino)ferrocene-palladium(II)dichloride DCM complex (0.05 g, 0.06 mmol). The tube was sealed and the mixture was heated at 100° C. for 2 h. After cooling, the reaction was quenched with 5% aqueous citric acid and extracted with EtOAc. The combined organic phases were filtered through diatomaceous earth, washed with saturated aqueous NaCl, dried over MgSO₄, filtered, and concentrated under reduced pressure giving the title compound (0.27 g, 100%) which was used with purification. ES-MS m/z 388 (M+H-BOC).

### Preparation 154

### (2S)-2-[(3R)-1-tert-Butoxycarbonylpyrrolidin-3-yl]-3-[3-(7-methoxybenzothiophen-2-yl)phenyl]propanoic acid

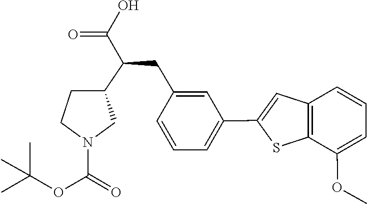 [SMILES: COc1cccc2cc(-c3cccc(C[C@H](C(=O)O)[C@H]4CCN(C(=O)OC(C)(C)C)C4)c3)sc12]

A sealed tube was charged with (2S)-2-[(3R)-1-tert-butoxycarbonylpyrrolidin-3-yl]-3-[3-(4,4,5,5-tetramethyl-1,3,2-dioxaborolan-2-yl)phenyl]propanoic acid (0.20 g, 0.45 mmol), 2-iodo-7-methoxy-benzothiophene (0.20 g, 0.68 mmol), and anhydrous DMF (4.5 mL). The reaction was purged with N₂ for 10 min, and then treated with aqueous potassium phosphate, dibasic (1 M, 2.25 mL, 2.25 mmol) and tetrakis(triphenylphosphine)palladium(0) (0.01 g, 0.01 mmol) were added. The tube was sealed and the reaction mixture stirred at 80° C. for 3 h. Reaction was allowed to cool to RT, purged with nitrogen for 5 min, and re-treated with tetrakis(triphenylphosphine)palladium(0) (0.01 g, 0.01 mmol) and the reaction stirred at 80° C. for 3 days. Reaction was allowed to cool to RT. The mixture was quenched with 5% aqueous citric acid and extracted with EtOAc. The organic layer was washed with saturated aqueous NaCl, dried over Na₂SO₄, filtered, and concentrated under reduced pressure. The residue was purified by silica gel chromatography eluted with a gradient of 10% to 40% acetone in hexanes giving the title compound (0.15 g, 69%) as a beige solid. ES-MS m/z 480 (M−H).

### Preparation 155

### (2S)-2-[(3R)-1-tert-Butoxycarbonylpyrrolidin-3-yl]-3-[3-[3-(2,2,2-trifluoroethoxy)phenyl]phenyl]propanoic acid

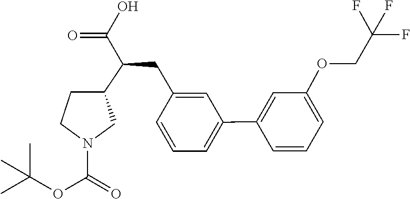 [SMILES: CC(C)(C)OC(=O)N1CC[C@H]([C@H](Cc2cccc(-c3cccc(OCC(F)(F)F)c3)c2)C(=O)O)C1]

To a stirred mixture of (2S)-3-(3-bromophenyl)-2-[(3R)-1-tert-butoxycarbonylpyrrolidin-3-yl]propanoic acid (ammonium salt, 0.25 g, 0.60 mmol), 3-(2,2,2-trifluoroethoxy)phenylboronic acid (0.20 g, 0.90 mmol), potassium carbonate (0.25 g, 1.81 mmol), 1,4-dioxane (4.2 mL), and water (0.60 mL) at 100° C. was added 1,1′-bis(diphenylphosphino)ferrocene-palladium(II) dichloride DCM complex (0.05 g, 0.06 mmol). The tube was sealed and the mixture was heated at 100° C. overnight. Stirred at RT for 24 h. The reaction was quenched with 5% aqueous citric acid and extracted with EtOAc. The combined organic phases were washed with saturated aqueous NaCl, dried over MgSO₄, filtered, and concentrated under reduced pressure giving the title compound (0.30 g, 100%) which was used without purification. ES-MS m/z 516 (M+Na).

### Preparation 156

### (2S)-3-[3-(1,3-Benzothiazol-2-yl)phenyl]-2-[(3R)-1-tert-butoxycarbonylpyrrolidin-3-yl]propanoic acid

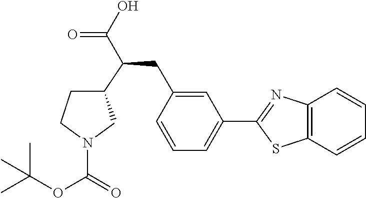 [SMILES: CC(C)(C)OC(=O)N1CC[C@H]([C@H](Cc2cccc(-c3nc4ccccc4s3)c2)C(=O)O)C1]

A pressure vial was charged with (2S)-2-[(3R)-1-tert-butoxycarbonylpyrrolidin-3-yl]-3-[3-(4,4,5,5-tetramethyl-1,3,2-dioxaborolan-2-yl)phenyl]propanoic acid (0.25 g, 0.56 mmol), 2-bromo-1,3-benzothiazole (0.19 g, 0.84 mmol), DMF (5.6 mL), and aqueous potassium phosphate, dibasic (1 M, 2.8 mL). The tube was warmed to 80° C. and tetrakis(triphenylphosphine)palladium(0) (0.013 g, 0.011 mmol) added. The tube was sealed and the mixture was heated at 80° C. for 90 min. The reaction was quenched with 5% aqueous citric acid and extracted with EtOAc. The combined organic phases were washed with saturated aqueous NaCl, dried over MgSO₄, filtered, and concentrated under reduced pressure to give the title compound (0.25 g, 100%) which was taken on to the next synthetic step without purification. ES-MS m/z 453 (M+H).

### Preparation 157

### (2S)-2-[(3R)-1-tert-Butoxycarbonylpyrrolidin-3-yl]-3-[3-(3-phenylisoxazol-5-yl)phenyl]propanoic acid

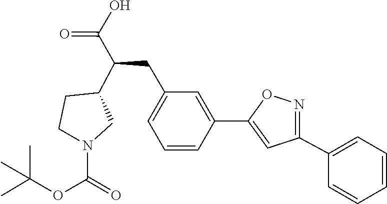 [SMILES: CC(C)(C)OC(=O)N1CC[C@H]([C@H](Cc2cccc(-c3cc(-c4ccccc4)no3)c2)C(=O)O)C1]

A tube was charged with (2S)-3-(3-bromophenyl)-2-[(3R)-1-tert-butoxycarbonylpyrrolidin-3-yl]propanoic acid (prepared essentially as described in WO 2020/247429, 100 mg, 0.25 mmol), (3-phenylisoxazol-5-yl)boronic acid (61.7 mg, 0.33 mmol), and tetrakis(triphenylphosphine)palladium(0) (23.9 mg, 0.02 mmol). The tube was purged with nitrogen and then 1,4-dioxane (1 mL) was added. Purged with nitrogen and added 2M aqueous K₂CO₃ (0.25 mL, 0.50 mmol). The tube was purged with nitrogen a third time, capped, and the reaction stirred at 100° C. for 18 h. Another 26 mg of (3-phenylisoxazol-5-yl)boronic acid was added and the reaction stirred at 100° C. for another 2 h. After cooling, the reaction was loaded onto an ACN and aqueous NH₄HCO₃ conditioned HLB cartridge and eluted with aqueous NH₄HCO₃ and ACN. The crude material was purified with reversed phase purification to give the title compound (116.1 mg, 100%). ES-MS m/z 463 (M+H).

### Preparation 158

### (2S)-3-(3-Aminophenyl)-2-[(3R)-1-tert-butoxycarbonylpyrrolidin-3-yl]propanoic acid

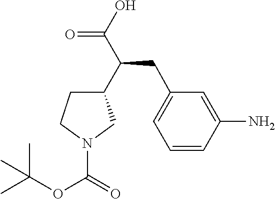 [SMILES: CC(C)(C)OC(=O)N1CC[C@H]([C@H](Cc2cccc(N)c2)C(=O)O)C1]

A mixture of (2S)-2-[(3R)-1-tert-butoxycarbonylpyrrolidin-3-yl]-3-(3-nitrophenyl)propanoic acid (prepared essentially as described in WO 2020/247429, 1.70 g, 4.67 mmol) and palladium (10% on activated carbon paste type 87 μL, 0.17 g) in MeOH (25 mL) was stirred under H₂ (balloon) at RT for 4 h. The catalyst was removed by filtration through diatomaceous earth and the filtrate concentrated under reduced pressure. The residue was purified by reversed phase chromatography eluted with a gradient of 10% to 40% ACN in water to give the title compound (1.00 g, 64%) as a white solid. ES-MS m/z 235 (M+H-BOC).

### Preparation 159

### (2S)-3-(3-Azidophenyl)-2-[(3R)-1-tert-butoxycarbonylpyrrolidin-3-yl]propanoic acid

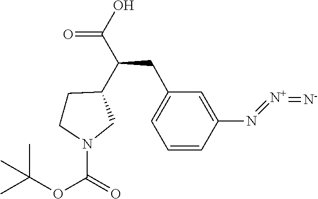 [SMILES: CC(C)(C)OC(=O)N1CC[C@H]([C@H](Cc2cccc(N=[N+]=[N-])c2)C(=O)O)C1]

The title compound was prepared by flow reaction, first by preparing the following solutions: Solution A—(2S)-3-(3-aminophenyl)-2-[(3R)-1-tert-butoxycarbonylpyrrolidin-3-yl]propanoic acid (0.80 g, 2.39 mmol) and DMF (80 mL) treated with azidotrimethylsilane (0.34 g, 2.87 mmol); and Solution B—tert-butyl nitrite (0.37 g, 3.59 mmol) in DMF (80 mL). Solution A and B were mixed by passing through a flow reactor (PTFE tubing, 15 mL volume) with Solution A at a flow rate of 0.1 mL/min and solution B at a flow rate of 0.13 mL/min at a temperature of 100° C. to give a solution (120 mL, 0.015 M) of the title compound which was used without purification.

### Preparation 160

### (2S)-3-[3-(4-Benzyltriazol-1-yl)phenyl]-2-[(3R)-1-tert-butoxycarbonylpyrrolidin-3-yl]propanoic acid

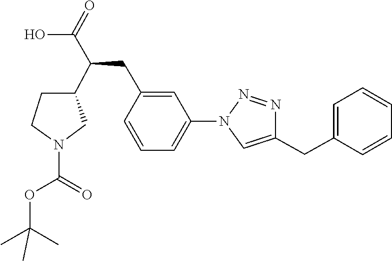 [SMILES: CC(C)(C)OC(=O)N1CC[C@H]([C@H](Cc2cccc(-n3cc(Cc4ccccc4)nn3)c2)C(=O)O)C1]

A solution of (2S)-3-(3-azidophenyl)-2-[(3R)-1-tert-butoxycarbonylpyrrolidin-3-yl]propanoic acid 0.015 M in DMF (10 mL, 0.15 mmol) was combined with a solution of prop-2-ynylbenzene (30 mg, 0.26 mmol), aqueous sodium ascorbate (0.2 M, 0.40 mL, 0.08 mmol), tris[(1-benzyl-1H-1,2,3-triazol-4-yl)methyl]amine (8.0 mg, 0.02 mmol), and aqueous copper(II) sulfate pentahydrate (0.05 M, 0.30 mL, 0.02 mmol). The reaction was stirred at 50° C. for 2 h. Analysis indicated mainly starting material. More tris[(1-benzyl-1H-1,2,3-triazol-4-yl)methyl]amine (8.0 mg, 0.02 mmol), aqueous sodium ascorbate (0.2 M, 0.40 mL, 0.08 mmol), and aqueous copper(II) sulfate pentahydrate (0.05 M, 0.30 mL, 0.02 mmol) were added. The reaction was stirred at 50′° C. Analysis indicated some starting material still present. Sodium ascorbate (0.2 M, 0.40 mL, 0.08 mmol) and aqueous copper(II) sulfate pentahydrate (0.05 M, 0.30 mL, 0.02 mmol) was added and the reaction was stirred at 50° C. for 2 h. After cooling, water was added. Aqueous layer was extracted with EtOAc and the organic layer was dried over Na₂SO₄. Filtrate was concentrated under reduced pressure and the residue was purified by reversed phase chromatography eluted with 2000 to 5000 ACN in water to give the title compound (60.7 mg, 850%) as a thick yellow oil. ES-MS m/z 477 (M+H).

The following compounds were prepared essentially as described in Preparation 160 using the appropriate reagents, adjusting the temperature, and the reaction times to determine completion of the reactions, and adjusting the purification system as appropriate.

| | | | ES/MS |
|---|---|---|---|
| Prep | | | m/z |
| # | Chemical Name | Structure | [M + H]⁺ |
| 161 | (2S)-2-[(3R)-1-tert- Butoxycarbonylpyrrolidin- 3-yl]-3-[3-[4-(3- methoxyphenyl)triazol-1- yl]phenyl]propanoic acid | 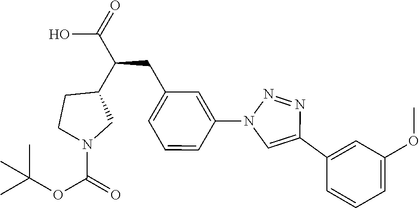 [SMILES: COc1cccc(-c2cn(-c3cccc(C[C@H](C(=O)O)[C@H]4CCN(C(=O)OC(C)(C)C)C4)c3)nn2)c1] | 393 (M + H − BOC) |
| 162 | (2S)-2-[(3R)-1-tert- Butoxycarbonylpyrrolidin- 3-yl]-3-[3-[4-(2,5- difluorophenyl)triazol-1- yl]phenyl]propanoic acid | 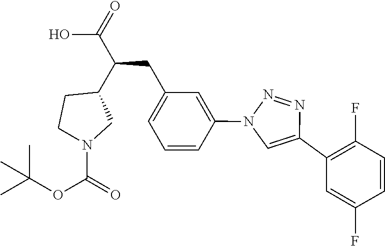 [SMILES: CC(C)(C)OC(=O)N1CC[C@H]([C@H](Cc2cccc(-n3cc(-c4cc(F)ccc4F)nn3)c2)C(=O)O)C1] | 499 (M + H) |
| 163 | (2S)-2-[(3R)-1-tert- Butoxycarbonylpyrrolidin- 3-yl]-3-[3-[4- (hydroxymethyl)triazol-1- yl]phenyl]propanoic acid | 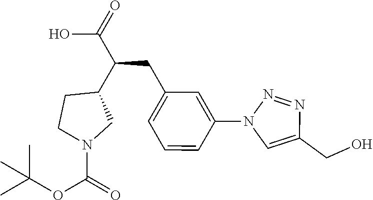 [SMILES: CC(C)(C)OC(=O)N1CC[C@H]([C@H](Cc2cccc(-n3cc(CO)nn3)c2)C(=O)O)C1] | 417 (M + H) |

### Preparation 164

### (2S)-2-[(3R)-1-tert-Butoxycarbonylpyrrolidin-3-yl]-3-(3-tetrahydropyran-4-ylphenyl)propanoic acid

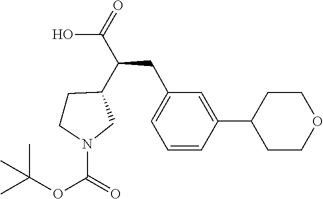 [SMILES: CC(C)(C)OC(=O)N1CC[C@H]([C@H](Cc2cccc(C3CCOCC3)c2)C(=O)O)C1]

A mixture of (2S)-3-(3-bromophenyl)-2-[(3R)-1-tert-butoxycarbonylpyrrolidin-3-yl]propanoic acid (prepared essentially as described in WO 2020/247429, 0.14 g, 0.35 mmol) and dichloro[1,3-bis(2,6-di-3-pentylphenyl)imidazol-2-ylidene](3-chloropyridyl)palladium(II) (0.01 g, 0.014 mmol) in dry 2-methyltetrahydrofuran (1.1 mL) was degassed for 5 min with nitrogen. Then, bromo(tetrahydropyran-4-yl)zinc (3 mL, 0.5 mol/L in THF, 1.40 mmol) was added, the reaction vessel was sealed, and the reaction heated to 100° C. for 30 min under microwave irradiation. Analysis still indicated starting material was present. 2-Methyltetrahydrofuran (1.1 mL) and dichloro[1,3-bis(2,6-di-3-pentylphenyl)imidazol-2-ylidene](3-chloropyridyl)palladium(II) (0.01 g, 0.014 mmol) were added followed by addition of bromo(tetrahydropyran-4-yl)zinc (4 mL, 0.5 mol/L in THF, 2.09 mmol) at RT under a nitrogen atmosphere. The reaction was heated again to 100° C. for 30 min under microwave irradiation. The crude material from a 50 mg reaction was added to this reaction. The mixture was filtered through a pad of diatomaceous earth and the filtrate evaporated under reduced pressure. The residue was suspended in DMSO (2 mL) and ACN (3 mL). The suspension was filtered through diatomaceous earth and the filtrate concentrated under reduced pressure. The remaining DMSO solution was purified by C18 reversed phase chromatography eluted with a gradient of 0% to 100% ACN in water. The resulting material was re-purified by reversed phase chromatography (XBridge® C18) eluted with a gradient of 5% to 40% ACN in aqueous 20 mM NH₄HCO₃ giving the title compound (10.5 mg, 7%) as a white solid. ES-MS m/z 402 (M−H).

### Preparation 165

### tert-Butyl (3R)-3-[(1S)-2-tert-butoxy-1-[(3-cyclobutylphenyl)methyl]-2-oxo-ethyl]pyrrolidine-1-carboxylate

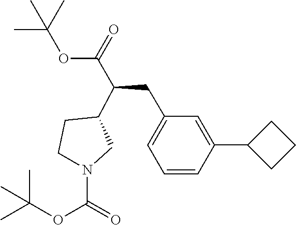 [SMILES: CC(C)(C)OC(=O)[C@@H](Cc1cccc(C2CCC2)c1)[C@H]1CCN(C(=O)OC(C)(C)C)C1]

tert-Butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 200 mg, 0.44 mmol), dichloro[1,3-bis(2,6-di-3-pentylphenyl)imidazol-2-ylidene](3-chloropyridyl)palladium(II) (7.36 mg, 0.01 mmol) in THE (1.76 mL) was degassed several times. Cyclobutylzinc bromide (0.5 mol/L in THF, 1 mL) was added dropwise and the reaction was stirred at 70° C. for 1 h. The reaction was evaporated under reduced pressure and the residue dissolved in DCM and filtered to remove inorganics. The filtrate was evaporated under reduced pressure to give a brown oil which was purified by silica gel chromatography eluted with a gradient of 0% to 15% EtOAc in hexane to give the title compound (0.11 g, 56%) as an amber oil. ES-MS m/z 318 (M+H-2(tBu)).

### Preparation 166

### (Z)-3-Amino-3-ethoxy-prop-2-enenitrile

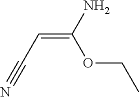

Malononitrile (0.51 g, 7.57 mmol) and EtOH (0.38 g, 8.32 mmol) were dissolved in 2M HCl in Et₂O (60.5 mL) at 0° C. under a nitrogen atmosphere. Reaction was allowed to warm to RT and stirred for 4 h. The resulting precipitate was removed by filtration and washed extensively with Et₂O. The solid was treated with saturated aqueous K₂CO₃ and extracted with Et₂O. The organic layer was collected, dried over Na₂SO₄, filtered, and concentrated under reduced pressure to give the title compound (0.51 g, 60%) as a white solid. ¹H NMR (400 MHz, DMSO-d₆) δ ppm 1.21 (t, J=6 Hz, 3H), 3.85 (d, J=6 Hz, 2H), 6.51 (s, 1H).

### Preparation 167

### tert-Butyl (3R)-3-[(1S)-1-[[3-[[(E)-1-amino-2-cyano-vinyl]amino]phenyl]methyl]-2-tert-butoxy-2-oxo-ethyl]pyrrolidine-1-carboxylate

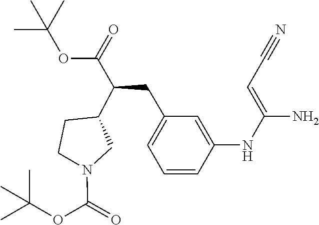 [SMILES: CC(C)(C)OC(=O)[C@@H](Cc1cccc(N/C(N)=C/C#N)c1)[C@H]1CCN(C(=O)OC(C)(C)C)C1]

A tube was charged with (Z)-3-amino-3-ethoxy-prop-2-enenitrile (50 mg, 0.45 mmol), tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 210 mg, 0.54 mmol) and EtOH (0.45 mL). The reaction was stirred under a nitrogen atmosphere at RT overnight. After 20 h, the reaction was concentrated under reduce pressure. The residue was purified by silica gel chromatography eluted with a gradient of 5% to 20% acetone in DCM. The resulting material was repurified by reversed phase chromatography eluted with a gradient of 50% to 80% ACN in aqueous NH₄HCO₃ giving the title compound (64 mg, 31%) as a beige solid which was a 60:40 mixture of the cis and trans isomers. ES-MS m/z 457 (M+H).

### Preparation 168

### tert-Butyl (3R)-3-[(1S)-2-[(4S)-4-benzyl-2-oxo-oxazolidin-3-yl]-1-[(3-benzylsulfanylphenyl)methyl]-2-oxo-ethyl]pyrrolidine-1-carboxylate

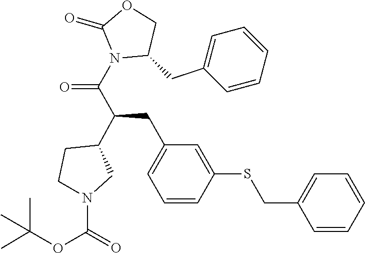 [SMILES: CC(C)(C)OC(=O)N1CC[C@H]([C@H](Cc2cccc(SCc3ccccc3)c2)C(=O)N2C(=O)OC[C@@H]2Cc2ccccc2)C1]

Nitrogen was bubbled through a suspension of tert-butyl (3R)-3-[(1S)-2-[(4S)-4-benzyl-2-oxo-oxazolidin-3-yl]-1-[(3-bromophenyl)methyl]-2-oxo-ethyl]pyrrolidine-1-carboxylate (prepared essentially as described in WO 2020/247429, 1.0 g, 1.79 mmol) in toluene (4 mL) and then tris(dibenzylideneacetone)dipalladium(0) (0.17 g, 0.18 mmol), 1,1′-bis(diphenylphosphino)ferrocene (0.21 g, 0.36 mmol), N,N-diisopropylethylamine (0.26 g, 1.97 mmol), and benzyl mercaptan (0.23 g, 1.79 mmol) were added. The reaction was stirred at 100° C. After 16 h, the reaction was allowed to cool and filtered. The filtrate was treated with water and extracted with EtOAc. The organic layer was collected, dried over MgSO₄, filtered, and concentrated under reduced pressure. The residue was purified by silica gel chromatography eluted with a gradient of 20% to 50% EtOAc in hexane. The resulting material was repurified by silica gel chromatography washing with DCM and then eluting with a gradient of 20% to 50% EtOAc in hexane to give the title compound (0.97 g, 71% pure, 64%). ES-MS m/z 501 (M+H-BOC).

### Preparation 169

### tert-Butyl (3R)-3-[(1S)-2-[(4S)-4-benzyl-2-oxo-oxazolidin-3-yl]-1-[(3-chlorosulfonylphenyl)methyl]-2-oxo-ethyl]pyrrolidine-1-carboxylate

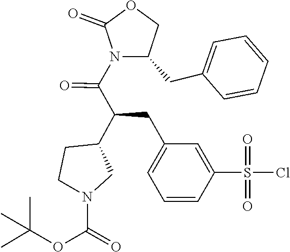 [SMILES: CC(C)(C)OC(=O)N1CC[C@H]([C@H](Cc2cccc(S(=O)(=O)Cl)c2)C(=O)N2C(=O)OC[C@@H]2Cc2ccccc2)C1]

A 0° C. solution of tert-butyl (3R)-3-[(1S)-2-[(4S)-4-benzyl-2-oxo-oxazolidin-3-yl]-1-[(3-benzylsulfanylphenyl)methyl]-2-oxo-ethyl]pyrrolidine-1-carboxylate (1.02 g, 1.21 mmol, 71 mass %) in ACN:acetic acid:water 40:1.5:1 (15 μL) was slowly treated with 1,3-dibromo-5,5-dimethylhydantoin (0.70 g, 2.41 mmol) in portions. The reaction was stirred at 0° C. for 10 min. The reaction was concentrated under reduced pressure. The residue was dissolved in DCM, cooled to 0° C., and treated with 5% aqueous NaHCO₃. After stirring for 5 min, the organic layer was collected, dried over MgSO₄, filtered, and concentrated under reduced pressure to give the title compound (1.1 g, 64 mass %, 100%) which was taken on to the next synthetic reaction without purification. ES-MS m/z 521 (M+H-BOC).

### Preparation 170

### tert-Butyl (3R)-3-[(1S)-2-[(4S)-4-benzyl-2-oxo-oxazolidin-3-yl]-1-[[3-(dimethylsulfamoyl)phenyl]methyl]-2-oxo-ethyl]pyrrolidine-1-carboxylate

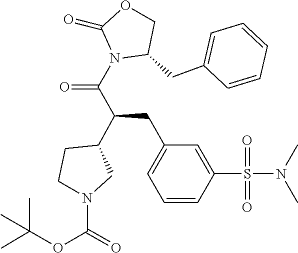 [SMILES: CN(C)S(=O)(=O)c1cccc(C[C@H](C(=O)N2C(=O)OC[C@@H]2Cc2ccccc2)[C@H]2CCN(C(=O)OC(C)(C)C)C2)c1]

A RT solution of tert-butyl (3R)-3-[(1S)-2-[(4S)-4-benzyl-2-oxo-oxazolidin-3-yl]-1-[(3-chlorosulfonylphenyl)methyl]-2-oxo-ethyl]pyrrolidine-1-carboxylate (0.54 g, 64 mass %, 0.60 mmol) in DCM (5 mL) was treated with TEA (0.12 g, 1.21 mmol) and dimethylamine (0.04 g, 40% in water, 0.90 mmol). The reaction was stirred at RT for 60 min. Water was added, the reaction was acidified with 1M aqueous HCl, and extracted with DCM. The organic layer was collected, dried over MgSO₄, collected, and concentrated under reduced pressure. The residue was purified by silica gel chromatography eluted with a gradient of 25% to 100% EtOAc in hexane to give the title compound (0.33 g, 93%) as an oil. ES-MS m/z 530 (M+H-^{t}Bu).

### Preparation 171

### (2S)-2-[(3R)-1-tert-Butoxycarbonylpyrrolidin-3-yl]-3-[3-(dimethylsulfamoyl)phenyl]propanoic acid

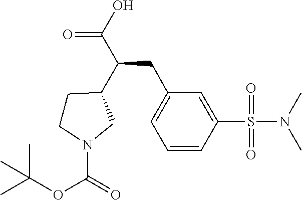 [SMILES: CN(C)S(=O)(=O)c1cccc(C[C@H](C(=O)O)[C@H]2CCN(C(=O)OC(C)(C)C)C2)c1]

A solution of tert-butyl (3R)-3-[(1S)-2-[(4S)-4-benzyl-2-oxo-oxazolidin-3-yl]-1-[[3-(dimethylsulfamoyl)phenyl]methyl]-2-oxo-ethyl]pyrrolidine-1-carboxylate (0.33 g, 0.56 mmol) in THE (5 mL) was treated with 1M aqueous lithium hydroxide (0.84 mL, 0.84 mmol) and hydrogen peroxide (0.44 mL, 35 mass %, 4.48 mmol). The reaction was stirred for 16 h. The reaction was treated with 1N aqueous NaOH and extracted with MTBE. The aqueous layer was collected, acidified with 1N aqueous HCl, and extracted with EtOAc. The organic layer was collected, dried over MgSO₄, filtered, and concentrated under reduced pressure. The residue was purified by reversed phase chromatography eluted with a gradient of 20% to 40% ACN in 20 mM aqueous NH₄HCO₃ to give the title compound (73 mg, 30%) as a white solid. ES-MS m/z 371 (M+H-tBu).

### Preparation 172

### tert-Butyl (3R)-3-[2-[(4S)-4-benzyl-2-oxo-oxazolidin-3-yl]-2-oxo-1-[[4-(trifluoromethyl)phenyl]methyl]ethyl]pyrrolidine-1-carboxylate

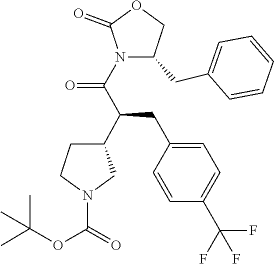 [SMILES: CC(C)(C)OC(=O)N1CC[C@H]([C@H](Cc2ccc(C(F)(F)F)cc2)C(=O)N2C(=O)OC[C@@H]2Cc2ccccc2)C1]

A solution of tert-butyl (3R)-3-[2-[(4S)-4-benzyl-2-oxo-oxazolidin-3-yl]-2-oxo-ethyl]pyrrolidine-1-carboxylate (prepared essentially as described in WO 2020/247429, 0.30 g, 0.77 mmol) in THE (7.72 mL) at −78° C. was treated with lithium bis(trimethylsilyl)amide (1.0 mol/L in THE (0.93 mL, 0.93 mmol) and the mixture was stirred for 90 min. The reaction was then treated with a solution of 4-(trifluoromethyl)benzyl bromide (0.20 g, 0.82 mmol) in THE (3.86 mL) and the resulting mixture was allowed to reach RT and stir overnight. The reaction was quenched with saturated aqueous NH₄Cl and extracted with EtOAc. The organic phases were washed with saturated aqueous NaCl, collected, dried over MgSO₄, filtered, and concentrated under reduced pressure. The residue was purified by reversed phase chromatography eluted with a gradient of 60% to 90% ACN in aqueous NH₄HCO₃ to give the title compound (0.20 g, 47%) as a colorless oil. ES-MS m/z 491 (M+H-^{t}Bu).

### Preparation 173

### (2S)-2-[(3R)-1-tert-Butoxycarbonylpyrrolidin-3-yl]-3-[4-(trifluoromethyl)phenyl]propanoic acid

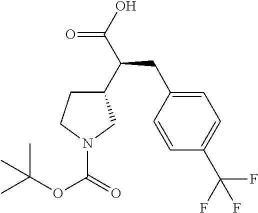 [SMILES: CC(C)(C)OC(=O)N1CC[C@H]([C@H](Cc2ccc(C(F)(F)F)cc2)C(=O)O)C1]

A solution of tert-butyl (3R)-3-[2-[(4S)-4-benzyl-2-oxo-oxazolidin-3-yl]-2-oxo-1-[[4-(trifluoromethyl)phenyl]methyl]ethyl]pyrrolidine-1-carboxylate (0.20 g, 0.37 mmol) in THE (2.5 mL) and water (0.48 mL) at 0° C. was treated with hydrogen peroxide (0.30 mL, 30 mass % in water, 2.93 mmol) followed by lithium hydroxide (0.02 g, 0.64 mmol) in water (0.73 mL). The reaction mixture was stirred at 0° C. After 30 min, the reaction was quenched with sodium thiosulfate (0.24 g, 1.46 mmol) in water (0.73 mL) at 0° C. and the reaction allowed to reach RT. The solution was extracted with EtOAc. The organic phase was concentrated under reduced pressure. The pH of the residue was adjusted to about 3 with 5% aqueous solution of citric acid and extracted with EtOAc. The combined organic phases were washed with saturated aqueous NaCl, dried over MgSO₄, filtered, and concentrated under reduced pressure. The residue was purified by reversed phase chromatography eluted with a gradient of 20% to 50% ACN in aqueous NH₄HCO₃ to give the title compound (60 mg, 40%) as a white solid. ES-MS m/z 288 (M+H-BOC).

### Preparation 174

### (2S)-2-[(3R)-1-tert-Butoxycarbonylpyrrolidin-3-yl]-3-[3-(1,1-dioxo-1,2-thiazolidin-2-yl)phenyl]propanoic acid

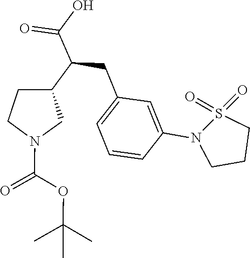 [SMILES: CC(C)(C)OC(=O)N1CC[C@H]([C@H](Cc2cccc(N3CCCS3(=O)=O)c2)C(=O)O)C1]

The title compound was prepared essentially as described in Preparation 23 using isothiazolidine 1,1-dioxide. Upon completion, the reaction was filtered through a pad of diatomaceous earth and the filtrate was concentrated to dryness. The residue was purified using an HLB cartridge eluting with 0 to 100% ACN in aqueous NH₄HCO₃ (pH 9). ES-MS m/z 383 (M+H-^{t}Bu).

### Preparation 175

### tert-Butyl (3R)-3-[(1S)-1-[[3-[(3-bromophenoxy)methyl]phenyl]methyl]-2-tert-butoxy-2-oxo-ethyl]pyrrolidine-1-carboxylate

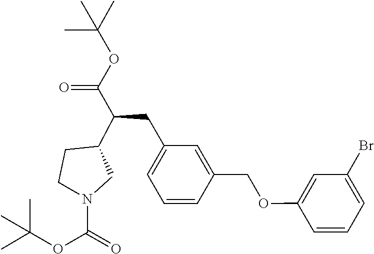 [SMILES: CC(C)(C)OC(=O)[C@@H](Cc1cccc(COc2cccc(Br)c2)c1)[C@H]1CCN(C(=O)OC(C)(C)C)C1]

A stirred solution of tert-butyl (3R)-3-[(1S)-2-tert-butoxy-1-[[3-(hydroxymethyl)phenyl]methyl]-2-oxo-ethyl]pyrrolidine-1-carboxylate (prepared essentially as described in WO 2020/247429; 100 mg, 0.25 mmol) in anhydrous THE (2.47 mL) was treated with 3-bromophenol (0.06 g, 0.37 mmol) and triphenylphosphine (0.10 g, 0.37 mmol). After purging the reaction with N₂, diisopropyl azodicarboxylate (0.07 g, 0.37 mmol) was added and the resulting mixture was allowed to stir at RT for 16 h. The reaction was concentrated under reduced pressure and the residue loaded onto diatomaceous earth. Residue was purified by silica gel chromatography eluted with a gradient of 10% to 40% MTBE in hexane giving the title compound (107 mg, 75%). ES-MS m/z 558/560 (M+H).

### Preparation 176

### tert-Butyl (3R)-3-[(1S)-2-tert-butoxy-2-oxo-1-[[3-[(3-phenylphenoxy)methyl]phenyl]methyl]ethyl]pyrrolidine-1-carboxylate

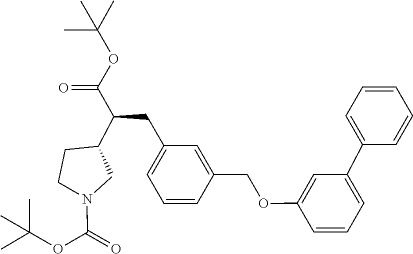 [SMILES: CC(C)(C)OC(=O)[C@@H](Cc1cccc(COc2cccc(-c3ccccc3)c2)c1)[C@H]1CCN(C(=O)OC(C)(C)C)C1]

A stirred suspension of tert-butyl (3R)-3-[(1S)-1-[[3-[(3-bromophenoxy)methyl]phenyl]methyl]-2-tert-butoxy-2-oxo-ethyl]pyrrolidine-1-carboxylate (0.11 g, 0.19 mmol), phenylboronic acid (0.03 g, 0.29 mmol), K₂CO₃ (0.08 g, 0.57 mmol) in 1,4-dioxane (1.9 mL) and water (0.19 mL) was treated with 1,1′-bis(diphenylphosphino)ferrocene-palladium(II)dichloride DCM complex (0.02 g, 0.02 mmol). The resulting mixture was degassed for 5 minutes and heated at 90° C. for 2 h. The reaction was allowed to cool, treated with water, and extracted with EtOAc. The organic layer was washed with saturated aqueous NaCl, collected, dried over MgSO₄, filtered, and concentrated under reduced pressure. The residue was purified by silica gel chromatography eluted with a gradient of 5% to 20% MTBE in hexane to give the title compound (91 mg, 85%) as a colorless oil. ES-MS m/z 458 (M+H-BOC).

### Preparation 177

### tert-Butyl (3R)-3-[(1S)-2-[(4S)-4-benzyl-2-oxo-oxazolidin-3-yl]-1-[(3-benzyloxyphenyl)methyl]-2-oxo-ethyl]pyrrolidine-1-carboxylate

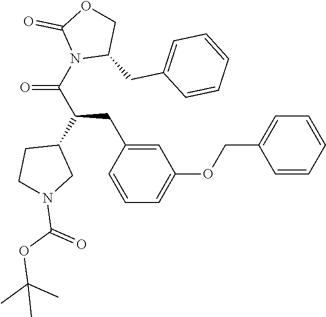 [SMILES: CC(C)(C)OC(=O)N1CC[C@H]([C@H](Cc2cccc(OCc3ccccc3)c2)C(=O)N2C(=O)OC[C@@H]2Cc2ccccc2)C1]

The title compound was prepared essentially as described in Preparation 11 using 1-benzyloxy-3-(bromomethyl)benzene and purifying the reaction product by silica gel chromatography using a gradient of 10 to 40% acetone in hexanes. ES-MS m/z 607 (M+Na).

### Preparation 178

### (2S)-3-(3-Benzyloxyphenyl)-2-[(3R)-1-tert-butoxycarbonylpyrrolidin-3-yl]propanoic acid

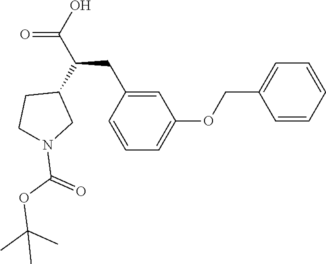 [SMILES: CC(C)(C)OC(=O)N1CC[C@H]([C@H](Cc2cccc(OCc3ccccc3)c2)C(=O)O)C1]

The title compound was prepared essentially as described in Preparation 12 using tert-butyl (3R)-3-[(1S)-2-[(4S)-4-benzyl-2-oxo-oxazolidin-3-yl]-1-[(3-benzyloxyphenyl)methyl]-2-oxo-ethyl]pyrrolidine-1-carboxylate, stirring the reaction for 3 h at RT then adding additional portions of hydrogen peroxide and lithium hydroxide and stirring the reaction an additional 2 h at 0° C. The reaction product was purified by silica gel chromatography using a gradient of 20 to 80% EtOAc in hexanes+1T acetic acid. ES-MS m/z 448 (M+Na).

### Preparation 179

### tert-Butyl (3R)-3-[(1S)-1-[(3-benzyloxyphenyl)methyl]-2-tert-butoxy-2-oxo-ethyl]pyrrolidine-1-carboxylate

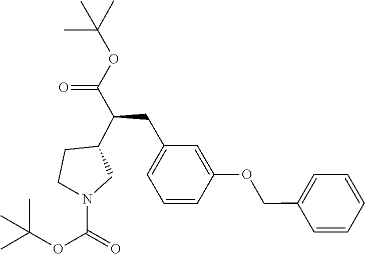 [SMILES: CC(C)(C)OC(=O)[C@@H](Cc1cccc(OCc2ccccc2)c1)[C@H]1CCN(C(=O)OC(C)(C)C)C1]

A solution of (2S)-3-(3-benzyloxyphenyl)-2-[(3R)-1-tert-butoxycarbonylpyrrolidin-3-yl]propanoic acid (4.86 g, 11.4 mmol) in toluene (103 mL) at 80° C. was treated with a solution of N,N-dimethylformamide di-tert-butyl acetal (12.9 g, 90 mass %, 57.1 mmol) in toluene (11.4 mL) over 1 h. After 3 h, another aliquot of N,N-dimethylformamide di-tert-butyl acetal (12.9 g, 90 mass %, 57.1 mmol) in toluene (11.4 mL) was added dropwise and stirring continued at 80° C. overnight. The reaction was cooled to RT, diluted with EtOAc, washed with water and saturated aqueous NaCl, collected, dried over Na₂SO₄, filtered, and concentrated under reduced pressure to give the title compound (5.7 g, 98%) as a yellow oil which was used without purification. ES-MS m/z 370 (M+H-2^{t}Bu).

### Preparation 180

### tert-Butyl (3R)-3-[(1S)-2-tert-butoxy-1-[(3-hydroxyphenyl)methyl]-2-oxo-ethyl]pyrrolidine-1-carboxylate

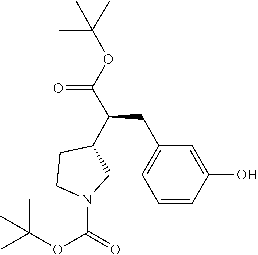 [SMILES: CC(C)(C)OC(=O)[C@@H](Cc1cccc(O)c1)[C@H]1CCN(C(=O)OC(C)(C)C)C1]

A mixture of tert-butyl (3R)-3-[(1S)-1-[(3-benzyloxyphenyl)methyl]-2-tert-butoxy-2-oxo-ethyl]pyrrolidine-1-carboxylate (4.45 g, 7.84 mmol) and palladium (10% on activated carbon paste type 87 μL, 0.45 g, 0.42 mmol) in EtOAc (39 mL) was stirred at RT under a hydrogen balloon over 4 days. The reaction was filtered through diatomaceous earth and the filtrate concentrated under reduced pressure. The residue was purified by silica gel chromatography eluted with a gradient of 10% to 40% acetone in hexane to give the title compound (2.85 g, 93%) as a white solid. ES-MS m/z 280 (M+H-2tBu).

### Preparation 181

### tert-Butyl (3R)-3-[(1S)-2-tert-butoxy-1-[[3-[(2,2-difluoro-1,3-benzodioxol-5-yl)methoxy]phenyl]methyl]-2-oxo-ethyl]pyrrolidine-1-carboxylate

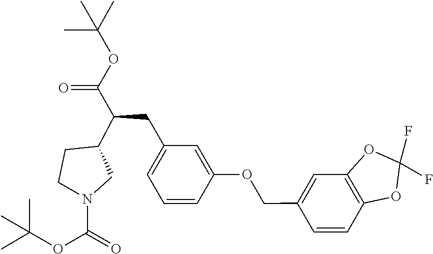 [SMILES: CC(C)(C)OC(=O)[C@@H](Cc1cccc(OCc2ccc3c(c2)OC(F)(F)O3)c1)[C@H]1CCN(C(=O)OC(C)(C)C)C1]

A tube was charged with tert-butyl (3R)-3-[(1S)-2-tert-butoxy-1-[(3-hydroxyphenyl)methyl]-2-oxo-ethyl]pyrrolidine-1-carboxylate (100 mg, 0.26 mmol), 5-(bromomethyl)-2,2-difluoro-1,3-benzodioxole (76.9 mg, 0.31 mmol), Cs₂CO₃ (100 mg, 0.31 mmol), and DMF (1.3 mL). The reaction was allowed to stir overnight at RT. The reaction temperature was raised to 45° C. and another ˜0.32 mg of 5-(bromomethyl)-2,2-difluoro-1,3-benzodioxole was added and the reaction stirred for another 3 h. The reaction was concentrated was under a stream of nitrogen and filtered through a cartridge of diatomaceous earth. The filtrate was diluted with EtOAc and washed with water, the organic layer collected, and concentrated under reduced pressure. The residue was purified by reversed phase chromatography under basic conditions giving the title compound (66.7 mg, 47%) as a white solid. ES-MS m/z 584 (M+Na).

The following compound was prepared essentially as described in Preparation 181 using the appropriate reagents, adjusting the temperature, and the reaction times to determine completion of the reactions, and adjusting the purification system as appropriate.

| Prep | | | ES/MS |
|---|---|---|---|
| # | Chemical Name | Structure | m/z |
| 182 | tert-Butyl (3R)-3-[(1S)-2- tert-butoxy-1-[[3-[[3-[(2,4- difluorophenyl)methoxy] phenyl]methoxy]phenyl] methyl]-2-oxo- ethyl]pyrrolidine-1- carboxylate | 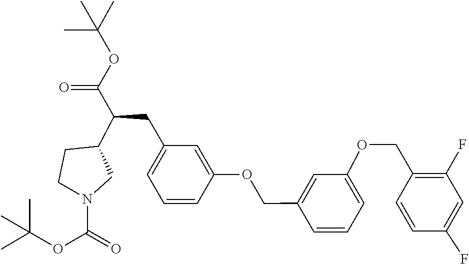 | 512 [M + H − 2tBu]⁺ |

### Preparation 183

### tert-Butyl (3R)-3-[(1S)-2-tert-butoxy-2-oxo-1-[[3-(2-pyridylmethylcarbamothioylamino)phenyl]methyl]ethyl]pyrrolidine-1-carboxylate

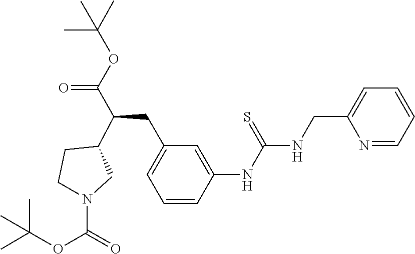 [SMILES: CC(C)(C)OC(=O)[C@@H](Cc1cccc(NC(=S)NCc2ccccn2)c1)[C@H]1CCN(C(=O)OC(C)(C)C)C1]

A 0° C. solution of 1,1′-thiocarbonyldiimidazole (0.11 g, 90 mass %, 0.58 mmol) in ACN (1.4 mL) was slowly treated with a solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 0.150 g, 0.38 mmol) in ACN (1.4 mL). The cooling bath was removed, the reaction allowed to reach RT, and stirred for 20 min. 2-(Aminomethyl)pyridine (0.08 g, 0.77 mmol) in ACN (1.4 mL) was added and the reaction stirred at RT for 1.5 h. Solvent was removed under reduced pressure and the residue purified by silica gel chromatography eluted with a gradient of 15% to 60% acetone in hexane giving the title compound (0.22 g, 107%) as a yellow solid. ES-MS m/z 541 (M+H).

### Preparation 184

### tert-Butyl (3R)-3-[(1S)-2-tert-butoxy-1-[[3-(imidazo[1,5-a]pyridin-3-ylamino)phenyl]methyl]-2-oxo-ethyl]pyrrolidine-1-carboxylate

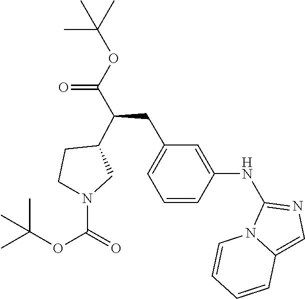 [SMILES: CC(C)(C)OC(=O)[C@@H](Cc1cccc(Nc2ncc3ccccn23)c1)[C@H]1CCN(C(=O)OC(C)(C)C)C1]

A sealed tube was charged with tert-butyl (3R)-3-[(1S)-2-tert-butoxy-2-oxo-1-[[3-(2-pyridylmethylcarbamothioylamino)phenyl]methyl]ethyl]pyrrolidine-1-carboxylate (0.22 g, 0.41 mmol), 1-(3-dimethylaminopropyl)-3-ethylcarbodiimide hydrochloride (0.11 g, 0.56 mmol), and toluene (3.7 mL). The tube was sealed and heated at 110° C. for 1 h. The reaction was cooled to RT and quenched with saturated aqueous NaHCO₃ and extracted with EtOAc (2×). The organic layers were combined, washed with saturated aqueous NaCl, collected, dried over Na₂SO₄, filtered, and concentrated under reduced pressure. The residue was purified by reversed phase chromatography eluted with a gradient of 60% to 90% ACN in aqueous NH₄HCO₃. The resulting material was repurified by reversed phase chromatography eluted with a gradient of 40% to 60% ACN (with 0.05% TFA) in water (with 0.05% TFA) to give the title compound (41.3 mg, 20%) as a white solid. ES-MS m/z 507 (M+H).

### Preparation 185

### tert-Butyl (R)-3-((S)-3-(3-(N-(benzo[d][1,3]dioxol-5-ylmethyl)sulfamoyl)phenyl)-1-(tert-butoxy)-1-oxopropan-2-yl)pyrrolidine-1-carboxylate

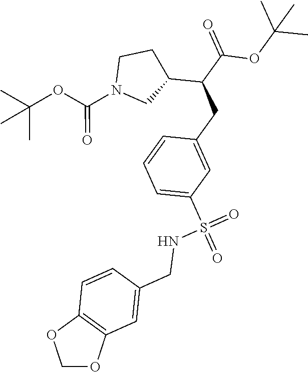 [SMILES: CC(C)(C)OC(=O)[C@@H](Cc1cccc(S(=O)(=O)NCc2ccc3c(c2)OCO3)c1)[C@H]1CCN(C(=O)OC(C)(C)C)C1]

### and tert-butyl (3R)-3-[(1S)-2-tert-butoxy-1-[[3-(2,3-dihydro-1,4-benzodioxin-6-ylmethylsulfamoyl)phenyl]methyl]-2-oxo-ethyl]pyrrolidine-1-carboxylate

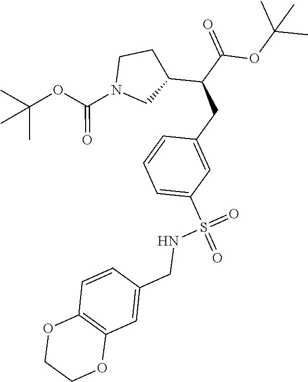 [SMILES: CC(C)(C)OC(=O)[C@@H](Cc1cccc(S(=O)(=O)NCc2ccc3c(c2)OCCO3)c1)[C@H]1CCN(C(=O)OC(C)(C)C)C1]

A RT solution of tert-butyl (3R)-3-[(1S)-2-tert-butoxy-1-[(3-chlorosulfonylphenyl)methyl]-2-oxo-ethyl]pyrrolidine-1-carboxylate (0.45 g, 64 mass %, 0.60 mmol) in DCM (3 mL) was treated with TEA (0.12 g, 1.21 mmol) and 1,3-benzodioxol-5-ylmethanamine;hydrate (0.11 g, 0.72 mmol). The reaction was stirred for 3 h. Analysis of the reaction showed only sulphonyl chloride. Added 2,3-dihydro-1,4-benzodioxin-6-ylmethylamine (0.12 g, 0.72 mmol) and continued stirring for 2 h. Diluted the reaction with 1N aqueous HCl (3 mL) and extracted with DCM. The organic layer was collected, dried over MgSO₄, filtered, and concentrated under reduced pressure. The residue was purified by silica gel chromatography eluted with a gradient of 25% to 75% EtOAc in hexane. The two compounds were separately purified by reversed phase chromatography eluted with a gradient of 60% to 80% ACN in aqueous 20 mM NH₄HCO₃ to give tert-butyl (3R)-3-[(1S)-1-[[3-(1,3-benzodioxol-5-ylmethylsulfamoyl)phenyl]methyl]-2-tert-butoxy-2-oxo-ethyl]pyrrolidine-1-carboxylate (20 mg, 6%, ES-MS m/z 489 (M+H-BOC)) and tert-butyl (3R)-3-[(1S)-1-[[3-(1,3-benzodioxol-5-ylmethylsulfamoyl)phenyl]methyl]-2-tert-butoxy-2-oxo-ethyl]pyrrolidine-1-carboxylate (27 mg, 7%, ES-MS m/z 503 (M+H-BOC)).

The following were prepared essentially as described in Preparation 121 using tert-butyl (3R)-3-[(1S)-1-[(3-bromophenyl)methyl]-2-tert-butoxy-2-oxo-ethyl]pyrrolidine-1-carboxylate and the appropriate amine. Before purification, the reaction mixtures were concentrated and loaded onto SCX resin, which was washed with MeOH (wash discarded), then eluted 2 M NH₃ in MeOH and concentrated the eluent.

| | | | Physical |
|---|---|---|---|
| | | | Data |
| Prep- | Chemical | | ES-MS |
| aration | name | Structure | m/z |
| 186 | tert-Butyl (3R)-3-[(1S)-2- tert-butoxy-2-oxo-1-[[3-(2- pyridylamino)phenyl]methyl] ethyl]pyrrolidine-1- carboxylate | 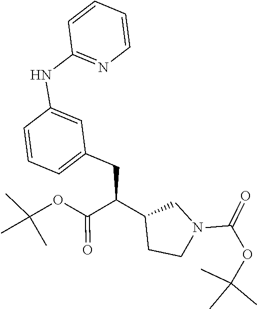 [SMILES: CC(C)(C)OC(=O)[C@@H](Cc1cccc(Nc2ccccn2)c1)[C@H]1CCN(C(=O)OC(C)(C)C)C1] | 468 (M + H) |
| 187 | tert-Butyl (3R)-3-[(1S)-2- tert-butoxy-1-[[3-(indan-5- ylamino)phenyl]methyl]-2- oxo-ethyl]pyrrolidine-1- carboxylate | 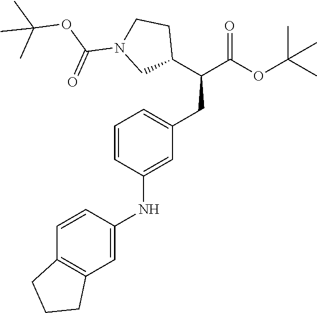 [SMILES: CC(C)(C)OC(=O)[C@@H](Cc1cccc(Nc2ccc3c(c2)CCC3)c1)[C@H]1CCN(C(=O)OC(C)(C)C)C1] | 407 (M − Boc) |
| 188 | tert-Butyl 4-[3-[3-[(2S)-3- tert-butoxy-2-[(3R)-1-tert- butoxycarbonylpyrrolidin- 3-yl]-3-oxo- propyl]anilino]phenyl] piperidine-1-carboxylate | 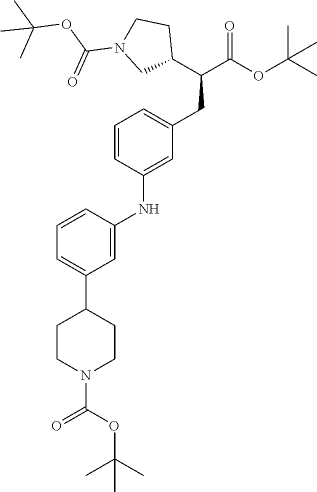 [SMILES: CC(C)(C)OC(=O)[C@@H](Cc1cccc(Nc2cccc(C3CCN(C(=O)OC(C)(C)C)CC3)c2)c1)[C@H]1CCN(C(=O)OC(C)(C)C)C1] | 550 (M + H − Boc) |

### Preparation 189

### tert-Butyl (3R)-3-[(1S)-2-tert-butoxy-1-[[3-[(4-oxazol-5-ylanilino)methyl]phenyl]methyl]-2-oxo-ethyl]pyrrolidine-1-carboxylate

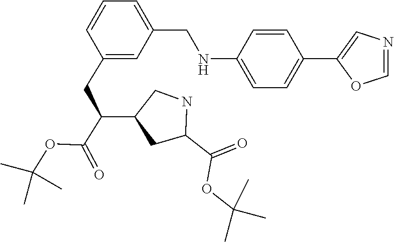

The title compound was prepared essentially as described in Preparation 20 using 4-oxazol-5-ylaniline. Upon completion, the reaction was concentrated and loaded onto SCX resin, which was washed with MeOH (wash discarded), then eluted with 2 M NH3 in MeOH and concentrated the eluent. The material was purified by reversed-phase chromatography [column XBridge® C18 21×100 mm, 5 μm; mobile phase 70% to 90% ACN in aqueous NH₄HCO₃ (20 mM, pH 9); flow rate 25 mL/min] to give the title compound (98 mg, 40%). ES-MS m/z 548 (M+H).

### Preparation 190

### tert-Butyl (3R)-3-[(1S)-2-tert-butoxy-1-[[3-[(3-methoxy-N-methyl-anilino)methyl]phenyl]methyl]-2-oxo-ethyl]pyrrolidine-1-carboxylate

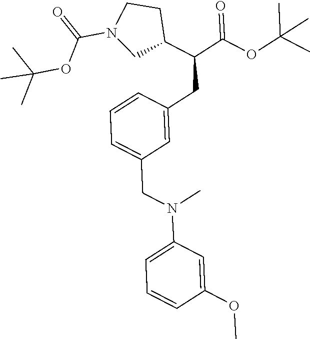 [SMILES: COc1cccc(N(C)Cc2cccc(C[C@H](C(=O)OC(C)(C)C)[C@H]3CCN(C(=O)OC(C)(C)C)C3)c2)c1]

The title compound was prepared essentially as described in Preparation 20 using 3-methoxy-N-methylaniline. Upon completion, the reaction was concentrated and loaded onto SCX resin, which was washed with MeOH (wash discarded), then eluted 2 M NH₃ in MeOH and concentrated the eluent. The residue was purified by reverse-phase HPLC [column: XBridge® C18 21×100 mm, 5 μm; mobile phase: 70% to 90% ACN in aqueous NH₄HCO₃ (20 mM, pH 9); flow rate 25 mL/min] to give the title compound (46.5 mg, 20%). ES-MS m/z 525 (M+H)

### Preparation 191

### tert-Butyl (3R)-3-[1-[(3-bromophenyl)methyl]-2-methoxy-2-oxo-ethyl]pyrrolidine-1-carboxylate (Mixture of Diastereomers)

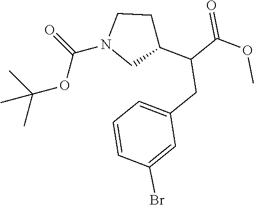 [SMILES: COC(=O)C(Cc1cccc(Br)c1)[C@H]1CCN(C(=O)OC(C)(C)C)C1]

A 500 mL round bottom flask equipped with a N₂ inlet was charged with THE (90 mL) and diisopropylamine (4.60 g, 45.2 mmol) and the mixture cooled to −70° C. N-Butyllithium (11 g, 2.5 mol/L in hexane, 41.10 mmol) was added dropwise and allowed to stir for 10 min. A solution of tert-butyl (3R)-3-(2-methoxy-2-oxo-ethyl)pyrrolidine-1-carboxylate (10 g, 41.1 mmol) in THE (30 mL) was added dropwise and the mixture stirred for 30 min. 3-Bromobenzyl bromide (11.3 g, 45.2 mmol) was added and the mixture allowed to warmed to RT. After stirring for 1 h at RT, the reaction was diluted with MeOH, concentrated under reduced pressure, and the residue purified by silica gel chromatography eluted with 20% EtOAc in hexane to give the title compound (11.5 g, 68%) as a colorless oil. ES-MS m/z 357 (M+H-^{t}Bu).

### Preparation 192

### 3-(3-Bromophenyl)-2-[(3R)-1-tert-butoxycarbonylpyrrolidin-3-yl]propanoic acid (Isomer 1 and Isomer 2)

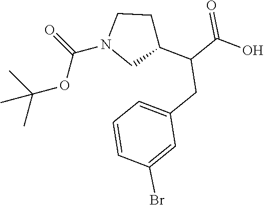 [SMILES: CC(C)(C)OC(=O)N1CC[C@H](C(Cc2cccc(Br)c2)C(=O)O)C1]

A solution of tert-butyl (3R)-3-[1-[(3-bromophenyl)methyl]-2-methoxy-2-oxo-ethyl]pyrrolidine-1-carboxylate (10 g, 24.25 mmol) in MeOH (218 mL) and THF (218 mL) was treated with 1M aqueous NaOH (243 mL). The resulting mixture was stirred at 50° C. for 2 h. After cooling to RT, the reaction was concentrated under reduced pressure.

The residue was dissolved in EtOAc, neutralized with aqueous citric acid to pH 4, and extracted with EtOAc (3×). The organic layers were combined, washed with saturated aqueous NaCl (2×), collected, dried over MgSO₄, filtered, and concentrated under reduced pressure to give the title compound (9.5 g, 98%) as a colorless oil. ES-MS m/z 343 [M+H-^{t}Bu]+.

Isomer 1 and Isomer 2 of tert-butyl (3R)-3-[rac-1-[(3-benzyloxyphenyl)methyl]-2-methoxy-2-oxo-ethyl]pyrrolidine-1-carboxylate (20.3 g, 51.0 mmol) were obtained by chiral SFC chromatography using a Chiralpak® AD column (5×25 cm, 5 μm) eluted with 20% CO₂ in IPA. The isomers obtained were separately dissolved in EtOAc and washed with 0.5 M aqueous HCl. The organic layers were collected, dried over Na₂SO₄, and concentrated under reduced pressure to obtain Isomer 1 (first-eluting isomer, 7.2 g, 36%) and Isomer 2 (second-eluting isomer, 8.1 g, 40%) as pale yellow solids. ES-MS m/z 344 (M+H-^{t}Bu).

### Preparation 193

### tert-Butyl (3R)-3-[1-[(3-bromophenyl)methyl]-2-methoxy-2-oxo-ethyl]pyrrolidine-1-carboxylate (Isomer 2)

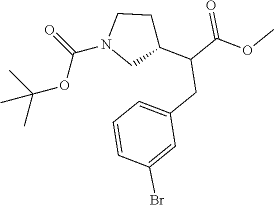 [SMILES: COC(=O)C(Cc1cccc(Br)c1)[C@H]1CCN(C(=O)OC(C)(C)C)C1]

A RT suspension of 3-(3-bromophenyl)-2-[(3R)-1-tert-butoxycarbonylpyrrolidin-3-yl]propanoic acid (Isomer 2, 7.7 g, 19 mmol) in MeOH (19 mL) was slowly treated with (trimethylsilyl)diazomethane (12 g, 2 M in hexanes, 33 mmol) and the reaction was stirred for 10 min. Two more aliquots of (trimethylsilyl)diazomethane (12 g, 2 M in hexanes, 33 mmol) were added and the reaction stirred for 10 min after each addition. The reaction was concentrated under reduced pressure. The residue was dissolved in DCM and filtered through a small pad of silica gel. The filtrate was concentrated under reduced pressure to give the title compound (8.0 g, 100%) as a pale brown oil. ES-MS m/z 356/358 (M+H-^{t}Bu).

### Preparation 194

### tert-Butyl (3R)-3-[1-(benzyloxymethyl)-1-[(3-bromophenyl)methyl]-2-methoxy-2-oxo-ethyl]pyrrolidine-1-carboxylate (Mixture of Diastereomers)

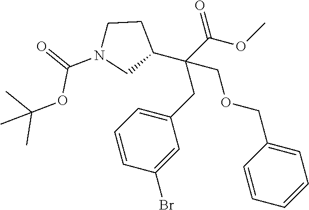 [SMILES: COC(=O)C(COCc1ccccc1)(Cc1cccc(Br)c1)[C@H]1CCN(C(=O)OC(C)(C)C)C1]

A solution of tert-butyl (3R)-3-[1-[(3-bromophenyl)methyl]-2-methoxy-2-oxo-ethyl]pyrrolidine-1-carboxylate (1.01 g, 2.45 mmol) in THE (24.5 mL) at −78° C. was treated with potassium bis(trimethylsilyl)amide (5.39 mL, 1M in THF, 5.39 mmol). After 30 min, chloromethoxymethylbenzene (0.77 g, 4.90 mmol) dissolved in 5 mL of THF was added. The mixture was allowed to warm to RT slowly and then stirred at RT overnight. Saturated aqueous NH₄Cl was added and the aqueous layer extracted with EtOAc. The organic layer was collected, dried over Na₂SO₄, filtered, and concentrated under reduced pressure. The residue was purified by normal phase chromatography eluted with a gradient of 7% to 30% EtOAc in hexane to give the title compound (1.20 g, 73 mass %, 67%) as a colorless oil. ES-MS m/z 476/478 (M+H-^{t}Bu).

### Preparation 195

### 2-(Benzyloxymethyl)-3-(3-bromophenyl)-2-[(3R)-1-tert-butoxycarbonylpyrrolidin-3-yl]propanoic acid (Mixture of Diastereomers)

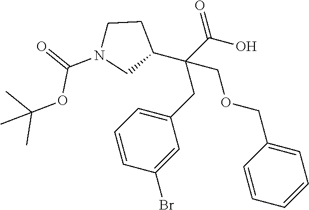 [SMILES: CC(C)(C)OC(=O)N1CC[C@H](C(COCc2ccccc2)(Cc2cccc(Br)c2)C(=O)O)C1]

tert-Butyl (3R)-3-[1-(benzyloxymethyl)-1-[(3-bromophenyl)methyl]-2-methoxy-2-oxo-ethyl]pyrrolidine-1-carboxylate (1.2 g, 2.3 mmol) was dissolved in THE (9 mL) and MeOH (9 mL) and treated with 1M aqueous NaOH (23 mL, 23 mmol). The reaction was stirred at 60° C. overnight. The reaction was treated again with 1M aqueous NaOH (23 mL, 23 mmol) and stirring continued at 60° C. overnight. The reaction was diluted with MeOH (9 mL) and the reaction heated at 80° C. for 5 h. Because of slow hydrolysis, the reaction was concentrated under reduced pressure and the residue dissolved in MeOH (15 mL) and THE (9 mL) and the solution treated with 5M aqueous NaOH (9 mL, 370 mmol). The reaction was stirred at 80° C. overnight. The reaction was cooled to RT and concentrated under reduced pressure. The residue was taken up in EtOAc and water. The pH of the aqueous layer was acidified with 1N aqueous HCl and extracted with EtOAc. The organic layer was collected, dried over Na₂SO₄, filtered, and concentrated under reduced pressure. The residue was purified by reversed phase chromatography eluted with a gradient of 40% to 70% ACN in water to give the title compound (0.58 g, 50%) as a white solid. ES-MS m/z 462/464 (M+H-^{t}Bu).

### Preparation 196

### 2-(Benzyloxymethyl)-2-[(3R)-1-tert-butoxycarbonylpyrrolidin-3-yl]-3-(3-phenylphenyl)propanoic acid (Isomer 1 and Isomer 2)

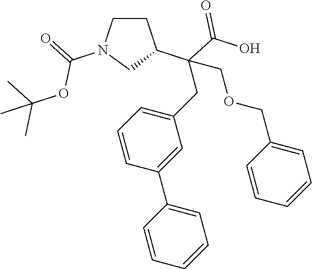 [SMILES: CC(C)(C)OC(=O)N1CC[C@H](C(COCc2ccccc2)(Cc2cccc(-c3ccccc3)c2)C(=O)O)C1]

A stirred mixture 2-(benzyloxymethyl)-3-(3-bromophenyl)-2-[(3R)-1-tert-butoxycarbonylpyrrolidin-3-yl]propanoic acid (0.58 g, 1.11 mmol), phenylboronic acid (0.209 g, 1.67 mmol), and potassium carbonate (0.46 g, 3.33 mmol, 100 mass %) in 1,4-dioxane (11.1 mL) and water (1.1 mL) at 100° C. was treated with 1,1′-bis(diphenylphosphino)ferrocene-palladium(ii)dichloride DCM complex (0.09 g, 0.11 mmol). The tube was sealed and the mixture was heated at 100° C. overnight. The reaction was allowed to cool to RT, diluted with EtOAc, filtered through diatomaceous earth, and concentrated under reduced pressure to dryness. The residue was purified by reversed phase chromatography eluted with a gradient of 40% to 70% ACN in water to give the title compound (700 mg).

The isomers were separated by SFC chromatography using a Chiralpak AD column (2×25 cm, 5 μm) eluted with 25% CO₂ in IPA (with 0.2% DMEA) to give Isomer 1 (130.3 mg, 23%) as a white solid. Isomer 2 was repurified by SFC chromatography using a Claricep C-series C18 column (20×250 mm, 5 μm) eluted with 25% CO₂ in IPA (with 0.2% DMEA) to give Isomer 2 (278.1 mg, 49%) as a white solid. ES-MS m/z 460 (M+H-^{t}Bu).

### Preparation 197a

### 2-[(3R)-1-tert-Butoxycarbonylpyrrolidin-3-yl]-2-(hydroxymethyl)-3-(3-phenylphenyl)propanoic acid (Isomer 2)

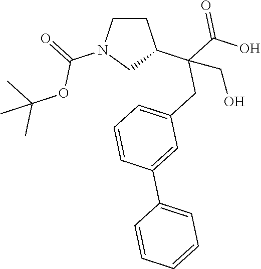 [SMILES: CC(C)(C)OC(=O)N1CC[C@H](C(CO)(Cc2cccc(-c3ccccc3)c2)C(=O)O)C1]

A mixture of 2-(benzyloxymethyl)-2-[(3R)-1-tert-butoxycarbonylpyrrolidin-3-yl]-3-(3-phenylphenyl)propanoic acid (Isomer 2, 100 mg, 0.19 mmol) and palladium (10% on activated carbon paste type 87 L, 0.1 g, 0.01 mmol) in EtOAc (1.9 mL) was stirred under hydrogen (balloon) at RT overnight. The reaction progress was slow, so it was filtered through diatomaceous earth and concentrated under reduced pressure. The residue was dissolved in EtOH (2.9 mL) and treated with palladium hydroxide on carbon (0.05 g) and stirred under hydrogen at 90-100 psi over the weekend. The reaction was filtered through diatomaceous earth and concentrated under reduced pressure. The residue was purified by reversed phase chromatography eluted with a gradient of 30% to 60% ACN in water to give the title compound (0.12 g, 70 mass %, 24%). ES-MS m/z 326 [M+H-Boc]+.

### Preparation 197b

### 2-[(3R)-1-tert-Butoxycarbonylpyrrolidin-3-yl]-2-(hydroxymethyl)-3-(3-phenylphenyl)propanoic acid (Isomer 1′ 1)

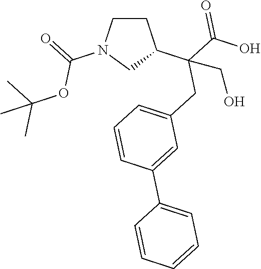 [SMILES: CC(C)(C)OC(=O)N1CC[C@H](C(CO)(Cc2cccc(-c3ccccc3)c2)C(=O)O)C1]

The title compound was prepared essentially as described in Preparation 197a using 2-(benzyloxymethyl)-2-[(3R)-1-tert-butoxycarbonylpyrrolidin-3-yl]-3-(3-phenylphenyl)propanoic acid (Isomer 1), stirring the starting material in EtOH with palladium hydroxide on carbon under H₂ (100 psi) over the weekend. ES-MS m/z 326 [M+H-Boc]+.

### Preparation 198

### tert-Butyl (3R)-3-[1-[(3-bromophenyl)methyl]-1-[(1,3-dioxoisoindolin-2-yl)methyl]-2-methoxy-2-oxo-ethyl]pyrrolidine-1-carboxylate (Mixture of Diastereomers)

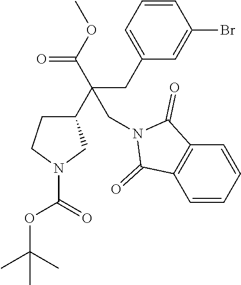 [SMILES: COC(=O)C(Cc1cccc(Br)c1)(CN1C(=O)c2ccccc2C1=O)[C@H]1CCN(C(=O)OC(C)(C)C)C1]

A solution of tert-butyl (3R)-3-[1-[(3-bromophenyl)methyl]-2-methoxy-2-oxo-ethyl]pyrrolidine-1-carboxylate (1.22 g, 2.96 mmol) in THE (29.6 mL) at −78° C. was treated with potassium bis(trimethylsilyl)amide (1 M in THF, 6.51 mL, 6.51 mmol). After 30 min, 2-(bromomethyl)isoindoline-1,3-dione (1.42 g, 5.92 mmol) dissolved in 10 mL of THE was added. The reaction was allowed to warm to RT slowly and stirred at RT overnight. The reaction was quenched with saturated aqueous NH₄Cl and extracted with EtOAc. The organic layer was collected, dried over Na₂SO₄, filtered, and concentrated under reduced pressure. The residue was purified by silica gel chromatography eluted with a gradient of 15% to 60% acetone in hexane to give the title compound (0.60 g, 94 mass %, 33%) as a white solid. ES-MS m/z 473 (M+H-BOC).

### Preparation 199

### 2-[[3-(3-Bromophenyl)-2-[(3R)-1-tert-butoxycarbonylpyrrolidin-3-yl]-2-carboxy-propyl]carbamoyl]benzoic acid (Mixture of Diastereomers)

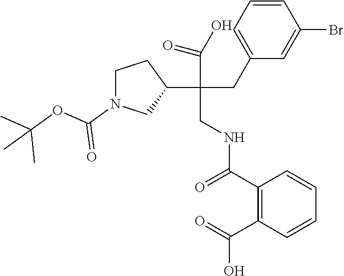 [SMILES: CC(C)(C)OC(=O)N1CC[C@H](C(CNC(=O)c2ccccc2C(=O)O)(Cc2cccc(Br)c2)C(=O)O)C1]

A solution of tert-butyl (3R)-3-[1-[(3-bromophenyl)methyl]-1-[(1,3-dioxoisoindolin-2-yl)methyl]-2-methoxy-2-oxo-ethyl]pyrrolidine-1-carboxylate (mixture of diastereomers, 0.60 g, 1 mmol) was dissolved in MeOH (7 mL) and THE (7 mL) was treated with 5M aqueous NaOH (4 mL, 20 mmol) and the reaction stirred at 80° C. overnight. The reaction was allowed to cool to RT and then concentrated under reduced pressure. The residue was suspended in water and washed with Et₂O. The aqueous layer was cooled to 0° C. and the pH adjusted to 3 with 1N aqueous HCl to obtain a solid which was removed by filtration and washed with water to give the title compound (0.58 g, 100%) as a white solid after drying at 40° C. overnight. ES-MS m/z 475/477 (M+H-BOC).

### Preparation 200

### 2-[[2-[(3R)-1-tert-Butoxycarbonylpyrrolidin-3-yl]-2-carboxy-3-(3-phenylphenyl)propyl]carbamoyl]benzoic acid (Mixture of Diastereomers)

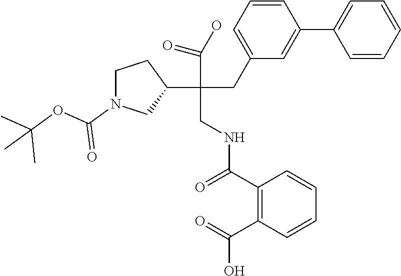 [SMILES: CC(C)(C)OC(=O)N1CC[C@H](C(CNC(=O)c2ccccc2C(=O)O)(Cc2cccc(-c3ccccc3)c2)C(=O)O)C1]

A pressure tube was charged with 2-[[3-(3-bromophenyl)-2-[(3R)-1-tert-butoxycarbonylpyrrolidin-3-yl]-2-carboxy-propyl]carbamoyl]benzoic acid (mixture of diastereomers, 78 mg, 0.14 mmol), phenylboronic acid (25.6 mg, 0.20 mmol), potassium carbonate (56.2 mg, 0.41 mmol), 1,4-dioxane (1.4 mL), and water (0.14 mL). The reaction was warmed to 100° C. and treated with 1,1′-bis(diphenylphosphino)ferrocene-palladium(II)dichloride DCM complex (11.3 mg, 0.01 mmol). The tube was sealed and the mixture heated at 100° C. overnight. Reaction was cooled to RT and filtered through diatomaceous earth. The filtrate was concentrated under reduced pressure. The residue was purified by reversed phase chromatography eluted with a gradient of 2000 to 5000 ACN in water to give the title compound (35 mg, 450%). ES-MS m/z 473 (M+H-BOC).

The following were prepared essentially as described in Preparation 11 using tert-butyl (3R)-3-[2-[(4S)-4-benzyl-2-oxo-oxazolidin-3-yl]-2-oxo-ethyl]pyrrolidine-1-carboxylate (prepared essentially as described in WO 2020/247429) and the appropriate alkyl bromide, stirring the reaction at 0° C. for 40-50 min prior to alkyl bromide addition, and purifying the product via silica gel chromatography using a gradient of EtOAc in petroleum ether (starting at 0% EtOAc and increasing up to 50-100% EtOAc).

| Prep- | Chemical | | Physical Data |
|---|---|---|---|
| aration | name | Structure | ¹H NMR |
| 201 | tert-Butyl (3R)-3-[(1S)- 2-[(4S)-4-benzyl-2-oxo- oxazolidin-3-yl]-1-[(5- bromopyrazin-2- yl)methyl]-2-oxo- ethyl]pyrrolidine-1- carboxylate | 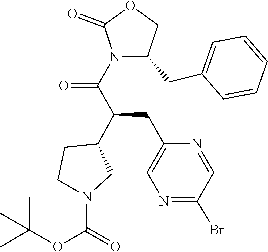 [SMILES: CC(C)(C)OC(=O)N1CC[C@H]([C@H](Cc2cnc(Br)cn2)C(=O)N2C(=O)OC[C@@H]2Cc2ccccc2)C1] | (400 MHz, methanol- d₄) δ ppm 8.62-8.55 (m,1H), 8.34-8.25 (m, 1H), 7.25-7.09 (m, 3H), 7.06-6.97 (m, 2H), 4.66-4.58 (m, 1H), 4.47-4.34 (m, 1H), 4.21-4.12 (m, 1H), 4.11-4.05 (m, 1H), 3.58-3.46 (m, 1H), 3.41-3.33 (m, 1H), 3.30-3.24 (m, 1H), 3.18-3.09 (m, 1H), 3.08-2.94 (m, 3H), 2.66-2.57 (m, 1H), 2.54-2.40 (m, |
| | | | 1H), 1.98-1.92 (m, |
| | | | 1H), 1.76-1.56 (m, |
| | | | 1H), 1.40-1.32 (s, 9H) |
| 202 | tert-Butyl (3R)-3-[(1S)- 2-[(4S)-4-benzyl-2-oxo- oxazolidin-3-yl]-1-[(5- bromopyrimidin-2- yl)methyl]-2-oxo- ethyl]pyrrolidine-1- carboxylate | 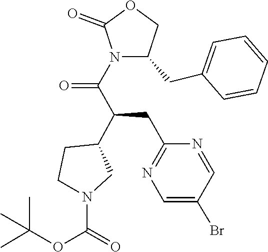 [SMILES: CC(C)(C)OC(=O)N1CC[C@H]([C@H](Cc2ncc(Br)cn2)C(=O)N2C(=O)OC[C@@H]2Cc2ccccc2)C1] | (400 MHz, methanol- d₄) δ ppm 8.70 (s, 2H), 7.22-7.13 (m, 5H), 4.75-4.59 (m, 1H), 4.43-4.34 (m, 1H), 4.17-4.07 (m, 2H), 3.51-3.49 (m, 1H), 3.40-3.35 (m, 2H), 3.20-2.99 (m, 4H), 2.70-2.66 (m, 1H), 2.54-2.40 (m, 1H), 1.95-1.85 (m, 1H), 1.70-1.60 (m, 1H), 1.35 (s, 9H) |
| 203 | tert-Butyl (3R)-3-[(1S)- 2-[(4S)-4-benzyl-2-oxo- oxazolidin-3-yl]-1-[(2- chloro-4- pyridyl)methyl]-2-oxo- ethyl]pyrrolidine-1- carboxylate | 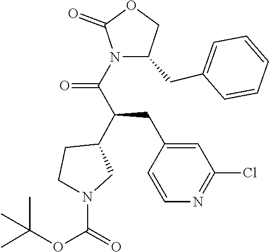 [SMILES: CC(C)(C)OC(=O)N1CC[C@H]([C@H](Cc2ccnc(Cl)c2)C(=O)N2C(=O)OC[C@@H]2Cc2ccccc2)C1] | (400 MHz, methanol- d₄) δ ppm 8.25-8.15 (m, 1H), 7.35- 6.75 (m, 7H), 4.70- 4.55 (m, 1H), 4.40- 3.95 (m, 4H), 3.55- 3.25 (m, 2H), 3.20- 2.25 (m, 6H), 1.55- 1.50 (m, 2H), 1.35 (s, 9H) |
| 204 | tert-Butyl (3R)-3-[(1S)- 2-[(4S)-4-benzyl-2-oxo- oxazolidin-3-yl]-1-[(3- bromo-5-methyl- phenyl)methyl]-2-oxo- ethyl]pyrrolidine-1- carboxylate | 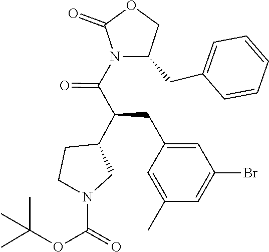 [SMILES: Cc1cc(Br)cc(C[C@H](C(=O)N2C(=O)OC[C@@H]2Cc2ccccc2)[C@H]2CCN(C(=O)OC(C)(C)C)C2)c1] | (400 MHz, methanol- d₄) δ ppm 7.23-7.10 (m, 5H), 7.06-7.00 (m, 1H), 6.91-6.81 (m, 2H), 4.67-4.60 (m, 1H), 4.42-4.28 (m, 1H), 4.19-4.09 (m, 1H), 3.59-3.46 (m, 1H), 3.44-3.35 (m, 1H), 3.22-2.80 (m, 6H), 2.49-2.32 (m, 2H), 2.23 (s, 3H), 1.95- 1.89 (m, 1H), 1.75- 1.57 (m, 1H), 1.40 (s, 9H) |
| 205 | tert-Butyl (3R)-3-[(1S)- 2-[(4S)-4-benzyl-2-oxo- oxazolidin-3-yl]-1-[(2- bromo-4-iodo- phenyl)methyl]-2-oxo- ethyl]pyrrolidine-1- carboxylate | 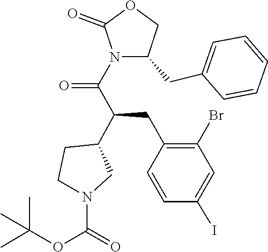 [SMILES: CC(C)(C)OC(=O)N1CC[C@H]([C@H](Cc2ccc(I)cc2Br)C(=O)N2C(=O)OC[C@@H]2Cc2ccccc2)C1] | (400 MHz, methanol- d₄) δ ppm 7.99-7.97 (m, 1H), 7.63-7.61 (m, 1H), 7.20- 7.11 (m, 4H), 6.85- 6.80 (m, 2H), 4.72- 4.59 (m, 2H), 4.23- 4.07 (m, 2H), 3.55- 3.25 (m, 2H), 3.23- 3.07 (m, 4H), 2.95- 2.80 (m, 1H), 2.60-2.50 (m, 2H), 2.01-1.90 (m, 1H), 1.80-1.55 (m, 1H), 1.46 (s, 9H) |
| 206 | tert-Butyl (3R)-3-[(1S)- 2-[(4S)-4-benzyl-2-oxo- oxazolidin-3-yl]-1-[[3- bromo-5- (trifluoromethoxy) phenyl]methyl]-2-oxo- ethyl]pyrrolidine-1- carboxylate | 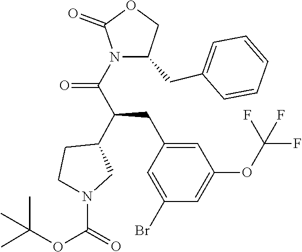 [SMILES: CC(C)(C)OC(=O)N1CC[C@H]([C@H](Cc2cc(Br)cc(OC(F)(F)F)c2)C(=O)N2C(=O)OC[C@@H]2Cc2ccccc2)C1] | (400 MHz, methanol- d₄) δ ppm 7.55-7.50 (m, 1H), 7.35 (m, 1H), 7.30-7.20 (m, 4H), 7.00-6.85 (m, 2H), 4.75-4.65 (m, 1H), 4.55-4.35 (m, 1H), 4.25-4.00 (m, 1H), 3.65-2.80 (m, 8H), 2.60-2.35 (m, 2H), 2.05-1.95 (m, 1H), 1.85-1.65 (m, 1H), 1.46 (s, 9H) |
| 207 | tert-Butyl (3R)-3-[(1S)- 2-[(4S)-4-benzyl-2-oxo- oxazolidin-3-yl]-1-[(2- bromo-4-cyano- phenyl)methyl]-2-oxo- ethyl]pyrrolidine-1- carboxylate | 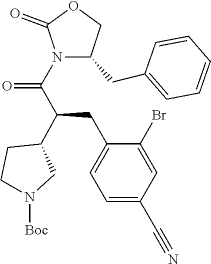 [SMILES: CC(C)(C)OC(=O)N1CC[C@H]([C@H](Cc2ccc(C#N)cc2Br)C(=O)N2C(=O)OC[C@@H]2Cc2ccccc2)C1] | (400 MHz, methanol- d₄) δ ppm 8.04 (m, 1H), 7.95-7.85 (m, 2H), 7.70-7.65 (m, 1H), 7.60-7.50 (m, 1H), 7.25-7.18 (m, 3H), 4.75-4.55 (m, 2H), 4.25-4.15 (m, 1H), 3.65-3.40 (m, 2H), 3.35-3.15 (m, 5H), 3.00-2.90 (m, 1H), 2.65-2.50 (m, 2H), 2.05-2.00 (m, 1H), 1.85-1.75 (m, 1H), 1.46 (s, 9H) |
| 208 | tert-butyl (3R)-3-[(1S)- 2-[(4S)-4-benzyl-2-oxo- oxazolidin-3-yl]-1-[(2- bromo-4- pyridyl)methyl]-2-oxo- ethyl]pyrrolidine-1- carboxylate | 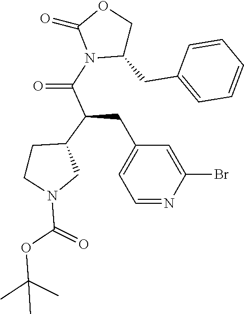 [SMILES: CC(C)(C)OC(=O)N1CC[C@H]([C@H](Cc2ccnc(Br)c2)C(=O)N2C(=O)OC[C@@H]2Cc2ccccc2)C1] | (400 MHz, DMSO-d₆) δ 8.33-8.32 (m, 1H), 7.59-7.57 (m, 1H), 7.37 (m, 1H), 7.21-7.20 (m, 3H), 6.86-6.84 (m, 2H), 4.70-4.60 (m, 1H), 4.35-4.25 (m, 2H), 4.15-4.05 (m, 1H), 3.50-3.30 (m, 1H), 3.20-2.75 (m, 6H), 2.60-2.40 (m, 2H), 1.95-1.85 (m, 1H), 1.65-1.55 (m, 1H), 1.39 (s, 9H) |
| 209 | tert-butyl (3R)-3-[(1S)- 2-[(4S)-4-benzyl-2-oxo- oxazolidin-3-yl]-1-[(2- bromo-5-iodo- phenyl)methyl]-2-oxo- ethyl]pyrrolidine-1- carboxylate | 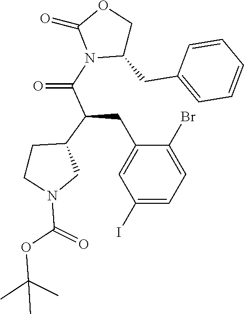 [SMILES: CC(C)(C)OC(=O)N1CC[C@H]([C@H](Cc2cc(I)ccc2Br)C(=O)N2C(=O)OC[C@@H]2Cc2ccccc2)C1] | (400 MHz, CDCl₃) δ ppm 7.44 (s, 1H), 7.29-7.28 (m, 1H), 7.25-7.15 (m, 4H), 7.00-6.95 (m, 2H), 4.60-4.40 (m, 2H), 4.05-3.85 (m, 2H), 3.75-3.30 (m, 2H), 3.20-2.90 (m, 5H), 2.50-2.40 (m, 1H), 2.25-2.15 (m, 1H), 1.85-1.75 (m, 1H), 1.70-1.60 (m, 1H), 1.36 (s, 9H) |

The following were prepared essentially as described in Preparation 12 using the appropriate starting material, stirring the reaction mixture at RT for 16 h following the addition of lithium hydroxide, and omitting any chromatography steps after reaction workup.

| Prep- | Chemical | | |
|---|---|---|---|
| aration | name | Structure | Physical Data |
| 210 | (2S)-3-(5- Bromopyrazin-2-yl)-2- [(3R)-1-tert- butoxycarbonylpyrrolidin- 3-yl]propanoic acid | 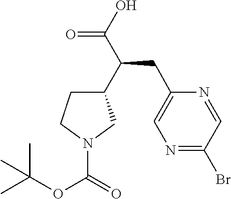 [SMILES: CC(C)(C)OC(=O)N1CC[C@H]([C@H](Cc2cnc(Br)cn2)C(=O)O)C1] | ¹H NMR (400 MHz, methanol-d₄) δ ppm 8.72-8.64 (m,1H), 8.37-8.30 (m, 1H), 3.67-3.59 (m, 1H), 3.49 (m, 1H), 3.35-2.85 (m, 5H), 2.53-2.45 (m,1H), 2.09-2.04 (m, 1H), 1.81-1.71 (m, 1H), 1.4 (s, 9H) |
| 211 | (2S)-3-(5- Bromopyrimidin-2-yl)- 2-[(3R)-1-tert- butoxycarbonylpyrrolidin- 3-yl]propanoic acid | 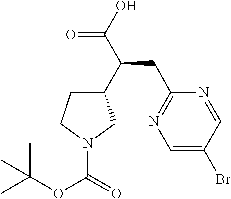 [SMILES: CC(C)(C)OC(=O)N1CC[C@H]([C@H](Cc2ncc(Br)cn2)C(=O)O)C1] | ¹H NMR (400 MHz, methanol-d₄) δ ppm 8.70 (s, 2H), 3.65-3.45 (m, 2H), 3.35-2.95 (m, 5H), 2.55-2.40 (m,1H), 2.10-2.05 (m, 1H), 1.85-1.65 (m, 1H), 1.47 (s, 9H) |
| 212 | (2S)-2-[(3R)-1-tert- Butoxycarbonylpyrrolidin- 3-yl]-3-(2-chloro-4- pyridyl)propanoic acid | 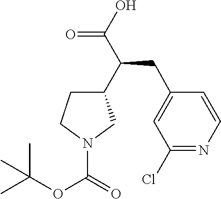 [SMILES: CC(C)(C)OC(=O)N1CC[C@H]([C@H](Cc2ccnc(Cl)c2)C(=O)O)C1] | ¹H NMR (400 MHz, methanol-d₄) δ ppm 8.15-8.13 (m, 1H), 7.26 (m, 1H), 7.16-7.15 (m, 1H), 3.60-3.45 (m, 2H), 3.20-3.10 (m, 1H), 3.05-2.95 (m, 1H), 2.85-2.75 (m, 2H), 2.65-2.50 (m, 1H), 2.40-2.25 (m, 1H), 1.95-1.85 (m, 1H), 1.70-1.55 (m, 1H), |
| | | | 1.47 (s, 9H) |
| 213 | (2S)-3-(3-Bromo-5- methyl-phenyl)-2-[(3R)- 1-tert- butoxycarbonylpyrrolidin- 3-yl]propanoic acid | 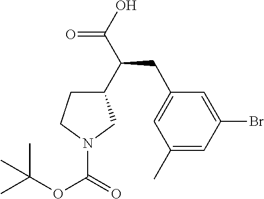 [SMILES: Cc1cc(Br)cc(C[C@H](C(=O)O)[C@H]2CCN(C(=O)OC(C)(C)C)C2)c1] | ¹H NMR (400 MHz, methanol-d₄) δ ppm 7.23-7.17 (m, 2H), 7.05-7.00 (m, 1H), 3.70-3.54 (m, 2H), 3.54-3.39 (m, 2H), 3.30-3.25 (m, 1H), 3.10-2.98 (m, 1H), 2.88-2.79 (m, 2H), 2.61-2.54 (m, 1H), 2.43-2.40 (m, 1H), 2.30 (s, 3H), 1.49 (s, 9H) |
| 214 | (2S)-3-(2-Bromo-4- iodo-phenyl)-2-[(3R)-1- tert-butoxy- carbonylpyrrolidin- 3-yl]propanoic acid | 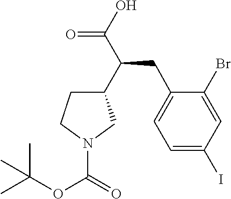 [SMILES: CC(C)(C)OC(=O)N1CC[C@H]([C@H](Cc2ccc(I)cc2Br)C(=O)O)C1] | ¹H NMR (400 MHz, methanol-d₄) δ ppm 7.93 (m, 1H), 7.63-7.61 (m, 1H), 7.27-7.25 (m, 1H), 3.66-3.40 (m, 2H), 3.35-3.12 (m, 2H), 3.05-2.85 (m, 3H), 2.75-2.65 (m, 1H), 2.05-1.95 (m, 1H), 1.80-1.70 (m, 1H), 1.48 (s, 9H) |
| 215 | (2S)-3-[3-Bromo-5- (trifluoromethoxy)phenyl]- 2-[(3R)-1-tert- butoxycarbonylpyrrolidin- 3-yl]propanoic acid | 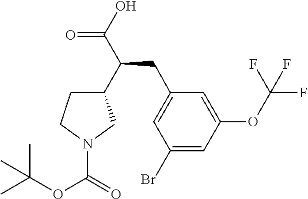 [SMILES: CC(C)(C)OC(=O)N1CC[C@H]([C@H](Cc2cc(Br)cc(OC(F)(F)F)c2)C(=O)O)C1] | ES-MS m/z 482 (M + H) |
| 216 | (2S)-3-(2-Bromo-4- pyridyl)-2-[(3R)-1-tert- butoxycarbonylpyrrolidin- 3-yl]propanoic acid | 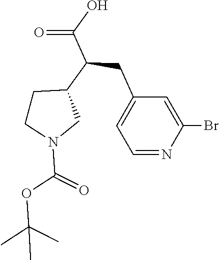 [SMILES: CC(C)(C)OC(=O)N1CC[C@H]([C@H](Cc2ccnc(Br)c2)C(=O)O)C1] | ¹H NMR (400 MHz, methanol-d₄) δ ppm 8.25-8.24 (m, 1H), 7.54 (m, 1H), 7.30 (m, 1H), 3.70-3.45 (m, 2H), 3.35-3.25 (m, 1H), 3.15-3.05 (m, 1H), 2.95-2.85 (m, 2H), 2.75-2.65 (m, 1H), 2.50-2.40 (m, 1H), 2.05-2.00 (m, 1H), 1.85-1.70 (m, 1H), 1.48 (s, 9H) |
| 217 | (2S)-3-(2-Bromo-5- iodo-phenyl)-2-[(3R)-1- tert-butoxy- carbonylpyrrolidin- 3-yl]propanoic acid | 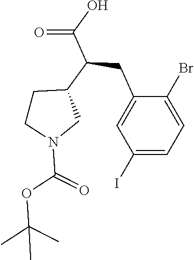 [SMILES: CC(C)(C)OC(=O)N1CC[C@H]([C@H](Cc2cc(I)ccc2Br)C(=O)O)C1] | ES-MS m/z 468/470 (M + H − (tBu)) |

### Preparation 218

### tert-Butyl (3R)-3-[(1S)-1-[(5-bromopyrazin-2-yl)methyl]-2-tert-butoxy-2-oxo-ethyl]pyrrolidine-1-carboxylate

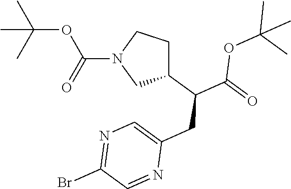 [SMILES: CC(C)(C)OC(=O)[C@@H](Cc1cnc(Br)cn1)[C@H]1CCN(C(=O)OC(C)(C)C)C1]

The title compound was prepared essentially as described in Preparation 79 using (2S)-3-(5-bromopyrazin-2-yl)-2-[(3R)-1-tert-butoxycarbonylpyrrolidin-3-yl]propanoic acid, using THF as solvent, and heating the reaction at 65° C. for 16 h. The reaction mixture was filtered and the filtrate was poured into saturated aqueous NaCl and extracted with EtOAc. The organics were dried over Na₂SO₄, filtered, and concentrated under reduced pressure. The residue was purified by silica gel chromatography using a gradient of 0 to 50% EtOAc in petroleum ether. R_{f}=0.6 on silica gel plate eluted with 17% EtOAc in petroleum ether and visualized with iodine. TLC (1:5 EtOAc/petroleum ether) R_{f}=0.6.

### Preparation 219

### tert-Butyl (3R)-3-[(1S)-1-[(3-bromo-5-methyl-phenyl)methyl]-2-tert-butoxy-2-oxo-ethyl]pyrrolidine-1-carboxylate

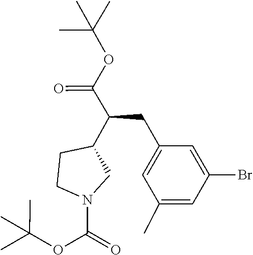 [SMILES: Cc1cc(Br)cc(C[C@H](C(=O)OC(C)(C)C)[C@H]2CCN(C(=O)OC(C)(C)C)C2)c1]

The title compound was prepared essentially as described in Preparation 79 using of (2S)-3-(3-bromo-5-methyl-phenyl)-2-[(3R)-1-tert-butoxycarbonylpyrrolidin-3-yl]propanoic acid, using THF as solvent, and heating the reaction at 65° C. for 16 h. The reaction mixture was filtered and the filtrate was poured into saturated aqueous NaCl and extracted with EtOAc. The organics were dried over Na₂SO₄, filtered, and concentrated under reduced pressure. The residue was purified by silica gel chromatography using a gradient of 0 to 50% EtOAc in petroleum ether. TLC (1:5 EtOAc/petroleum ether) R_{f}=0.6.

### Preparation 220

### Methyl 5-[(2S)-3-tert-butoxy-2-[(3R)-1-tert-butoxycarbonylpyrrolidin-3-yl]-3-oxo-propyl]pyrazine-2-carboxylate

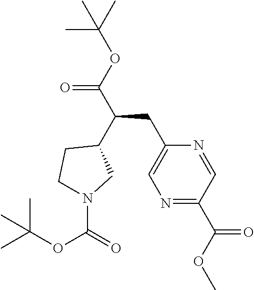 [SMILES: COC(=O)c1cnc(C[C@H](C(=O)OC(C)(C)C)[C@H]2CCN(C(=O)OC(C)(C)C)C2)cn1]

A mixture of tert-butyl (3R)-3-[(1S)-1-[(5-bromopyrazin-2-yl)methyl]-2-tert-butoxy-2-oxo-ethyl]pyrrolidine-1-carboxylate (150 mg, 0.30 mmol, 90 mass %) in MeOH (6 mL) was treated with TEA (0.5 mL, 4 mmol) and [1,1′-bis(diphenylphosphino)ferrocene]dichloropalladium(II) (26 mg, 0.03 mmol) at 25° C. in one portion. The reaction was stirred at 100° C. under CO (2 MPa) for 16 h. The reaction was cooled to RT, filtered, and concentrated under reduced pressure. The residue was purified by silica gel chromatography eluted with a gradient of 0% to 30% EtOAc in petroleum ether to give the title compound (100 mg, 62%) as a yellow oil. ES-MS m/z 436 (M+H).

### Preparation 221

### 5-[(2S)-3-tert-Butoxy-2-[(3R)-1-tert-butoxycarbonylpyrrolidin-3-yl]-3-oxo-propyl]pyrazine-2-carboxylic acid

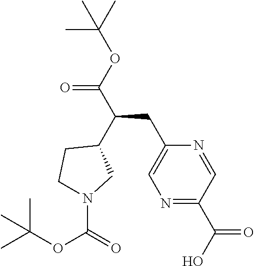 [SMILES: CC(C)(C)OC(=O)[C@@H](Cc1cnc(C(=O)O)cn1)[C@H]1CCN(C(=O)OC(C)(C)C)C1]

A mixture of methyl 5-[(2S)-3-tert-butoxy-2-[(3R)-1-tert-butoxycarbonylpyrrolidin-3-yl]-3-oxo-propyl]pyrazine-2-carboxylate (100 mg, 80 mass %, 0.18 mmol) in THE (2 mL), MeOH (1 mL) and water (0.5 mL) was treated with lithium hydroxide (50 mg, 1.98 mmol) at 25° C. in one portion. The reaction mixture was stirred at 25° C. for 3 h. The pH of the reaction was adjusted to 7 with 1N aqueous HCl. The reaction was extracted with EtOAc (3×10 mL). The organic phases were combined, dried over Na₂SO₄, filtered, and concentrated under reduced pressure giving the title compound (90 mg, 105%) as a yellow oil which was taken on to the next synthetic step without purification. ES-MS m/z 310 (M+H-2^{t}Bu).

### Preparation 222

### tert-Butyl (3R)-3-[(1S)-1-[(2-bromo-4-iodo-phenyl)methyl]-2-tert-butoxy-2-oxo-ethyl]pyrrolidine-1-carboxylate

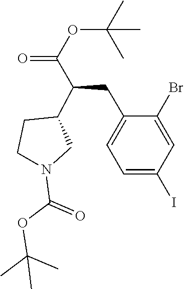 [SMILES: CC(C)(C)OC(=O)[C@@H](Cc1ccc(I)cc1Br)[C@H]1CCN(C(=O)OC(C)(C)C)C1]

The title compound was prepared essentially as described in Preparation 79 using (2S)-3-(2-bromo-4-iodo-phenyl)-2-[(3R)-1-tert-butoxycarbonylpyrrolidin-3-yl]propanoic acid, heating the reaction to 65° C. for 16 h. The reaction mixture was filtered and the filtrate was poured into saturated aqueous NaCl and extracted with EtOAc. The organics were dried over Na₂SO₄, filtered, and concentrated under reduced pressure. The residue was purified by silica gel chromatography using a gradient of 0 to 50% EtOAc in petroleum ether. TLC (1:3 EtOAc/petroleum ether) R_{f}=0.6.

### Preparation 223

### tert-Butyl (3R)-3-[(1S)-1-[(2-bromo-4-cyano-phenyl)methyl]-2-tert-butoxy-2-oxo-ethyl]pyrrolidine-1-carboxylate

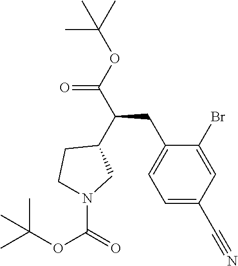 [SMILES: CC(C)(C)OC(=O)[C@@H](Cc1ccc(C#N)cc1Br)[C@H]1CCN(C(=O)OC(C)(C)C)C1]

A solution of tert-butyl (3R)-3-[(1S)-1-[(2-bromo-4-iodo-phenyl)methyl]-2-tert-butoxy-2-oxo-ethyl]pyrrolidine-1-carboxylate (320 mg, 80 mass %, 0.44 mmol) in DMF (4 mL) was treated with zinc cyanide (165 mg, 1.36 mmol) and tetrakis(triphenylphosphine)palladium(0) (110 mg, 0.09 mmol). The reaction was stirred at 80° C. for 3 h. The reaction was cooled to RT, diluted with water (100 mL), and extracted with EtOAc (3×40 mL). The organic layers were combined, washed with saturated aqueous NaCl (50 mL), dried over anhydrous Na₂SO₄, filtered, and concentrated under reduced pressure. The residue was purified by silica gel chromatography eluted with a gradient of 0% to 30% EtOAc in petroleum ether to give the title compound (230 mg, 98%) as a yellow oil. ¹H NMR (400 MHz, methanol-d₄) δ ppm 7.99 (m, 1H), 7.67-7.65 (m, 1H), 7.46-7.43 (m, 1H), 3.70-3.60 (m, 1H), 3.50-3.40 (m, 1H), 3.35-3.00 (m, 4H), 2.75-2.65 (m, 1H), 2.50-2.40 (m, 1H), 2.00-1.90 (m, 1H), 1.80-1.65 (m, 1H), 1.47 (s, 9H), 1.30 (s, 9H).

### Preparation 224

### tert-Butyl (3R)-3-[(1S)-1-[(3-azidophenyl)methyl]-2-tert-butoxy-2-oxo-ethyl]pyrrolidine-1-carboxylate

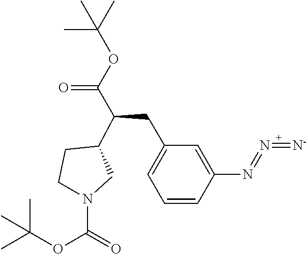

A mixture of tert-butyl nitrite (280 mg, 2.58 mmol) and azidotrimethylsilane (160 mg, 1.32 mmol) in THE (10 mL) was treated in portions with a solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 100 mg, 0.26 mmol) in THE (10 mL) at 0° C. The reaction was stirred at 25° C. for 16 h. The reaction was poured into saturated aqueous NaCl (15 mL) and extracted with EtOAc (3×15 mL). The organics were combined, dried over Na₂SO₄, filtered, and concentrated under reduced pressure to give the title compound (60 mg, 85 mass %, 48%) as a yellow oil that was taken on to the next synthetic step without purification. ¹H NMR (400 MHz, CDCl₃) δ ppm 7.10-7.06 (m, 1H), 6.82-6.76 (m, 1H), 6.75-6.70 (m, 1H), 6.69-6.64 (m, 1H), 3.56-3.42 (m, 2H), 3.37-3.30 (m, 1H), 3.15-3.05 (m, 1H), 2.90-2.80 (m, 1H), 2.74-2.64 (m, 1H), 2.63-2.56 (m, 1H), 2.33-2.27 (m, 1H), 2.25-2.17 (m, 1H), 1.86-1.73 (m, 1H), 1.32 (s, 9H), 1.16 (s, 9H).

### Preparation 225

### tert-Butyl (2S)-3-(3-methylsulfanylphenyl)-2-[(3R)-pyrrolidin-3-yl]propanoate

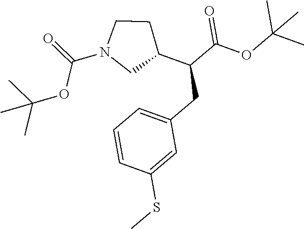 [SMILES: CSc1cccc(C[C@H](C(=O)OC(C)(C)C)[C@H]2CCN(C(=O)OC(C)(C)C)C2)c1]

A solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 200 mg, 0.51 mmol) in methyl disulfide (4 mL) was treated with copper(II) chloride (7 mg, 0.05 mmol) and isoamyl nitrite (60 mg, 0.50 mmol) at 25° C. The reaction was stirred under N₂ (15 psi) at 60° C. When the starting material had been consumed, the reaction was poured into saturated aqueous NaCl (100 mL) and extracted with DCM (3×200 mL). The organics were combined, dried over Na₂SO₄, filtered, and concentrated under reduced pressure. The residue was purified by silica gel chromatography eluted with a gradient of 0% to 10% MeOH in DCM to give the title compound (120 mg, 39%) as a brown solid. ES-MS m/z 322 [M+H-Boc]+.

### Preparation 226

### tert-Butyl (3R)-3-[(1S)-2-tert-butoxy-1-[[3-(1-ethoxyvinyl)phenyl]methyl]-2-oxo-ethyl]pyrrolidine-1-carboxylate

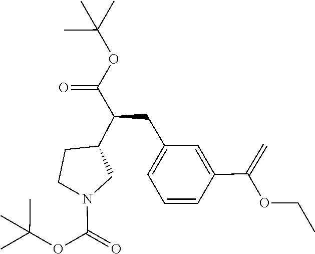 [SMILES: C=C(OCC)c1cccc(C[C@H](C(=O)OC(C)(C)C)[C@H]2CCN(C(=O)OC(C)(C)C)C2)c1]

A solution of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 501 mg, 1.05 mmol) in DMF (5 mL) was treated with tributyl(1-ethoxyvinyl)tin (1.35 g, 3.63 mmol) and tetrakis(triphenylphosphine)palladium(0) (246 mg, 0.21 mmol). The reaction was stirred at 100° C. for 3 h. The reaction mixture was quenched with saturated aqueous KF (5 mL), stirred at 20° C. for 16 h, and filtered. The filtrate was diluted with water (50 mL) and extracted with EtOAc (3×40 mL). The combined organics were washed with saturated aqueous NaCl (50 mL), dried over anhydrous Na₂SO₄, filtered, and concentrated under reduced pressure. The residue was purified by silica gel chromatography eluted with a gradient of 0% to 30% EtOAc in petroleum ether to give the title compound (200 mg, 90 mass %, 39%) as a yellow oil. ¹H NMR (400 MHz, methanol-d₄) δ ppm 7.46-7.45 (m, 2H), 7.30-7.12 (m, 2H), 4.64-4.63 (d, 1H, J=2.4 Hz), 4.22-4.21 (d, 1H, J=2.4 Hz), 3.91 (c, 2H, J=6.8 Hz), 3.70-3.55 (m, 1H), 3.50-3.40 (m, 1H), 3.30-3.20 (m, 1H), 3.10-3.00 (m, 1H), 2.85-2.80 (m, 2H), 2.60-2.50 (m, 1H), 2.40-2.30 (m, 1H), 2.00-1.90 (m, 1H), 1.75-1.60 (m, 1H), 1.47 (s, 9H), 1.41 (t, 3H, J=6.8 Hz), 1.28-1.27 (m, 9H).

### Preparation 227

### tert-Butyl (3R)-3-[(1S)-2-tert-butoxy-2-oxo-1-[[3-(2-trimethylsilylethynyl)phenyl]methyl]ethyl]pyrrolidine-1-carboxylate

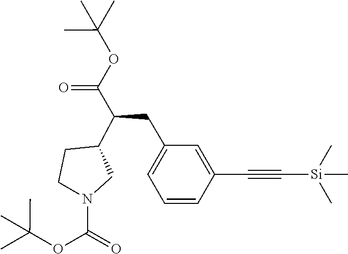 [SMILES: CC(C)(C)OC(=O)[C@@H](Cc1cccc(C#C[Si](C)(C)C)c1)[C@H]1CCN(C(=O)OC(C)(C)C)C1]

A mixture of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 200 mg, 0.44 mmol) and trimethylsilylacetylene (180 mg, 1.80 mmol) in TEA (6 mL) was treated with 1,3-bis(diphenylphosphino)propane (40 mg, 0.1 mmol), tris(dibenzylideneacetone)dipalladium(0) (41 mg, 0.04 mmol) and copper(I) iodide (8.5 mg, 0.05 mmol) at 25° C. in one portion. The reaction was stirred at 120° C. for 16 h under N₂ (15 psi). The reaction was poured into saturated aqueous NaCl (20 mL) and extracted with EtOAc (3×25 mL). The combined organics were washed with water (2×20 mL), dried over Na₂SO₄, filtered, and concentrated under reduced pressure. The residue was purified by silica gel chromatography eluted with a gradient of 0% to 30% EtOAc in petroleum ether. The resulting material was repurified by reversed phase chromatography eluted with a gradient of 75% to 95% ACN in water (with NH₃ and NH₄HCO₃) to give the title compound (50 mg, 90 mass %, 22%) as a yellow oil. ES-MS m/z 472 (M+H).

### Preparation 228

### tert-Butyl (3R)-3-[(1S)-2-tert-butoxy-1-[(3-formyl-5-methyl-phenyl)methyl]-2-oxo-ethyl]pyrrolidine-1-carboxylate

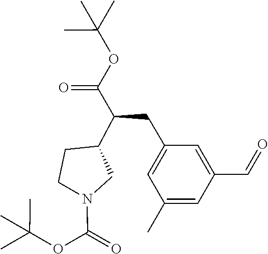 [SMILES: Cc1cc(C=O)cc(C[C@H](C(=O)OC(C)(C)C)[C@H]2CCN(C(=O)OC(C)(C)C)C2)c1]

A mixture of tert-butyl (3R)-3-[(1S)-1-[(3-bromo-5-methyl-phenyl)methyl]-2-tert-butoxy-2-oxo-ethyl]pyrrolidine-1-carboxylate (400 mg, 90 mass %, 0.77 mmol) in THE (4 mL) was treated in portions with N-butyllithium (0.80 mL, 2.0 mmol, 2.5 mol/L in hexanes) at −78° C. The reaction was stirred at −78° C. for 1 h. Then, the reaction was treated with a solution of DMF (0.30 mL, 3.9 mmol) in THE (1 mL) and the reaction was stirred at 25° C. for 16 h. The reaction was poured into saturated aqueous NaCl (20 mL) and extracted with EtOAc (4×30 mL). The combined organics were collected, dried over Na₂SO₄, filtered, and concentrated under reduced pressure. The residue was purified by silica gel chromatography eluted with a gradient of 0% to 20% EtOAc in petroleum ether to give the title compound (60 mg, 60 mass %, 11%) as a yellow oil. ES-MS m/z 318 (M+H-BOC).

### Preparation 229

### tert-Butyl (3R)-3-[(1S)-2-tert-butoxy-1-[(3-methoxycarbonylphenyl)methyl]-2-oxo-ethyl]pyrrolidine-1-carboxylate

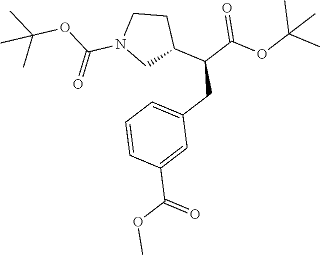 [SMILES: COC(=O)c1cccc(C[C@H](C(=O)OC(C)(C)C)[C@H]2CCN(C(=O)OC(C)(C)C)C2)c1]

A mixture of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 400 mg, 0.88 mmol), TEA (1.3 mL, 9.3 mmol) and [1,1′-bis(diphenylphosphino)ferrocene]dichloropalladium(II) (68 mg, 0.09 mmol) in MeOH (5 mL) was degassed with a CO atmosphere 3 times and stirred at 110° C. overnight under a CO atmosphere (2 Mpa) for 72 h. The reaction was cooled, poured into water (50 mL), and extracted with EtOAc (3×30 mL). The combined organics were washed with saturated aqueous NaCl (30 mL), dried over anhydrous Na₂SO₄, filtered, and concentrated under reduced pressure. The residue was purified by silica gel chromatography eluted with a gradient of 0% to 100% EtOAc in petroleum ether to give the title compound (270 mg, 90 mass %, 83%) as a yellow oil. ES-MS m/z 334 (M+H).

### Preparation 230

### tert-Butyl (3R)-3-[(1S)-2-tert-butoxy-1-[[3-[(tert-butoxycarbonylamino)methyl]-5-methyl-phenyl]methyl]-2-oxo-ethyl]pyrrolidine-1-carboxylate

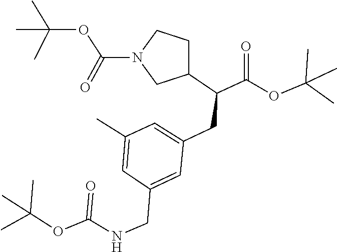 [SMILES: Cc1cc(CNC(=O)OC(C)(C)C)cc(C[C@H](C(=O)OC(C)(C)C)C2CCN(C(=O)OC(C)(C)C)C2)c1]

A mixture of tert-butyl (3R)-3-[(1S)-1-[(3-bromo-5-methyl-phenyl)methyl]-2-tert-butoxy-2-oxo-ethyl]pyrrolidine-1-carboxylate (500 mg, 1.07 mmol), potassium (((tert-butoxycarbonyl)amino)methyl)trifluoroborate (400 mg, 1.61 mmol), PdCl₂(1,1′-bis(di-tert-butylphosphino)ferrocene) (140 mg, 0.213 mmol) and K₂CO₃ (470 mg, 3.23 mmol) in 1,4-dioxane (4 mL) and water (1 mL) was degassed and purged with N₂ three times. The mixture was stirred at 100° C. overnight under N₂ atmosphere. The reaction mixture was combined with a reaction run essentially as described above using 100 mg (0.214 mmol) of the starting aryl bromide. The reaction mixture was diluted with water (30 mL) and extracted with EtOAc (50 mL×3). The organics were washed with saturated aqueous NaCl (50 mL), dried over anhydrous Na₂SO₄, filtered, and concentrated. The residue was purified by silica gel chromatography using a gradient of 0 to 50% EtOAc in petroleum ether to give the title compound (300 mg, 40%) as a yellow oil. ES-MS m/z 419 (M+H-Boc).

### Preparation 231

### tert-Butyl (3R)-3-[(1S)-2-tert-butoxy-1-[[3-methyl-5-(4,4,5,5-tetramethyl-1,3,2-dioxaborolan-2-yl)phenyl]methyl]-2-oxo-ethyl]pyrrolidine-1-carboxylate

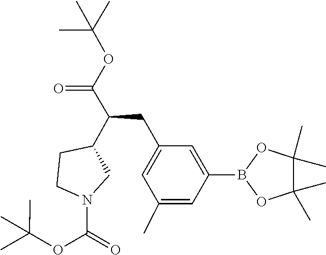 [SMILES: Cc1cc(C[C@H](C(=O)OC(C)(C)C)[C@H]2CCN(C(=O)OC(C)(C)C)C2)cc(B2OC(C)(C)C(C)(C)O2)c1]

A mixture of tert-butyl (3R)-3-[(1S)-1-[(3-bromo-5-methyl-phenyl)methyl]-2-tert-butoxy-2-oxo-ethyl]pyrrolidine-1-carboxylate (1.0 g, 2.14 mmol) and pinacolborane (0.85 g, 6.44 mmol) in 1,2-dichloroethane (10 mL) was treated with dichlorobis(triphenylphosphine)palladium(II) (80 mg, 0.11 mmol) and TEA (1.5 mL, 11 mmol) at 25° C. The reaction was stirred at 90° C. for 16 h under a N₂ (15 Psi) atmosphere. The reaction was concentrated under reduced pressure. The residue was purified by reversed phase HPLC eluted with a gradient of 60% to 100% ACN in water (with NH₃ and NH₄HCO₃) to give the title compound (0.35 g, 90 mass %, 29%) as a yellow oil. ES-MS m/z 516 [M+H]⁺.

### Preparation 232

### tert-Butyl (3R)-3-[(1S)-2-tert-butoxy-1-[(3-hydroxy-5-methyl-phenyl)methyl]-2-oxo-ethyl]pyrrolidine-1-carboxylate

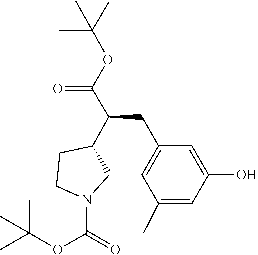 [SMILES: Cc1cc(O)cc(C[C@H](C(=O)OC(C)(C)C)[C@H]2CCN(C(=O)OC(C)(C)C)C2)c1]

A solution of tert-butyl (3R)-3-[(1S)-2-tert-butoxy-1-[[3-methyl-5-(4,4,5,5-tetramethyl-1,3,2-dioxaborolan-2-yl)phenyl]methyl]-2-oxo-ethyl]pyrrolidine-1-carboxylate (250 mg, 0.49 mmol) in THE (6 mL) was treated with sodium hydroxide (40 mg, 0.99 mmol) and hydrogen peroxide solution (110 mg, 30 mass % in water, 0.97 mmol) at 0° C. The reaction was stirred at 25° C. for 3 h. The reaction was combined with a 50 mg reaction for work up. The reaction was poured into saturated aqueous NaCl (50 mL) and extracted with EtOAc (4×100 mL). The organics were combined, dried over Na₂SO₄, filtered, and concentrated under reduced pressure. The residue was purified by silica gel chromatography eluted with a gradient of 0% to 100% EtOAc in petroleum ether to give the title compound (240 mg, 80 mass %, 98%) as a yellow oil. ES-MS m/z 406.4 [M+H]⁺.

### Preparation 233

### (2S)-3-(2-Bromo-4-carbamoyl-phenyl)-2-[(3R)-1-tert-butoxycarbonylpyrrolidin-3-yl]propanoic acid

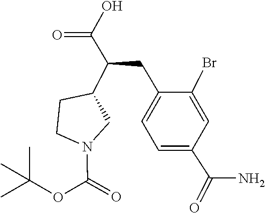 [SMILES: CC(C)(C)OC(=O)N1CC[C@H]([C@H](Cc2ccc(C(N)=O)cc2Br)C(=O)O)C1]

A mixture of tert-butyl (3R)-3-[(1S)-2-[(4S)-4-benzyl-2-oxo-oxazolidin-3-yl]-1-[(2-bromo-4-cyano-phenyl)methyl]-2-oxo-ethyl]pyrrolidine-1-carboxylate (430 mg, 80 mass %, 0.59 mmol) in THE (8 mL) was treated with hydrogen peroxide (140 mg, 30% in water, 1.23 mmol) at 0° C. in one portion. Then, a solution of lithium hydroxide (30 mg, 1.24 mmol) in water (2 mL) was added to the mixture at 0° C. in portions. The reaction was stirred at 20° C. for 3 h. A solution of saturated aqueous sodium bisulfite (30 mL) was added to the reaction over 5 min, raising the temperature to 30° C. The pH was adjusted to >12 with 5N aqueous NaOH and extracted with EtOAc (3×30 mL). The pH of the aqueous layer was adjusted to 2 with 2N aqueous HCl and extracted with EtOAc (3×30 mL). The organics from the acidic extraction were combined, dried over Na₂SO₄, filtered, and concentrated under reduced pressure giving the title compound (160 mg, 70 mass %, 43%) as a colorless oil. TLC (1:1 EtOAc/petroleum ether) R_{f}=0.2.

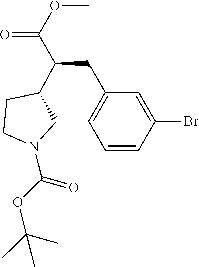 [SMILES: COC(=O)[C@@H](Cc1cccc(Br)c1)[C@H]1CCN(C(=O)OC(C)(C)C)C1]

A mixture of (2S)-3-(3-bromophenyl)-2-[(3R)-1-tert-butoxycarbonylpyrrolidin-3-yl]propanoic acid (400 mg, ammonium salt, 0.96 mmol) in DMF (5 mL) was treated with sodium bicarbonate (250 mg, 2.98 mmol) and iodomethane (420 mg, 2.93 mmol) at 25° C. The reaction was stirred at 25° C. for 16 h. The reaction was poured into saturated aqueous NaCl (15 mL) and extracted with EtOAc (3×20 mL). The organics were combined, dried over Na₂SO₄, filtered, and concentrated under reduced pressure. The residue was purified by silica gel chromatography eluted with a gradient of 0% to 20% EtOAc in petroleum ether to give the title compound (300 mg, 76%) as a yellow oil which was used in the next synthetic step without characterization.

### Preparation 235

### Methyl (2S)-3-(3-bromophenyl)-2-[(3R)-pyrrolidin-3-yl]propanoate

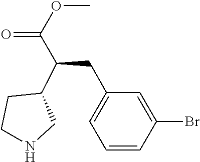 [SMILES: COC(=O)[C@@H](Cc1cccc(Br)c1)[C@H]1CCNC1]

tert-Butyl (3R)-3-[(1S)-1-[(3-bromophenyl)methyl]-2-methoxy-2-oxo-ethyl]pyrrolidine-1-carboxylate (390 mg, 0.95 mmol) was treated with HCl (5 mL, 4 M in 1,4-dioxane, 20 mmol) at 25° C. The reaction was stirred at 25° C. for 3 h. The pH of the reaction was adjusted to 8 with saturated aqueous NaHCO₃ and extracted with EtOAc (4×30 mL). The organics were collected, dried over Na₂SO₄, filtered, and concentrated under reduced pressure. The residue was purified by silica gel chromatography eluted with a gradient of 0% to 80% EtOAc in petroleum ether to give the title compound (230 mg, 90 mass %, 70%) as a colorless oil. ES-MS m/z 312.1 (M+H).

### Preparation 236

### Methyl (2S)-2-[(3R)-pyrrolidin-3-yl]-3-(3-vinylphenyl)propanoate

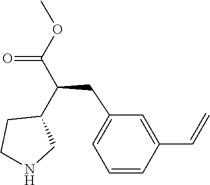 [SMILES: C=Cc1cccc(C[C@H](C(=O)OC)[C@H]2CCNC2)c1]

A mixture of methyl (2S)-3-(3-bromophenyl)-2-[(3R)-pyrrolidin-3-yl]propanoate (230 mg, 0.74 mmol) and vinylboronic acid pinacol ester (180 mg, 1.11 mmol) in water (1 mL) and 1,2-dimethoxyethane (10 mL) was treated with potassium carbonate (200 mg, 1.45 mmol) and [1,1′-bis(diphenylphosphino)ferrocene]dichloropalladium(II) (58 mg, 0.08 mmol) at 25° C. The reaction was stirred at 90° C. for 16 h under N₂ (15 psi). The reaction mixture was combined with a 20 mg reaction and concentrated under reduced pressure. The residue was purified by silica gel chromatography eluted with a gradient of 0% to 20% MeOH in DCM to give the title compound (120 mg, 45%) as a yellow oil. TLC (DCM/MeOH=10:1) R_{f}=0.5, observed by I₂ staining.

### Preparation 237

### tert-Butyl (3R)-3-[(1S)-2-tert-butoxy-1-[[3-(hydroxymethyl)-5-methyl-phenyl]methyl]-2-oxo-ethyl]pyrrolidine-1-carboxylate

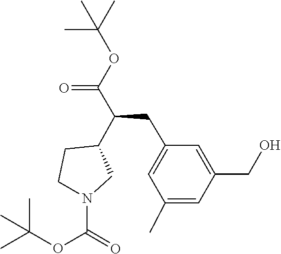 [SMILES: Cc1cc(CO)cc(C[C@H](C(=O)OC(C)(C)C)[C@H]2CCN(C(=O)OC(C)(C)C)C2)c1]

A solution of tert-butyl (3R)-3-[(1S)-1-[(3-bromo-5-methyl-phenyl)methyl]-2-tert-butoxy-2-oxo-ethyl]pyrrolidine-1-carboxylate (500 mg, 1.07 mmol), tributylstannylmethanol (412 mg, 1.28 mmol), and (2-dicyclohexylphosphino-2′,4′,6′-triisopropyl-1,1′-biphenyl)[2-(2′-amino-1,1′-biphenyl)]palladium(II) methanesulfonate (100 mg, 0.12 mmol) in 1,4-dioxane (5 mL, 58.57 mmol) was degassed and purged with N₂ 3 times. The reaction was stirred at 100° C. overnight under a N₂ atmosphere. The reaction was combined with a 100 mg reaction for work up. The reaction was diluted with H₂O (30 mL) and extracted with EtOAc (3×50 mL). The combined organics were washed with saturated aqueous NaCl (50 mL), dried over anhydrous Na₂SO₄, filtered, and concentrated under gradient of 0% to 50% EtOAc in petroleum ether to give the title compound (400 mg, 90 mass %, 80%) as a yellow oil. ES-MS m/z 442 (M+Na).

### Preparation 238

### tert-Butyl (3R)-3-[(1S)-2-tert-butoxy-2-oxo-1-[[3-(4,4,5,5-tetramethyl-1,3,2-dioxaborolan-2-yl)phenyl]methyl]ethyl]pyrrolidine-1-carboxylate

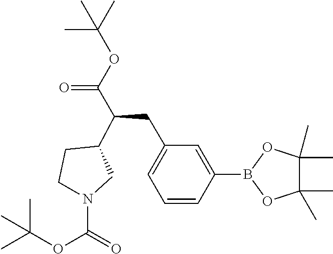 [SMILES: CC(C)(C)OC(=O)[C@@H](Cc1cccc(B2OC(C)(C)C(C)(C)O2)c1)[C@H]1CCN(C(=O)OC(C)(C)C)C1]

A mixture of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 200 mg, 0.44 mmol), bis(pinacolato)diboron (170 mg, 0.66 mmol), [1,1′-bis(diphenylphosphino)ferrocene]dichloropalladium(II) (34 mg, 0.04 mmol), and potassium acetate (132 mg, 1.33 mmol) in 1,4-dioxane (3 mL) was degassed and purged with N₂ 3 times, and then the mixture was stirred at 100° C. overnight. The reaction was diluted with H₂O (15 mL) and extracted with EtOAc (2×20 mL). The organic layers were washed with saturated aqueous NaCl (20 mL), dried over anhydrous Na₂SO₄, filtered, and concentrated under reduced pressure. The residue was purified by silica gel chromatography eluted with a gradient of 0% to 100% EtOAc in petroleum ether to give the title compound (190 mg, 80 mass %, 69%) as a pale yellow oil. ES-MS m/z 502 (M+H).

### Preparation 239

### tert-Butyl (3R)-3-[(1S)-2-tert-butoxy-2-oxo-1-[[3-[1-(2-trimethylsilylethoxymethyl)pyrazol-3-yl]phenyl]methyl]ethyl]pyrrolidine-1-carboxylate

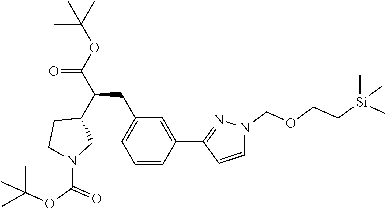 [SMILES: CC(C)(C)OC(=O)[C@@H](Cc1cccc(-c2ccn(COCC[Si](C)(C)C)n2)c1)[C@H]1CCN(C(=O)OC(C)(C)C)C1]

A mixture of [1-(2-trimethylsilylethoxymethyl)pyrazol-3-yl]boronic acid (150 mg, 0.62 mmol), tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 200 mg, 0.44 mmol), Na₂CO₃ (132 mg, 1.25 mmol), and [1,1′-bis(diphenylphosphino)ferrocene]dichloropalladium(II) (46 mg, 0.06 mmol) in 1,4-dioxane (4 mL) and water (1 mL) was purged with N₂ (3 times) and the reaction stirred at 100° C. overnight. The reaction was diluted with H₂O (25 mL) and extracted with EtOAc (2×20 mL). The organic layers were washed with saturated aqueous NaCl (20 mL), dried over anhydrous Na₂SO₄, filtered, and concentrated under reduced pressure. The residue was purified by silica gel chromatography eluted with a gradient of 0% to 100% EtOAc in petroleum ether to give the title compound (180 mg, 80 mass %, 41%) as a pale yellow oil. ES-MS m/z 572 (M+H).

### Preparation 240

### tert-Butyl (3R)-3-[(1S)-2-tert-butoxy-2-oxo-1-[[3-(1H-pyrazol-3-yl)phenyl]methyl]ethyl]pyrrolidine-1-carboxylate

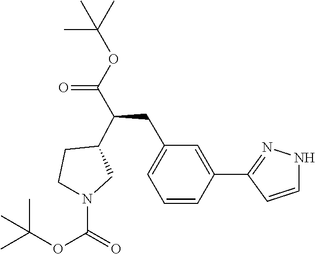 [SMILES: CC(C)(C)OC(=O)[C@@H](Cc1cccc(-c2cc[nH]n2)c1)[C@H]1CCN(C(=O)OC(C)(C)C)C1]

A solution of tert-butyl (3R)-3-[(1S)-2-tert-butoxy-2-oxo-1-[[3-[1-(2-trimethylsilylethoxymethyl)pyrazol-3-yl]phenyl]methyl]ethyl]pyrrolidine-1-carboxylate (180 mg, 80 mass %, 0.25 mmol) in THE (4 mL) was treated with tetrabutylammonium fluoride (1.3 mL, 1 N in THF, 1.3 mmol) and the reaction stirred at 50° C. for 16 h. The reaction was diluted with water (20 mL) and extracted with EtOAc (3×20 mL). The combined organics were washed with saturated aqueous NaCl (30 mL), dried over anhydrous Na₂SO₄, filtered, the filtrate concentrated under reduced pressure to give the title compound (150 mg, 80 mass %, 108%) that was taken on to the next synthetic step without purification. ES-MS m/z 442.3 (M+H).

### Preparation 241

### tert-Butyl (3R)-3-[(1S)-2-tert-butoxy-1-[[3-[1-[3-[(2S)-3-tert-butoxy-2-[(3R)-1-tert-butoxycarbonylpyrrolidin-3-yl]-3-oxo-propyl]phenyl]pyrazol-3-yl]phenyl]methyl]-2-oxo-ethyl]pyrrolidine-1-carboxylate

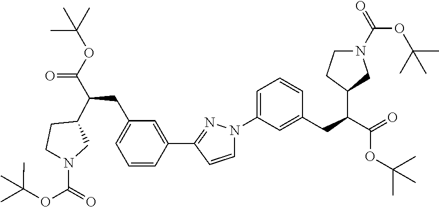 [SMILES: CC(C)(C)OC(=O)[C@@H](Cc1cccc(-c2ccn(-c3cccc(C[C@H](C(=O)OC(C)(C)C)[C@H]4CCN(C(=O)OC(C)(C)C)C4)c3)n2)c1)[C@H]1CCN(C(=O)OC(C)(C)C)C1]

A solution of tert-butyl (3R)-3-[(1S)-2-tert-butoxy-2-oxo-1-[[3-(4,4,5,5-tetramethyl-1,3,2-dioxaborolan-2-yl)phenyl]methyl]ethyl]pyrrolidine-1-carboxylate (100 mg, 80 mass % 0.16 mmol) and tert-butyl (3R)-3-[(1S)-2-tert-butoxy-2-oxo-1-[[3-(1H-pyrazol-3-yl)phenyl]methyl]ethyl]pyrrolidine-1-carboxylate (140 mg, 80 mass %, 0.25 mmol) in pyridine (4 mL) was treated with copper(II) acetate (60 mg, 0.33 mmol). The reaction was stirred at 70° C. under O₂ overnight. The reaction was diluted with water (10 mL) and extracted with EtOAc (3×30 mL). The organic layers were combined, washed with saturated aqueous NaCl (30 mL), collected, dried over anhydrous Na₂SO₄, filtered, and concentrated under reduced pressure. The residue was purified by silica gel chromatography eluted with a gradient of 0% to 20% EtOAc in petroleum leaving an unpure product. A second purification was done by SFC [Column: Daicel CHIRALPAK IC™ (250 mm×30 mm, 10 m); mobile phase: solvent A=CO₂, solvent B=EtOH+0.1% NH₄OH); isocratic 35% solvent B in solvent A; flow rate 80 mL/min] giving the title compound (50 mg, 90 mass %, 35%) as a white solid. ES-MS m/z 815.7 (M+H).

### Preparation 243

### 3-[(2S)-3-tert-Butoxy-2-[(3R)-1-tert-butoxycarbonylpyrrolidin-3-yl]-3-oxo-propyl]benzoic acid

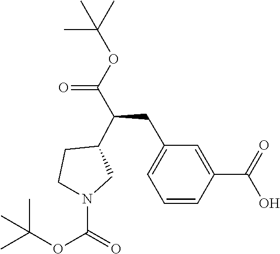 [SMILES: CC(C)(C)OC(=O)[C@@H](Cc1cccc(C(=O)O)c1)[C@H]1CCN(C(=O)OC(C)(C)C)C1]

A solution of tert-butyl (3R)-3-[(1S)-2-tert-butoxy-1-[(3-methoxycarbonylphenyl)methyl]-2-oxo-ethyl]pyrrolidine-1-carboxylate (390 mg, 95 mass %, 0.85 mmol) in THE (4 mL) and MeOH (2 mL) was treated with lithium hydroxide (150 mg, 3.50 mmol) in water (1 mL) at 25° C. The reaction was stirred at 25° C. for 12 h. The reaction was concentrated under reduced pressure. The pH of the mixture was adjusted to 6 with HCl (1N). The mixture was extracted with EtOAc (3×30 mL). The combined organics were dried over Na₂SO₄, filtered, and concentrated under reduced pressure to give the title compound (370 mg, 98%) as a white solid. ES-MS m/z 320 (M+H-BOC).

### Preparation 244

### tert-Butyl (3R)-3-[(1S)-2-tert-butoxy-1-[[3-[[3-[(2S)-3-tert-butoxy-2-[(3R)-1-tert-butoxycarbonylpyrrolidin-3-yl]-3-oxo-propyl]phenyl]carbamoyl]phenyl]methyl]-2-oxo-ethyl]pyrrolidine-1-carboxylate

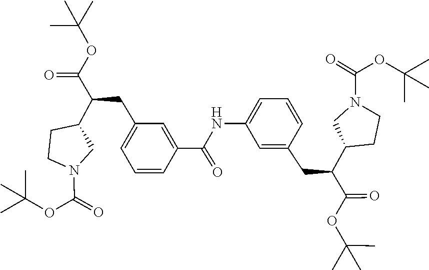 [SMILES: CC(C)(C)OC(=O)[C@@H](Cc1cccc(NC(=O)c2cccc(C[C@H](C(=O)OC(C)(C)C)[C@H]3CCN(C(=O)OC(C)(C)C)C3)c2)c1)[C@H]1CCN(C(=O)OC(C)(C)C)C1]

A mixture of 3-[(2S)-3-tert-butoxy-2-[(3R)-1-tert-butoxycarbonylpyrrolidin-3-yl]-3-oxo-propyl]benzoic acid (320 mg, 0.72 mmol) and tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 290 mg, 0.74 mmol) in DMF (15 mL, 194 mmol, 100 mass %) was treated with N,N-diisopropylethylamine (380 mg, 2.94 mmol) and HATU (420 mg, 1.10 mmol) at 25° C. The reaction was stirred at 25° C. for 15 h. The reaction was poured into saturated aqueous NaCl (30 mL) and extracted with EtOAc (3×50 mL). The combined organics were dried over Na₂SO₄, filtered, and concentrated under reduced pressure. The residue was purified by silica gel chromatography eluted with a gradient of 0% to 100% EtOAc in petroleum ether to give the title compound (460 mg, 85 mass %, 68%) as a yellow oil. ES-MS m/z 692 (M+H-BOC).

### Preparation 245

### tert-Butyl (3R)-3-[(1S)-2-tert-butoxy-1-[[3-[[3-[(2S)-3-tert-butoxy-2-[(3R)-1-tert-butoxycarbonylpyrrolidin-3-yl]-3-oxo-propyl]phenyl]sulfonylamino]phenyl]methyl]-2-oxo-ethyl]pyrrolidine-1-carboxylate

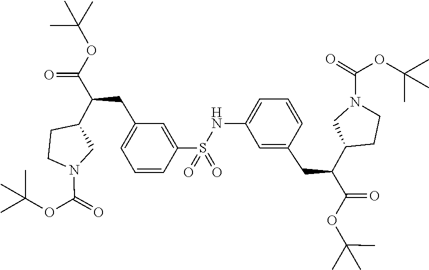 [SMILES: CC(C)(C)OC(=O)[C@@H](Cc1cccc(NS(=O)(=O)c2cccc(C[C@H](C(=O)OC(C)(C)C)[C@H]3CCN(C(=O)OC(C)(C)C)C3)c2)c1)[C@H]1CCN(C(=O)OC(C)(C)C)C1]

A mixture of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 270 mg, 0.69 mmol) in THE (5 mL) and pyridine (36 mg, 0.45 mmol) was stirred at RT for 0.5 h and treated with tert-butyl (3R)-3-[(1S)-2-tert-butoxy-1-[(3-chlorosulfonylphenyl)methyl]-2-oxo-ethyl]pyrrolidine-1-carboxylate (300 mg, 90 mass %, 0.56 mmol) was stirred at RT for 2 h. The reaction was diluted with H₂O (10 mL) and extracted with EtOAc (2×20 mL). The combined organic layers were washed with saturated aqueous NaCl (20 mL), dried over anhydrous Na₂SO₄, filtered, and concentrated under reduced pressure. The residue was purified by silica gel chromatography eluted with a gradient of 0% to 60% EtOAc in petroleum ether to give the title compound (330 mg, 90 mass %, 63%) as a pale yellow solid. ES-MS m/z 829 (M+H).

### Preparation 246

### tert-Butyl (3R)-3-[(1S)-2-tert-butoxy-1-[(3-ethynylphenyl)methyl]-2-oxo-ethyl]pyrrolidine-1-carboxylate

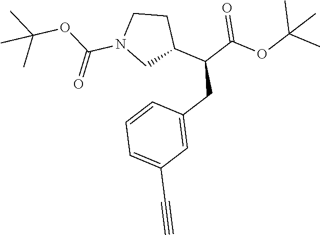 [SMILES: C#Cc1cccc(C[C@H](C(=O)OC(C)(C)C)[C@H]2CCN(C(=O)OC(C)(C)C)C2)c1]

A mixture of tert-butyl (3R)-3-[(1S)-2-tert-butoxy-2-oxo-1-[[3-(2-trimethylsilylethynyl)phenyl]methyl]ethyl]pyrrolidine-1-carboxylate (300 mg, 80 mass %, 0.51 mmol) in THE (2 mL) was treated with tetrabutylammonium fluoride (940 mg, 3.60 mmol) at 25° C. When the reaction was complete [monitoring by TLC (1:5 EtOAc:petroleum ether)], the mixture was poured into NH₄Cl (20 mL) and extracted with EtOAc (3×25 mL). The combined organic phases were washed with water (2×20 mL), dried over Na₂SO₄, filtered, and concentrated under reduced pressure to give the title compound (240 mg, 77 mass %, 91%) as a yellow oil. ES-MS m/z 422 (M+Na).

### Preparation 247

### tert-Butyl (3R)-3-[(1S)-2-tert-butoxy-1-[[3-[1-[3-[(2S)-3-tert-butoxy-2-[(3R)-1-tert-butoxycarbonylpyrrolidin-3-yl]-3-oxo-propyl]phenyl]triazol-4-yl]phenyl]methyl]-2-oxo-ethyl]pyrrolidine-1-carboxylate

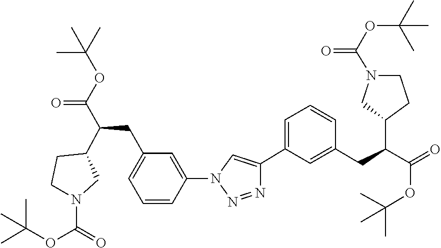 [SMILES: CC(C)(C)OC(=O)[C@@H](Cc1cccc(-c2cn(-c3cccc(C[C@H](C(=O)OC(C)(C)C)[C@H]4CCN(C(=O)OC(C)(C)C)C4)c3)nn2)c1)[C@H]1CCN(C(=O)OC(C)(C)C)C1]

To a mixture of tert-butyl (3R)-3-[(1S)-1-[(3-azidophenyl)methyl]-2-tert-butoxy-2-oxo-ethyl]pyrrolidine-1-carboxylate (180 mg, 0.432 mmol) and tert-butyl (3R)-3-[(1S)-2-tert-butoxy-1-[(3-ethynylphenyl)methyl]-2-oxo-ethyl]pyrrolidine-1-carboxylate (180 mg, 0.451 mmol) in tert-butanol (2 mL) and water (4 mL) was added cupric sulfate (7 mg, 0.044 mmol), sodium ascorbate (26 mg, 0.13 mmol) and benzoic acid (30 mg, 0.25 mmol) at 25° C. in one portion. The reaction mixture was stirred at 25° C. for 16 h then concentrated to dryness under reduced pressure. The residue was purified by silica gel chromatography using a gradient of 0 to 30% EtOAc in petroleum ether to give the title compound (200 mg, 51%) as a yellow oil. ES-MS m/z 816.5.

### Preparation 248

### (R)-tert-Butyl 3-((S)-1-(tert-butoxy)-3-(3-(5-((S)-3-(tert-butoxy)-2-((R)-1-(tert-butoxycarbonyl)pyrrolidin-3-yl)-3-oxopropyl)pyrazine-2-carboxamido)phenyl)-1-oxopropan-2-yl)pyrrolidine-1-carboxylate

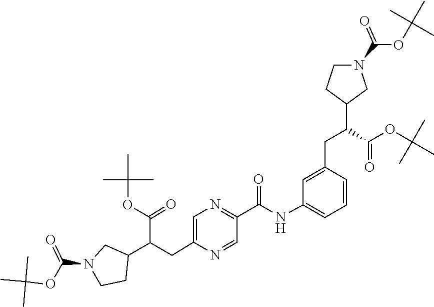

To a mixture of 5-[(2S)-3-tert-butoxy-2-[(3R)-1-tert-butoxycarbonylpyrrolidin-3-yl]-3-oxo-propyl]pyrazine-2-carboxylic acid (130 mg, 0.308 mmol) and tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 120 mg, 0.307 mmol) in DMF (4 mL) was added N,N-diisopropylethylamine (160 mg, 1.24 mmol) and HATU (180 mg, 0.473 mmol) at 25° C. in one portion. The reaction mixture was stirred at 25° C. for 5 h, then combined with a reaction mixture which was run on 20 mg (0.038 mmol) of the starting carboxylic acid. The mixture was poured into saturated aqueous NaCl (15 mL) and extracted with EtOAc (25 mL×4). The combined organic phases were dried over Na₂SO₄, filtered and concentrated to dryness under reduced pressure. The residue was purified by silica gel chromatography using a gradient of 0 to 10% EtOAc in petroleum ether to give the title compound (200 mg, 69%) as a yellow oil. ES-MS m/z 694.6.

### Preparation 249

### (2S)-2-[(3R)-1-tert-Butoxycarbonylpyrrolidin-3-yl]-3-[2-[3-[4-[(2S)-2-[(3R)-1-tert-butoxycarbonylpyrrolidin-3-yl]-2-carboxy-ethyl]-2-pyridyl]-2-oxo-imidazolidin-1-yl]-4-pyridyl]propanoic acid

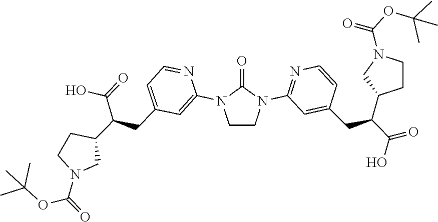 [SMILES: CC(C)(C)OC(=O)N1CC[C@H]([C@H](Cc2ccnc(N3CCN(c4cc(C[C@H](C(=O)O)[C@H]5CCN(C(=O)OC(C)(C)C)C5)ccn4)C3=O)c2)C(=O)O)C1]

A mixture of (2S)-3-(2-bromo-4-pyridyl)-2-[(3R)-1-tert-butoxycarbonylpyrrolidin-3-yl]propanoic acid (2.1 g, 4.7 mmol), imidazolidin-2-one (330 mg, 3.83 mmol), [(2-di-tert-butylphosphino-2′,4′,6′-triisopropyl-1,1′-biphenyl)-2-(2′-amino-1,1′-biphenyl)]palladium(II) methanesulfonate (400 mg, 0.493 mmol), sodium tert-butoxide (1.4 g, 14 mmol) in 1,4-dioxane (15 mL) was stirred under N₂ (15 Psi) at 100° C. for 16 h. The reaction mixture was concentrated and the residue was purified by HPLC [column: Welch Xtimate® C18 250×50 mm, 10 μm; mobile phase: solvent A=aqueous formic acid, solvent B=ACN; gradient 20 to 100% solvent B in solvent A] to give the title compound (100 mg, 3%) as a yellow oil. ES-MS m/z 723.5.

### Preparation 250

### tert-Butyl (3R)-3-[(1S)-1-[(2-bromo-5-iodo-phenyl)methyl]-2-tert-butoxy-2-oxo-ethyl]pyrrolidine-1-carboxylate

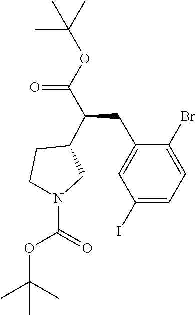 [SMILES: CC(C)(C)OC(=O)[C@@H](Cc1cc(I)ccc1Br)[C@H]1CCN(C(=O)OC(C)(C)C)C1]

To a mixture of (2S)-3-(2-bromo-5-iodo-phenyl)-2-[(3R)-1-tert-butoxycarbonylpyrrolidin-3-yl]propanoic acid (4.9 g, 6.5 mmol) in THE (60 mL) was added 2-tert-butyl-1,3-diisopropylisourea (4 g, 19.6 mmol) at 25° C. in one portion. The reaction mixture was stirred at 70° C. for 16 h, then diluted with water (150 mL) and extracted with EtOAc (200 mL×3). The combined organic layers were washed with saturated aqueous NaCl (100 mL×2), dried over anhydrous Na₂SO₄, filtered and concentrated under reduced pressure. The residue was purified by silica gel chromatography using a gradient of 0 to 100% EtOAc in petroleum ether to give the title compound (2 g, 48%) as a yellow oil. TLC (1:3 EtOAc/petroleum ether) R_{f}=0.7.

### Preparation 251

### tert-Butyl (3R)-3-[(1S)-1-[(2-bromo-5-cyano-phenyl)methyl]-2-tert-butoxy-2-oxo-ethyl]pyrrolidine-1-carboxylate

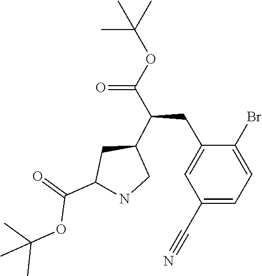

To a solution of tert-butyl (3R)-3-[(1S)-1-[(2-bromo-5-iodo-phenyl)methyl]-2-tert-butoxy-2-oxo-ethyl]pyrrolidine-1-carboxylate (2 g, 3.1 mmol) in DMF (30 mL) was added zinc cyanide (380 mg, 3.14 mmol) and tetrakis(triphenylphosphine)palladium(0) (730 mg, 0.625 mmol) at 25° C., the mixture was stirred at 80° C. for 1 h under N₂. The reaction mixture was diluted with water (100 mL) extracted with EtOAc (100 mL×3). The combined organic layers were washed with saturated aqueous NaCl (100 mL×2), dried over anhydrous Na₂SO₄, filtered and concentrated under reduced pressure. The residue was purified by silica gel chromatography using a gradient of 0 to 100% EtOAc in petroleum ether to give the title compound (1.38 g, 88%) as a yellow oil. TLC (1:3 EtOAc/petroleum ether) R_{f}=0.6.

### Preparation 252

### tert-Butyl (3R)-3-[(1R)-2-tert-butoxy-1-[[3-[2-[(2S)-3-tert-butoxy-2-[(3R)-1-tert-butoxycarbonylpyrrolidin-3-yl]-3-oxo-propyl]-4-cyano-anilino]phenyl]methyl]-2-oxo-ethyl]pyrrolidine-1-carboxylate

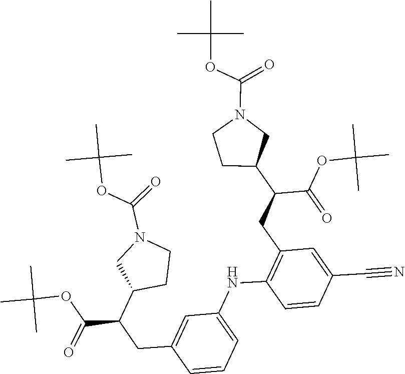 [SMILES: CC(C)(C)OC(=O)[C@@H](Cc1cc(C#N)ccc1Nc1cccc(C[C@@H](C(=O)OC(C)(C)C)[C@H]2CCN(C(=O)OC(C)(C)C)C2)c1)[C@H]1CCN(C(=O)OC(C)(C)C)C1]

To a solution of tert-butyl (3R)-3-[(1S)-1-[(2-bromo-5-cyano-phenyl)methyl]-2-tert-butoxy-2-oxo-ethyl]pyrrolidine-1-carboxylate (180 mg, 0.376 mmol) in 1,4-dioxane (15 mL) was added potassium tert-butoxide (130 mg, 1.12 mmol), [1,1′-bis(diphenylphosphino)ferrocene]dichloropalladium(II) (60 mg, 0.082 mmol) and tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 180 mg, 0.461 mmol). The reaction mixture was stirred at 100° C. for 4 h under N₂. The reaction mixture was poured into water (100 mL) and the aqueous layer was extracted with EtOAc (100 mL×3). The organic layer was dried over anhydrous Na₂SO₄, filtered and the filtrate was concentrated under reduced pressure. The residue was purified by silica gel chromatography using a gradient of 0 to 100% EtOAc in petroleum ether to give the title compound (20 mg, 5%) as yellow solid. ES-MS m/z 633.3 (M-Boc-tert-butyl).

### Preparation 253

### tert-Butyl (3R)-3-[(1S)-2-tert-butoxy-1-[[3-[[[3-[(2S)-3-tert-butoxy-2-[(3R)-1-tert-butoxycarbonylpyrrolidin-3-yl]-3-oxo-propyl]-5-methyl-phenyl]methylamino]methyl]-5-methyl-phenyl]methyl]-2-oxo-ethyl]pyrrolidine-1-carboxylate

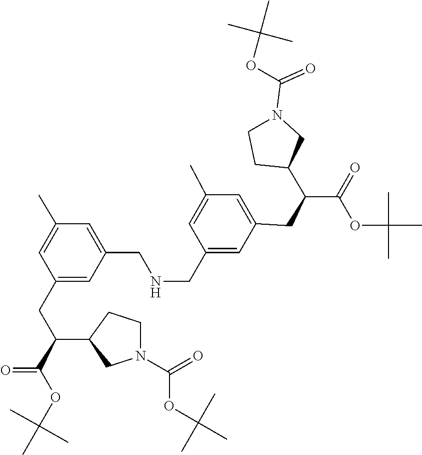 [SMILES: Cc1cc(CNCc2cc(C)cc(C[C@H](C(=O)OC(C)(C)C)[C@H]3CCN(C(=O)OC(C)(C)C)C3)c2)cc(C[C@H](C(=O)OC(C)(C)C)[C@H]2CCN(C(=O)OC(C)(C)C)C2)c1]

To a solution of tert-butyl (3R)-3-[(1S)-2-tert-butoxy-1-[(3-formyl-5-methyl-phenyl)methyl]-2-oxo-ethyl]pyrrolidine-1-carboxylate (300 mg, 0.719 mmol) in THE (3 mL) was added ammonium acetate (250 mg, 3.24 mmol) and the mixture was stirred at 20° C. for 30 min. Then to the mixture was added sodium triacetoxyborohydride (460 mg, 2.17 mmol) and the mixture was stirred at 20° C. for 2 h. To the reaction mixture was added saturated aqueous NaCl (300 mL) and the mixture was extracted with EtOAc (200 mL×3). The combined organic phase was washed with water (200 mL×2, then 150 mL×3), then dried over Na₂SO₄, filtered, and concentrated under reduced pressure. The residue was purified by silica gel chromatography using a gradient of 0 to 50% EtOAc in petroleum ether to give the title compound (150 mg, 25%). TLC (3:1 petroleum ether. EtOAc) R_{f}=0.2.

### Preparation 254

### tert-Butyl (3R)-3-[(1S)-1-[[3-[[bis[[3-[(2S)-3-tert-butoxy-2-[(3R)-1-tert-butoxycarbonylpyrrolidin-3-yl]-3-oxo-propyl]-5-methyl-phenyl]methyl]amino]methyl]-5-methyl-phenyl]methyl]-2-tert-butoxy-2-oxo-ethyl]pyrrolidine-1-carboxylate

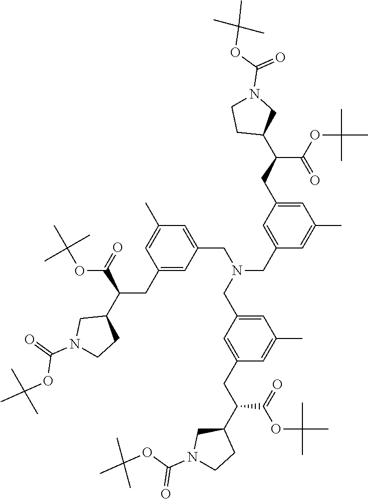 [SMILES: Cc1cc(C[C@H](C(=O)OC(C)(C)C)[C@H]2CCN(C(=O)OC(C)(C)C)C2)cc(CN(Cc2cc(C)cc(C[C@H](C(=O)OC(C)(C)C)[C@H]3CCN(C(=O)OC(C)(C)C)C3)c2)Cc2cc(C)cc(C[C@H](C(=O)OC(C)(C)C)[C@H]3CCN(C(=O)OC(C)(C)C)C3)c2)c1]

To a solution of tert-butyl (3R)-3-[(1S)-2-tert-butoxy-1-[(3-formyl-5-methyl-phenyl)methyl]-2-oxo-ethyl]pyrrolidine-1-carboxylate (150 mg, 0.359 mmol) and tert-butyl (3R)-3-[(1S)-2-tert-butoxy-1-[[3-[[[3-[(2S)-3-tert-butoxy-2-[(3R)-1-tert-butoxycarbonylpyrrolidin-3-yl]-3-oxo-propyl]-5-methyl-phenyl]methylamino]methyl]-5-methyl-phenyl]methyl]-2-oxo-ethyl]pyrrolidine-1-carboxylate (150 mg, 0.183 mmol) in THE (3 mL) was added acetic acid (60 mg, 1.00 mmol) and the mixture was stirred at 20° C. for 3 h, then sodium triacetoxyborohydride (120 mg, 0.566 mmol) was added. The mixture was stirred at 20° C. for 6 h then poured into saturated aqueous NaCl (20 mL) and extracted with EtOAc (20 mL×4). The combined organic phase was dried over Na₂SO₄, filtered and concentrated to dryness under reduced pressure. The residue was purified by silica gel chromatography using a gradient of 0 to 30% EtOAc in petroleum ether to give the title compound (60 mg, 19%). ES-MS m/z (M-Boc+2H)/2

### Preparation 255

### tert-Butyl (3R)-3-[(1S)-2-tert-butoxy-1-[[3-[[[3-[(2S)-3-tert-butoxy-2-[(3R)-1-tert-butoxycarbonylpyrrolidin-3-yl]-3-oxo-propyl]-5-methyl-phenyl]methyl-[(3-fluoro-5-methoxy-phenyl)methyl]amino]methyl]-5-methyl-phenyl]methyl]-2-oxo-ethyl]pyrrolidine-1-carboxylate

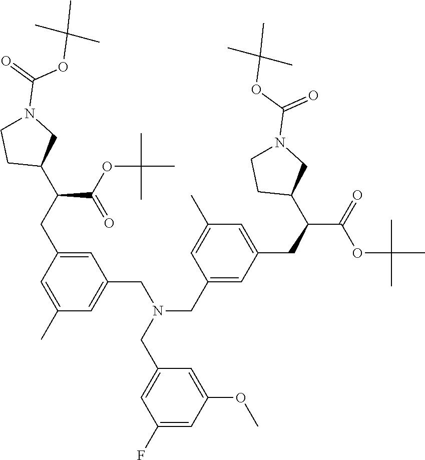 [SMILES: COc1cc(F)cc(CN(Cc2cc(C)cc(C[C@H](C(=O)OC(C)(C)C)[C@H]3CCN(C(=O)OC(C)(C)C)C3)c2)Cc2cc(C)cc(C[C@H](C(=O)OC(C)(C)C)[C@H]3CCN(C(=O)OC(C)(C)C)C3)c2)c1]

To a solution of tert-butyl (3R)-3-[(1S)-2-tert-butoxy-1-[[3-[[[3-[(2S)-3-tert-butoxy-2-[(3R)-1-tert-butoxycarbonylpyrrolidin-3-yl]-3-oxo-propyl]-5-methyl-phenyl]methylamino]methyl]-5-methyl-phenyl]methyl]-2-oxo-ethyl]pyrrolidine-1-carboxylate (50 mg, 0.061 mmol) in THE (3 mL) was added acetic acid (20 mg, 0.33 mmol) and the mixture was stirred at 20° C. for 30 min, then sodium triacetoxyborohydride (40 mg, 0.19 mmol) was added. The mixture was stirred at 20° C. under N₂ for 1 h, then diluted with H₂O (20 mL) and extracted with EtOAc (15 mL×3). The combined organic layers were washed with saturated aqueous NaCl (15 mL), dried over Na₂SO₄, filtered and concentrated under reduced pressure to give the title compound (100 mg, quantitative yield) which was used without further purification. ES-MS m/z 959.5 (M+H).

### Preparation 256

### tert-Butyl (3R)-3-[(1S)-1-[(3-allylphenyl)methyl]-2-tert-butoxy-2-oxo-ethyl]pyrrolidine-1-carboxylate

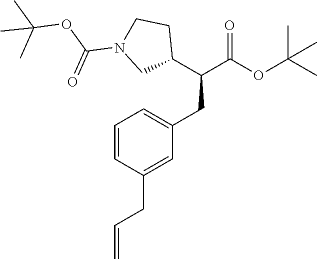 [SMILES: C=CCc1cccc(C[C@H](C(=O)OC(C)(C)C)[C@H]2CCN(C(=O)OC(C)(C)C)C2)c1]

To a mixture of tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 500 mg, 1.10 mmol) and 2-allyl-4,4,5,5-tetramethyl-1,3,2-dioxaborolane (560 mg, 3.33 mmol) in water (1 mL) and 1,2-dimethoxyethane (10 mL) was added potassium carbonate (460 mg, 3.33 mmol) and [1,1′-bis(diphenylphosphino)ferrocene]dichloropalladium(II) (90 mg, 0.12 mmol) and the reaction mixture was stirred at 90° C. for 16 h under N₂. To the reaction mixture was added saturated aqueous NaCl (20 mL). Then, the mixture was extracted with EtOAc (30 mL×3) and the combined organic phase was washed with water (20 mL×2), dried over Na₂SO₄, filtered and concentrated to dryness under reduced pressure. The residue was purified by silica gel chromatography using a gradient of 0 to 20% EtOAc in petroleum ether to give the title compound (300 mg, 59%) as a yellow oil. TLC (1:5 EtOAc/petroleum ether) R_{f}=0.7 (I₂ staining).

### Preparation 257

### tert-Butyl (3R)-3-[(1S)-2-tert-butoxy-1-[[3-[3-[3-[(2S)-3-tert-butoxy-2-[(3R)-1-tert-butoxycarbonylpyrrolidin-3-yl]-3-oxo-propyl]phenyl]propyl]phenyl]methyl]-2-oxo-ethyl]pyrrolidine-1-carboxylate

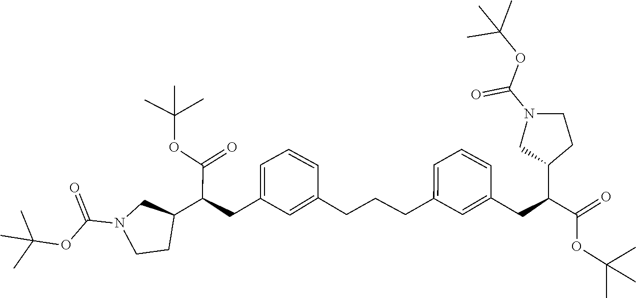 [SMILES: CC(C)(C)OC(=O)[C@@H](Cc1cccc(CCCc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

To a mixture of tert-butyl (3R)-3-[(1S)-1-[(3-allylphenyl)methyl]-2-tert-butoxy-2-oxo-ethyl]pyrrolidine-1-carboxylate (120 mg, 0.289 mmol) and tert-butyl (3R)-3-[(1S)-1-[(3-bromophenyl)methyl]-2-tert-butoxy-2-oxo-ethyl]pyrrolidine-1-carboxylate (prepared essentially as described in WO 2020/247429; 200 mg, 0.440 mmol) in 1,4-dioxane (4 mL) was added [1,1′-bis(diphenylphosphino)ferrocene]dichloropalladium(II) (25 mg, 0.034 mmol), TEA (80 mg, 0.80 mmol), and the reaction mixture was stirred at 70° C. under N₂ for 16 h. The mixture was combined with a reaction which was run essentially as described above using 200 mg (0.481 mmol) of the starting alkene, and the resulting mixture was concentrated to dryness under reduced pressure. The residue was purified by silica gel chromatography using a gradient of 0 to 10% EtOAc in petroleum ether to give tert-butyl (3R)-3-[(1S)-2-tert-butoxy-1-[[3-[(E)-3-[3-[(2S)-3-tert-butoxy-2-[(3R)-1-tert-butoxycarbonylpyrrolidin-3-yl]-3-oxo-propyl]phenyl]allyl]phenyl]methyl]-2-oxo-ethyl]pyrrolidine-1-carboxylate (150 mg, 59%) as a yellow oil.

To a mixture of tert-butyl (3S)-3-[(1S)-2-tert-butoxy-1-[[3-[(E)-3-[3-[(2S)-3-tert-butoxy-2-[(3R)-1-tert-butoxycarbonylpyrrolidin-3-yl]-3-oxo-propyl]phenyl]allyl]phenyl]methyl]-2-oxo-ethyl]pyrrolidine-1-carboxylate (130 mg, 0.148 mmol) in MeOH (10 mL) was added palladium (10 wt % on carbon, 30 mg, 0.0282 mmol) at 25° C. The reaction mixture was stirred at 25° C. under H₂ (45 PSI) for 16 h. The reaction mixture was combined with a mixture run on 20 mg (0.022 mmol) of the starting olefin. The mixture was filtered and the filtrate was concentrated to dryness under reduced pressure to give the title compound (110 mg, 54%) as a yellow oil. TLC (1:5 EtOAc:petroleum ether) R_{f}=0.6.

### Preparation 258

### (2S)-2-[(3R)-1-tert-Butoxycarbonylpyrrolidin-3-yl]-3-[3-[2-(cyclohexoxy)-3-pyridyl]phenyl]propanoic acid

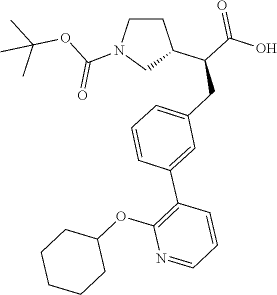 [SMILES: CC(C)(C)OC(=O)N1CC[C@H]([C@H](Cc2cccc(-c3cccnc3OC3CCCCC3)c2)C(=O)O)C1]

(2S)-3-(3-Bromophenyl)-2-[(3R)-1-tert-butoxycarbonylpyrrolidin-3-yl]propanoic acid (prepared essentially as described in WO 2020/247429, 100 mg, 0.25 mmol), 2-(cyclohexoxy)-3-(4,4,5,5-tetramethyl-1,3,2-dioxaborolan-2-yl)pyridine (114 mg, 0.38 mmol), [1,1′-bis(diphenylphosphino)ferrocene]dichloropalladium(II) (19 mg, 0.03 mmol), 2M aqueous potassium carbonate (0.38 mL), and 1,4-dioxane (1.7 mL) were transferred to a reaction vessel, purged with N₂, sealed and stirred at 100° C. for 2 h. The reaction was concentrated under a stream of nitrogen. The residue was taken up in EtOAc and extracted with water. The aqueous layer was acidified with 500 acetic acid and extracted with EtOAc. The organic layer was concentrated under a stream of nitrogen and purified by reversed phase chromatography giving the title compound (57.4 mg, 46%) as a white solid. ES-MS m/z 495 (M+H).

The following were prepared essentially as described in Preparation 258 using the appropriate boronic acid or boronic ester.

| Prep- | Chemical | | ES-MS |
|---|---|---|---|
| aration | name | Structure | m/z |
| 259 | (2S)-2-[(3R)-1-tert- Butoxycarbonylpyrrolidin- 3-yl]-3-[3-[6- (trifluoromethyl)-3- pyridyl]phenyl]propanoic acid | 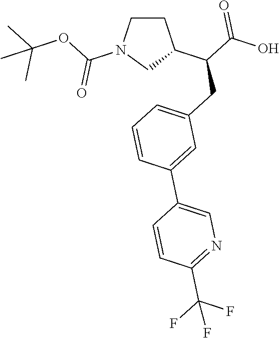 [SMILES: CC(C)(C)OC(=O)N1CC[C@H]([C@H](Cc2cccc(-c3ccc(C(F)(F)F)nc3)c2)C(=O)O)C1] | 409 (M − tBu) |
| 260 | (2S)-2-[(3R)-1-tert- Butoxycarbonylpyrrolidin- 3-yl]-3-[3-(1- methylindazol-3- yl)phenyl]propanoic acid | 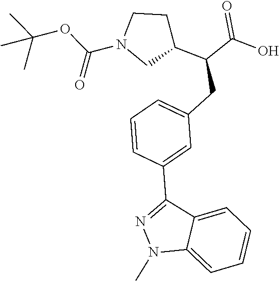 [SMILES: Cn1nc(-c2cccc(C[C@H](C(=O)O)[C@H]3CCN(C(=O)OC(C)(C)C)C3)c2)c2ccccc21] | 450 (M + H) |
| 261 | (2S)-2-[(3R)-1-tert- Butoxycarbonylpyrrolidin- 3-yl]-3-[3-(1- cyclohexylpyrazol-4- yl)phenyl]propanoic acid | 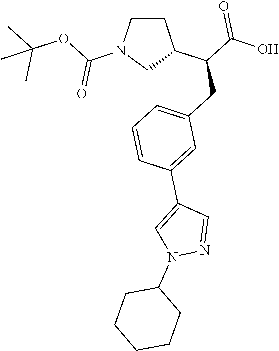 [SMILES: CC(C)(C)OC(=O)N1CC[C@H]([C@H](Cc2cccc(-c3cnn(C4CCCCC4)c3)c2)C(=O)O)C1] | 468 (M + H) |

The following were prepared essentially as described in Preparation 10 using 2-[(3R)-1-tert-butoxycarbonylpyrrolidin-3-yl]acetic acid (prepared essentially as described in WO 2020/247429) and the appropriate heterocyclic starting material. The compounds in the table below were purified by HPLC (column: Phenomenex® C18 75×30 mm, 3 am; mobile phase: solvent A—aqueous NH₄OH+NH₄HCO₃, solvent B—ACN; flow rate: 25 mL/min).

| Prep- | | | |
|---|---|---|---|
| aration | Chemical name | Structure | ES-MS m/z |
| 262 | tert-Butyl (3R)-3-[2- oxo-2-[(4S)-2-oxo-4- phenyl-oxazolidin-3- yl]ethyl]pyrrolidine- 1-carboxylate | 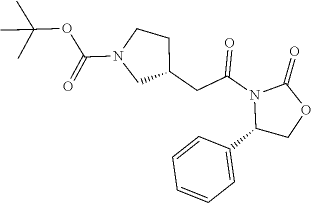 [SMILES: CC(C)(C)OC(=O)N1CC[C@H](CC(=O)N2C(=O)OC[C@@H]2c2ccccc2)C1] | 319 (M − tBu) |
| 263 | tert-Butyl (3R)-3-[2- [(4S)-4-isopropyl-2- oxo-oxazolidin-3-yl]- 2-oxo- ethyl]pyrrolidine-1- carboxylate | 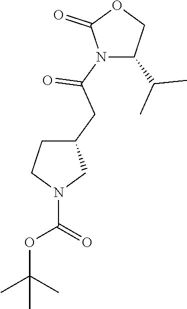 [SMILES: CC(C)[C@H]1COC(=O)N1C(=O)C[C@H]1CCN(C(=O)OC(C)(C)C)C1] | 285 (M − tBu) |
| 264 | tert-Butyl (3R)-3-[2- [(4S,5R)-4-methyl-2- oxo-5-phenyl- oxazolidin-3-yl]-2- oxo- ethyl]pyrrolidine-1- carboxylate | 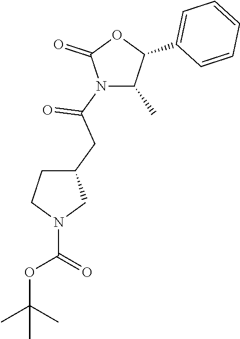 [SMILES: C[C@H]1[C@@H](c2ccccc2)OC(=O)N1C(=O)C[C@H]1CCN(C(=O)OC(C)(C)C)C1] | 333 (M − tBu) |
| 265 | tert-Butyl (3R)-3-[2- [(4R)-4-(4- chlorophenyl)-2-oxo- oxazolidin-3-yl]-2- oxo- ethyl]pyrrolidine-1- carboxylate | 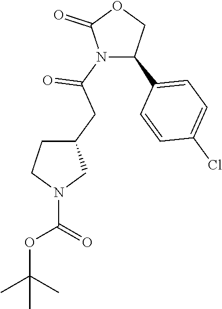 [SMILES: CC(C)(C)OC(=O)N1CC[C@H](CC(=O)N2C(=O)OC[C@H]2c2ccc(Cl)cc2)C1] | 353 (M − tBu) |
| 266 | tert-Butyl (3R)-3-[2- oxo-2-[(4S,5R)-2- oxo-4,5-diphenyl- oxazolidin-3- yl]ethyl]pyrrolidine- 1-carboxylate | 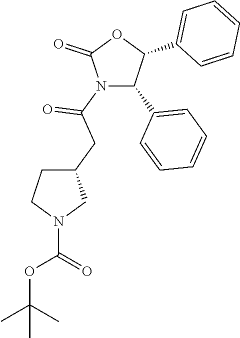 [SMILES: CC(C)(C)OC(=O)N1CC[C@H](CC(=O)N2C(=O)O[C@H](c3ccccc3)[C@@H]2c2ccccc2)C1] | 451 (M + H) |
| 267 | tert-Butyl (3R)-3-[2- [(4S)-4-[[4- (dimethylamino) phenyl]methyl]-2-oxo- oxazolidin-3-yl]-2- oxo- ethyl]pyrrolidine-1- carboxylate | 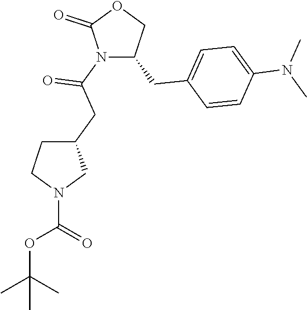 [SMILES: CN(C)c1ccc(C[C@H]2COC(=O)N2C(=O)C[C@H]2CCN(C(=O)OC(C)(C)C)C2)cc1] | 432 (M + H) |
| 268 | tert-Butyl (3R)-3-[2- [(4S)-4-benzyl-2- oxo-thiazolidin-3-yl]- 2-oxo- ethyl]pyrrolidine-1- carboxylate | 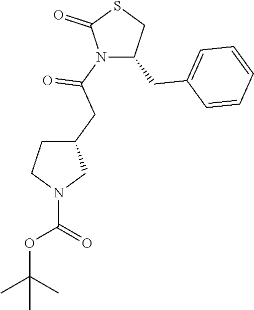 [SMILES: CC(C)(C)OC(=O)N1CC[C@H](CC(=O)N2C(=O)SC[C@@H]2Cc2ccccc2)C1] | 305 (M − Boc + H) |
| 269 | tert-Butyl (3R)-3-[2- [(4S)-4-benzyl-5,5- dimethyl-2-oxo- oxazolidin-3-yl]-2- oxo- ethyl]pyrrolidine-1- carboxylate | 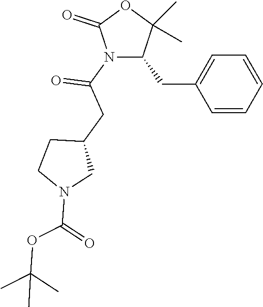 [SMILES: CC(C)(C)OC(=O)N1CC[C@H](CC(=O)N2C(=O)OC(C)(C)[C@@H]2Cc2ccccc2)C1] | 361 (M − tBu) |
| 270 | tert-Butyl (3R)-3-[2- [(4S)-4-benzyl-2- thioxo-thiazolidin-3- yl]-2-oxo- ethyl]pyrrolidine-1- carboxylate | 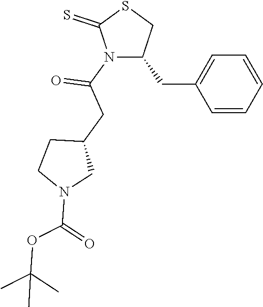 [SMILES: CC(C)(C)OC(=O)N1CC[C@H](CC(=O)N2C(=S)SC[C@@H]2Cc2ccccc2)C1] | 365 (M − tBu) |
| 271 | tert-Butyl (3R)-3-[2- [(4S,5R)-5-[3,5- bis(trifluoromethyl) phenyl]-4-methyl-2- oxo-oxazolidin-3-yl]- 2-oxo- ethyl]pyrrolidine-1- carboxylate | 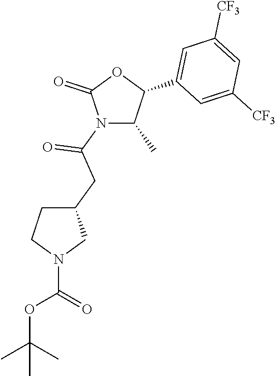 [SMILES: C[C@H]1[C@@H](c2cc(C(F)(F)F)cc(C(F)(F)F)c2)OC(=O)N1C(=O)C[C@H]1CCN(C(=O)OC(C)(C)C)C1] | 469 (M − tBu) |
| 272 | tert-Butyl (3R)-3-[2- [(4S)-4-benzyl-2- thioxo-oxazolidin-3- yl]-2-oxo- ethyl]pyrrolidine-1- carboxylate | 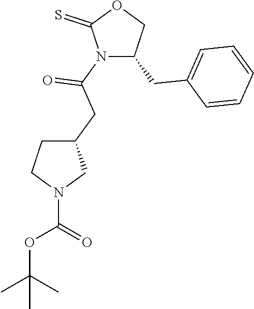 [SMILES: CC(C)(C)OC(=O)N1CC[C@H](CC(=O)N2C(=S)OC[C@@H]2Cc2ccccc2)C1] | 349 (M − tBu) |

### EXAMPLE 1A

### 3-(3-Ethynylphenyl)-2-[(3R)-pyrrolidin-3-yl]propanoic acid;hydrochloride—Isomer 1

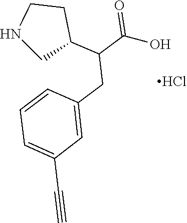 [SMILES: C#Cc1cccc(CC(C(=O)O)[C@H]2CCNC2)c1 ; Cl]

5 To 2-[(3R)-1-tert-butoxycarbonylpyrrolidin-3-yl]-3-(3-ethynylphenyl) propanoic acid Isomer 1 (454 mg, 1.32 mmol) was added HCl in Et₂O (2.0 M, 13 mL, 26 mmol) and the mixture was stirred at RT for 6 h. The solvent was removed and the residue was dried in vacuo at 40° C. overnight to give the title compound (370 mg, 100%) as a white powder. ES-MS m/z 244 (M+H).

### EXAMPLE 1

### 3-(3-Ethynylphenyl)-2-[(3R)-pyrrolidin-3-yl]propanoic acid;hydrochloride—Isomer 2

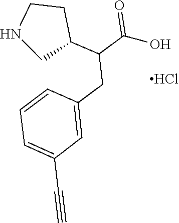 [SMILES: C#Cc1cccc(CC(C(=O)O)[C@H]2CCNC2)c1 ; Cl]

The title compound was prepared essentially as described in Example 1a using 2-[(3R)-1-tert-butoxycarbonylpyrrolidin-3-yl]-3-(3-ethynylphenyl) propanoic acid Isomer 2. ES-MS m/z 244 (M+H).

### EXAMPLE 2a

### 3-(6-Chloro-3-pyridyl)-2-[(3R)-pyrrolidin-3-yl]propanoic acid; hydrochloride—Isomer 1

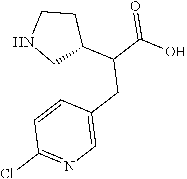 [SMILES: O=C(O)C(Cc1ccc(Cl)nc1)[C@H]1CCNC1]

To a solution of 2-[(3R)-1-tert-butoxycarbonylpyrrolidin-3-yl]-3-(6-chloro-3-pyridyl) propanoic acid-Isomer 1 (20 mg, 0.056 mmol) in DCM (2 mL) was added HCl (4 M in 1,4-dioxane, 2 mL) and the reaction was stirred at RT for 1 h. The solvent was removed in vacuo, then the residue was dissolved in water and loaded onto 5 g SCX resin. The resin was eluted with ACN and the eluate was discarded. The resin was then eluted with 2 M NH3 in MeOH and the eluate was concentrated in vacuo to give the title compound (13 mg, 91%) as a white solid. ES-MS m/z 255 (M+H).

### EXAMPLE 2b

### 3-(6-Chloro-3-pyridyl)-2-[(3R)-pyrrolidin-3-yl]propanoic acid;hydrochloride—Isomer 2

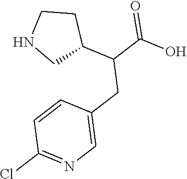 [SMILES: O=C(O)C(Cc1ccc(Cl)nc1)[C@H]1CCNC1]

The title compound was prepared essentially as described in Example 2a using 2-[(3R)-1-tert-butoxycarbonylpyrrolidin-3-yl]-3-(6-chloro-3-pyridyl) propanoic acid-Isomer 2. ES-MS m/z 255 (M+H).

### EXAMPLE 3

### (2S)-3-(3-Methyl-5-ureido-phenyl)-2-[(3R)-pyrrolidin-3-yl]propanoic acid; hydrochloride

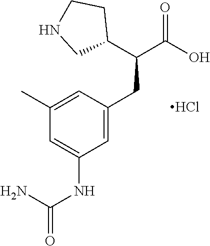 [SMILES: Cc1cc(C[C@H](C(=O)O)[C@H]2CCNC2)cc(NC(N)=O)c1 ; Cl]

The title compound was prepared essentially as described in Example 1a using (2S)-2-[(3R)-1-tert-butoxycarbonylpyrrolidin-3-yl]-3-(3-methyl-5-ureido-phenyl)propanoic acid. ES-MS m/z 292 (M+H).

### EXAMPLE 4

### (2S)-3-[3-[(3-fluoro-5-methoxy-anilino)methyl]phenyl]-2-[(3R)-pyrrolidin-3-yl]propanoic acid;dihydrochloride

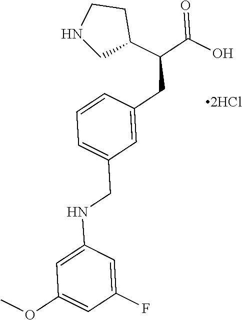 [SMILES: COc1cc(F)cc(NCc2cccc(C[C@H](C(=O)O)[C@H]3CCNC3)c2)c1]

To a solution of tert-butyl (3R)-3-[(1S)-2-tert-butoxy-1-[[3-[(3-fluoro-5-methoxy-anilino)methyl]phenyl]methyl]-2-oxo-ethyl]pyrrolidine-1-carboxylate (433 mg, 0.82 mmol) in DCM (10 mL) was added hydrochloric acid (4 M in 1,4-dioxane, 10 mL, 40 mmol) and the mixture was stirred at RT for 4 h. The solvent was eliminated in vacuo. The residue was dissolved in water and concentrated to eliminate residual solvents to obtain the title compound (350 mg, 99%). ES/MS m/z 373 (M+H).

The following Examples were prepared essentially as described in Example 4 using the appropriate starting material, which is either a Boc-protected 2-(pyrrolidine-3-yl)propanoic acid (Preparations 12, 23-33, and 50) or a Boc-protected tert-butyl-2-(pyrrolidine-3-yl)propanoate (Preparations 13-22, 34-40, 43-46, and 51). Examples 5a and 5b were prepared using (2S)-3-(3-Bromophenyl)-2-(1-tert-butoxycarbonyl-4,4-difluoro-pyrrolidin-3-yl)propanoic acid—Isomer 1 and Isomer 2 respectively (Preparation 12) as starting material.

| Ex- | | | | | |
|---|---|---|---|---|---|
| ample | Chemical | | Procedure | Purification | Physical Data |
| # | name | Structure | modifications | modifications | ES-MS m/z |
| 5a | (2S)-3-(3- Bromophenyl)-2-(4,4- difluoropyrrolidin-3- yl)propanoic acid;hydrochloride- Isomer 1 | 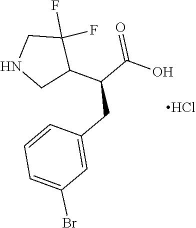 [SMILES: O=C(O)[C@@H](Cc1cccc(Br)c1)C1CNCC1(F)F ; Cl] | B | | 334/336 (M + H) |
| 5b | (2S)-3-(3- Bromophenyl)-2-(4,4- difluoropyrrolidin-3- yl)propanoic acid;hydrochloride- Isomer 2 | 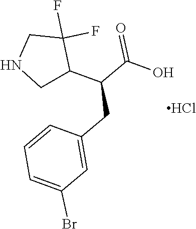 [SMILES: O=C(O)[C@@H](Cc1cccc(Br)c1)C1CNCC1(F)F ; Cl] | B | | 334/336 (M + H) |
| 6 | (2S)-3-[3-[[(3-Fluoro- 5-methoxy- phenyl)methyl]amino] phenyl]-2-[(3R)- pyrrolidin-3- yl]propanoic acid;hydrochloride | 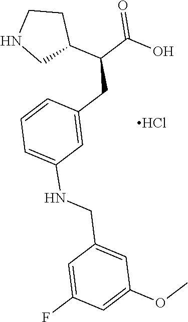 [SMILES: COc1cc(F)cc(CNc2cccc(C[C@H](C(=O)O)[C@H]3CCNC3)c2)c1 ; Cl] | B | | 373 (M + H) |
| 7 | (2S)-3-[3- [(Cyclobutylmethyl) amino]phenyl]-2- [(3R)-pyrrolidin-3- yl]propanoic acid;dihydrochloride | 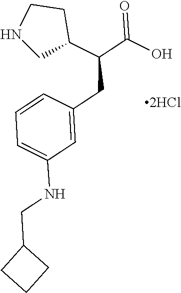 [SMILES: O=C(O)[C@@H](Cc1cccc(NCC2CCC2)c1)[C@H]1CCNC1] | B | | 303 (M + H) |
| 8 | (2S)-3-[3-[[(4,4- Difluorocyclohexyl) methyl]amino]phenyl]- 2-[(3R)-pyrrolidin-3- yl]propanoic acid;hydrochloride | 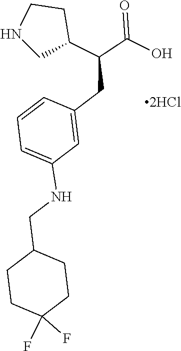 [SMILES: O=C(O)[C@@H](Cc1cccc(NCC2CCC(F)(F)CC2)c1)[C@H]1CCNC1] | B | | 367 (M + H) |
| 9 | (2S)-3-[3-[(2- Cyclopropylethyl) amino]phenyl]-2- [(3R)-pyrrolidin-3- yl]propanoic acid;dihydrochloride | 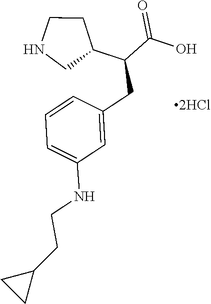 [SMILES: O=C(O)[C@@H](Cc1cccc(NCCC2CC2)c1)[C@H]1CCNC1] | B | A | 303 (M + H) |
| 10 | (2S)-3-[3- [(Cyclopropylmethyl) amino]phenyl]-2- [(3R)-pyrrolidin-3- yl]propanoic acid;dihydrochloride | 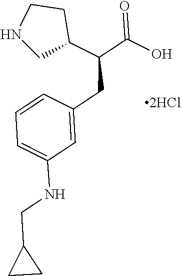 [SMILES: O=C(O)[C@@H](Cc1cccc(NCC2CC2)c1)[C@H]1CCNC1] | B | | 289 (M + H) |
| 11 | (2S)-3-[3- [(Cyclohexylmethyl) amino]phenyl]-2- [(3R)-pyrrolidin-3- yl]propanoic acid;dihydrochloride | 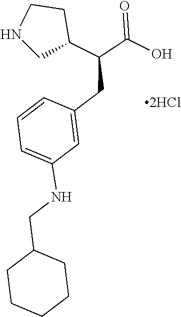 [SMILES: O=C(O)[C@@H](Cc1cccc(NCC2CCCCC2)c1)[C@H]1CCNC1] | B | | 331 (M + H) |
| 12 | (2S)-2-[(3R)- Pyrrolidin-3-yl]-3-[3- [(3,3,3- trifluoropropyl)amino] phenyl]propanoic acid;dihydrochloride | 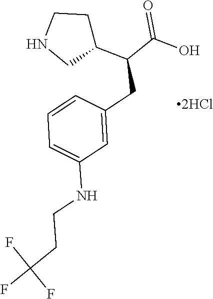 [SMILES: O=C(O)[C@@H](Cc1cccc(NCCC(F)(F)F)c1)[C@H]1CCNC1] | B | | 331 (M + H) |
| 13 | (2S)-3-[3-[[[(3-Fluoro- 5-methoxy- phenyl)methyl]amino] methyl]phenyl]-2- [(3R)-pyrrolidin-3- yl]propanoic acid;dihydrochloride | 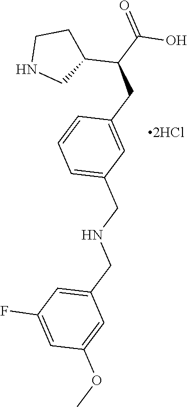 [SMILES: COc1cc(F)cc(CNCc2cccc(C[C@H](C(=O)O)[C@H]3CCNC3)c2)c1] | | E | 387 (M + H) |
| 14 | (2S)-3-[3-[(1H- Benzimidazol-2- ylamino)methyl] phenyl]-2-[(3R)- pyrrolidin- 3-yl]propanoic acid;hydrochloride | 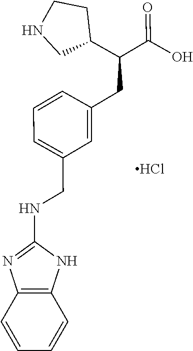 [SMILES: O=C(O)[C@@H](Cc1cccc(CNc2nc3ccccc3[nH]2)c1)[C@H]1CCNC1 ; Cl] | B | | 365 (M + H) |
| 15 | (2S)-3-[3-[(2-Fluoro- 3-methoxy-phenyl) carbamoylamino] phenyl]-2-[(3R)- pyrrolidin-3- yl]propanoic acid;hydrochloride | 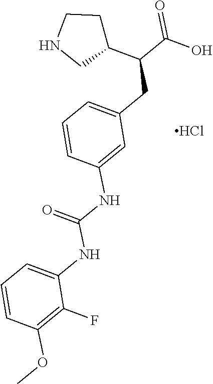 [SMILES: COc1cccc(NC(=O)Nc2cccc(C[C@H](C(=O)O)[C@H]3CCNC3)c2)c1F ; Cl] | | | 402 (M + H) |
| 16 | (2S)-3-[3-[(4- Methylthiazol-2- yl)amino]phenyl]-2- [(3R)-pyrrolidin-3- yl]propanoic acid;hydrochloride | 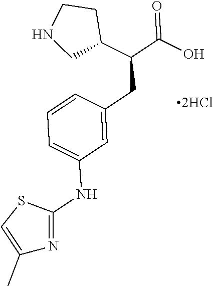 [SMILES: Cc1csc(Nc2cccc(C[C@H](C(=O)O)[C@H]3CCNC3)c2)n1] | | | 332 (M + H) |
| 17 | (2S)-3-[3-(tert- Butylcarbamoylamino) phenyl]-2-[(3R)- pyrrolidin-3- yl]propanoic acid;hydrochloride | 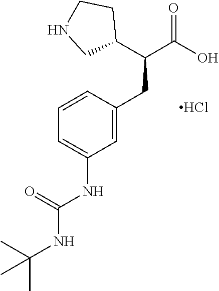 [SMILES: CC(C)(C)NC(=O)Nc1cccc(C[C@H](C(=O)O)[C@H]2CCNC2)c1 ; Cl] | C | | 334 (M + H) |
| 18 | (2S)-3-[3-[[Methyl (phenyl)carbamoyl] amino]phenyl]- 2-[(3R)-pyrrolidin-3- yl]propanoic acid;hydrochloride | 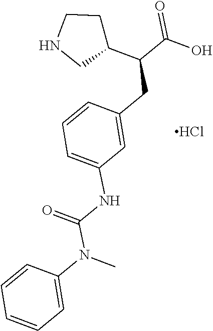 [SMILES: CN(C(=O)Nc1cccc(C[C@H](C(=O)O)[C@H]2CCNC2)c1)c1ccccc1 ; Cl] | C | | 368 (M + H) |
| 19 | (2S)-3-[3-[(1-Benzyl- 4-piperidyl) carbamoylamino] phenyl]-2-[(3R)- pyrrolidin-3- yl]propanoic acid;hydrochloride | 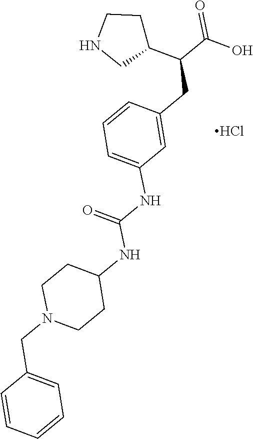 [SMILES: O=C(Nc1cccc(C[C@H](C(=O)O)[C@H]2CCNC2)c1)NC1CCN(Cc2ccccc2)CC1 ; Cl] | C | | 451 (M + H) |
| 20 | (2S)-2-[(3R)- Pyrrolidin-3-yl]-3-[3- (8-quinolyl- carbamoylamino) phenyl]propanoic acid;hydrochloride | 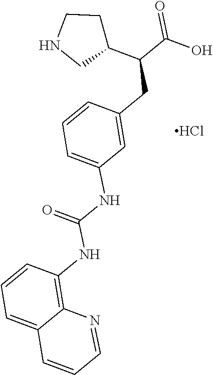 [SMILES: O=C(Nc1cccc(C[C@H](C(=O)O)[C@H]2CCNC2)c1)Nc1cccc2cccnc12 ; Cl] | C | | 405 (M + H) |
| 21 | (2S)-3-[3-[(3,4- Dichlorophenyl) carbamoylamino] phenyl]-2-[(3R)- pyrrolidin-3- yl]propanoic acid;hydrochloride | 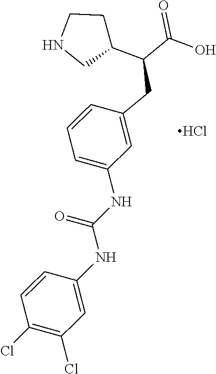 [SMILES: O=C(Nc1cccc(C[C@H](C(=O)O)[C@H]2CCNC2)c1)Nc1ccc(Cl)c(Cl)c1 ; Cl] | C | | 422/424 (M + H) |
| 22 | (2S)-3-[3-[(4- Benzyloxyphenyl) carbamoylamino] phenyl]-2-[(3R)- pyrrolidin-3- yl]propanoic acid;hydrochloride | 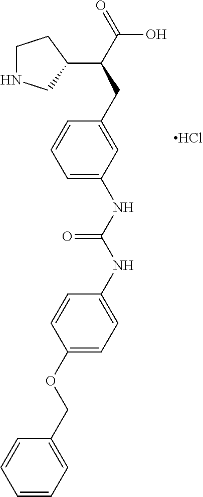 [SMILES: O=C(Nc1ccc(OCc2ccccc2)cc1)Nc1cccc(C[C@H](C(=O)O)[C@H]2CCNC2)c1 ; Cl] | C | | 460 (M + H) |
| 23 | (2S)-3-[3-(Isopropyl- carbamoylamino) phenyl]-2-[(3R)- pyrrolidin-3- yl]propanoic acid;hydrochloride | 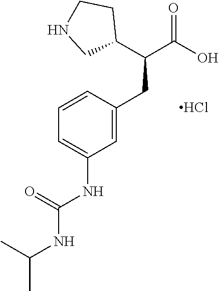 [SMILES: CC(C)NC(=O)Nc1cccc(C[C@H](C(=O)O)[C@H]2CCNC2)c1 ; Cl] | C | | 320 (M + H) |
| 24 | (2S)-3-[3-(2-Methoxy- ethylcarbamoylamino) phenyl]-2- [(3R)-pyrrolidin-3- yl]propanoic acid;hydrochloride | 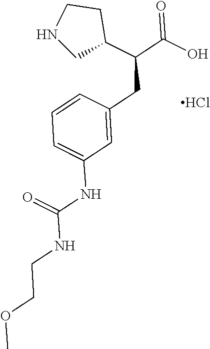 [SMILES: COCCNC(=O)Nc1cccc(C[C@H](C(=O)O)[C@H]2CCNC2)c1 ; Cl] | C | | 336 (M + H) |
| 25 | (2S)-3-[3-[(3-Phenyl- 1,2,4-oxadiazol-5- yl)carbamoylamino] phenyl]-2-[(3R)- pyrrolidin-3- yl]propanoic acid;hydrochloride | 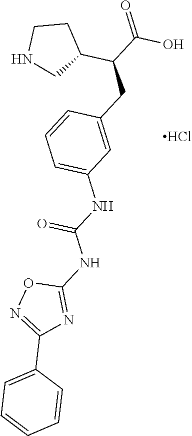 [SMILES: O=C(Nc1cccc(C[C@H](C(=O)O)[C@H]2CCNC2)c1)Nc1nc(-c2ccccc2)no1 ; Cl] | C | | 422 (M + H) |
| 26 | (2S)-3-[3-[[2-(2- Bromophenyl)acetyl] amino]phenyl]-2-[(3R)- pyrrolidin-3- yl]propanoic acid;hydrochloride | 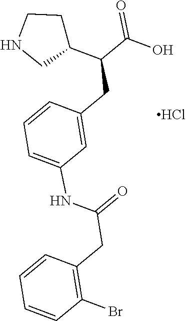 [SMILES: O=C(Cc1ccccc1Br)Nc1cccc(C[C@H](C(=O)O)[C@H]2CCNC2)c1 ; Cl] | C | | 431/433 (M + H) |
| 27 | (2S)-3-[3-(3-Hydroxy- propanoylamino) phenyl]-2-[(3R)- pyrrolidin-3- yl]propanoic acid;hydrochloride | 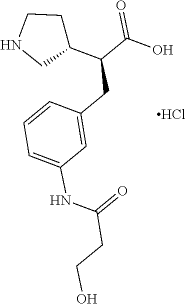 [SMILES: O=C(CCO)Nc1cccc(C[C@H](C(=O)O)[C@H]2CCNC2)c1 ; Cl] | B | | 307 (M + H) |
| 28 | (2S)-3-[3-[(2- Hydroxyacetyl)amino] phenyl]-2-[(3R)- pyrrolidin-3- yl]propanoic acid;hydrochloride | 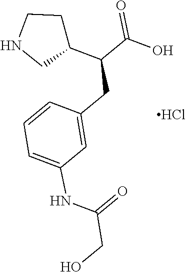 [SMILES: O=C(CO)Nc1cccc(C[C@H](C(=O)O)[C@H]2CCNC2)c1 ; Cl] | B | | 293 (M + H) |
| 29 | (2S)-3-[3-(3-Hydroxy- propylcarbamoyl- amino)phenyl]-2- [(3R)-pyrrolidin-3- yl]propanoic acid;hydrochloride | 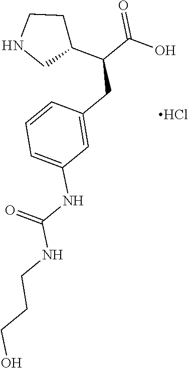 [SMILES: O=C(NCCCO)Nc1cccc(C[C@H](C(=O)O)[C@H]2CCNC2)c1 ; Cl] | B | | 336 (M + H) |
| 30 | (2R)-2-[(3R)- Pyrrolidin-3-yl]-3-[3- (2,2,2-trifluoro- ethylsulfonylamino) phenyl]propanoic acid;hydrochloride | 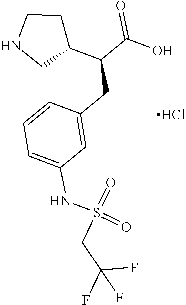 [SMILES: O=C(O)[C@@H](Cc1cccc(NS(=O)(=O)CC(F)(F)F)c1)[C@H]1CCNC1 ; Cl] | | A | 381 (M + H) |
| 31 | (2S)-3-[3- [(Cyclohexylmethyl) sulfonylamino]phenyl]- 2-[(3R)-pyrrolidin-3- yl]propanoic acid;hydrochloride | 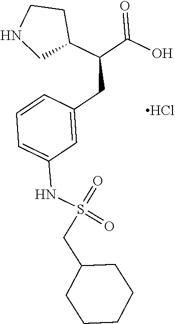 [SMILES: O=C(O)[C@@H](Cc1cccc(NS(=O)(=O)CC2CCCCC2)c1)[C@H]1CCNC1 ; Cl] | | A | 395 (M + H) |
| 32 | (2S)-3-[3- [(Cyclopropylsulfonyl) amino]phenyl]-2- [(3R)-pyrrolidin-3- yl]propanoic acid;hydrochloride | 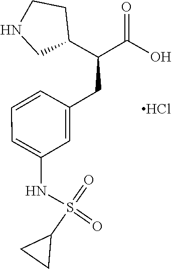 [SMILES: O=C(O)[C@@H](Cc1cccc(NS(=O)(=O)C2CC2)c1)[C@H]1CCNC1 ; Cl] | B + D | A | 339 (M + H) |
| 33 | (2S)-3-[3-[(3- Chlorobenzyl) sulfamoyl] phenyl]-2-[(3R)- pyrrolidin-3- yl]propanoic acid;hydrochloride | 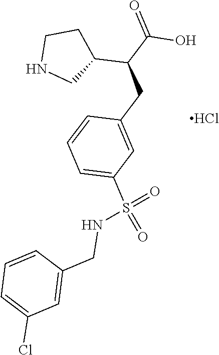 [SMILES: O=C(O)[C@@H](Cc1cccc(S(=O)(=O)NCc2cccc(Cl)c2)c1)[C@H]1CCNC1 ; Cl] | | | 423 (M + H) |
| 34 | (2S)-3-[3-[(Carboxy- methyl)sulfamoyl] phenyl]-2-[(3R)- pyrrolidin-3- yl]propanoic acid;hydrochloride | 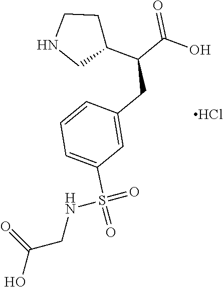 [SMILES: O=C(O)CNS(=O)(=O)c1cccc(C[C@H](C(=O)O)[C@H]2CCNC2)c1 ; Cl] | | | 357 (M + H) |
| 35 | (2S)-3-[3-[[2-(3- Methoxyphenyl)ethyl] sulfamoyl]phenyl]-2- [(3R)-pyrrolidin-3- yl]propanoic acid;hydrochloride | 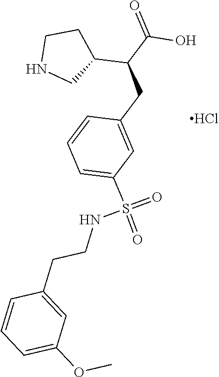 [SMILES: COc1cccc(CCNS(=O)(=O)c2cccc(C[C@H](C(=O)O)[C@H]3CCNC3)c2)c1 ; Cl] | | | 433 (M + H) |
| 36 | (2S)-3-[3-[(3,5- Dimethoxybenzyl) sulfamoyl]phenyl]-2- [(3R)-pyrrolidin-3- yl]propanoic acid;hydrochloride | 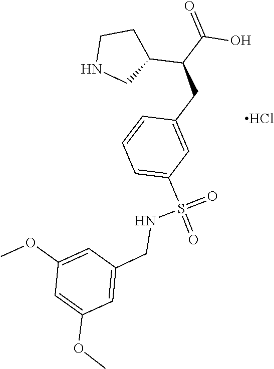 [SMILES: COc1cc(CNS(=O)(=O)c2cccc(C[C@H](C(=O)O)[C@H]3CCNC3)c2)cc(OC)c1 ; Cl] | | | 449 (M + H) |
| 37 | 3-[3-(Prop-2-yn-1- ylamino)phenyl]-2- [(3R)-pyrrolidin-3- yl]propanoic acid;dihydrochloride | 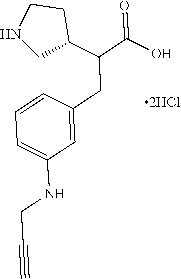 [SMILES: C#CCNc1cccc(CC(C(=O)O)[C@H]2CCNC2)c1] | B | | 273 (M + H) |
| 38 | (2S)-3-[3-(Carboxy- methyl)phenyl]- 2-[(3R)-pyrrolidin- 3-yl]propanoic acid;hydrochloride | 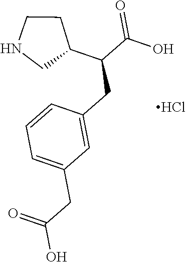 [SMILES: O=C(O)Cc1cccc(C[C@H](C(=O)O)[C@H]2CCNC2)c1 ; Cl] | | | 278 (M + H) |

A. RP-HPLC/MS [column: XBridge ™ C18; mobile phase: solvent A = 20 mM aqueous NH₄HCO₃ (pH 9), solvent B = ACN]. After purification, the compound was dissolved in water and 1N aqueous HCl was added, then evaporated to dryness and further dried in vacuo
B. HCl (2M) in Et₂O was used in place of HCl (4M in 1,4-dioxane)
C. Hydrochloric acid (4M in 1,4-dioxane) used as solvent
D. Water was used as solvent
E. RP-HPLC/MS [column: Kinetex EVO 30 × 100 mm, 5 μm; mobile phase: solvent A = 10 mM aqueous NH₄HCO₃ (pH 10) + 5% MeOH, solvent B: ACN; flow rate: 80 mL/min]. After purification, the compound was dissolved in water and 1N aqueous HCl was added, then evaporated to dryness and further dried in vacuo.

### EXAMPLE 39

### (2S)-3-[3-[[3-[(2S)-2-Carboxy-2-[(3R)-pyrrolidin-3-yl]ethyl]phenyl]carbamoylamino]phenyl]-2-[(3R)-pyrrolidin-3-yl]propanoic acid;dihydrochloride

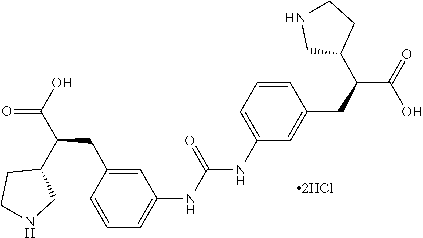 [SMILES: O=C(Nc1cccc(C[C@H](C(=O)O)[C@H]2CCNC2)c1)Nc1cccc(C[C@H](C(=O)O)[C@H]2CCNC2)c1]

A solution of HCl (4.0 M in 1,4-dioxane, 170 mL, 694 mmol) was added to a mixture of tert-butyl (3R)-3-[(1S)-2-tert-butoxy-1-[[3-[[3-[(2S)-3-tert-butoxy-2-[(3R)-1-tert-butoxycarbonylpyrrolidin-3-yl]-3-oxo-propyl]phenyl]carbamoylamino]phenyl]methyl]-2-oxo-ethyl]pyrrolidine-1-carboxylate (56 g, 69.4 mmol) in 1,4-dioxane (56 mL). The mixture was stirred at RT overnight. The resulting solid was filtered, washed with 1,4-dixoane, and dried under reduced pressure at 40° C. The solid was triturated in tert-amyl alcohol (600 mL) overnight. The solid was collected, washed with tert-amyl alcohol, then dried under reduced pressure at 40° C. The solid was stirred with tert-amyl alcohol (400 mL) and water (60 mL) resulting in a solution, which was concentrated in vacuo. The solid obtained was stirred at RT in tert-amyl alcohol (400 mL) for 72 h, then filtered and washed with tert-amyl alcohol and dried in vacuo at 40° C. to give the title compound (39.0 g, 99%). ES/MS (m/z): 494 (M+H). Analytical chiral HPLC [column: XBridge® C18 2.1×50 mm, 3.5 μm, mobile phase: ACN in aqueous NH₄HCO₃ (10 mM, pH 9.0); flow rate: 1.2 mL/min; column temperature: 50° C.] shows de >98%.

The following Examples were prepared essentially as described in Example 39 using the appropriate starting material, which is either a Boc-protected 2-(pyrrolidine-3-yl)propanoic acid or a Boc-protected tert-butyl-2-(pyrrolidine-3-yl)propanoate.

| | | | Pro- | Purifi- | Physical |
|---|---|---|---|---|---|
| Ex- | | | cedure | cation | Data |
| ample | Chemical | | modifi- | modifi- | ES-MS |
| # | name | Structure | cations | cations | m/z |
| 40 | (2R)-3-[3-[[3-[(2R)-2- Carboxy-2-[(3S)- pyrrolidin-3- yl]ethyl]phenyl] carbamoylamino] phenyl]-2-[(3S)- pyrrolidin-3-yl]propanoic acid;dihydrochloride | 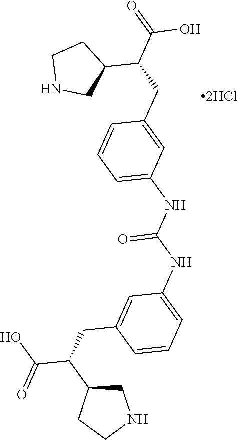 [SMILES: O=C(Nc1cccc(C[C@@H](C(=O)O)[C@@H]2CCNC2)c1)Nc1cccc(C[C@@H](C(=O)O)[C@@H]2CCNC2)c1] | B | A, N | 495 (M + H). |
| 41 | (2S)-3-[3-[[3-[(2S)-2- Carboxy-2-[(3R)- pyrrolidin-3- ylethyl]phenyl] carbamoylamino] methyl]phenyl]- 2-[(3R)-pyrrolidin-3- yl]propanoic acid;dihydrochloride | 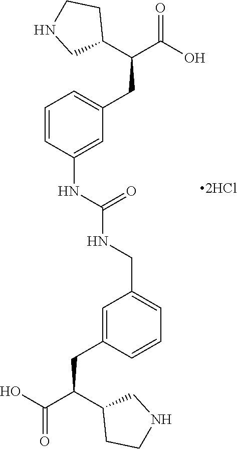 [SMILES: O=C(NCc1cccc(C[C@H](C(=O)O)[C@H]2CCNC2)c1)Nc1cccc(C[C@H](C(=O)O)[C@H]2CCNC2)c1 ; "·2HCl"] | B | | 509 (M + H). |
| 42 | (2S)-3-[3-[3-[3-[(2S)-2- Carboxy-2-[(3R)- pyrrolidin-3- yl]ethyl]phenyl]-2-oxo- hexahydropyrimidin-1- yl]phenyl]-2-[(3R)- pyrrolidin-3-yl]propanoic acid; dihydrochloride | 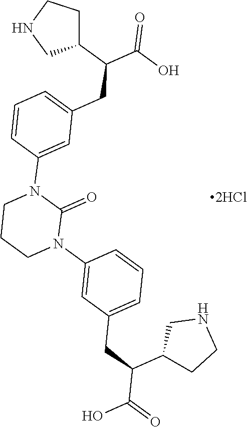 [SMILES: O=C(O)[C@@H](Cc1cccc(N2CCCN(c3cccc(C[C@H](C(=O)O)[C@H]4CCNC4)c3)C2=O)c1)[C@H]1CCNC1 ; "·2HCl"] | B | C | 535 (M + H). |
| 43 | ((2S)-3-[3-[4-[3-[(2S)-2- Carboxy-2-[(3R)- pyrrolidin-3- yl]ethyl]phenyl]-2,5- dioxo-piperazin-1- yl]phenyl]-2-[(3R)- pyrrolidin-3-yl]propanoic acid; dihydrochloride | 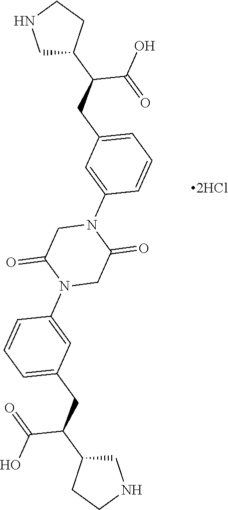 [SMILES: O=C(O)[C@@H](Cc1cccc(N2CC(=O)N(c3cccc(C[C@H](C(=O)O)[C@H]4CCNC4)c3)CC2=O)c1)[C@H]1CCNC1] | B | C | 549 (M + H) |
| 44 | (2S)-3-[3-[[N- Carbamoyl-3-[(2S)-2- carboxy-2-[(3R)- pyrrolidin-3- yl]ethyl]anilino]methyl] phenyl]-2-[(3R)- pyrrolidin-3-yl]propanoic acid; dihydrochloride | 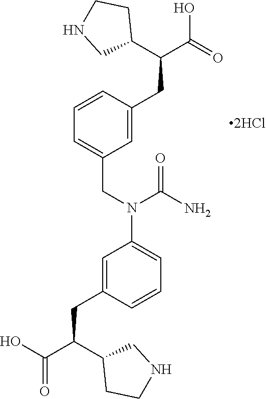 [SMILES: NC(=O)N(Cc1cccc(C[C@H](C(=O)O)[C@H]2CCNC2)c1)c1cccc(C[C@H](C(=O)O)[C@H]2CCNC2)c1 ; "·2HCl"] | B | M | 509 (M + H) |
| 45 | (2S)-3-[3-[3-[3-[(2S)-2- Carboxy-2-[(3R)- pyrrolidin-3- yl]ethyl]phenyl]-2-oxo- benzimidazol-1- yl]phenyl]-2-[(3R)- pyrrolidin-3-yl]propanoic acid; dihydrochloride | 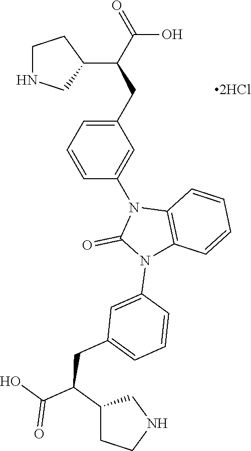 | B | L, K | 569 (M + H) |
| 46 | (2S)-3-[3-[3-[3-[(2S)-2- Carboxy-2-[(3R)- pyrrolidin-3- yl]ethyl]phenyl]-2-oxo- imidazolidin-1- yl]phenyl]-2-[(3R)- pyrrolidin-3-yl]propanoic acid; di-hydrochloride | 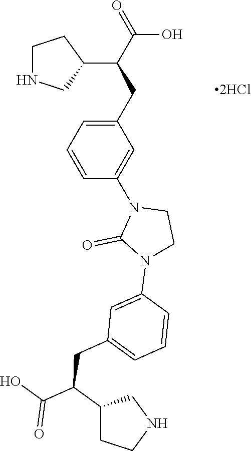 | B | C | 521 (M + H) |
| 47 | (2S)-3-[3-[3-[(2S)-2- Carboxy-2-[(3R)- pyrrolidin-3- yl]ethyl]phenyl]phenyl]- 2-[(3R)-pyrrolidin-3- yl]propanoic acid; dihydrochloride | 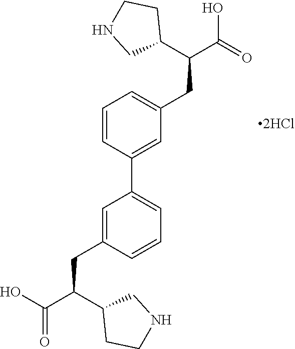 [SMILES: O=C(O)[C@@H](Cc1cccc(-c2cccc(C[C@H](C(=O)O)[C@H]3CCNC3)c2)c1)[C@H]1CCNC1 ; "·2HCl"] | B | C | 437 (M + H) |
| 48 | (2R)-3-[3-[3-[(2R)-2- Carboxy-2-[(3S)- pyrrolidin-3- yl]ethyl]phenyl]phenyl]- 2-[(3S)-pyrrolidin-3- yl]propanoic acid; dihydrochloride | 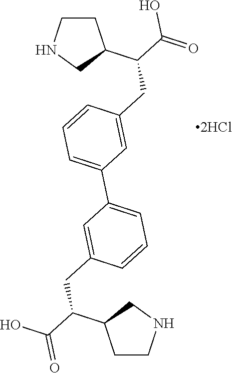 [SMILES: O=C(O)[C@H](Cc1cccc(-c2cccc(C[C@@H](C(=O)O)[C@@H]3CCNC3)c2)c1)[C@@H]1CCNC1] | B | C, J | 437 (M + H) |
| 49 | (2S)-3-[3-[3-[(2S)-2- Carboxy-2-[(3R)- pyrrolidin-3-yl]ethyl]-5- fluoro- phenyl]carbamoylamino]- 5-fluoro-phenyl]-2- [(3R)-pyrrolidin-3- yl]propanoic acid; di- hydrochloride | 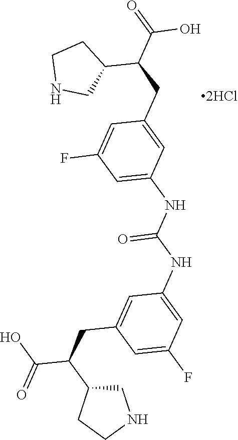 [SMILES: O=C(Nc1cc(F)cc(C[C@H](C(=O)O)[C@H]2CCNC2)c1)Nc1cc(F)cc(C[C@H](C(=O)O)[C@H]2CCNC2)c1] | B | C | 531 (M + H) |
| 50 | (2S)-3-[3-[5-[(2S)-2- Carboxy-2-[(3R)- pyrrolidin-3-yl]ethyl]-1- (2- phenylethyl)benzimidazol- 2-yl]phenyl]-2-[(3R)- pyrrolidin-3-yl]propanoic acid; trihydrochloride | 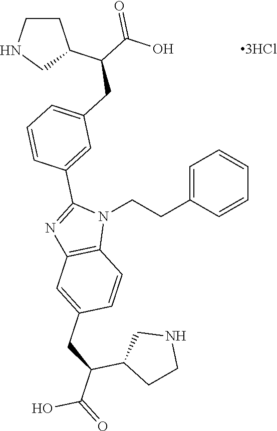 [SMILES: O=C(O)[C@@H](Cc1cccc(-c2nc3cc(C[C@H](C(=O)O)[C@H]4CCNC4)ccc3n2CCc2ccccc2)c1)[C@H]1CCNC1 ; "·3HCl"] | | C | 581 (M + 1) |
| 51 | (2S)-3-[3-[3-[(2S)-2- Carboxy-2-[(3R)- pyrrolidin-3-yl]ethyl]-5- (trifluoromethyl)anilino] phenyl]-2-[(3R)- pyrrolidin-3-yl]propanoic acid; dihydrochloride | 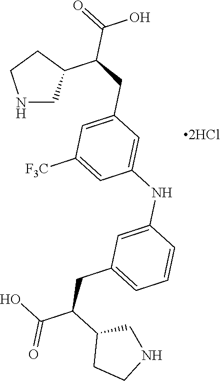 | B | C | 520 (M + H) |
| 52 | (2S)-3-[4-[3-[(2S)-2- Carboxy-2-[(3R)- pyrrolidin-3- yl]ethyl]anilino]phenyl]- 2-[(3R)-pyrrolidin-3- yl]propanoic acid; dihydrochloride | 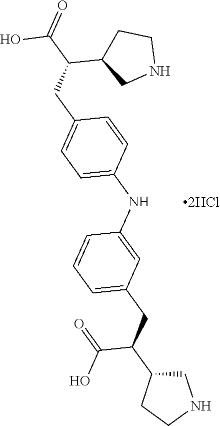 [SMILES: O=C(O)[C@@H](Cc1ccc(Nc2cccc(C[C@H](C(=O)O)[C@H]3CCNC3)c2)cc1)[C@H]1CCNC1] | B | C | 452 (M + H) |
| 53 | (2S)-3-[3-[[3-[[3-[(2S)-2- Carboxy-2-[(3R)- pyrrolidin-3- yl]ethyl]phenyl]methoxy] phenoxy]methyl]phenyl]- 2-[(3R)-pyrrolidin-3- yl]propanoic acid;dihydrochloride | 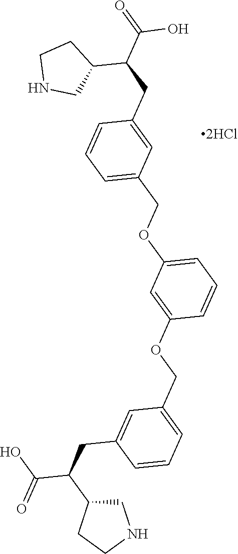 [SMILES: O=C(O)[C@@H](Cc1cccc(COc2cccc(OCc3cccc(C[C@H](C(=O)O)[C@H]4CCNC4)c3)c2)c1)[C@H]1CCNC1 ; "·2HCl"] | B | | 573 (M + H) |
| 54 | (2S)-3-[3-[[3-[(2S)-2- Carboxy-2-[(3R)- pyrrolidin-3-yl]ethyl]-N- [[3-[(2S)-2-carboxy-2- [(3R)-pyrrolidin-3- ylethyl]phenyl]carbamoyl] anilino]methyl]phenyl]- 2-[(3R)-pyrrolidin-3- yl]propanoic acid;trihydrochloride | 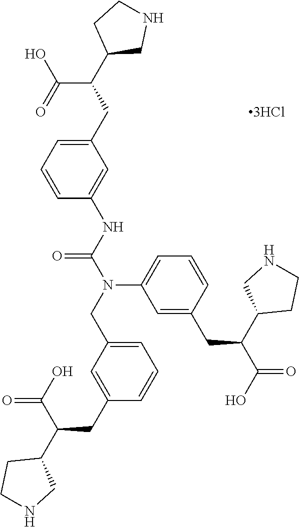 [SMILES: O=C(O)[C@@H](Cc1cccc(CN(C(=O)Nc2cccc(C[C@H](C(=O)O)[C@H]3CCNC3)c2)c2cccc(C[C@H](C(=O)O)[C@H]3CCNC3)c2)c1)[C@H]1CCNC1 ; "·3HCl"] | B | C | 726 (M + H) |
| 55 | (2S)-3-[3-[3-[3-[(2S)-2- Carboxy-2-(4,4- difluoropyrrolidin-3- yl)ethyl]phenyl]-2-oxo- imidazolidin-1- yl]phenyl]-2-(4,4- difluoropyrrolidin-3- yl)propanoic acid; dihydrochloride (Isomer 2) | 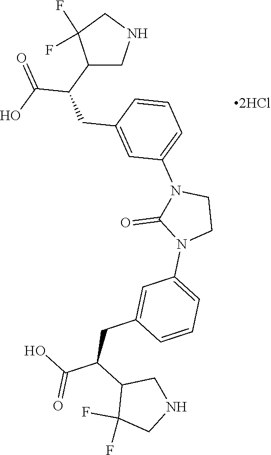 [SMILES: O=C(O)[C@@H](Cc1cccc(N2CCN(c3cccc(C[C@H](C(=O)O)C4CNCC4(F)F)c3)C2=O)c1)C1CNCC1(F)F ; "·2HCl"] | E, B | H | 593 (M + H) |
| 56 | 3-[3-[[3-[2-Carboxy-2- (4,4-difluoropyrrolidin-3- yl)ethyl]phenyl] carbamoylamino] phenyl]-2-(4,4- difluoropyrrolidin-3- yl)propanoic acid;2,2,2- trifluoroacetic acid (1/2) (Isomer 1) | 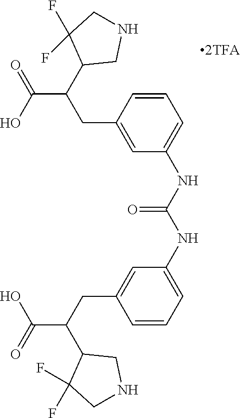 [SMILES: O=C(Nc1cccc(CC(C(=O)O)C2CNCC2(F)F)c1)Nc1cccc(CC(C(=O)O)C2CNCC2(F)F)c1 ; "·2TFA"] | D | A | 567 (M + H) |
| 57 | 3-[3-[[3-[2-Carboxy-2- (4,4-difluoropyrrolidin-3- yl)ethyl]phenyl] carbamoylamino] phenyl]-2-(4,4- difluoropyrrolidin-3- yl)propanoic acid;dihydrochloride (Isomer 2) | 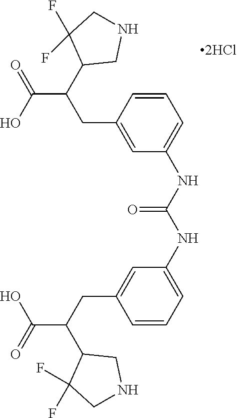 [SMILES: O=C(Nc1cccc(CC(C(=O)O)C2CNCC2(F)F)c1)Nc1cccc(CC(C(=O)O)C2CNCC2(F)F)c1 ; "·2HCl"] | E, B | M, N | 567 (M + H) |
| 58 | 3-[3-[[3-[2-Carboxy-2- (4,4-difluoropyrrolidin-3- yl)ethyl]phenyl] carbamoylamino] phenyl]-2-(4,4- difluoropyrrolidin-3- yl)propanoic acid;dihydrochloride (Isomer 3) | 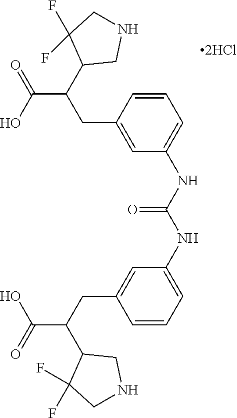 [SMILES: O=C(Nc1cccc(CC(C(=O)O)C2CNCC2(F)F)c1)Nc1cccc(CC(C(=O)O)C2CNCC2(F)F)c1 ; "·2HCl"] | F | M, N | 567 (M + H) |
| 59 | 3-[3-[[3-[2-Carboxy-2- (4,4-difluoropyrrolidin-3- yl)ethyl]phenyl] carbamoylamino] phenyl]-2-(4,4- difluoropyrrolidin-3- yl)propanoic acid; dihydrochloride (Isomer 4) | 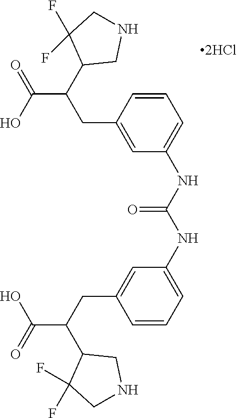 [SMILES: O=C(Nc1cccc(CC(C(=O)O)C2CNCC2(F)F)c1)Nc1cccc(CC(C(=O)O)C2CNCC2(F)F)c1 ; "·2HCl"] | G | C | 567 (M + H) |
| 60 | (2S)-3-[3-[N-Carbamoyl- 3-[(2S)-2-carboxy-2- [(3R)-pyrrolidin-3- ylethyl]anilino]phenyl]- 2-[(3R)-pyrrolidin-3- yl]propanoic acid; dihydrochloride | 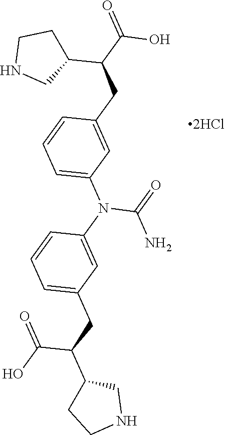 [SMILES: NC(=O)N(c1cccc(C[C@H](C(=O)O)[C@H]2CCNC2)c1)c1cccc(C[C@H](C(=O)O)[C@H]2CCNC2)c1 ; "·2HCl"] | B | C | 495 (M + H) |
| 61 | 2-[3-[[3-[Carboxy-[(3S)- pyrrolidin-3- yl]methoxy]phenyl] carbamoylamino] phenoxy]-2- [(3S)-pyrrolidin-3- yl]acetic acid; dihydrochloride, (Isomer 2) | 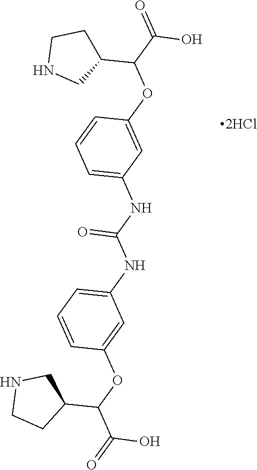 [SMILES: O=C(Nc1cccc(OC(C(=O)O)[C@H]2CCNC2)c1)Nc1cccc(OC(C(=O)O)[C@H]2CCNC2)c1 ; "·2HCl"] | | C | 499 (M + H) |
| 62 | 3-[3-[[3-[2-Carboxy-2- hydroxy-2-[(3S)- pyrrolidin-3- yl]ethyl]phenyl] carbamoylamino] phenyl]-2- hydroxy-2-[(3S)- pyrrolidin-3-yl]propanoic acid; dihydrochloride (Isomer 1) | 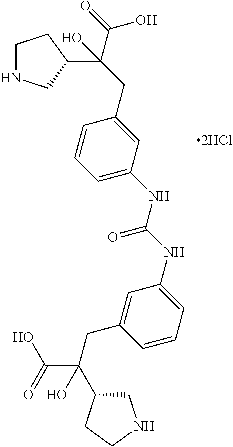 [SMILES: O=C(Nc1cccc(CC(O)(C(=O)O)[C@H]2CCNC2)c1)Nc1cccc(CC(O)(C(=O)O)[C@H]2CCNC2)c1 ; "·2HCl"] | | C | 527 (M + H) |
| 63 | 2-[3-[[3-[Carboxy-[(3S)- pyrrolidin-3- yl]methyl]sulfanylphenyl] carbamoylamino]phenyl] sulfanyl-2-[(3S)- pyrrolidin-3-yl]acetic acid; dihydrochloride (Isomer 1) | 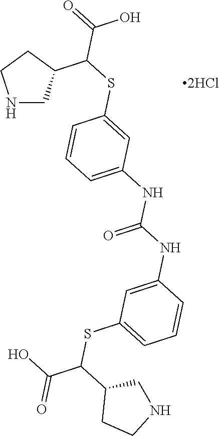 [SMILES: O=C(Nc1cccc(SC(C(=O)O)[C@H]2CCNC2)c1)Nc1cccc(SC(C(=O)O)[C@H]2CCNC2)c1 ; "·2HCl"] | B | L, N | 531 (M + H) |
| 64 | 2-[3-[[3-[Carboxy-[(3S)- pyrrolidin-3- yl]methyl]sulfanylphenyl] carbamoylamino]phenyl] sulfanyl-2-[(3S)- pyrrolidin-3-yl]acetic acid; dihydrochloride (Isomer 2) | 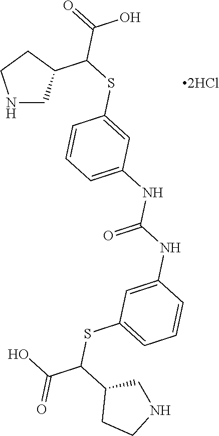 [SMILES: O=C(Nc1cccc(SC(C(=O)O)[C@H]2CCNC2)c1)Nc1cccc(SC(C(=O)O)[C@H]2CCNC2)c1 ; "·2HCl"] | B | M, N | 531 (M + H) |
| 65 | (2S)-3-[3-[[3-[(2S)-2- Carboxy-2-[(3R,5R)-5- methylpyrrolidin-3- yl]ethyl]phenyl] carbamoylamino] phenyl]-2- [(3R,5R)-5- methylpyrrolidin-3- yl]propanoic acid; dihydrochloride | 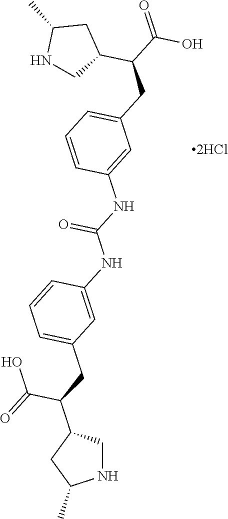 [SMILES: C[C@@H]1C[C@H]([C@H](Cc2cccc(NC(=O)Nc3cccc(C[C@H](C(=O)O)[C@@H]4CN[C@H](C)C4)c3)c2)C(=O)O)CN1 ; "·2HCl"] | | C | 523.2 (M + H) |
| 66 | 3-[3-[[3-[2-Carboxy-2- [(3R)-pyrrolidin-3- yl]propyl]phenyl] carbamoylamino] phenyl]-2- methyl-2-[(3R)- pyrrolidin-3-yl]propanoic acid; dihydrochloride (Isomer 2) | 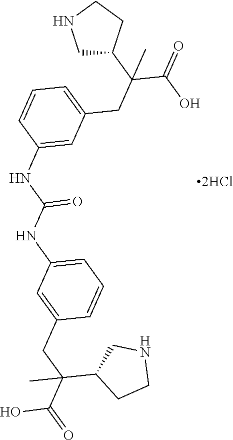 [SMILES: CC(Cc1cccc(NC(=O)Nc2cccc(CC(C)(C(=O)O)[C@H]3CCNC3)c2)c1)(C(=O)O)[C@H]1CCNC1 ; "·2HCl"] | B, E | I | 523 (M + H). |
| 67 | (2S)-3-[3-[[[3-[(2S)-2- Carboxy-2-[(3R)- pyrrolidin-3- yl]ethyl]phenyl] carbamoyl- [[3-[(2S)-2-carboxy-2- [(3R)-pyrrolidin-3- yl]ethyl]phenyl]methyl] amino]methyl]phenyl]-2- [(3R)-pyrrolidin-3- yl]propanoic acid; tri- hydrochloride | 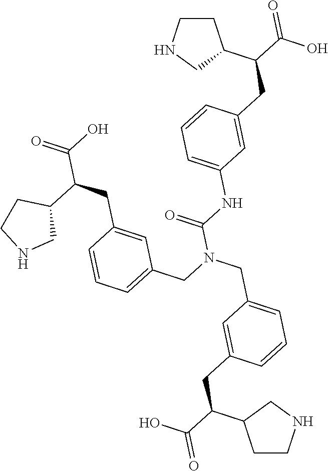 [SMILES: O=C(O)[C@@H](Cc1cccc(CN(Cc2cccc(C[C@H](C(=O)O)[C@H]3CCNC3)c2)C(=O)Nc2cccc(C[C@H](C(=O)O)[C@H]3CCNC3)c2)c1)C1CCNC1] | B | C | 740 (M + H) |
| 68 | (2S)-3-[3-[[2-[Bis[[3- [(2S)-2-carboxy-2-[(3R)- pyrrolidin-3- yl]ethyl]phenyl]methyl] amino]ethyl-[3-[(2S)-2- carboxy-2-[(3R)- pyrrolidin-3- yl]ethyl]phenyl]methyl] amino]methyl]phenyl]-2- [(3R)-pyrrolidin-3- yl]propanoic acid; hexahydrochloride | 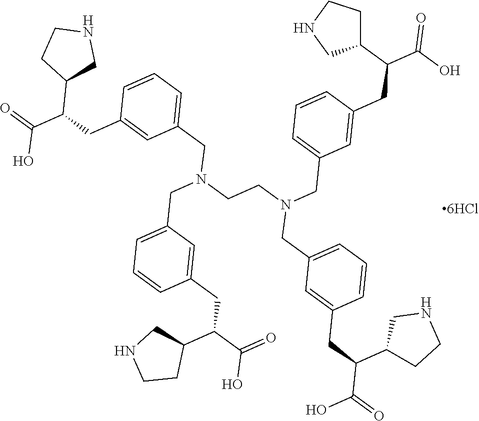 [SMILES: O=C(O)[C@@H](Cc1cccc(CN(CCN(Cc2cccc(C[C@H](C(=O)O)[C@H]3CCNC3)c2)Cc2cccc(C[C@H](C(=O)O)[C@H]3CCNC3)c2)Cc2cccc(C[C@H](C(=O)O)[C@H]3CCNC3)c2)c1)[C@H]1CCNC1 ; "·6HCl"] | B | C | 493 (M + 2H)/2 |
| 71 | (2S)-3-(Biphenyl-3-yl)-2- [(3R)-pyrrolidin-3- yl]propanoic acid hydrochloride | 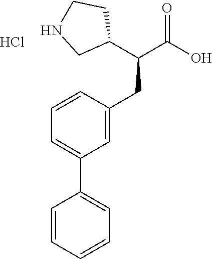 [SMILES: Cl ; O=C(O)[C@@H](Cc1cccc(-c2ccccc2)c1)[C@H]1CCNC1] | | C | 296 (M + H) |
| 72 | (2S)-3-{3-[2-(3- Cyclopropylphenyl) ethoxy] phenyl}-2-[(3R)- pyrrolidin-3-yl]propanoic acid hydrochloride | 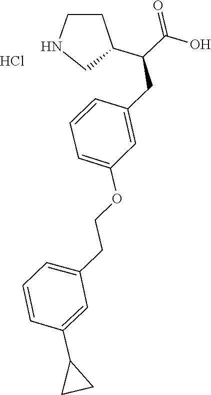 [SMILES: Cl ; O=C(O)[C@@H](Cc1cccc(OCCc2cccc(C3CC3)c2)c1)[C@H]1CCNC1] | B | C | 380 (M + H) |
| 73 | (2S)-3-[3- (Dihydroxyboranyl) phenyl]- 2-[(3R)-pyrrolidin-3- yl]propanoic acid hydrochloride | 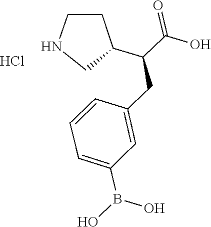 [SMILES: Cl ; O=C(O)[C@@H](Cc1cccc(B(O)O)c1)[C@H]1CCNC1] | | C | 264 (M + H) |
| 74 | (2S)-3-[3-(5- Chlorothiophen-2- yl)phenyl]-2-[(3R)- pyrrolidin-3-yl]propanoic acid hydrochloride | 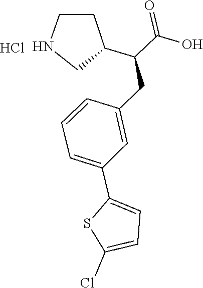 [SMILES: Cl ; O=C(O)[C@@H](Cc1cccc(-c2ccc(Cl)s2)c1)[C@H]1CCNC1] | | C | 336 (M + H) |
| 75 | 3-(3-Bromophenyl)-2- methyl-2-[(3R)- pyrrolidin-3-yl]propanoic acid;hydrochloride (Isomer 1) | 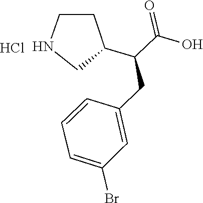 [SMILES: Cl ; O=C(O)[C@@H](Cc1cccc(Br)c1)[C@H]1CCNC1] | B, D | C | 312/314 (M + H) |
| 76 | 3-(3-Bromophenyl)-2- methyl-2-[(3R)- pyrrolidin-3-yl]propanoic acid;hydrochloride (Isomer 2) | 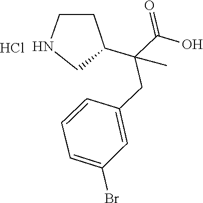 [SMILES: Cl ; CC(Cc1cccc(Br)c1)(C(=O)O)[C@H]1CCNC1] | B, E | C | 312/314 (M + H) |
| 77 | (2S)-3-(3-Fluoro-5- thiazol-4-yl-phenyl)-2- [(3R)-pyrrolidin-3- yl]propanoic acid; hydrochloride | 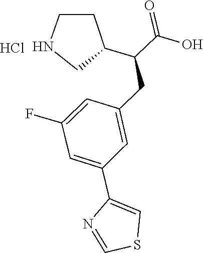 [SMILES: Cl ; O=C(O)[C@@H](Cc1cc(F)cc(-c2cscn2)c1)[C@H]1CCNC1] | | C | 321 (M + H) |
| 78 | (2S)-3-[3-(3,3- Difluoropyrrolidin-1- yl)phenyl]-2-[(3R)- pyrrolidin-3-yl]propanoic acid hydrochloride | 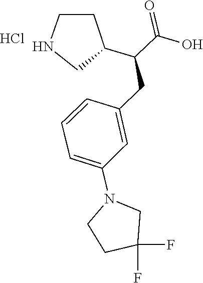 [SMILES: Cl ; O=C(O)[C@@H](Cc1cccc(N2CCC(F)(F)C2)c1)[C@H]1CCNC1] | B | C | 325 (M + H) |
| 79 | (2S)-3-[3-(3,3- Dimethylindolin-1- yl)phenyl]-2-[(3R)- pyrrolidin-3-yl]propanoic acid hydrochloride | 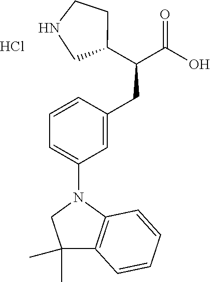 [SMILES: Cl ; CC1(C)CN(c2cccc(C[C@H](C(=O)O)[C@H]3CCNC3)c2)c2ccccc21] | B | I | 365 (M + H) |
| 80 | (2S)-3-[3-(2,2′-Dioxo- 1′,2′-dihydro-3,3′- spirobi[indol]-1(2H)- yl)phenyl]-2-[(3R)- pyrrolidin-3-yl]propanoic acid hydrochloride | 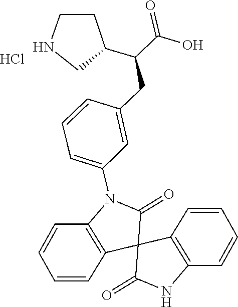 [SMILES: Cl ; O=C(O)[C@@H](Cc1cccc(N2C(=O)C3(C(=O)Nc4ccccc43)c3ccccc32)c1)[C@H]1CCNC1] | B | A, K | 468 (M + H) |
| 81 | (2S)-3-[3-(5-Methyl-2- oxo-indolin-1-yl)phenyl]- 2-[(3R)-pyrrolidin-3- yl]propanoic acid hydrochloride | 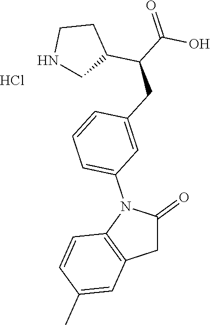 | B | I | 365 (M + H) |
| 82 | (2S)-3-[3-(2-Oxo-2,3- dihydro-1H- benzimidazol-1- yl)phenyl]-2-[(3R)- pyrrolidin-3-yl]propanoic acid hydrochloride | 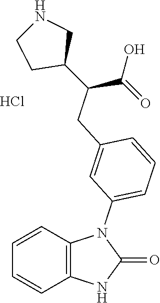 [SMILES: Cl ; O=C(O)[C@@H](Cc1cccc(-n2c(=O)[nH]c3ccccc32)c1)[C@H]1CCNC1] | | C | 352.2 (M + H) |
| 83 | (2S)-3-{3-[2-Oxo-3- (2,2,2- trifluoroethyl)imidazolidin- 1-yl]phenyl}-2-[(3R)- pyrrolidin-3-yl]propanoic acid hydrochloride | 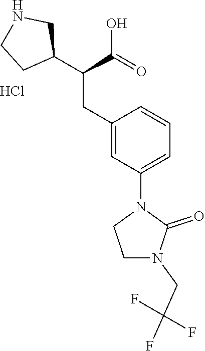 [SMILES: O=C(O)[C@@H](Cc1cccc(N2CCN(CC(F)(F)F)C2=O)c1)[C@H]1CCNC1 ; Cl] | O | C | 386 (M + H) |
| 84 | (2S)-3-[3-Fluoro-5-(2- oxo-2,3-dihydro-1H- indol-1-yl)phenyl]-2- [(3R)-pyrrolidin-3- yl]propanoic acid hydrochloride | 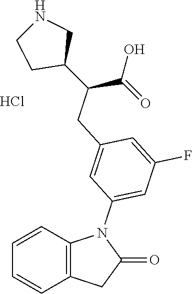 [SMILES: Cl ; O=C(O)[C@@H](Cc1cc(F)cc(N2C(=O)Cc3ccccc32)c1)[C@H]1CCNC1] | | C | 369 (M + H) |
| 85 | (2S)-3-[4-(2,5- Dioxoimidazolidin-1- yl)phenyl]-2-[(3R)- pyrrolidin-3-yl]propanoic acid hydrochloride | 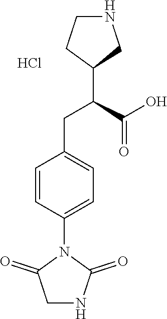 [SMILES: O=C(O)[C@@H](Cc1ccc(N2C(=O)CNC2=O)cc1)[C@H]1CCNC1 ; Cl] | | C | 318 (M + H) |
| 86 | (2S)-3-[3-(4- Phenylpiperidin-1- yl)phenyl]-2-[(3R)- pyrrolidin-3-yl]propanoic acid | 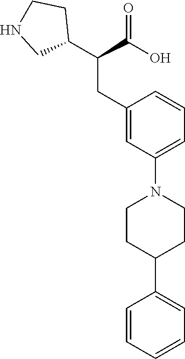 [SMILES: O=C(O)[C@@H](Cc1cccc(N2CCC(c3ccccc3)CC2)c1)[C@H]1CCNC1] | | M | 379 (M + H) |
| 87 | (2S)-3-[3-(1-Methyl-1H- indazol-3-yl)phenyl]-2- [(3R)-pyrrolidin-3- yl]propanoic acid hydrochloride | 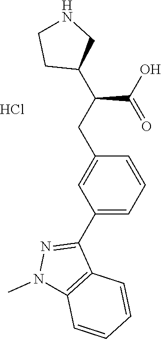 [SMILES: Cl ; Cn1nc(-c2cccc(C[C@H](C(=O)O)[C@H]3CCNC3)c2)c2ccccc21] | B | M, K | 350 (M + H) |
| 88 | (2S)-3-{3-[2- (Cyclohexyloxy)pyridin- 3-yl]phenyl}-2-[(3R)- pyrrolidin-3-yl]propanoic acid dihydrochloride | 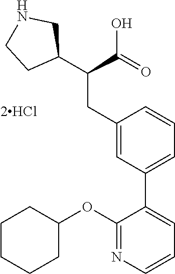 [SMILES: O=C(O)[C@@H](Cc1cccc(-c2cccnc2OC2CCCCC2)c1)[C@H]1CCNC1] | B | M, K | 395 (M + H) |
| 89 | (2S)-2-[(3R)-Pyrrolidin- 3-yl]-3-{3-[6- (trifluoromethyl)pyridin- 3-yl]phenyl}propanoic acid hydrochloride | 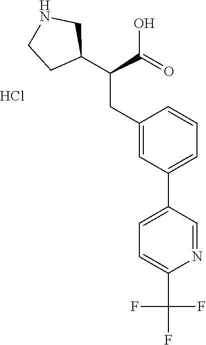 [SMILES: Cl ; O=C(O)[C@@H](Cc1cccc(-c2ccc(C(F)(F)F)nc2)c1)[C@H]1CCNC1] | B | M, K | 365 (M + H) |
| 90 | (2S)-3-(2′-Fluoro-3′- methoxybiphenyl-3-yl)- 2-[(3R)-pyrrolidin-3- yl]propanoic acid hydrochloride | 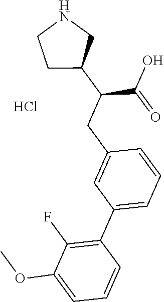 [SMILES: COc1cccc(-c2cccc(C[C@H](C(=O)O)[C@H]3CCNC3)c2)c1F ; Cl] | | L, N | 344 (M + H) |
| 91 | (2S)-3-[3-(7-Methoxy-1- benzothiophen-2- yl)phenyl]-2-[(3R)- pyrrolidin-3-yl]propanoic acid hydrochloride | 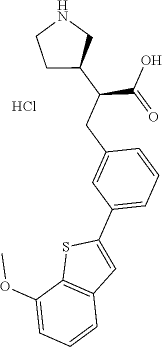 [SMILES: COc1cccc2cc(-c3cccc(C[C@H](C(=O)O)[C@H]4CCNC4)c3)sc12 ; Cl] | | C | 382 (M + H) |
| 92 | (2S)-2-[(3R)-Pyrrolidin- 3-yl]-3-[3′-(2,2,2- trifluoroethoxy)biphenyl- 3-yl]propanoic acid hydrochloride | 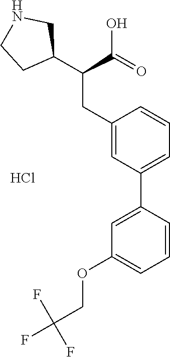 [SMILES: O=C(O)[C@@H](Cc1cccc(-c2cccc(OCC(F)(F)F)c2)c1)[C@H]1CCNC1 ; Cl] | | L, N | 394 (M + H) |
| 93 | (2S)-3-[3-(1,3- Benzothiazol-2- yl)phenyl]-2-[(3R)- pyrrolidin-3-yl]propanoic acid hydrochloride | 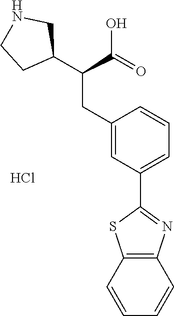 [SMILES: O=C(O)[C@@H](Cc1cccc(-c2nc3ccccc3s2)c1)[C@H]1CCNC1 ; Cl] | | L, N | 353 (M + H) |
| 94 | (2S)-3-[3-(3-Phenyl-1,2- oxazol-5-yl)phenyl]-2- [(3R)-pyrrolidin-3- yl]propanoic acid hydrochloride | 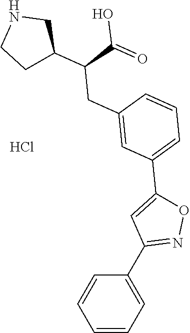 [SMILES: O=C(O)[C@@H](Cc1cccc(-c2cc(-c3ccccc3)no2)c1)[C@H]1CCNC1 ; Cl] | | C | 363 (M + H) |
| 95 | 3-{3-[4- (Hydroxymethyl)-1H- 1,2,3-triazol-1- yl]phenyl}-2-[(3R)- pyrrolidin-3-yl]propanoic acid hydrochloride | 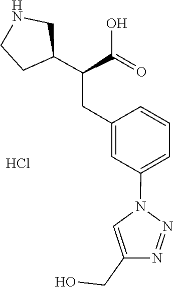 [SMILES: O=C(O)[C@@H](Cc1cccc(-n2cc(CO)nn2)c1)[C@H]1CCNC1 ; Cl] | B | C | 317 (M + H) |
| 96 | 3-[3-(4-Benzyl-1H-1,2,3- triazol-1-yl)phenyl]-2- [(3R)-pyrrolidin-3- yl]propanoic acid hydrochloride | 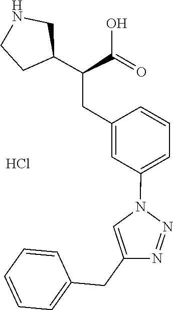 [SMILES: O=C(O)[C@@H](Cc1cccc(-n2cc(Cc3ccccc3)nn2)c1)[C@H]1CCNC1 ; Cl] | B | C | 377 (M + H) |
| 97 | 3-{3-[4-(3- Methoxyphenyl)-1H- 1,2,3-triazol-1- yl]phenyl}-2-[(3R)- pyrrolidin-3-yl]propanoic acid hydrochloride | 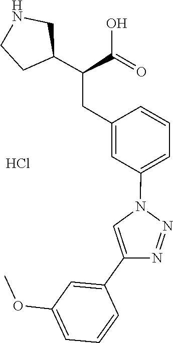 [SMILES: COc1cccc(-c2cn(-c3cccc(C[C@H](C(=O)O)[C@H]4CCNC4)c3)nn2)c1 ; Cl] | B | C | 393 (M + H) |
| 98 | 3-{3-[4-(2,5- Difluorophenyl)-1H- 1,2,3-triazol-1- yl]phenyl}-2-[(3R)- pyrrolidin-3-yl]propanoic acid hydrochloride | 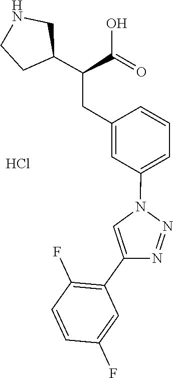 [SMILES: O=C(O)[C@@H](Cc1cccc(-n2cc(-c3cc(F)ccc3F)nn2)c1)[C@H]1CCNC1 ; Cl] | B | C | 399 (M + H) |
| 99 | (2S)-2-[(3R)-Pyrrolidin- 3-yl]-3-[3-(tetrahydro- 2H-pyran-4- yl)phenyl]propanoic acid hydrochloride | 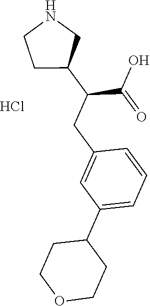 [SMILES: Cl ; O=C(O)[C@@H](Cc1cccc(C2CCOCC2)c1)[C@H]1CCNC1] | | C | 304 (M + H) |
| 100 | (2S)-3-(3- Cyclobutylphenyl)-2- [(3R)-pyrrolidin-3- yl]propanoic acid hydrochloride | 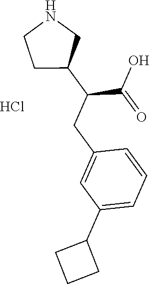 [SMILES: Cl ; O=C(O)[C@@H](Cc1cccc(C2CCC2)c1)[C@H]1CCNC1] | | C | 274 (M + H) |
| 101 | (2S)-3-{3-[(2- Cyanoethanimidoyl)amino] phenyl}-2-[(3R)- pyrrolidin-3-yl]propanoic acid dihydrochloride | 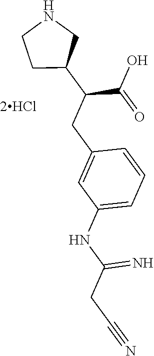 [SMILES: "2·HCl" ; N#CCC(=N)Nc1cccc(C[C@H](C(=O)O)[C@H]2CCNC2)c1] | P, B | C | 301 (M + H) |
| 102 | (2S)-3-[3- (Dimethylsulfamoyl) phenyl]-2-[(3R)-pyrrolidin- 3-yl]propanoic acid hydrochloride | 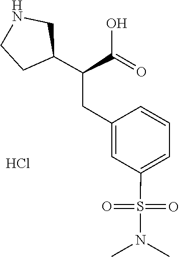 [SMILES: CN(C)S(=O)(=O)c1cccc(C[C@H](C(=O)O)[C@H]2CCNC2)c1 ; Cl] | | C | 327 (M + H) |
| 103 | (2S)-2-[(3R)-Pyrrolidin- 3-yl]-3-[4- (trifluoromethyl)phenyl] propanoic acid hydrochloride | 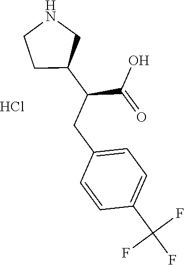 [SMILES: Cl ; O=C(O)[C@@H](Cc1ccc(C(F)(F)F)cc1)[C@H]1CCNC1] | | Q, N | 288 (M + H) |
| 104 | (2S)-2-[(3R)-Pyrrolidin- 3-yl]-3-{3-[(3- sulfopropyl)amino]phenyl} propanoic acid hydrochloride | 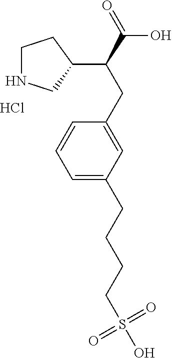 [SMILES: Cl ; O=C(O)[C@@H](Cc1cccc(CCCCS(=O)(=O)O)c1)[C@H]1CCNC1] | U | A, K | 357 (M + H) |
| 105 | (2S)-3-{3-[(Biphenyl-3- yloxy)methyl]phenyl}-2- [(3R)-pyrrolidin-3- yl]propanoic acid hydrochloride | 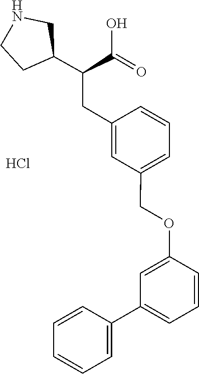 [SMILES: O=C(O)[C@@H](Cc1cccc(COc2cccc(-c3ccccc3)c2)c1)[C@H]1CCNC1 ; Cl] | | C | 402 (M + H) |
| 106 | (2S)-3-{3-[(2,2-Difluoro- 1,3-benzodioxol-5- yl)methoxy]phenyl}-2- [(3R)-pyrrolidin-3- yl]propanoic acid hydrochloride | 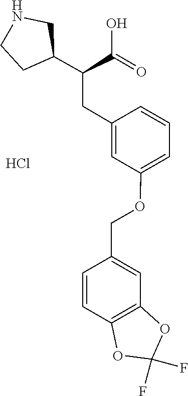 [SMILES: O=C(O)[C@@H](Cc1cccc(OCc2ccc3c(c2)OC(F)(F)O3)c1)[C@H]1CCNC1 ; Cl] | | C | 406 (M + H) |
| 107 | (2S)-3-[3-({3-[(2,4- Difluorobenzyl)oxy]benzyl} oxy)phenyl]-2-[(3R)- pyrrolidin-3-yl]propanoic acid hydrochloride | 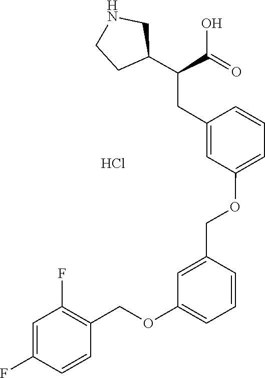 [SMILES: O=C(O)[C@@H](Cc1cccc(OCc2cccc(OCc3ccc(F)cc3F)c2)c1)[C@H]1CCNC1 ; Cl] | | C | 468 (M + H) |
| 108 | (2S)-3-[3-(Imidazo[1,5- alpyridin-3- ylamino)phenyl]-2-[(3R)- pyrrolidin-3-yl]propanoic acid hydrochloride | 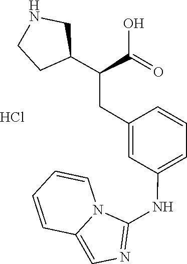 [SMILES: Cl ; O=C(O)[C@@H](Cc1cccc(Nc2ncc3ccccn23)c1)[C@H]1CCNC1] | B | C | 351 (M + H) |
| 109 | (2S)-3-{3-[(1,3- Benzodioxol-5- ylmethyl)sulfamoyl]phenyl}- 2-[(3R)-pyrrolidin-3- yl]propanoic acid hydrochloride | 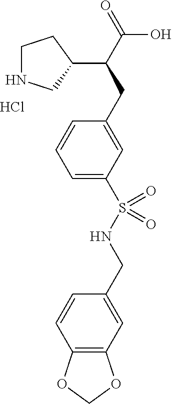 [SMILES: Cl ; O=C(O)[C@@H](Cc1cccc(S(=O)(=O)NCc2ccc3c(c2)OCO3)c1)[C@H]1CCNC1] | | C | 434 (M + H) |
| 110 | (2S)-3-{3-[(2,3-Dihydro- 1,4-benzodioxin-6- ylmethyl)sulfamoyl]phenyl}- 2-[(3R)-pyrrolidin-3- yl]propanoic acid hydrochloride | 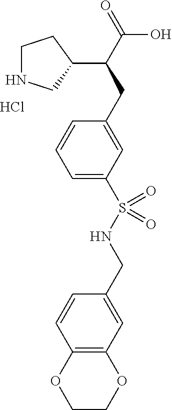 [SMILES: Cl ; O=C(O)[C@@H](Cc1cccc(S(=O)(=O)NCc2ccc3c(c2)OCCO3)c1)[C@H]1CCNC1] | | C | 446 (M + H) |
| 111 | (2S)-3-{3-[(Pyridin-2- yl)amino]phenyl}-2- [(3R)-pyrrolidin-3- yl]propanoic acid hydrochloride | 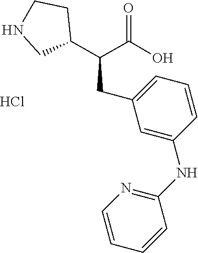 [SMILES: Cl ; O=C(O)[C@@H](Cc1cccc(Nc2ccccn2)c1)[C@H]1CCNC1] | | A, K | 312 (M + H) |
| 112 | (2S)-3-{3-[(2,3-Dihydro- 1H-inden-5- yl)amino]phenyl}-2- [(3R)-pyrrolidin-3- yl]propanoic acid hydrochloride | 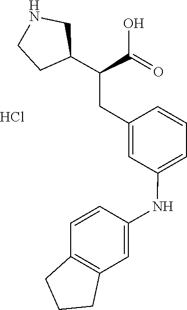 [SMILES: Cl ; O=C(O)[C@@H](Cc1cccc(Nc2ccc3c(c2)CCC3)c1)[C@H]1CCNC1] | | A, K | 351 (M + H) |
| 113 | (2S)-3-{3-[3-(Piperidin- 4-yl)anilino]phenyl}-2- [(3R)-pyrrolidin-3- yl]propanoic acid dihydrochloride | 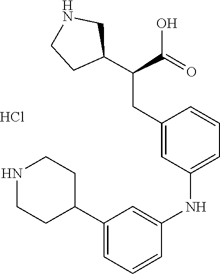 [SMILES: Cl ; O=C(O)[C@@H](Cc1cccc(Nc2cccc(C3CCNCC3)c2)c1)[C@H]1CCNC1] | | A, K | 394 (M + H) |
| 114 | (2S)-3-(3-{[4-(1,3- Oxazol-5- yl)anilino]methyl}phenyl)- 2-[(3R)-pyrrolidin-3- yl]propanoic acid hydrochloride | 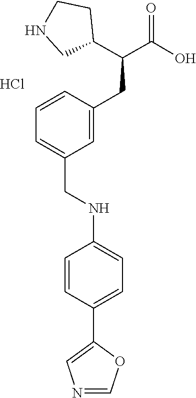 [SMILES: Cl ; O=C(O)[C@@H](Cc1cccc(CNc2ccc(-c3cnco3)cc2)c1)[C@H]1CCNC1] | | C | 392 (M + H) |
| 115 | (2S)-3-(3-{[(3- Methoxyphenyl)(methyl) amino]methyl}phenyl)-2- [(3R)-pyrrolidin-3- yl]propanoic acid hydrochloride | 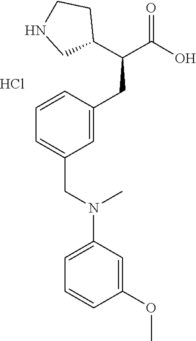 [SMILES: Cl ; COc1cccc(N(C)Cc2cccc(C[C@H](C(=O)O)[C@H]3CCNC3)c2)c1] | | C | 369 (M + H) |
| 116 | 3-(Benzyloxy)-2- (biphenyl-3-ylmethyl)-2- [(3R)-pyrrolidin-3- yl]propanoic acid hydrochloride (Isomer 1) | 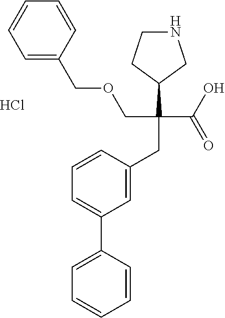 [SMILES: Cl ; O=C(O)C(COCc1ccccc1)(Cc1cccc(-c2ccccc2)c1)[C@H]1CCNC1] | D | C | 416 (M + H) |
| 117 | 3-(Benzyloxy)-2- (biphenyl-3-ylmethyl)-2- [(3R)-pyrrolidin-3- yl]propanoic acid hydrochloride (Isomer 2) | 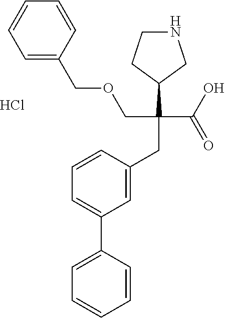 [SMILES: Cl ; O=C(O)C(COCc1ccccc1)(Cc1cccc(-c2ccccc2)c1)[C@H]1CCNC1] | E | C | 416 (M + H) |
| 118 | 3-(Biphenyl-3-yl)-2- (hydroxymethyl)-2- [(3R)-pyrrolidin-3- yl]propanoic acid hydrochloride (Isomer 2) | 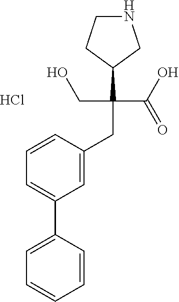 [SMILES: Cl ; O=C(O)C(CO)(Cc1cccc(-c2ccccc2)c1)[C@H]1CCNC1] | E | C | 326 (M + H) |
| 119 | 3-(Biphenyl-3-yl)-2- (hydroxymethyl)-2- [(3R)-pyrrolidin-3- yl]propanoic acid hydrochloride (Isomer 1) | 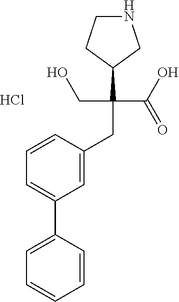 [SMILES: Cl ; O=C(O)C(CO)(Cc1cccc(-c2ccccc2)c1)[C@H]1CCNC1] | D | C | 326 (M + H) |
| 120 | 5-{(2S)-2-Carboxy-2- [(3R)-pyrrolidin-3- yl]ethyl}pyrazine-2- carboxylic acid hydrochloride | 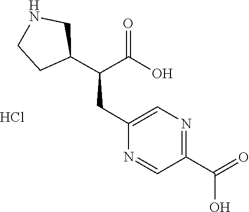 [SMILES: Cl ; O=C(O)c1cnc(C[C@H](C(=O)O)[C@H]2CCNC2)cn1] | | R | 266 (M + H) |
| 121 | (2S)-3-(5- Bromopyrimidin-2-yl)-2- [(3R)-pyrrolidin-3- yl]propanoic acid hydrochloride | 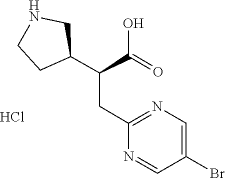 [SMILES: Cl ; O=C(O)[C@@H](Cc1ncc(Br)cn1)[C@H]1CCNC1] | | R | 302.2 (M + H) |
| 122 | (2S)-3-(2-Chloropyridin- 4-yl)-2-[(3R)-pyrrolidin- 3-yl]propanoic acid hydrochloride | 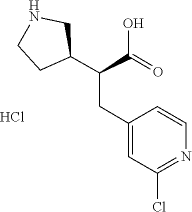 [SMILES: Cl ; O=C(O)[C@@H](Cc1ccnc(Cl)c1)[C@H]1CCNC1] | | R | 255.3 (M + H) |
| 123 | (2S)-3-(2-Bromo-4- cyanophenyl)-2-[(3R)- pyrrolidin-3-yl]propanoic acid hydrochloride | 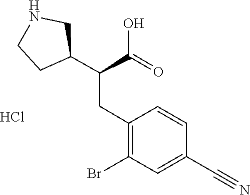 [SMILES: Cl ; N#Cc1ccc(C[C@H](C(=O)O)[C@H]2CCNC2)c(Br)c1] | | R | 323.2 (M + H) |
| 124 | (2S)-3-[3-Bromo-5- (trifluoromethoxy)phenyl]- 2-[(3R)-pyrrolidin-3- yl]propanoic acid hydrochloride | 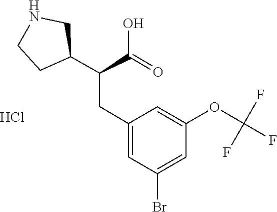 [SMILES: Cl ; O=C(O)[C@@H](Cc1cc(Br)cc(OC(F)(F)F)c1)[C@H]1CCNC1] | | R | 382.2 (M + H) |
| 125 | (2S)-3-(3-Azidophenyl)- 2-[(3R)-pyrrolidin-3- yl]propanoic acid hydrochloride | 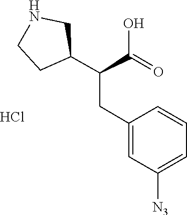 [SMILES: Cl ; [N-]=[N+]=Nc1cccc(C[C@H](C(=O)O)[C@H]2CCNC2)c1] | | R | 261.2 (M + H) |
| 126 | (2S)-3-[3- (Methylsulfanyl)phenyl]- 2-[(3R)-pyrrolidin-3- yl]propanoic acid hydrochloride | 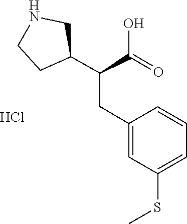 [SMILES: Cl ; CSc1cccc(C[C@H](C(=O)O)[C@H]2CCNC2)c1] | | S | 266.0 (M + H) |
| 127 | (2S)-3-(3-Acetylphenyl)- 2-[(3R)-pyrrolidin-3- yl]propanoic acid hydrochloride | 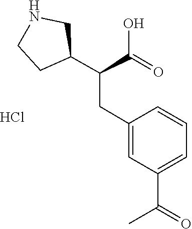 [SMILES: Cl ; CC(=O)c1cccc(C[C@H](C(=O)O)[C@H]2CCNC2)c1] | V | R | 262.0 (M + H) |
| 128 | (2S)-3-[3- (Aminomethyl)-5- methylphenyl]-2-[(3R)- pyrrolidin-3-yl]propanoic acid dihydrochloride | 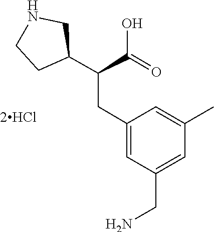 [SMILES: "2·HCl" ; Cc1cc(CN)cc(C[C@H](C(=O)O)[C@H]2CCNC2)c1] | | R | 263.4 (M + H) |
| 129 | (2S)-3-(3-Formyl-5- methylphenyl)-2-[(3R)- pyrrolidin-3-yl]propanoic acid hydrochloride | 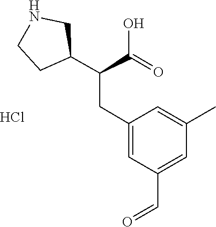 [SMILES: Cl ; Cc1cc(C=O)cc(C[C@H](C(=O)O)[C@H]2CCNC2)c1] | | R | 262.1 (M + H) |
| 130 | (2S)-3-[3- (Methoxycarbonyl)phenyl]- 2-[(3R)-pyrrolidin-3- yl]propanoic acid hydrochloride | 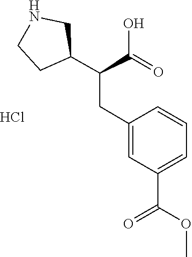 [SMILES: Cl ; COC(=O)c1cccc(C[C@H](C(=O)O)[C@H]2CCNC2)c1] | | C | 278.1 (M + H) |
| 131 | (2S)-3-(3-Bromo-5- methylphenyl)-2-[(3R)- pyrrolidin-3-yl]propanoic acid hydrochloride | 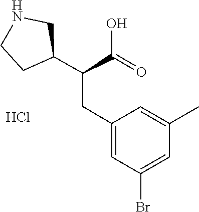 [SMILES: Cc1cc(Br)cc(C[C@H](C(=O)O)[C@H]2CCNC2)c1 ; Cl] | | R | 312.2 (M + H) |
| 132 | (2S)-3-(3-Hydroxy-5- methylphenyl)-2-[(3R)- pyrrolidin-3-yl]propanoic acid hydrochloride | 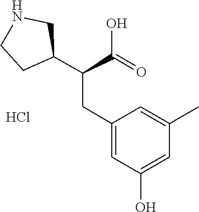 [SMILES: Cc1cc(O)cc(C[C@H](C(=O)O)[C@H]2CCNC2)c1 ; Cl] | | R | 250.4 (M + H) |
| 133 | (2S)-3-(3-Borono-5- methylphenyl)-2-[(3R)- pyrrolidin-3-yl]propanoic acid hydrochloride | 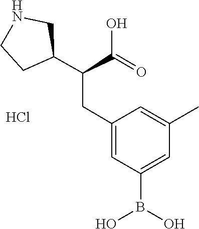 [SMILES: Cc1cc(C[C@H](C(=O)O)[C@H]2CCNC2)cc(B(O)O)c1 ; Cl] | W | R | 278.3 (M + H) |
| 134 | (2S)-3-(2-Bromo-4- carbamoylphenyl)-2- [(3R)-pyrrolidin-3- yl]propanoic acid hydrochloride | 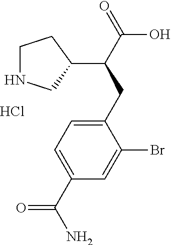 [SMILES: Cl ; NC(=O)c1ccc(C[C@H](C(=O)O)[C@H]2CCNC2)c(Br)c1] | | R | 342.9 (M + H) |
| 135 | (2S)-3-[3- (Hydroxymethyl)-5- methylphenyl]-2-[(3R)- pyrrolidin-3- yl]propanoate ammonium salt | 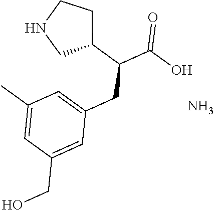 | | T | 264.4 (M + H) |
| 136 | (2S,2′S)-3,3′-[1H- Pyrazole-1,3-diyldi(3,1- phenylene)]bis{2-[(3R)- pyrrolidin-3-yl]propanoic acid} dihydrochloride | 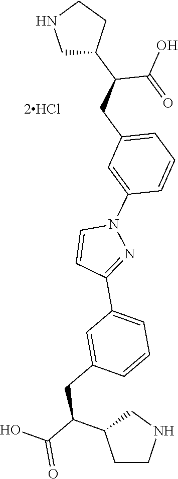 [SMILES: O=C(O)[C@@H](Cc1cccc(-c2ccn(-c3cccc(C[C@H](C(=O)O)[C@H]4CCNC4)c3)n2)c1)[C@H]1CCNC1] | | R | 503.1 (M + H) |
| 137 | (2S)-3-[3-(3-{(2S)-2- Carboxy-2-[(3R)- pyrrolidin-3- yl]ethyl}benzamido)phenyl]- 2-[(3R)-pyrrolidin-3- yl]propanoic acid dihydrochloride | 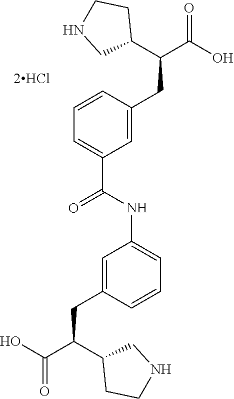 [SMILES: O=C(Nc1cccc(C[C@H](C(=O)O)[C@H]2CCNC2)c1)c1cccc(C[C@H](C(=O)O)[C@H]2CCNC2)c1 ; "2·HCl"] | | R | 480.3 (M + H) |
| 138 | (2S)-3-{3-[(3-{(2S)-2- Carboxy-2-[(3R)- pyrrolidin-3- yl]ethyl}benzene-1- sulfonyl)amino]phenyl}- 2-[(3R)-pyrrolidin-3- yl]propanoic acid dihydrochloride | 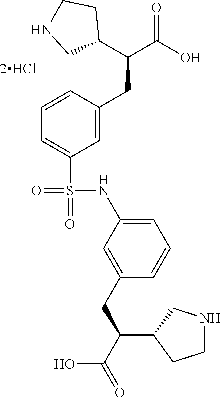 [SMILES: "2·HCl" ; O=C(O)[C@@H](Cc1cccc(NS(=O)(=O)c2cccc(C[C@H](C(=O)O)[C@H]3CCNC3)c2)c1)[C@H]1CCNC1] | | R | 516.4 (M + H) |
| 139 | (2S,2′S)-3,3′-[1H-1,2,3- Triazole-1,4-diyldi(3,1- phenylene)]bis{2-[(3R)- pyrrolidin-3-yl]propanoic acid} dihydrochloride | 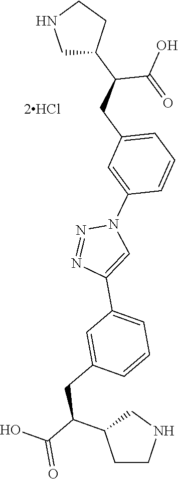 [SMILES: O=C(O)[C@@H](Cc1cccc(-c2cn(-c3cccc(C[C@H](C(=O)O)[C@H]4CCNC4)c3)nn2)c1)[C@H]1CCNC1] | | R | 504.4 (M + H) |
| 140 | (2S,2′S)-3,3′-[Propane- 1,3-diyldi(3,1- phenylene)]bis{2-[(3R)- pyrrolidin-3-yl]propanoic acid} dihydrochloride | 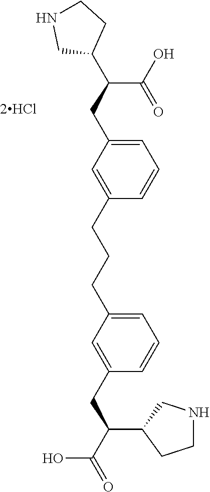 [SMILES: O=C(O)[C@@H](Cc1cccc(CCCc2cccc(C[C@H](C(=O)O)[C@H]3CCNC3)c2)c1)[C@H]1CCNC1] | | R | 479.4 (M + H) |
| 141 | (2S)-3-{5-[(3-{(2S)-2- Carboxy-2-[(3R)- pyrrolidin-3- yl]ethyl}phenyl)carbamoyl] pyrazin-2-yl}-2-[(3R)- pyrrolidin-3-yl]propanoic acid dihydrochloride | 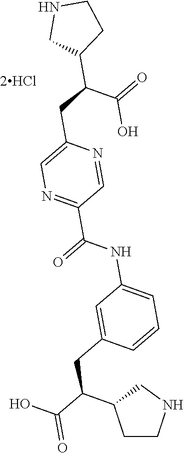 | | R | 482.2 (M + H) |
| 142 | (2S,2′S)-3,3′-[(2- Oxoimidazolidine-1,3- diyl)di(pyridine-2,4- diyl)]bis{2-[(3R)- pyrrolidin-3-yl]propanoic acid} dihydrochloride | 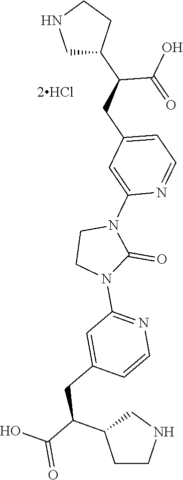 [SMILES: O=C(O)[C@@H](Cc1ccnc(N2CCN(c3cc(C[C@H](C(=O)O)[C@H]4CCNC4)ccn3)C2=O)c1)[C@H]1CCNC1] | | R | 523.4 (M + H) |
| 143 | (2S)-3-[2-(3-{(2S)-2- Carboxy-2-[(3R)- pyrrolidin-3- yl]ethyl}anilino)-5- cyanophenyl]-2-[(3R)- pyrrolidin-3-yl]propanoic acid hydrochloride | 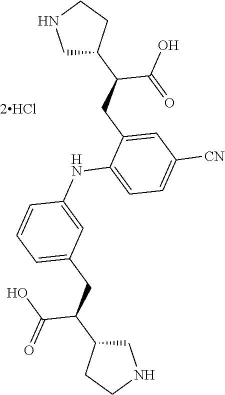 [SMILES: N#Cc1ccc(Nc2cccc(C[C@H](C(=O)O)[C@H]3CCNC3)c2)c(C[C@H](C(=O)O)[C@H]2CCNC2)c1] | | R | 477.3 (M + H) |
| 144 | (2S)-3-[3-[[Bis[[3-[(2S)- 2-carboxy-2-[(3R)- pyrrolidin-3-yl]ethyl]-5- methyl- phenyl]methyl]amino] methyl]-5-methyl-phenyl]- 2-[(3R)-pyrrolidin-3- yl]propanoic acid tetrahydrochloride | 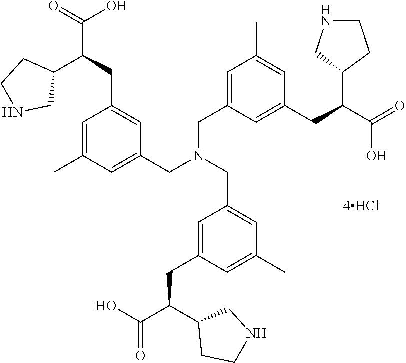 [SMILES: Cc1cc(C[C@H](C(=O)O)[C@H]2CCNC2)cc(CN(Cc2cc(C)cc(C[C@H](C(=O)O)[C@H]3CCNC3)c2)Cc2cc(C)cc(C[C@H](C(=O)O)[C@H]3CCNC3)c2)c1] | | R | 753.4 (M + H) |
| 145 | (2S)-3-[3-[[[3-[(2S)-2- Carboxy-2-[(3R)- pyrrolidin-3-yl]ethyl]-5- methyl-phenyl]methyl- [(3-fluoro-5-methoxy- phenyl)methyl]amino] methyl]-5-methyl-phenyl]- 2-[(3R)-pyrrolidin-3- yl]propanoic acid trihydrochloride | 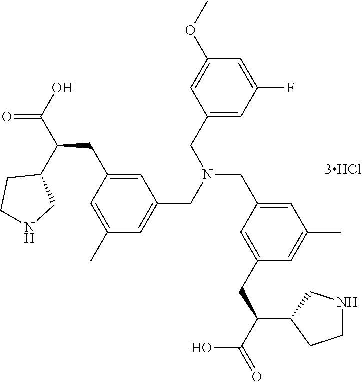 [SMILES: COc1cc(F)cc(CN(Cc2cc(C)cc(C[C@H](C(=O)O)[C@H]3CCNC3)c2)Cc2cc(C)cc(C[C@H](C(=O)O)[C@H]3CCNC3)c2)c1] | | R | 646.4 (M + H) |
| 154 | (2S)-3-[3-(1- Cyclohexylpyrazol-4- yl)phenyl]-2-[(3R)- pyrrolidin-3-yl]propanoic acid hydrochloride | 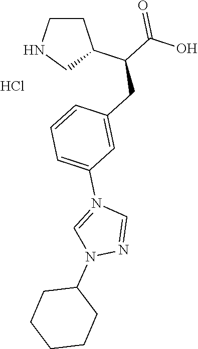 | | C | 368 (M + H) |

A. Reverse-phase HPLC purification [column: Bonus RP 21 × 100 mm, 5 μm; mobile phase: solvent A-aqueous TFA (0.05%) pH 2.5, solvent B − ACN + 0.05% TFA]
B. HCl (2 M) in Et₂O was used in place of HCl (4 M in 1,4-dioxane), and DCM was used as reaction solvent.
C. Upon complete deprotection, product isolated by concentrating reaction mixture to dryness
D. Isomer 1 of the starting material was used
E. Isomer 2 of the starting material was used
F. Isomer 3 of the starting material was used
G. Isomer 4 of the starting material was used
H. The product was triturated with ACN + water
I. Upon complete deprotection, product isolated by adding MTBE and collecting the solid by filtration
J. The product was triturated with MTBE
K. After purification, the HCl salt was prepared by mixing the free base with water and 2 M HCl in Et₂O, then evaporated to dryness and further dried in vacuo.
L. Reverse-phase flash chromatography eluting with a gradient of ACN in aqueous NH₄HCO₃
M. Reverse-phase HPLC purification [column: XBridge ™ C18; mobile phase: solvent A = 20 mM aqueous NH₄HCO₃ (pH 9), solvent B = ACN].
N. After purification, the HCl salt was prepared by mixing the free base with water and 1N aqueous HCl, then evaporated to dryness and further dried in vacuo.
O. HCl (5.5 M) in IPA was used in place of HCl (4 M in 1,4-dioxane)
P. Starting material used: tert-Butyl (3R)-3-[(1S)-1-[[3-[[(E)-1-amino-2-cyano-vinyl]amino]phenyl]methyl]-2-tert-butoxy-2-oxo-ethyl]pyrrolidine-1-carboxylate
Q. Strong cation exchange (SCX) purification, eluted with NH₃ in MeOH
R. Reverse-phase HPLC purification [column: Welch Xtimate ® C18 150 × 25 mm, 5 μm; mobile phase − solvent A = aqueous HCl, solvent B = ACN]
S. Reverse-phase HPLC purification [column: Phenomenex ® Gemini ® NX C18 75 × 30 mm, 3 μm; mobile phase: solvent A = 10 mM aqueous NH₄HCO₃, solvent B = ACN]
T. Reverse-phase HPLC purification [column: Welch Xtimate ® C18 150 × 25 mm, 5 μm; mobile phase − solvent A = aqueous NH₄HCO₃, solvent B = ACN]
U. Starting material used: (2S)-2-[(3R)-1-tert-butoxycarbonylpyrrolidin-3-yl]-3-[3-(1,1-dioxo-1,2-thiazolidin-2-yl)phenyl]propanoic acid; 1,2-thiazolidine ring opened under these conditions
V. Starting material used: tert-Butyl (3R)-3-[(1S)-2-tert-butoxy-1-[[3-(1-ethoxyvinyl)phenyl]methyl]-2-oxo-ethyl]pyrrolidine-1-carboxylate
W. Starting material used: tert-Butyl (3R)-3-[(1S)-2-tert-butoxy-1-[[3-methyl-5-(4,4,5,5-tetramethyl-1,3,2-dioxaborolan-2-yl)phenyl]methyl]-2-oxo-ethyl]pyrrolidine-1-carboxylate

### EXAMPLE 69

### (2S)-3-[3-[[3-[(2S)-2-Carboxy-2-[(3R)-pyrrolidin-3-yl]ethyl]-5-(trifluoromethyl)phenyl]carbamoylamino]-5-(trifluoromethyl)phenyl]-2-[(3R)-pyrrolidin-3-yl]propanoic acid;dihydrochloride

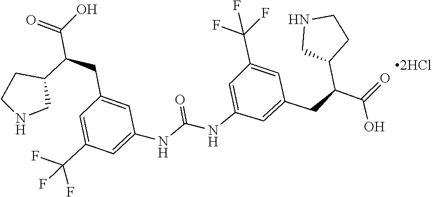 [SMILES: O=C(Nc1cc(C[C@H](C(=O)O)[C@H]2CCNC2)cc(C(F)(F)F)c1)Nc1cc(C[C@H](C(=O)O)[C@H]2CCNC2)cc(C(F)(F)F)c1]

A mixture of tert-butyl (3R)-3-[(1S)-1-[[3-amino-5-(trifluoromethyl)phenyl]methyl]-2-tert-butoxy-2-oxo-ethyl]pyrrolidine-1-carboxylate (346 mg, 0.755 mmol) and 1,1′-carbonyldiimidazole (61 mg, 0.38 mmol) in THE (7.5 mL) was stirred for 16 h. The reaction mixture was acidified with 1 M aqueous HCl and extracted with EtOAc. The organic layer was separated, and the organic layer was dried over MgSO₄, filtered, and concentrated. The crude material was dissolved in DCM (2 mL) and 4 M HCl in dioxane (3 mL) was added. The reaction was stirred for 16 h. The solvent was evaporated in vacuo. The residue was purified via reverse phase flash chromatography using a gradient of 20 to 60% ACN in aqueous NH₄CO₃ (pH9) giving the free-base product. This material was treated with 1 M aqueous HCl (1 mL) and the solvent evaporated to obtain the title compound (30 mg, 5%). ES/MS (m/z): 631 (M+1).

### EXAMPLE 70

### (2S)-3-[4-[4-[(2S)-2-Carboxy-2-[(3R)-pyrrolidin-3-yl]ethyl]anilino]phenyl]-2-[(3R)-pyrrolidin-3-yl]propanoic acid;dihydrochloride

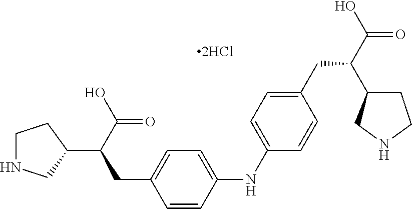 [SMILES: O=C(O)[C@@H](Cc1ccc(Nc2ccc(C[C@H](C(=O)O)[C@H]3CCNC3)cc2)cc1)[C@H]1CCNC1]

tert-Butyl (3R)-3-[(1S)-1-[(4-bromophenyl)methyl]-2-tert-butoxy-2-oxo-ethyl]pyrrolidine-1-carboxylate (620 mg, 1.36 mmol), tert-butyl (3R)-3-[(1S)-1-[(4-aminophenyl)methyl]-2-tert-butoxy-2-oxo-ethyl]pyrrolidine-1-carboxylate (799 mg, 2.05 mmol), [tBuBrettPhos Pd(allyl)]OTf (80 mg, 0.10 mmol), and cesium carbonate (1.33 g, 4.09 mmol) were combined in a microwave vessel, followed by argon-sparged dioxane (14 mL). The reaction mixture was heated to 100° C. for 12 h in a microwave reactor. The reaction mixture was then concentrated to dryness under reduced pressure, and the residue purified by silica gel chromatography eluting with a 0-50% gradient of EtOAc in hexanes. The residue was dissolved in 4 N HCl-dioxane (24 mL), stirred until desired product was observed, concentrated to a solid, and purified via reverse phase HPLC [column: Kinetex Evo 100×30 mm 5 μm; mobile phase: solvent A=10 mM aqueous NH₄HCO₃+5% MeOH, solvent B=ACN; gradient: 0 to 10% solvent B in solvent A]. The isolated product was dissolved in HCl (1 N aqueous solution) and lyophilized to give the title compound (130 mg, 18%) as a yellow solid. ES/MS (m/z): 452 (M+H).

### EXAMPLE 146

### (2S)-3-[3-(Pyrimidin-5-yl)phenyl]-2-[(3R)-pyrrolidin-3-yl]propanoic acid hydrochloride

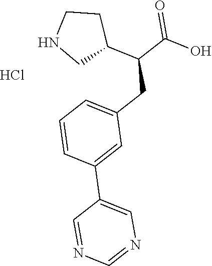 [SMILES: Cl ; O=C(O)[C@@H](Cc1cccc(-c2cncnc2)c1)[C@H]1CCNC1]

A mixture of pyrimidin-5-ylboronic acid (40 mg, 0.33 mmol), tetrakis(triphenylphosphine)palladium(0) (24 mg, 20 mmol), (2S)-3-(3-bromophenyl)-2-[(3R)-1-tert-butoxycarbonylpyrrolidin-3-yl]propanoic acid (prepared essentially as described in WO 2020/247429, 100 mg, 251 mmol), K₂CO₃ (2 M solution in water, 251 L, 0.502 mmol), and 1,4-dioxane (1 mL) was purged with N₂ and incubated at 100° C. for 18 h. The reaction mixture was concentrated under N₂ then the residue was purified on a hydrophobic lipophilic balance (HLB) column eluted with a gradient of 0 to 100% ACN in aqueous NH₄HCO₃ (pH9). To the residue was added HCl (4 M in 1,4-dioxane, 627 μL, 2.51 mmol) and the reaction was stirred 3 h at RT. The mixture was concentrated to give the title compound (71 mg, 85%). ES-MS m/z 298 (M+H).

### EXAMPLE 147

### (2S)-3-(3-{[3-(Hydrazinecarbonyl)phenoxy]methyl}phenyl)-2-[(3R)-pyrrolidin-3-yl]propanoic acid hydrochloride

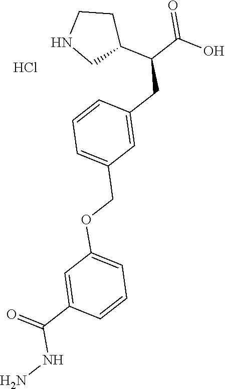 [SMILES: NNC(=O)c1cccc(OCc2cccc(C[C@H](C(=O)O)[C@H]3CCNC3)c2)c1 ; Cl]

To a reaction vessel containing tert-butyl (3R)-3-[(1S)-2-tert-butoxy-1-[[3-(hydroxymethyl)phenyl]methyl]-2-oxo-ethyl]pyrrolidine-1-carboxylate (prepared essentially as described in WO 2020/247429; 125 mg, 0.308 mmol) was added sequentially: 3-(1,3,4-oxadiazol-2-yl)phenol (75 mg, 0.462 mmol), triphenylphosphine (123 mg, 0.462 mmol), THE (1 mL), and finally diethyl azodicarboxylate (73 μL, 81 mg, 0.462 mmol). The reaction mixture was stirred at RT for 24 h, then the mixture was evaporated to dryness. The residue was taken up in DCM and passed through a plug of silica gel eluting with 20/80 acetone/hexane. The filtrate was concentrated and the residue was purified by reverse-phase HPLC [column: XBridge® C18 19×100 mm, 5 μm; mobile phase: solvent A=20 mM NH₄HCO₃ (pH 9), solvent B=ACN, gradient: 62-93% solvent B in solvent A; flow rate 25 mL/min]. The product was treated with HCl (4 M solution in 1,4-dioxane, 77 L) at 50° C. overnight, then the solvent was evaporated to give the title compound (12 mg, 7%). ES-MS m/z 384 (M+H).

### EXAMPLE 148

### (2S)-3-{3-[3-(3-Hydroxyphenyl)-2-oxoimidazolidin-1-yl]phenyl}-2-[(3R)-pyrrolidin-3-yl]propanoic acid hydrochloride

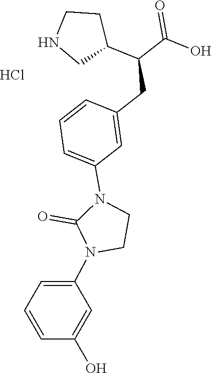 [SMILES: Cl ; O=C(O)[C@@H](Cc1cccc(N2CCN(c3cccc(O)c3)C2=O)c1)[C@H]1CCNC1]

To a solution of (2S)-3-[3-[3-(3-benzyloxyphenyl)-2-oxo-imidazolidin-1-yl]phenyl]-2-[(3R)-1-tert-butoxycarbonylpyrrolidin-3-yl]propanoic acid (1.10 g, 1.88 mmol) in toluene (18.8 mL) at 70° C. was added N,N-dimethylformamide di-tert-butyl acetal (3.39 g, 4.02 mL 15.0 mmol) and the reaction was heated at 70° C. for 3 days. The reaction was concentrated and the residue was purified on silica gel using a gradient of 0 to 50% EtOAc in hexanes to give tert-butyl (3R)-3-[(1S)-1-[[3-[3-(3-benzyloxyphenyl)-2-oxo-imidazolidin-1-yl]phenyl]methyl]-2-tert-butoxy-2-oxo-ethyl]pyrrolidine-1-carboxylate (345 mg, 27%), of which a portion (120 mg, 0.187 mmol) was diluted with DCM (6 mL) and cooled to 0° C. Boron tribromide (1 M solution in heptane, 0.56 mL, 0.56 mmol) was added dropwise and the reaction was stirred at 0° C. for 30 min. MeOH was added and the reaction was concentrated. The residue was purified by HPLC [column: Kinetex Evo C18 3×100 mm, 5 μm; mobile phase: solvent A=0.1% aqueous formic acid in water, solvent B=ACN, gradient from 5-38% solvent B in solvent A; column temperature: 50° C.] to give the title compound (42 mg, 51%) as a white solid. ES-MS m/z 396.2 (M+H)

### EXAMPLE 149

### 3-Amino-2-(biphenyl-3-ylmethyl)-2-[(3R)-pyrrolidin-3-yl]propanoic acid dihydrochloride (Mixture of Diastereomers)

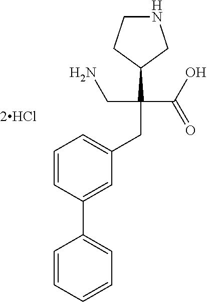 [SMILES: NCC(Cc1cccc(-c2ccccc2)c1)(C(=O)O)[C@H]1CCNC1]

A mixture of 2-[[2-[(3R)-1-tert-butoxycarbonylpyrrolidin-3-yl]-2-carboxy-3-(3-phenylphenyl)propyl]carbamoyl]benzoic acid (mixture of diastereomers, 28 mg, 0.049 mmol) and concentrated aqueous HCl (0.489 mL) was heated at 100° C. overnight. The mixture was cooled to RT and loaded onto SCX resin (previously washed with CH₃CN—H₂O 50%). The column was eluted with CH₃CN—H₂O 50%, then with NH₃ (2 M in MeOH, then 7 M in MeOH). The MeOH—NH₃ fraction was collected and concentrated under reduced pressure. The residue was dried under reduced pressure at 40° C. overnight, then treated with HCl (1 N aqueous solution, 0.462 mL, 0.462 mmol) for 1 h and then the solvent was eliminated under a stream of nitrogen. The residue was dried under reduced pressure at 40° C. overnight to give the title compound (9 mg, 73%) as pale brown solid. ES-MS m/z 325 (M+H).

### EXAMPLE 150

### (2S)-3-(3-Ethynylphenyl)-2-[(3R)-pyrrolidin-3-yl]propanoic acid hydrochloride

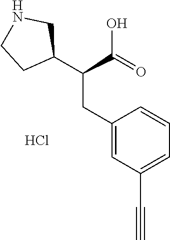 [SMILES: C#Cc1cccc(C[C@H](C(=O)O)[C@H]2CCNC2)c1 ; Cl]

To a mixture of tert-butyl (3R)-3-[(1S)-2-tert-butoxy-2-oxo-1-[[3-(2-trimethylsilylethynyl)phenyl]methyl]ethyl]pyrrolidine-1-carboxylate (40 mg, 0.085 mmol) in DCM (1 mL) was added TFA (2 mL) at 25° C., and the mixture was stirred at 0° C. for 16 h. The reaction mixture was concentrated to dryness and purified by HPLC [column: Welch Xtimate® C18 150×30 mm, 5 μm; mobile phase: solvent A=aqueous HCL, solvent B=ACN, gradient 0-100% solvent B in solvent A, flow rate: 25 mL/min] to give the title compound (5.18 mg, 24%) as a white solid. ES-MS m/z 244.0 (M+H).

### EXAMPLE 151

### (2S)-3-(3-Hydrazinylphenyl)-2-[(3R)-pyrrolidin-3-yl]propanoic acid hydrochloride

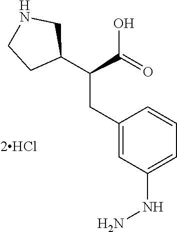 [SMILES: "2·HCl" ; NNc1cccc(C[C@H](C(=O)O)[C@H]2CCNC2)c1]

To a solution of tert-butyl (3R)-3-[(1S)-1-[(3-aminophenyl)methyl]-2-tert-butoxy-2-oxo-ethyl]pyrrolidine-1-carboxylate (prepared essentially as described in WO 2020/247429; 200 mg, 0.512 mmol) in water (9.6 mL) and concentrated HCl (3.2 mL) was added sodium nitrite (53 mg, 0.77 mmol) in water (2.4 mL) slowly at 0° C. The reaction mixture was stirred at 0° C. for 0.1 h, then stannous chloride (200 mg, 1.03 mmol) in HCl (1.2 mL) was added at 0° C., and the mixture was stirred under N₂ 0-25° C. for 16 h. The reaction mixture was purified by HPLC [column: Phenomenex® Gemini© NX C18 75×30 mm, 3 μm; mobile phase: solvent A=aqueous HCl, solvent B=ACN, gradient 0-100% solvent B in solvent A] to give the title compound (22 mg, 16%) as a white solid. ES-MS m/z 250.2 (M+H).

### EXAMPLE 152

### 3-{(2S)-2-Carboxy-2-[(3R)-pyrrolidin-3-yl]ethyl}benzoic acid hydrochloride

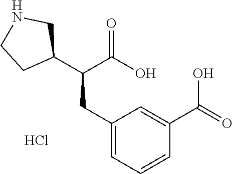 [SMILES: O=C(O)c1cccc(C[C@H](C(=O)O)[C@H]2CCNC2)c1 ; Cl]

To a solution of (2S)-3-[3-(methoxycarbonyl)phenyl]-2-[(3R)-pyrrolidin-3-yl]propanoic acid hydrochloride (120 mg, 0.351 mmol) in THE (1 mL) and water (1 mL) was added LiOH (37 mg, 1.47 mmol) at 25° C. The resulting mixture was stirred at 40° C. for 2 h and the reaction mixture was purified by HPLC [column: Phenomenex® Gemini® NX C18 75×30 mm, 3 μm; mobile phase: solvent A=aqueous HCl, solvent B=ACN, gradient 0-100% solvent B in solvent A] to give the title compound (16 mg, 18%) as a yellow oil. ES-MS m/z 264.1 (M+H).

### EXAMPLE 153

### (2S)-3-(3-Ethenylphenyl)-2-[(3R)-pyrrolidin-3-yl]propanoic acid ammonium salt

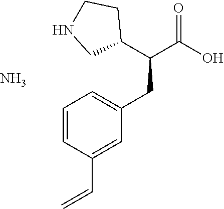

To a mixture of methyl (2S)-2-[(3R)-pyrrolidin-3-yl]-3-(3-vinylphenyl)propanoate (290 mg, 1.12 mmol) in THE (4 mL), MeOH (2 mL) and water (1 mL) was added LiOH (290 mg, 11.5 mmol) at 25° C. in one portion. The reaction mixture was stirred at 25° C. for 3 h, then the pH was adjusted to 7 by the addition of 1 N aqueous HCl. The mixture was concentrated to dryness under reduced pressure and the residue was purified by HPLC [column: Phenomenex® Gemini® NX C18 75×30 mm, 3 μm; mobile phase: solvent A=aqueous HCl, solvent B=ACN, gradient 0-100% solvent B in solvent A], and re-purified by HPLC [column: same; mobile phase: solvent A=aqueous NH₄OH+NH₄HCO₃, solvent B=ACN; gradient 0-100% solvent B in solvent A] to give the title compound (18.1 mg, 6%) as a white solid. ES-MS m/z 246.2 (M+H).

### Biological Assays

### In Vitro Apo(a) Binding Assay

The in vitro binding affinity of compounds to the intended target human Apo(a) protein were tested in a competitive binding assay. Human Apo(a) protein containing 17 Kringle repeats was affinity purified from conditioned media of transiently transfected HEK-293F cells. All reagents were prepared in assay buffer containing 50 mM Tris-HCl pH 7.4, 0.1% BSA. The binding assay was conducted by adding to each well of a clear-bottom plate 50 μL each of (1) test compound in dilution series (final concentration 0.32˜10000 nM), (2) Apo(a) protein (6 ng/well), (3) re-suspended Wheat Germ Agglutinin Polyvinyltoluene SPA beads (20 mg/mL), and (4) the radioligand, tritium-labeled (2S)-3-[3-[2-oxo-3-[3-(tritritiomethoxy)phenyl]imidazolidin-1-yl]phenyl]-2-[(3R)-pyrrolidin-3-yl]propanoic acid hydrochloride (prepared essentially as described in WO 2020/247429; final concentration 0.52 nM). Plates were incubated for 60 min at RT and counted on TRILUX LSC. Non-specific binding, defined as binding in the presence of 10 M cold (i.e. non-radiolabeled) ligand: (2S)-3-[3-[2-oxo-3-[3-(methoxy)phenyl]imidazolidin-1-yl]phenyl]-2-[(3R)-pyrrolidin-3-yl]propanoic acid hydrochloride (prepared essentially as described in WO 2020/247429) was subtracted to determine specific binding. Data were analyzed by fitting to a standard single site binding model and the IC₅₀ for the Example test compound was determined.

In this assay, the compound of Example 6 has an IC₅₀ of 4.68 nM (SEM 2.26 nM, n=2), indicating that it binds to human Apo(a) protein. Inhibition of the assembly of the LDL particle with apo(a) through binding to the Apo(a) protein supports a reduction in Lp(a) levels.

### In Vitro Lp(a) Assembly Assay

The ability of compounds to inhibit the formation of Lp(a) particles in vitro was assessed by a cell-free assembly assay. Conditioned media (DMEM supplemented with 10% FBS, 20 mM HEPES, and 1× penicillin/streptomycin) was collected from confluent wild-type HepG2 cells (a source of endogenously expressed ApoB) and from a HEK293 stable cell line expressing human Apo(a) protein containing 17 Kringle repeats (selected on 1 mg/ml geneticin) after 24 h of culture at 37° C. and 5% CO₂. An in vitro assembly assay was conducted by combining equal parts of HepG2 and HEK293 conditioned media with the test compounds added in dilution series (final concentration 0.01˜100 nM). The reaction was incubated at 37° C. for 2 hrs and then stopped with the addition of 6-aminocaproic acid (EACA) to a final concentration of 150 mM. Lp(a) was detected using a sandwich ELISA with an anti-Lp(a) capture antibody and an HRP-conjugated anti-ApoB detection antibody. The ELISA was developed using TMB, stopped using 1 N sulfuric acid, and the signal was read at 450 nm on a Molecular Devices plate reader. The % inhibition of Lp(a) formed for each test condition was determined with an assembly reaction having no inhibitor present (with matched DMSO concentration at 1%) set to 0% inhibition, and an assembly reaction with a minimal amount of the HepG2 conditioned media present (50-fold dilution) set to 100% inhibition. Data were fitted to a 4-parameter curve to determine the IC₅₀ values summarized in Table 1. Addition of the Example test compound to conditioned media containing ApoB and Apo(a) lead to concentration-dependent inhibition of Lp(a) formation in vitro, as summarized in Table 1. The results indicate that these compounds inhibit the assembly of Lp(a) from Apo(a) and the LDL particle.

TABLE 1

| Example | IC₅₀ nM (SEM, n) | |
|---|---|---|
| 1a | 103 | (23.6, n = 3) |
| 2b | 695 | (255, n = 2) |
| 3 | 40.8 | (5.93, n = 4) |
| 4 | 18.8 | (3.79, n = 14) |
| 5a | 5630 | (69.6, n = 2) |
| 5b | 1260 | (355, n = 4) |
| 6 | 83.3 | (11.9, n = 4) |
| 7 | 107 | (22.8, n = 4) |
| 8 | 76 | (13.1, n = 4) |
| 9 | 169 | (12.8, n = 4) |
| 10 | 237 | (52.9, n = 4) |
| 11 | 55.8 | (10.6, n = 4) |
| 12 | 171 | (64.1, n = 4) |
| 13 | 211 | (33, n = 4) |
| 14 | 30.4 | (6.04, n = 4) |
| 15 | 11 | (7.31, n = 2) |
| 16 | 24.9 | (7.93, n = 4) |
| 17 | 22.2 | (0, n = 2) |
| 18 | 4.1 | (0, n = 2) |
| 19 | 115 | (0.25, n = 2) |
| 20 | 16 | (0.185, n = 3) |
| 21 | 17.4 | (0, n = 2) |
| 22 | 31.2 | (0, n = 2) |
| 23 | 102 | (0.4, n = 2) |
| 24 | 41.3 | (0, n = 2) |
| 25 | 21.5 | (0.2, n = 2) |
| 26 | 226 | (10.6, n = 2) |
| 27 | 81.5 | (60.7, n = 6) |
| 28 | 388 | (93, n = 2) |
| 29 | 83.7 | (4.82, n = 4) |
| 30 | 146 | (17, n = 4) |
| 31 | 100 | (19.7, n = 4) |
| 32 | 200 | (46.7, n = 4) |
| 33 | 116 | (21.9, n = 4) |
| 34 | 59.8 | (3.33, n = 2) |
| 35 | 187 | (36.4, n = 4) |
| 36 | 7.45 | (2.32, n = 6) |
| 37 | 16.4 | (0.00726, n = 2) |
| 38 | 684 | (149, n = 4) |
| 39 | 0.184 | (0.0794, n = 8) |
| 40 | 0.861 | (0.767, n = 4) |
| 41 | 0.536 | (0.0845, n = 2) |
| 42 | 0.323 | (0.0804, n = 2) |
| 43 | <1.52 | (n = 1) |
| 44 | 0.809 | (0.0004519, n = 2) |
| 45 | 0.0883 | (0.017, n = 4) |
| 46 | 0.206 | (0.144, n = 5) |
| 47 | 0.427 | (0.0639, n = 4) |
| 48 | 0.629 | (0.235, n = 2) |
| 49 | <1.52 | (n = 1) |
| 50 | 23.8 | (2.09, n = 4) |
| 51 | 0.294 | (0.162, n = 4) |
| 52 | 0.193 | (0.109, n = 4) |
| 53 | 0.247 | (0.0552, n = 4) |
| 54 | 0.072 | (0.00632, n = 2) |
| 55 | 21.6 | (15.7, n = 4) |
| 56 | 4170 | (484, n = 3) |
| 57 | 36.6 | (15.1, n = 4) |
| 58 | 6300 | (2390, n = 2) |
| 59 | 34 | (21.8, n = 4) |
| 60 | 1.1 | (0.089, n = 2) |
| 61 | 22.2 | (8.51, n = 4) |
| 62 | 1.16 | (0.361, n = 2) |
| 63 | 0.313 | (0.0364, n = 2) |
| 64 | 34.2 | (2.9, n = 3) |
| 65 | 3450 | (278, n = 2) |
| 66 | 0.94 | (0.118, n = 2) |
| 67 | 0.074 | (0.0152, n = 9) |
| 68 | 0.0889 | (0.00844, n = 2) |
| 69 | <1.52 | (n = 1) |
| 70 | <1.52 | (n = 1) |
| 71 | 94.6 | (27.5, n = 3) |
| 72 | 110 | (n = 1) |
| 73 | 649 | (97.7, n = 2) |
| 74 | 42.3 | (22.7, n = 6) |
| 76 | 1070 | (150, n = 2) |
| 77 | 101 | (2.03, n = 2) |
| 78 | 25.6 | (8.35, n = 4) |
| 79 | 16.8 | (13.1, n = 6) |
| 80 | 2.03 | (0.458, n = 4) |
| 81 | 22.5 | (6.26, n = 4) |
| 82 | 29.6 | (7.08, n = 2) |
| 83 | 62.9 | (n = 1) |
| 84 | 7.86 | (5.27, n = 4) |
| 85 | 41.2 | (4.25, n = 4) |
| 86 | 17 | (1.66, n = 8) |
| 87 | 18.2 | (6.17, n = 10) |
| 88 | 58 | (22.9, n = 2) |
| 89 | 69.6 | (15.1, n = 2) |
| 90 | 78.1 | (n = 1) |
| 91 | 30.4 | (5, n = 3) |
| 92 | 92.9 | (n = 1) |
| 93 | 37.7 | (n = 1) |
| 94 | 17.9 | (6.53, n = 4) |
| 95 | 34.4 | (n = 1) |
| 96 | 195 | (n = 1) |
| 97 | 47.7 | (n = 1) |
| 98 | 45.7 | (n = 1) |
| 99 | 81.5 | (26.3, n = 4) |
| 100 | 219 | (38.1, n = 4) |
| 101 | 12.6 | (5.19, n = 4) |
| 102 | 356 | (23.3, n = 3) |
| 103 | 473 | (277, n = 2) |
| 104 | 16.3 | (2.58, n = 4) |
| 105 | 164 | (n = 1) |
| 106 | 228 | (n = 1) |
| 107 | 658 | (n = 1) |
| 108 | 1.86 | (1.03, n = 4) |
| 109 | 33.7 | (4.79, n = 4) |
| 110 | 41.4 | (10.1, n = 4) |
| 111 | 46.4 | (14.9, n = 4) |
| 112 | 6.3 | (3.56, n = 4) |
| 113 | 9.23 | (2.46, n = 4) |
| 114 | 18.3 | (0.852, n = 2) |
| 115 | 5.12 | (0.479, n = 2) |
| 116 | 3770 | (n = 1) |
| 117 | 4700 | (1560, n = 2) |
| 118 | 6890 | (n = 1) |
| 119 | 4940 | (518, n = 2) |
| 120 | 1340 | (31.4, n = 2) |
| 121 | 1360 | (22.7, n = 2) |
| 122 | 1340 | (91.4, n = 2) |
| 123 | 1940 | (169, n = 2) |
| 124 | 60 | (137, n = 3) |
| 125 | 84.7 | (8.98, n = 3) |
| 126 | 376 | (18.7, n = 3) |
| 127 | 179 | (9.67, n = 3) |
| 128 | 1430 | (201, n = 2) |
| 129 | 24.8 | (3.5, n = 3) |
| 130 | 282 | (28.3, n = 3) |
| 131 | 591 | (96.8, n = 3) |
| 132 | 729 | (126, n = 3) |
| 133 | 337 | (28.5, n = 2) |
| 134 | 1810 | (450, n = 2) |
| 135 | 556 | (19.2, n = 3) |
| 136 | 0.0848 | (0.0211, n = 3) |
| 137 | 0.118 | (0.0196, n = 6) |
| 138 | 0.273 | (0.0661, n = 3) |
| 139 | 0.106 | (0.0178, n = 6) |
| 140 | 0.14 | (0.0151, n = 3) |
| 141 | 0.298 | (0.0534, n = 5) |
| 142 | 0.194 | (0.0172, n = 3) |
| 143 | 15.6 | (9.13, n = 3) |
| 144 | 0.0364 | (0.00214, n = 3) |
| 145 | 0.0988 | (0.00873, n = 6) |
| 146 | 44.3 | (19.9, n = 4) |
| 147 | 4.83 | (1.07, n = 4) |
| 148 | 28.1 | (2.99, n = 2) |
| 149 | 1140 | (n = 1) |
| 150 | 22.5 | (1.77, n = 3) |
| 151 | 4.56 | (0.671, n = 3) |
| 152 | 319 | (44.1, n = 3) |
| 153 | 10.2 | (2.54, n = 3) |
| 154 | 31.4 | (n = 1) |

1. A compound of formula: 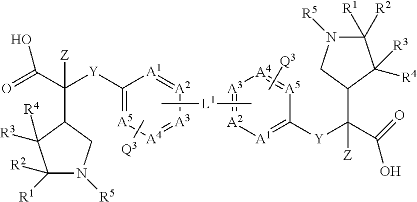
wherein
L¹ is attached at A¹, A² or A³ and is selected from a bond, —(CH₂)ₚNHC(O)NH(CH₂)ₚ—, —(CH₂)ₚC(O)NH(CH₂)ₚ—, —(CH₂)ₚS(O)₂NH(CH₂)ₚ—, —(CH₂)_{q}—, —(CH₂)ₚNH(CH₂)ₚ—, 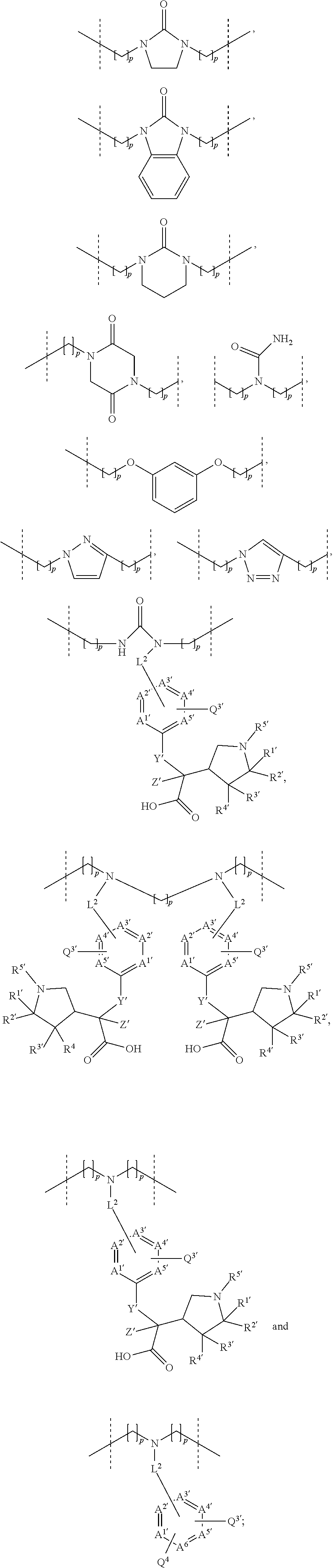
or L¹ together with the carbons at positions A² and A³ on one ring form the fused ring: 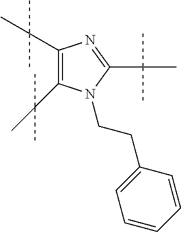
R¹, R^{1′}, R² and R^{2′} at each occurrence are independently selected from H, C₁₋₄ alkyl and F;
R³, R^{3′}, R⁴ and R^{4′} at each occurrence are independently either H or F;
R⁵ and R^{5′} at each occurrence are independently H, C₁₋₄ alkyl, or cyclopropyl;
Z and Z′ at each occurrence are independently H, C₁₋₄ alkyl, OH, cyclopropyl, CH₂OH, CH₂NH₂ or CH₂OCH₂phenyl;
Y and Y′ at each occurrence are independently CH₂, CH(CH₃), O or S;
A¹, A^{1′}, A², A^{2′}, A³, A^{3′}, A⁴, A^{4′}, A⁵, A^{5′} and A⁶ at each occurrence are independently C or N, wherein no more than two of A¹, A², A³, A⁴ and A⁵ on each ring are N or no more than two of A¹, A^{2′}, A^{3′}, A^{4′}, A^{5′} and A⁶ on each ring are N;
Q³ and Q^{3′} at each occurrence are independently H; —(CH₂)ₙO(CH₂)ₙR¹⁰; —(CH₂)ₙNR¹⁵(CH₂)ₙR¹⁰; —CN; —(CH₂)ₙCO₂R¹⁰; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; NO₂; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or 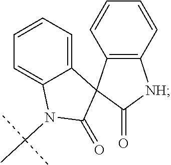
Q⁴ is R¹¹, CF₃, O—R¹¹, OCF₃, halo, or CN;
m at each occurrence is independently 0, 1, or 2;
n at each occurrence is independently 0, 1, 2, or 3;
R¹⁰ at each occurrence is independently H; halo; OH; carboxyl; —S(O)₂OH; C₁₋₄ alkyl optionally substituted with one to four OH or with OCH₃; C₃₋₆ cycloalkyl optionally substituted with one or two halo; C₁₋₄ haloalkyl; —C₂₋₆ alkynyl;1-benzyl-4-piperidyl; 2-tert-butoxy-2-oxo-ethyl; benzyloxyphenyl optionally substituted with one or two halo; O(C₁₋₂alkyl)ᵣOCH₃; NH₂; 2,3-dihydro-1H-indene; 2,3-dihydrobenzo[b][1,4]dioxine; benzo[d][1,3]dioxole optionally substituted with one or two halo; indoline optionally substituted with C(O)CH₃; 5- or 6-membered heteroaryl or 9- or 10-membered bicyclic heteroaryl optionally substituted with C₁₋₄ alkyl, C₁₋₄ alkoxy, halo or phenyl; phenyl optionally substituted with one to three substituents independently selected from: halo, C₁₋₄ alkoxy, hydroxy, C₁₋₄ alkyl, CF₃, CN, —(CH₂)₂C(O)OH, —C(O)NHNH₂, —OCF₃, —N(CH₃)₂, C₃₋₆ cycloalkyl, —OC₃₋₆ cycloalkyl, —OCH₂C₃₋₆ cycloalkyl, 5- or 6-membered heteroaryl, —(CH₂)ₙ(5- or 6-membered heterocyclyl) or —(CH₂)ₙphenyl wherein the phenyl is optionally substituted one or two halo;
R¹¹ is H, C₁₋₄ alkyl, or cyclopropyl;
L² is attached at A^{1′}, A^{2′} or A^{3′} and is C₁₋₃ alkylene or a bond; and
p at each occurrence is independently 0 to 3;
q is 1 to 5;
r is 1, 2, or 3;
R¹¹ is H or C₁₋₃ alkyl;
or a pharmaceutically acceptable salt thereof,
wherein the compound is not of the formula: 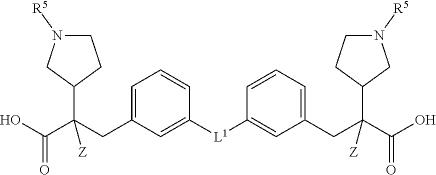
wherein
L¹ is selected from the group consisting of —CH₂NHCH₂—, —CH₂NH—, —NH—, 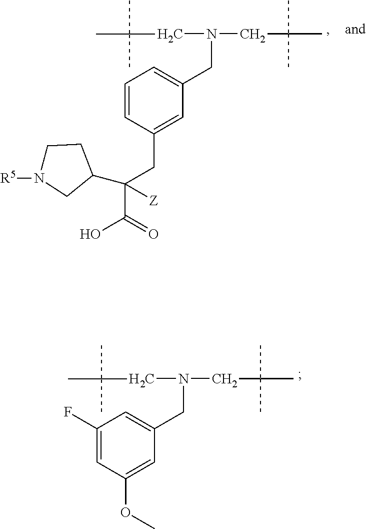
R⁵ is H or CH₃; and
Z is H or CH₃.

2. The compound according to claim 1 wherein L¹ is selected from a bond, —(CH₂)ₚNHC(O)NH(CH₂)ₚ—, —(CH₂)ₚC(O)NH(CH₂)ₚ—, —(CH₂)ₚS(O)₂NH(CH₂)ₚ—, —(CH₂)_{q}—, 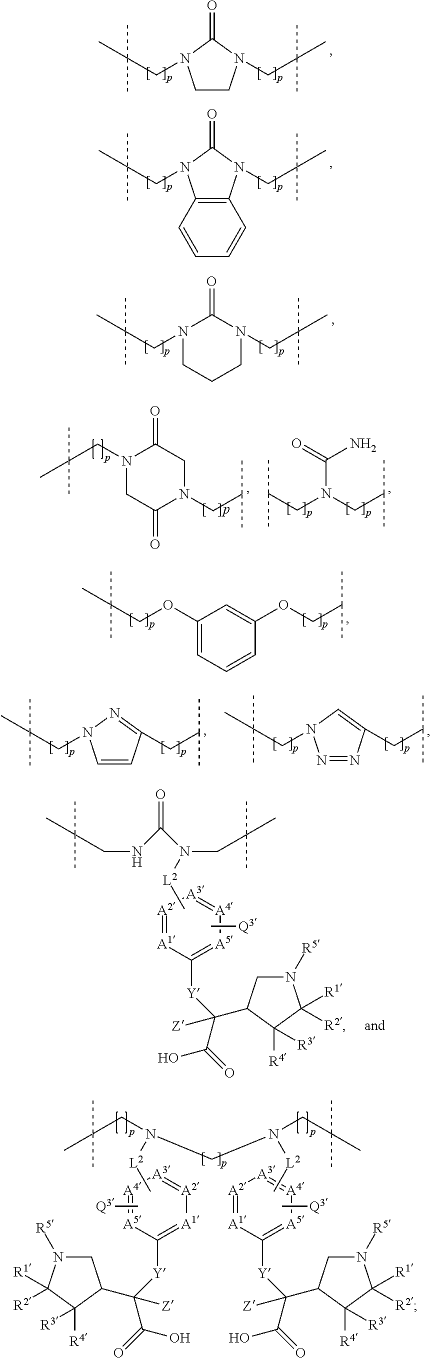
or L¹ together with the carbons at positions A² and A³ on one ring form the fused ring: 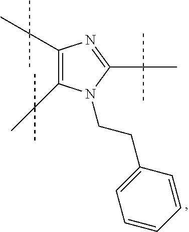
or a pharmaceutically acceptable salt thereof.

3. The compound according to claim 1 wherein L¹ is —(CH₂)ₚNH(CH₂)ₚ—, 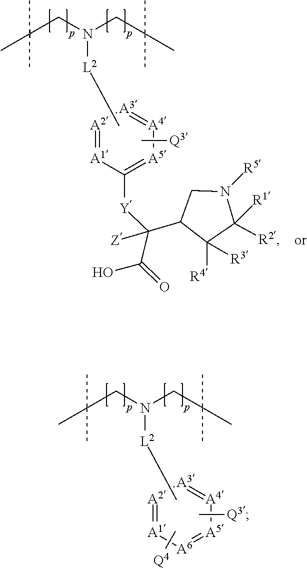
and at least one Q³ is other than H, or a pharmaceutically acceptable salt thereof.

4. The compound according to claim 1 wherein L¹ is attached at A² on each ring; at A² on one ring and A³ on the other; at A³ on each ring; or at A¹ on one ring and A² on the other, or a pharmaceutically acceptable salt thereof.

5. The compound according to claim 4 wherein L¹ is attached at A² on each ring, or a pharmaceutically acceptable salt thereof.

6. The compound according to claim 1 wherein L¹ is selected from: a bond, —(CH₂)ₚNHC(O)NH(CH₂)ₚ—, —(CH₂)ₚC(O)NH(CH₂)ₚ—, —(CH₂)ₚS(O)₂NH(CH₂)ₚ—, —(CH₂)_{q}—, —(CH₂)ₚNH(CH₂)ₚ—, 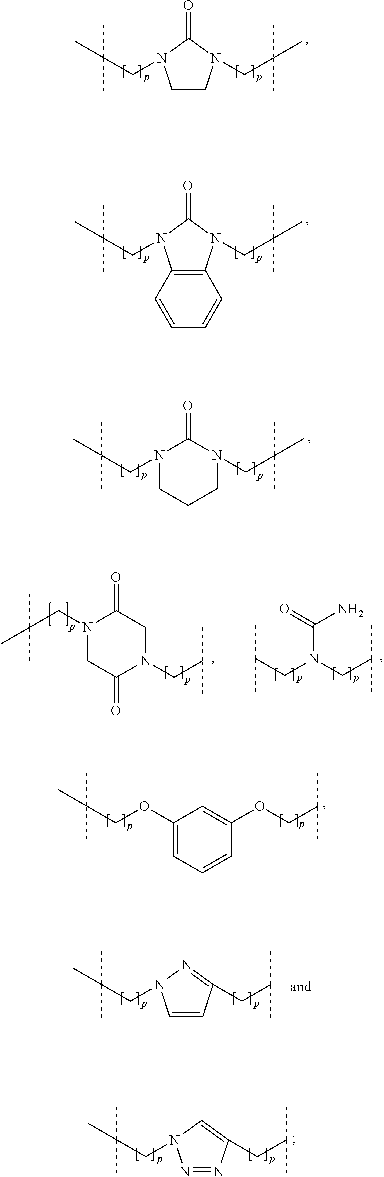
or L¹ together with the carbons at positions A² and A³ on one ring form the fused ring: 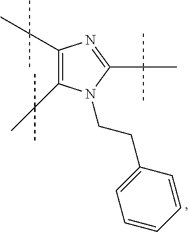
or a pharmaceutically acceptable salt thereof.

7. The compound according to claim 6 wherein L¹ is selected from: a bond, —NHC(O)NH—, —NHC(O)NHCH₂—, —C(O)NH—, —S(O)₂NH—, —CH₂CH₂CH₂—, —NH—, 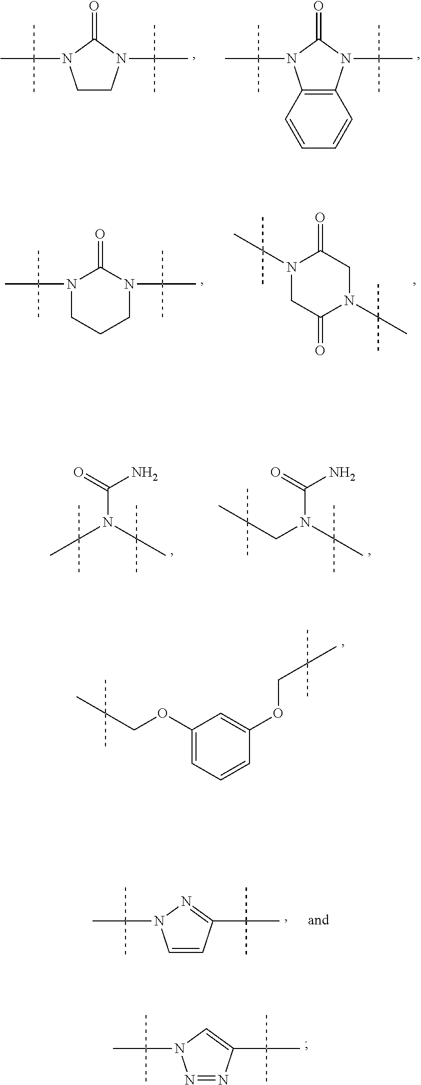
L¹ together with the carbons at positions A² and A³ on one ring form the fused ring: 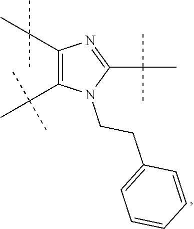
or a pharmaceutically acceptable salt thereof.

8. The compound according to claim 1 wherein L¹ is selected from: 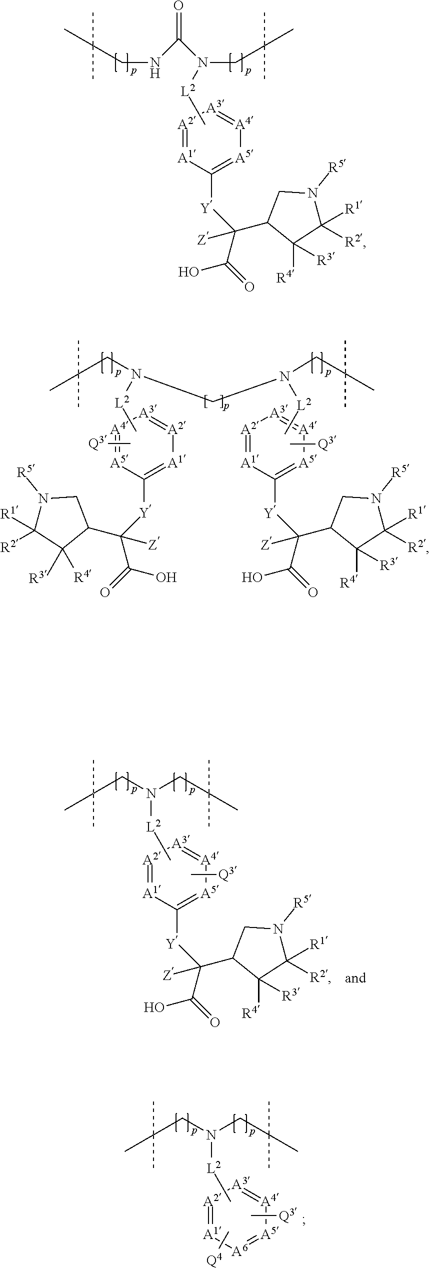
or a pharmaceutically acceptable salt thereof.

9. The compound according to claim 8 wherein L¹ is selected from: 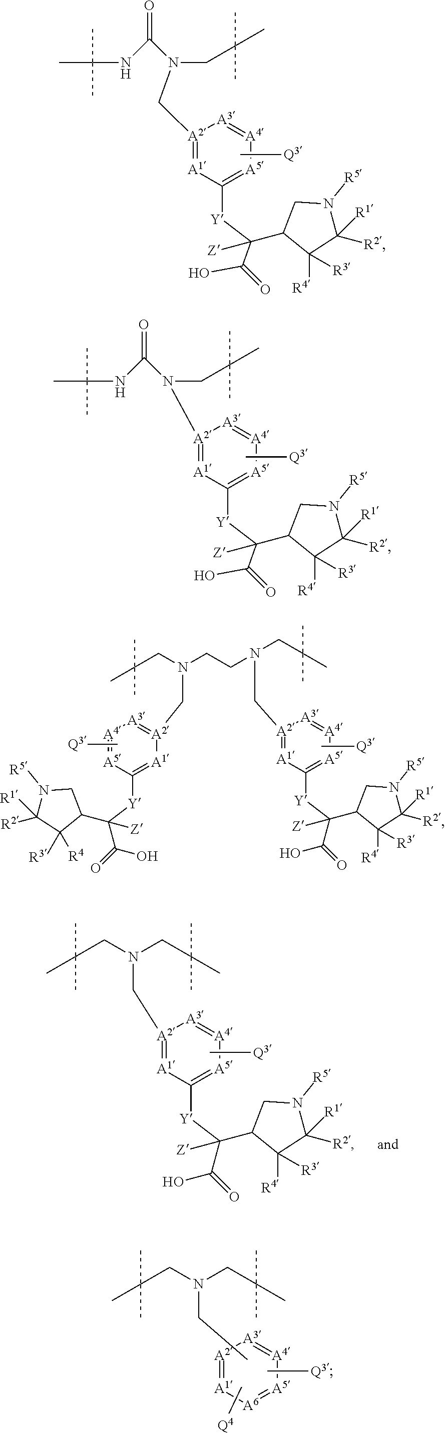
or a pharmaceutically acceptable salt thereof.

10. The compound according to claim 9 wherein L¹ is selected from: 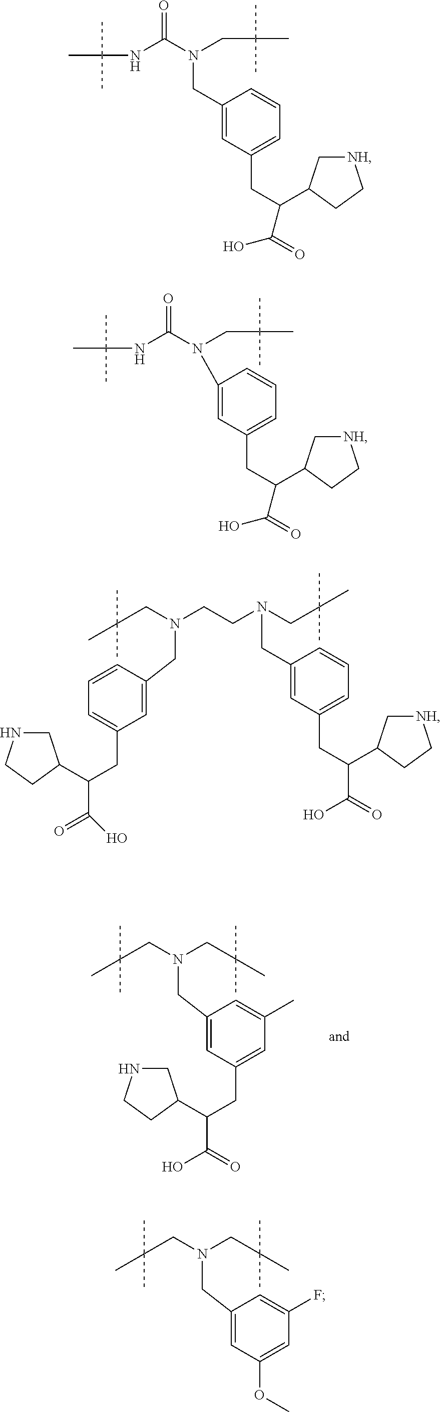
or a pharmaceutically acceptable salt thereof.

11. The compound according to claim 1 wherein on each ring R¹ and R² are both H or one of R¹ and R² is H and the other is CH₃, or a pharmaceutically acceptable salt thereof.

12. The compound according to claim 1 wherein on each ring R³ and R⁴ are both H or R³ and R⁴ are both F, or a pharmaceutically acceptable salt thereof.

13. The compound according to claim 1 wherein at each occurrence R⁵ is H, or a pharmaceutically acceptable salt thereof.

14. The compound according to claim 1 wherein at each occurrence Z is H, CH₃ or OH, or a pharmaceutically acceptable salt thereof.

15. The compound according to claim 1 wherein on each ring A¹, A², A³, A⁴ and A⁵ are all C, or a pharmaceutically acceptable salt thereof.

16. The compound according to claim 1 wherein at each occurrence Q³ is H, F, CF₃ or CN, or a pharmaceutically acceptable salt thereof.

17. A compound of formula: 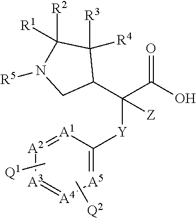
wherein
R¹ and R² are independently selected from H, C₁₋₄ alkyl, and F;
R³ and R⁴ are independently either H or F;
R⁵ is H, C₁₋₄ alkyl, or cyclopropyl;
Z is H, C₁₋₄ alkyl, OH, cyclopropyl, CH₂OH, CH₂NH₂ or CH₂OCH₂phenyl;
Y is CH₂, CH(CH₃), O or S;
A¹, A², A³, A⁴, and A⁵ are each independently C or N, wherein no more than two of A¹, A², A³, A⁴, or A⁵ are N;
Q¹ is —(CH₂)ₙO(CH₂)ₙR¹⁰; —(CH₂)ₙNR¹⁵(CH₂)ₙR¹⁰; —CN; —(CH₂)ₙCO₂R¹⁰; —B(OR¹⁰)₂; a boronic acid ethylene glycol ester; a boronic acid pinacol ester; a boronic acid propylene-1,3-diol ester; a boronic acid 2,2-dimethyl-propylene-1,3-diol ester; —N₃; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; NO₂; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or 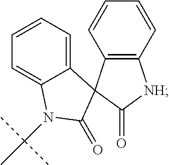
Q² is R¹¹, CF₃, O—R¹¹, OCF₃, halo, or CN;
m is 0, 1, or 2;
n is 0, 1, 2, or 3;
R¹⁰ is H; halo; OH; carboxyl; —S(O)₂OH; C₁₋₄ alkyl optionally substituted with one to four OH or with OCH₃; C₃₋₆ cycloalkyl optionally substituted with one or two halo; C₁₋₄ haloalkyl; —C₂₋₆ alkynyl; 1-benzyl-4-piperidyl; 2-tert-butoxy-2-oxo-ethyl; benzyloxyphenyl optionally substituted with one or two halo; O(C₁₋₂alkyl)ᵣOCH₃; NH₂; 2,3-dihydro-1H-indene; 2,3-dihydrobenzo[b][1,4]dioxine; benzo[d][1,3]dioxole optionally substituted with one or two halo; indoline optionally substituted with C(O)CH₃; 5- or 6-membered heteroaryl or 9- or 10-membered bicyclic heteroaryl optionally substituted with C₁₋₄ alkyl, C₁₋₄ alkoxy, halo or phenyl; phenyl optionally substituted with one to three substituents independently selected from: halo, C₁₋₄ alkoxy, hydroxy, C₁₋₄ alkyl, CF₃, CN, —(CH₂)₂C(O)OH, —C(O)NHNH₂, —OCF₃, —N(CH₃)₂, C₃₋₆ cycloalkyl, —OC₃₋₆ cycloalkyl, —OCH₂C₃₋₆ cycloalkyl, 5- or 6-membered heteroaryl, —(CH₂)ₙ(5- or 6-membered heterocyclyl) or —(CH₂)ₙphenyl wherein the phenyl is optionally substituted one or two halo;
R¹¹ is H, C₁₋₄ alkyl, or cyclopropyl;
r is 1, 2 or 3; and
R¹⁵ is H or C₁₋₃alkyl;
or a pharmaceutically acceptable salt thereof.

18. The compound according to claim 17 which is of the formula: 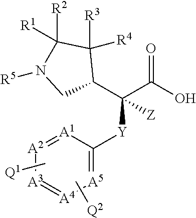
or a pharmaceutically acceptable salt thereof.

19. The compound according to claim 17 selected from: 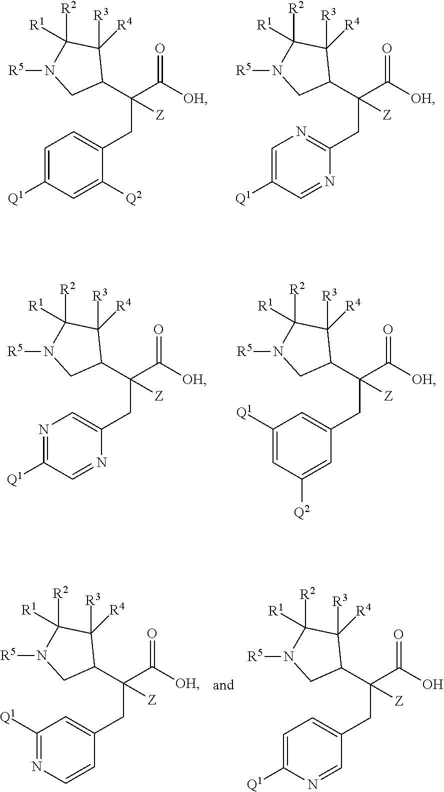
or a pharmaceutically acceptable salt thereof.

20. The compound according to claim 17 wherein Q¹ is selected from: —(CH₂)ₙO(CH₂)ₙR¹⁰; —(CH₂)ₙNR¹⁵(CH₂)ₙR¹⁰; —CN; —(CH₂)ₙCO₂R¹⁰; —N₃; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; CF₃; C₃₋₆cycloalkyl; —NH(C═NH)CH₂CN; 5- or 6-membered heterocyclyl optionally substituted with one to four halo or with a phenyl; indoline optionally substituted with one or two CH₃; imidazolidinone or imidazolidin-2,5-dione optionally substituted with (CH₂)CF₃, or (CH₂)ₙphenyl, which phenyl is optionally substituted with OH; indolin-2-one or benzimidazol-2-one optionally substituted with CH₃; phenyl, 5- or 6-membered heteroaryl or 9-membered bicyclic heteroaryl wherein phenyl and heteroaryl are optionally substituted with one or two substituents independently selected from halo, OC₁₋₄haloalkyl, C₃₋₆cycloalkyl, —OC₃₋₆cycloalkyl, C₁₋₄alkoxy, C₁₋₆alkyl optionally substituted with OH or halo, phenyl or benzyl which phenyl or benzyl is optionally substituted with one to three substituents independently selected from —OCH₃ and halo; or 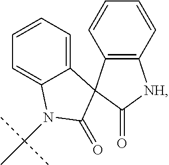
or a pharmaceutically acceptable salt thereof.

21. The compound according to claim 17, wherein Q² is H, CH₃, F or Br, or a pharmaceutically acceptable salt thereof.

22. The compound according to claim 17, wherein R⁵ is H, or a pharmaceutically acceptable salt thereof.

23. The compound according to claim 17, wherein R¹, R², R³, and R⁴ are all H, or a pharmaceutically acceptable salt thereof.

24. The compound according to claim 23, wherein R¹ and R² are H and R³ and R⁴ are F, or a pharmaceutically acceptable salt thereof.

25. The compound according to claim 17, wherein Z is selected from H, methyl, CH₂OH, CH₂NH₂ and CH₂OCH₂phenyl, or a pharmaceutically acceptable salt thereof.

26. A compound of the formula: 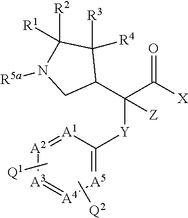
wherein
R¹ and R² are independently selected from H, C₁₋₄ alkyl, and F;
R³ and R⁴ are independently either H or F;
R^{5a} is H, C₁₋₄ alkyl, cyclopropyl, or a protecting group;
X is OH, —OR⁶, or 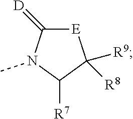
R⁶ is C₁₋₄ alkyl;
D and E are each independently O or S;
R⁷ is H, C₁₋₄ alkyl, phenyl or benzyl, wherein the phenyl and benzyl are optionally substituted with one or two substituents independently selected from: halo, C₁₋₄ alkyl, trifluoromethyl, amino, C₁₋₄ alkylamino, and di-C₁₋₄ alkylamino;
R⁸ and R⁹ are each independently H, C₁₋₄ alkyl or phenyl optionally substituted with one or two substituents independently selected from: halo, C₁₋₄ alkyl, trifluoromethyl, amino, C₁₋₄ alkylamino and di-C₁₋₄ alkylamino;
Z is H, C₁₋₄ alkyl, OH, cyclopropyl, CH₂OH, CH₂NH₂ or CH₂OCH₂phenyl;
Y is CH₂, CH(CH₃), O or S;
A¹, A², A³, A⁴, and A⁵ are each independently C or N, wherein no more than two of A¹, A², A³, A⁴, or A⁵ are N;
Q¹ is —(CH₂)ₙO(CH₂)ₙR¹⁰; —(CH₂)ₙNR¹⁵(CH₂)ₙR¹⁰; —CN; —(CH₂)ₙCO₂R¹⁰; —B(OR¹⁰)₂; a boronic acid ethylene glycol ester; a boronic acid pinacol ester; a boronic acid propylene-1,3-diol ester; a boronic acid 2,2-dimethyl-propylene-1,3-diol ester; —N₃; C₁₋₆ alkyl; —C₂₋₆ alkenyl; —C₂₋₆ alkynyl; halo; —C(O)—R¹⁰; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; NO₂; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or 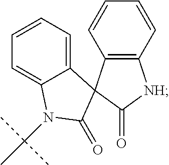
Q² is R¹¹, CF₃, O—R¹¹, OCF₃, halo, or CN;
m is 0, 1, or 2;
n is 0, 1, 2, or 3;
R¹⁰ is H; halo; OH; carboxyl; —S(O)₂OH; C₁₋₄ alkyl optionally substituted with one to four OH or with OCH₃; C₃₋₆ cycloalkyl optionally substituted with one or two halo; C₁₋₄ haloalkyl; —C₂₋₆ alkynyl; 1-benzyl-4-piperidyl; 2-tert-butoxy-2-oxo-ethyl; benzyloxyphenyl optionally substituted with one or two halo; O(C₁₋₂alkyl)ᵣOCH₃; NH₂; 2,3-dihydro-1H-indene; 2,3-dihydrobenzo[b][1,4]dioxine; benzo[d][1,3]dioxole optionally substituted with one or two halo; indoline optionally substituted with C(O)CH₃; 5- or 6-membered heteroaryl or 9- or 10-membered bicyclic heteroaryl optionally substituted with C₁₋₄ alkyl, C₁₋₄ alkoxy, halo or phenyl; phenyl optionally substituted with one to three substituents independently selected from: halo, C₁₋₄ alkoxy, hydroxy, C₁₋₄ alkyl, CF₃, CN, —(CH₂)₂C(O)OH, —C(O)NHNH₂, —OCF₃, —N(CH₃)₂, C₃₋₆ cycloalkyl, —OC₃₋₆ cycloalkyl, —OCH₂C₃₋₆ cycloalkyl, 5- or 6-membered heteroaryl, —(CH₂)ₙ(5- or 6-membered heterocyclyl) or —(CH₂)ₙphenyl wherein the phenyl is optionally substituted one or two halo; R¹¹ is H, C₁₋₄ alkyl, or cyclopropyl;
r is 1, 2 or 3; and
R¹⁵ is H or C₁₋₃ alkyl;
or a salt thereof,
wherein if X is OH then R^{5a} must be a protecting group; and
wherein the compound is not:
tert-butyl-3-[1-[(3-bromophenyl)methyl]-2-methoxy-2-oxo-ethyl]pyrrolidine-1-carboxylate;
3-(3-bromophenyl)-2-[1-tert-butoxycarbonylpyrrolidin-3-yl]-2-methyl-propanoic acid;
tert-butyl-3-[2-[4-benzyl-2-oxo-oxazolidin-3-yl]-1-[(3-bromophenyl)methyl]-2-oxo-ethyl]pyrrolidine-1-carboxylate;
tert-butyl-3-[(2-[4-benzyl-2-oxo-oxazolidin-3-yl]-1-[(3-nitrophenyl)methyl]-2-oxo-ethyl]pyrrolidine-1-carboxylate;
3-(3-bromophenyl)-2-[1-tert-butoxycarbonylpyrrolidin-3-yl]propanoic acid;
2-[(1-tert-butoxycarbonylpyrrolidin-3-yl]-3-(3-nitrophenyl) propanoic acid;
tert-butyl-3-[1-[(3-bromophenyl)methyl]-2-tert-butoxy-2-oxo-ethyl]pyrrolidine-1-carboxylate;
tert-butyl-3-[1-[(3-bromophenyl)methyl]-2-tert-butoxy-1-methyl-2-oxo-ethyl]pyrrolidine-1-carboxylate;
tert-butyl-3-[(2-tert-butoxy-1-[(3-nitrophenyl)methyl]-2-oxo-ethyl]pyrrolidine-1-carboxylate;
tert-butyl-3-[2-tert-butoxy-1-[(3-formylphenyl)methyl]-2-oxo-ethyl]pyrrolidine-1-carboxylate;
tert-butyl-3-[2-tert-butoxy-1-[(3-formylphenyl)methyl]-1-methyl-2-oxo-ethyl]pyrrolidine-1-carboxylate;
tert-butyl-3-[1-[[3-(aminomethyl)phenyl]methyl]-2-tert-butoxy-2-oxo-ethyl]pyrrolidine-1-carboxylate;
tert-butyl-3-[1-[(3-aminophenyl)methyl]-2-tert-butoxy-2-oxo-ethyl]pyrrolidine-1-carboxylate;
tert-butyl-3-[2-tert-butoxy-1-[[3-(hydroxymethyl)phenyl]methyl]-2-oxo-ethyl]pyrrolidine-1-carboxylate;
[3-[3-tert-butoxy-2-[1-tert-butoxycarbonylpyrrolidin-3-yl]-3-oxo-propyl]phenyl]boronic acid;
tert-butyl-3-[2-tert-butoxy-1-[(3-hydroxyphenyl)methyl]-2-oxo-ethyl]pyrrolidine-1-carboxylate.

27. The compound according to claim 26 wherein Q¹ is —CN; —(CH₂)ₙCO₂R¹⁰; a boronic acid ethylene glycol ester; a boronic acid pinacol ester; a boronic acid propylene-1,3-diol ester; a boronic acid 2,2-dimethyl-propylene-1,3-diol ester; —N₃; C₁₋₆ alkyl; —C₂₋₆ alkenyl; —C₂₋₆ alkynyl; —C(O)NR¹⁵R¹⁰; —S(O)ₘ(CH₂)ₙR¹⁰; —(CH₂)ₙNR¹⁵S(O)ₘ(CH₂)ₙR¹⁰; —(CH₂)ₙS(O)ₘNR¹⁵(CH₂)ₙR¹⁰; —(CH₂)ₙNHCONR¹⁵R¹⁰; —NHCO(CH₂)ₙR¹⁰; —NHCOOR¹⁰; CF₃; C₃₋₆ cycloalkyl; —NH(C═NH)CH₂CN; 5- or 6-membered heterocyclyl optionally substituted with one to four halo or with a phenyl; indoline optionally substituted with one or two CH₃; imidazolidinone, pyrrolidinone, imidazolidin-2,5-dione, pyrrolidin-2,5-dione or oxazolidin-2-one optionally substituted with (CH₂)CF₃, CH₃, or (CH₂)ₙphenyl, which phenyl is optionally substituted with OH or OCH₃; indolin-2-one, isoindolin-1-one or benzimidazol-2-one optionally substituted with one or two substituents independently selected from: OCH₃, CH₃ and halo; phenyl, 5- or 6-membered heteroaryl or 9- or 10-membered bicyclic heteroaryl wherein phenyl and heteroaryl are optionally substituted with one or two substituents independently selected from halo, OC₁₋₄ haloalkyl, C₃₋₆cycloalkyl, —OC₃₋₆ cycloalkyl, OH, C₁₋₄ alkoxy, C₁₋₆ alkyl optionally substituted with OH or one to four halo, NH₂, C₁₋₆ alkylCOOC₁₋₂alkyl, phenyl or benzyl which phenyl or benzyl is optionally substituted with one to three substituents independently selected from —OCH₃, CF₃, CH₃, CN and halo, or pyridine optionally substituted with CH₂OH; or 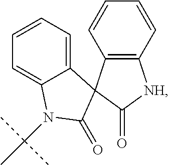
or a salt thereof.

28. The compound according to claim 26 wherein R^{5a} is a protecting group and the protecting group is selected from: tert-butyloxycarbonyl, carboxybenzyl, 9-fluorenylmethoxycarbonyl, allyloxycarbonyl, trimethylsilylethoxycarbonyl, trichloroethoxycarbonyl, trifluoroacetamide, benzamide, benzylamine, triphenylmethylamine, and p-toluenesulfonamide, or a salt thereof.

29. The compound according to claim 26, wherein X is selected from: 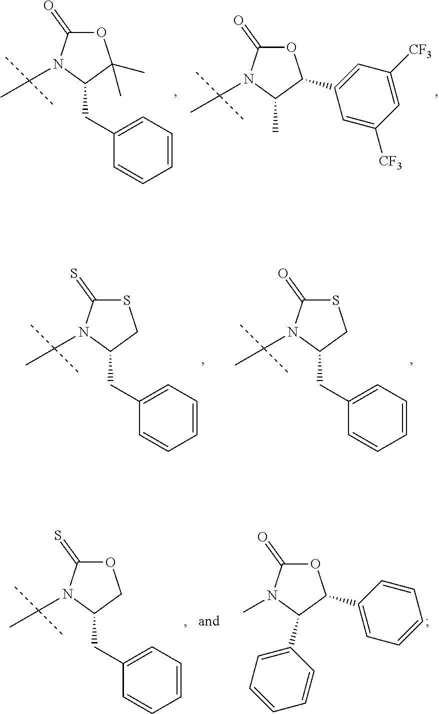
wherein R¹² at each occurrence is independently H or C₁₋₄ alkyl, or a salt thereof.

30. A compound of the formula: 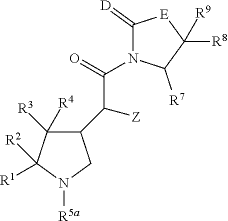
wherein
R¹ and R² are independently selected from H, C₁₋₄ alkyl, and F;
R³ and R⁴ are independently either H or F;
R^{5a} is H, C₁₋₄ alkyl, cyclopropyl, or a protecting group;
D and E are each independently O or S;
R⁷ is H, C₁₋₄ alkyl, phenyl or benzyl, wherein the phenyl and benzyl are optionally substituted with one or two substituents independently selected from: halo, C₁₋₄ alkyl, trifluoromethyl, amino, C₁₋₄ alkylamino, and di-C₁₋₄ alkylamino;
R⁸ and R⁹ are each independently H, C₁₋₄ alkyl or phenyl optionally substituted with one or two substituents independently selected from: halo, C₁₋₄ alkyl, trifluoromethyl, amino, C₁₋₄ alkylamino and di-C₁₋₄ alkylamino;
Z is H, C₁₋₄ alkyl, OH, cyclopropyl, CH₂OH, CH₂NH₂ or CH₂OCH₂phenyl;
or a salt thereof,
wherein the compound is not: 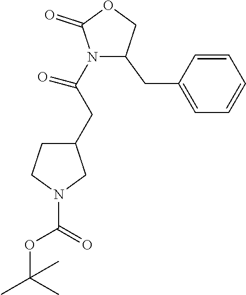 [SMILES: CC(C)(C)OC(=O)N1CCC(CC(=O)N2C(=O)OCC2Cc2ccccc2)C1]
